SEQUENCE LISTING (680 sequences)

SEQ ID NO: 1 - protein, 117 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 2 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 3 - protein, 117 aa, Artificial Sequence; identical to SEQ ID NO: 1
  Description of Artificial Sequence Synthetic polypeptide

SEQ ID NO: 4 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGGGTKVEIK

SEQ ID NO: 5 - protein, 117 aa, Artificial Sequence; identical to SEQ ID NO: 1
  Description of Artificial Sequence Synthetic polypeptide

SEQ ID NO: 6 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFYTFGGGTKVEIK

SEQ ID NO: 7 - protein, 117 aa, Artificial Sequence; identical to SEQ ID NO: 1
  Description of Artificial Sequence Synthetic polypeptide

SEQ ID NO: 8 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQSNSYYTFGGGTKVEIK

SEQ ID NO: 9 - protein, 117 aa, Artificial Sequence; identical to SEQ ID NO: 1
  Description of Artificial Sequence Synthetic polypeptide

SEQ ID NO: 10 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 11 - protein, 117 aa, Artificial Sequence
  Description of Artificial Sequence Synthetic polypeptide
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWGFDPWGQGTLVTVSS

SEQ ID NO: 12 - protein, 107 aa, Artificial Sequence
  Description of Artificial Sequence Synthetic polypeptide
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 13 - protein, 117 aa, Artificial Sequence; identical to SEQ ID NO: 1
  Description of Artificial Sequence Synthetic polypeptide

SEQ ID NO: 14 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 15 - protein, 117 aa, Artificial Sequence; identical to SEQ ID NO: 1
  Description of Artificial Sequence Synthetic polypeptide

SEQ ID NO: 16 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTDFTLTISSLQPDDFATYYCQQSKEVPWTFGQGTKVEIK

SEQ ID NO: 17 - protein, 117 aa, Artificial Sequence; identical to SEQ ID NO: 1
  Description of Artificial Sequence Synthetic polypeptide

SEQ ID NO: 18 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSFPTFGGGTKVEIK

SEQ ID NO: 19 - protein, 117 aa, Artificial Sequence; identical to SEQ ID NO: 1
  Description of Artificial Sequence Synthetic polypeptide

SEQ ID NO: 20 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYDIYPTFGGGTKVEIK

SEQ ID NO: 21 - protein, 117 aa, Artificial Sequence; identical to SEQ ID NO: 1
  Description of Artificial Sequence Synthetic polypeptide

SEQ ID NO: 22 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSYPTFGGGTKVEIK

SEQ ID NO: 23 - protein, 117 aa, Artificial Sequence; identical to SEQ ID NO: 1
  Description of Artificial Sequence Synthetic polypeptide

SEQ ID NO: 24 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYGSFPTFGGGTKVEIK

SEQ ID NO: 25 - protein, 117 aa, Artificial Sequence; identical to SEQ ID NO: 1
  Description of Artificial Sequence Synthetic polypeptide

SEQ ID NO: 26 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSFPTFGGGTKVEIK

SEQ ID NO: 27 - protein, 117 aa, Artificial Sequence; identical to SEQ ID NO: 1
  Description of Artificial Sequence Synthetic polypeptide

SEQ ID NO: 28 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYSSFSTFGGGTKVEIK

SEQ ID NO: 29 - protein, 117 aa, Artificial Sequence; identical to SEQ ID NO: 1
  Description of Artificial Sequence Synthetic polypeptide

SEQ ID NO: 30 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYESYSTFGGGTKVEIK

SEQ ID NO: 31 - protein, 117 aa, Artificial Sequence; identical to SEQ ID NO: 1
  Description of Artificial Sequence Synthetic polypeptide

SEQ ID NO: 32 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDSFITFGGGTKVEIK

SEQ ID NO: 33 - protein, 117 aa, Artificial Sequence; identical to SEQ ID NO: 1
  Description of Artificial Sequence Synthetic polypeptide

SEQ ID NO: 34 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYQSYPTFGGGTKVEIK

SEQ ID NO: 35 - protein, 117 aa, Artificial Sequence; identical to SEQ ID NO: 1
  Description of Artificial Sequence Synthetic polypeptide

SEQ ID NO: 36 - protein, 106 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYHSFPTFGGGTKVEIK

SEQ ID NO: 37 - protein, 117 aa, Artificial Sequence; identical to SEQ ID NO: 1
  Description of Artificial Sequence Synthetic polypeptide

SEQ ID NO: 38 - protein, 107 aa, Artificial Sequence
  Description of Artificial Sequence Synthetic polypeptide
DIQMTQSPSTLSASVGDRVTITCRASQSIGSWLAWYQQKPGKAPKLLIYKASSLESGVPSRFSGSGSGTEFTLTISSLQPDDFATYYCQQYELYSYTFGGGTKVEIK

SEQ ID NO: 39 - protein, 117 aa, Artificial Sequence; identical to SEQ ID NO: 1
  Description of Artificial Sequence Synthetic polypeptide

SEQ ID NO: 40 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYDTFITFGGGTKVEIK

SEQ ID NO: 41 - protein, 125 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDSSIRHAYYYYGMDVWGQGTTVTVSS

SEQ ID NO: 42 - protein, 113 aa, Artificial Sequence
  Description of Artificial Sequence Synthetic polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGGGTKVEIK

SEQ ID NO: 43 - protein, 9 aa, Artificial Sequence
  Description of Artificial Sequence Synthetic peptide
GTFSSYAIS

SEQ ID NO: 44 - protein, 17 aa, Artificial Sequence
  Description of Artificial Sequence Synthetic peptide
GIIPIFGTANYAQKFQG

SEQ ID NO: 45 - protein, 18 aa, Artificial Sequence
  Description of Artificial Sequence Synthetic peptide
ARGDSSIRHAYYYYGMDV

SEQ ID NO: 46 - protein, 17 aa, Artificial Sequence
  Description of Artificial Sequence Synthetic peptide
KSSQSVLYSSNNKNYLA

SEQ ID NO: 47 - protein, 7 aa, Artificial Sequence
  Description of Artificial Sequence Synthetic peptide
WASTRES

SEQ ID NO: 48 - protein, 9 aa, Artificial Sequence
  Description of Artificial Sequence Synthetic peptide
QQYYSTPIT

SEQ ID NO: 49 - protein, 121 aa, Artificial Sequence
  Description of Artificial Sequence Synthetic polypeptide
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSDRFHPYFDYWGQGTLVTVSS

SEQ ID NO: 50 - protein, 107 aa, Artificial Sequence
  Description of Artificial Sequence Synthetic polypeptide
EIVLTQSPATLSLSPGERATLSCRASQSVSRYLAWYQQKPGQAPRLLIYDASNRATGIPARFSGSGSGTDFTLTISSLEPEDFAVYYCQQFDTWPPTFGGGTKVEIK

SEQ ID NO: 51 - protein, 11 aa, Artificial Sequence
  Description of Artificial Sequence Synthetic peptide
GSISSSSYYWG

SEQ ID NO: 52 - protein, 16 aa, Artificial Sequence
  Description of Artificial Sequence Synthetic peptide
SIYYSGSTYYNPSLKS

SEQ ID NO: 53 - protein, 13 aa, Artificial Sequence
  Description of Artificial Sequence Synthetic peptide
ARGSDRFHPYFDY

SEQ ID NO: 54 - protein, 11 aa, Artificial Sequence
  Description of Artificial Sequence Synthetic peptide
RASQSVSRYLA

SEQ ID NO: 55 - protein, 7 aa, Artificial Sequence
  Description of Artificial Sequence Synthetic peptide
DASNRAT

SEQ ID NO: 56 - protein, 9 aa, Artificial Sequence
  Description of Artificial Sequence Synthetic peptide
QQFDTWPPT

SEQ ID NO: 57 - protein, 117 aa, Artificial Sequence; identical to SEQ ID NO: 1
  Description of Artificial Sequence Synthetic polypeptide

SEQ ID NO: 58 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCEQYDSYPTFGGGTKVEIK

SEQ ID NO: 59 - protein, 126 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGRKASGSFYYYYGMDVWGQGTTVTVSS

SEQ ID NO: 60 - protein, 113 aa, Artificial Sequence [sequence omitted]

SEQ ID NO: 61 - protein, 126 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGAPNYGDTTHDYYYMDVWGKGTTVTVSS

SEQ ID NO: 62 - protein, 107 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYDDWPFTFGGGTKVEIK

SEQ ID NO: 63 - protein, 9 aa, Artificial Sequence
  Description of Artificial Sequence Synthetic peptide
YTFTSYYMH

SEQ ID NO: 64 - protein, 17 aa, Artificial Sequence
  Description of Artificial Sequence Synthetic peptide
IINPSGGSTSYAQKFQG

SEQ ID NO: 65 - protein, 19 aa, Artificial Sequence
  Description of Artificial Sequence Synthetic peptide
ARGAPNYGDTTHDYYYMDV

SEQ ID NO: 66 - protein, 11 aa, Artificial Sequence
  Description of Artificial Sequence Synthetic peptide
RASQSVSSNLA

SEQ ID NO: 67 - protein, 7 aa, Artificial Sequence
  Description of Artificial Sequence Synthetic peptide
GASTRAT

SEQ ID NO: 68 - protein, 9 aa, Artificial Sequence
  Description of Artificial Sequence Synthetic peptide
QQYDDWPFT

SEQ ID NO: 69 - protein, 124 aa, Artificial Sequence
  Description of Artificial Sequence Synthetic polypeptide
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDTGEYYDTDDHGMDVWGQGTTVTVSS

SEQ ID NO: 70 - protein, 107 aa, Artificial Sequence
  Description of Artificial Sequence Synthetic polypeptide
EIVLTQSPGTLSLSPGERATLSCRASQSVSSNLAWYQQKPGQAPRLLIYGASTRATGIPARFSGSGSGTEFTLTISSLQSEDFAVYYCQQDDYWPPTFGGGTKVEIK

SEQ ID NO: 71 - protein, 9 aa, Artificial Sequence
  Description of Artificial Sequence Synthetic peptide
YTFTGYYMH

SEQ ID NO: 72 - protein, 17 aa, Artificial Sequence
  Description of Artificial Sequence Synthetic peptide
WINPNSGGTNYAQKFQG

SEQ ID NO: 73 - protein, 17 aa, Artificial Sequence
  Description of Artificial Sequence Synthetic peptide
ARDTGEYYDTDDHGMDV

SEQ ID NO: 74 - protein, 11 aa, Artificial Sequence
  Description of Artificial Sequence Synthetic peptide
RASQSVSSNLA

SEQ ID NO: 75 - protein, 7 aa, Artificial Sequence
  Description of Artificial Sequence Synthetic peptide
GASTRAT

SEQ ID NO: 76 - protein, 9 aa, Artificial Sequence
  Description of Artificial Sequence Synthetic peptide
QQDDYWPPT

SEQ ID NO: 77 - protein, 121 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSS

SEQ ID NO: 78 - protein, 107 aa, Artificial Sequence
  Description of Artificial Sequence Synthetic polypeptide
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIK

SEQ ID NO: 79 - protein, 9 aa, Artificial Sequence
  Description of Artificial Sequence Synthetic peptide
FTFSSYAMS

SEQ ID NO: 80 - protein, 17 aa, Artificial Sequence
  Description of Artificial Sequence Synthetic peptide
AISGSGGSTYYADSVKG

SEQ ID NO: 81 - protein, 14 aa, Artificial Sequence
  Description of Artificial Sequence Synthetic peptide
AKDGGYYDSGAGDY

SEQ ID NO: 82 - protein, 11 aa, Artificial Sequence
  Description of Artificial Sequence Synthetic peptide
RASQGIDSWLA

SEQ ID NO: 83 - protein, 7 aa, Artificial Sequence
  Description of Artificial Sequence Synthetic peptide
AASSLQS

SEQ ID NO: 84 - protein, 9 aa, Artificial Sequence
  Description of Artificial Sequence Synthetic peptide
QQGVSYPRT

SEQ ID NO: 85 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 86 - protein, 107 aa, Artificial Sequence
  Description of Artificial Sequence Synthetic polypeptide
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 87 - protein, 9 aa, Artificial Sequence
  Description of Artificial Sequence Synthetic peptide
FTFSSYSMN

SEQ ID NO: 88 - protein, 17 aa, Artificial Sequence
  Description of Artificial Sequence Synthetic peptide
SISSSSSYIYYADSVKG

SEQ ID NO: 89 - protein, 15 aa, Artificial Sequence
  Description of Artificial Sequence Synthetic peptide
ARGAPMGAAAGWFDP

SEQ ID NO: 90 - protein, 11 aa, Artificial Sequence
  Description of Artificial Sequence Synthetic peptide
RASQGISSWLA

SEQ ID NO: 91 - protein, 7 aa, Artificial Sequence
  Description of Artificial Sequence Synthetic peptide
AASSLQS

SEQ ID NO: 92 - protein, 9 aa, Artificial Sequence
  Description of Artificial Sequence Synthetic peptide
QQGVSFPRT

SEQ ID NO: 93 - protein, 125 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREGAGFAYGMDYYYMDVWGKGTTVTVSS

SEQ ID NO: 94 - protein, 107 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSDNWPFTFGGGTKVEIK

SEQ ID NO: 95 - protein, 9 aa, Artificial Sequence
  Description of Artificial Sequence Synthetic peptide
YTFTSYYMH

SEQ ID NO: 96 - protein, 17 aa, Artificial Sequence
  Description of Artificial Sequence Synthetic peptide
IINPSGGSTSYAQKFQG

SEQ ID NO: 97 - protein, 18 aa, Artificial Sequence
  Description of Artificial Sequence Synthetic peptide
AREGAGFAYGMDYYYMDV

SEQ ID NO: 98 - protein, 11 aa, Artificial Sequence
  Description of Artificial Sequence Synthetic peptide
RASQSVSSYLA

SEQ ID NO: 99 - protein, 7 aa, Artificial Sequence
  Description of Artificial Sequence Synthetic peptide
DASNRAT

SEQ ID NO: 100 - protein, 9 aa, Artificial Sequence
  Description of Artificial Sequence Synthetic peptide
QQSDNWPFT

SEQ ID NO: 101 - protein, 121 aa, Homo sapiens
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 102 - protein, 110 aa, Homo sapiens
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 103 - protein, 115 aa, Homo sapiens
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 104 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 105 - protein, 9 aa, Artificial Sequence
  Description of Artificial Sequence Synthetic peptide
GSFSGYYWS

SEQ ID NO: 106 - protein, 16 aa, Artificial Sequence
  Description of Artificial Sequence Synthetic peptide
EIDHSGSTNYNPSLKS

SEQ ID NO: 107 - protein, 11 aa, Artificial Sequence
  Description of Artificial Sequence Synthetic peptide
ARARGPWSFDP

SEQ ID NO: 108 - protein, 9 aa, Artificial Sequence
  Description of Artificial Sequence Synthetic peptide
GTFSSYAIS

SEQ ID NO: 109 - protein, 17 aa, Artificial Sequence
  Description of Artificial Sequence Synthetic peptide
GIIPIFGTANYAQKFQG

SEQ ID NO: 110 - protein, 19 aa, Artificial Sequence
  Description of Artificial Sequence Synthetic peptide
ARRGRKASGSFYYYYGMDV

SEQ ID NO: 111 - protein, 17 aa, Artificial Sequence
  Description of Artificial Sequence Synthetic peptide
ESSQSLLNSGNQKNYLT

SEQ ID NO: 112 - protein, 7 aa, Artificial Sequence
  Description of Artificial Sequence Synthetic peptide
WASTRES

SEQ ID NO: 113 - protein, 9 aa, Artificial Sequence
  Description of Artificial Sequence Synthetic peptide
QNDYSYPYT

SEQ ID NO: 114 - protein, 118 aa, Artificial Sequence
  Description of Artificial Sequence Synthetic polypeptide
QVQLQQSGAELMKPGASVKLSCKATGYTFTGYWIDWIKQRPGHGLEWVGEILPGSDNINYNEKFRGKATFTADTSSNTAYIQLSSLTTEDSAIYFCARCGTGPWFTYWGQGTLVTVSA

SEQ ID NO: 115 - protein, 113 aa, Artificial Sequence
  Description of Artificial Sequence Synthetic polypeptide
NIMMTQSPSSLAVSAGEKVTMSCKSSQSVLYSSNQKNYLAWYQQKPGQSPRLLIYWASTRASGVPDRFTGSGSGTDFTLTITNIQPEDLAVYYCHQFLSSTWTFGGGTKLEIK

SEQ ID NO: 116 - protein, 5 aa, Artificial Sequence
  Description of Artificial Sequence Synthetic peptide
GYWID

SEQ ID NO: 117 - protein, 17 aa, Artificial Sequence
  Description of Artificial Sequence Synthetic peptide
EILPGSDNINYNEKFRG

SEQ ID NO: 118 - protein, 9 aa, Artificial Sequence
  Description of Artificial Sequence Synthetic peptide
CGTGPWFTY

SEQ ID NO: 119 - protein, 17 aa, Artificial Sequence
  Description of Artificial Sequence Synthetic peptide
KSSQSVLYSSNQKNYLA

SEQ ID NO: 120 - protein, 7 aa, Artificial Sequence
  Description of Artificial Sequence Synthetic peptide
WASTRAS

SEQ ID NO: 121 - protein, 9 aa, Artificial Sequence
  Description of Artificial Sequence Synthetic peptide
HQFLSSTWT

SEQ ID NO: 122 - protein, 117 aa, Artificial Sequence
  Description of Artificial Sequence Synthetic polypeptide
QLQLVQSGPELMRPGETVKISCKASGYTFTTYGMNWVKQAPGKGLKWVGWINTYSGVPTYADDFKGRFAFSLESSASTAFLQINNLKDEDTATYFCARFGNYGFDCWGQGTTLTVSS

SEQ ID NO: 123 - protein, 113 aa, Artificial Sequence
  Description of Artificial Sequence Synthetic polypeptide
DIVMTQSPSSLSVSAGEKVTMSCKSSQSLLNSGNQKNYLAWYQQKPGQPPKLLIYGASTRESGVPDRFTGSGSGTDFTLTISSVQAEDLAVYYCQNDHIYPYTFGGGTKLEIK

SEQ ID NO: 124 - protein, 5 aa, Artificial Sequence
  Description of Artificial Sequence Synthetic peptide
TYGMN

SEQ ID NO: 125 - protein, 17 aa, Artificial Sequence
  Description of Artificial Sequence Synthetic peptide
WINTYSGVPTYADDFKG

SEQ ID NO: 126 - protein, 8 aa, Artificial Sequence
  Description of Artificial Sequence Synthetic peptide
FGNYGFDC

SEQ ID NO: 127 - protein, 17 aa, Artificial Sequence
  Description of Artificial Sequence Synthetic peptide
KSSQSLLNSGNQKNYLA

SEQ ID NO: 128 - protein, 7 aa, Artificial Sequence
  Description of Artificial Sequence Synthetic peptide
GASTRES

SEQ ID NO: 129 - protein, 9 aa, Artificial Sequence
  Description of Artificial Sequence Synthetic peptide
QNDHIYPYT

SEQ ID NO: 130 - protein, 120 aa, Artificial Sequence
  Description of Artificial Sequence Synthetic polypeptide
EVQLQQSGAELVRPGASVKLSCTASGFNIKDDYIHWVKQWPEQGLEWIGWIDSENGDTEYASKFQGKATMTADTSSNTAYLQLSGLTSEDTAVYYCTTSSYYSYDLFVYWGQGTLVTVSA

SEQ ID NO: 131 - protein, 111 aa, Artificial Sequence
  Description of Artificial Sequence Synthetic polypeptide
DVLMTQTPLTLSVPIGQPASISCKSSQSLLHSNGKTYLNWLLQRPGQSPKLLIYLVSKLESGVPDRFSGSGSGTDFTLKISRVEAEDLGVYYCLQTTHLYTFGGGTKLEIK

SEQ ID NO: 132 - protein, 5 aa, Artificial Sequence
  Description of Artificial Sequence Synthetic peptide
DDYIH

SEQ ID NO: 133 - protein, 17 aa, Artificial Sequence
  Description of Artificial Sequence Synthetic peptide
WIDSENGDTEYASKFQG

SEQ ID NO: 134 - protein, 11 aa, Artificial Sequence
  Description of Artificial Sequence Synthetic peptide
SSYYSYDLFVY

SEQ ID NO: 135 - protein, 16 aa, Artificial Sequence
  Description of Artificial Sequence Synthetic peptide
KSSQSLLHSNGKTYLN

SEQ ID NO: 136 - protein, 7 aa, Artificial Sequence
  Description of Artificial Sequence Synthetic peptide
LVSKLES

SEQ ID NO: 137 - protein, 8 aa, Artificial Sequence
  Description of Artificial Sequence Synthetic peptide
LQTTHLYT

SEQ ID NO: 138 - protein, 117 aa, Artificial Sequence
  Description of Artificial Sequence Synthetic polypeptide
QIQLVQSGPELKKPGETVKISCKASGYTFTTYGVNWVKQAPGKGLKWMGWINTYSGVPTYADDFKGRFAFSLETIATTAYLQINNLKNEDTATYFCARFGNYGFDYWGQGTTLTVSS

SEQ ID NO: 139 - protein, 113 aa, Artificial Sequence
  Description of Artificial Sequence Synthetic polypeptide
DIVMTQSPSSLSVSAGEKVTMSCKSSQSLVNSGNQKNYLAWYQQKPGQPPKLLISGASTRESGVPDRFTGSGSGTDFTLTISSVQAEDLAVYYCQNDHNYPYTFGGGTKLEIK

SEQ ID NO: 140 - protein, 5 aa, Artificial Sequence
  Description of Artificial Sequence Synthetic peptide
TYGVN

SEQ ID NO: 141 - protein, 17 aa, Artificial Sequence
  Description of Artificial Sequence Synthetic peptide
WINTYSGVPTYADDFKG

SEQ ID NO: 142 - protein, 8 aa, Artificial Sequence
  Description of Artificial Sequence Synthetic peptide
FGNYGFDY

SEQ ID NO: 143 - protein, 17 aa, Artificial Sequence
  Description of Artificial Sequence Synthetic peptide
KSSQSLVNSGNQKNYLA

SEQ ID NO: 144 - protein, 7 aa, Artificial Sequence
  Description of Artificial Sequence Synthetic peptide
GASTRES

SEQ ID NO: 145 - protein, 9 aa, Artificial Sequence
  Description of Artificial Sequence Synthetic peptide
QNDHNYPYT

SEQ ID NO: 146 - protein, 118 aa, Artificial Sequence
  Description of Artificial Sequence Synthetic polypeptide
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSS

SEQ ID NO: 147 - protein, 108 aa, Artificial Sequence
  Description of Artificial Sequence Synthetic polypeptide
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIKR

SEQ ID NO: 148 - protein, 7 aa, Artificial Sequence
  Description of Artificial Sequence Synthetic peptide
GYTFTNY

SEQ ID NO: 149 - protein, 6 aa, Artificial Sequence
  Description of Artificial Sequence Synthetic peptide
NTYTGE

SEQ ID NO: 150 - protein, 9 aa, Artificial Sequence
  Description of Artificial Sequence Synthetic peptide
IGDSSPSDY

SEQ ID NO: 151 - protein, 8 aa, Artificial Sequence
  Description of Artificial Sequence Synthetic peptide
QSVSNDVV

SEQ ID NO: 152 - protein, 7 aa, Artificial Sequence
  Description of Artificial Sequence Synthetic peptide
YASNRYT

SEQ ID NO: 153 - protein, 9 aa, Artificial Sequence
  Description of Artificial Sequence Synthetic peptide
QQDYTSPWT

SEQ ID NO: 154 - protein, 121 aa, Artificial Sequence
  Description of Artificial Sequence Synthetic polypeptide
QVQLQQSGADLVRPGTSVKVSCKASGYAFTNYLIEWIKQRPGQGLEWIGVMNPGSGGTHYSEKFRGKATLTADKSSSTAYMQLISLTSDDSAVYFCARSDYDYVTYAMDYWGQGTSVTVSS

SEQ ID NO: 155 - protein, 107 aa, Artificial Sequence
  Description of Artificial Sequence Synthetic polypeptide
DIKMTQSPSSMYASLGERVTITCKASQDINSYLIWFQQKPGKSPKTLIYRTNRLVDGVPSRFSGSGSGQDYSLTISSLEYGDMGIYYCLQYDEFPFTFGSGTKLEIK

SEQ ID NO: 156 - protein, 5 aa, Artificial Sequence
  Description of Artificial Sequence Synthetic peptide
NYLIE

SEQ ID NO: 157 - protein, 17 aa, Artificial Sequence
  Description of Artificial Sequence Synthetic peptide
VMNPGSGGTHYSEKFRG

SEQ ID NO: 158 - protein, 12 aa, Artificial Sequence
  Description of Artificial Sequence Synthetic peptide
SDYDYVTYAMDY

SEQ ID NO: 159 - protein, 11 aa, Artificial Sequence
  Description of Artificial Sequence Synthetic peptide
KASQDINSYLI

SEQ ID NO: 160 - protein, 7 aa, Artificial Sequence
  Description of Artificial Sequence Synthetic peptide
RTNRLVD

SEQ ID NO: 161 - protein, 9 aa, Artificial Sequence
  Description of Artificial Sequence Synthetic peptide
LQYDEFPFT

SEQ ID NO: 162 - protein, 118 aa, Artificial Sequence
  Description of Artificial Sequence Synthetic polypeptide
EVQLQQSGPELVKPGASVKMSCRASGYTFTDYYMKWVKQSHGKSLEWIGDINPNNGDTFYNQKFKGKATLTIDKSSSTAYMQLNSLTSDDSAVYYCARDGNYAYFDYWGQGTTLTVSS

SEQ ID NO: 163 - protein, 111 aa, Artificial Sequence
  Description of Artificial Sequence Synthetic polypeptide
DIVLTQSPASLAVSLGQRATISCRASKSVSTSGYSYMHWYQQKPGQPPKLLIFLASNLESGVPARFSGSGSGTDFTLNIHPVEEEDAATYYCQHSRHLPWTFGGGTKLEIK

SEQ ID NO: 164 - protein, 5 aa, Artificial Sequence
  Description of Artificial Sequence Synthetic peptide
DYYMK

SEQ ID NO: 165 - protein, 17 aa, Artificial Sequence
  Description of Artificial Sequence Synthetic peptide
DINPNNGDTFYNQKFKG

SEQ ID NO: 166 - protein, 9 aa, Artificial Sequence
  Description of Artificial Sequence Synthetic peptide
DGNYAYFDY

SEQ ID NO: 167 - protein, 15 aa, Artificial Sequence
  Description of Artificial Sequence Synthetic peptide
RASKSVSTSGYSYMH

SEQ ID NO: 168 - protein, 7 aa, Artificial Sequence
  Description of Artificial Sequence Synthetic peptide
LASNLES

SEQ ID NO: 169 - protein, 9 aa, Artificial Sequence
  Description of Artificial Sequence Synthetic peptide
QHSRHLPWT

SEQ ID NO: 170 - protein, 119 aa, Artificial Sequence
  Description of Artificial Sequence Synthetic polypeptide
QVQLQQSGGDLMKPGASVKISCKAAGYTFSNYYIEWVKQRPGHGLEWIGEILPGSGSTTYNEKFKGKASFTADTSSNTAYMQLSSLTSEDSAVYYCARWGAREPGFPYWGQGTLVTVSA

SEQ ID NO: 171 - protein, 107 aa, Artificial Sequence
  Description of Artificial Sequence Synthetic polypeptide
DIVMTQSQKFMSTSVGDRVSVTCKASQNVGTNVAWYQQKPGQSPKALIYSASYRYSGVPDRFTGSGSGTDFTLTISNVQSEDLAEYFCQQYNNYPLTFGAGTKLELK

SEQ ID NO: 172 - protein, 5 aa, Artificial Sequence
  Description of Artificial Sequence Synthetic peptide
NYYIE

SEQ ID NO: 173 - protein, 17 aa, Artificial Sequence
  Description of Artificial Sequence Synthetic peptide
EILPGSGSTTYNEKFKG

SEQ ID NO: 174 - protein, 10 aa, Artificial Sequence
  Description of Artificial Sequence Synthetic peptide
WGAREPGFPY

SEQ ID NO: 175 - protein, 11 aa, Artificial Sequence
  Description of Artificial Sequence Synthetic peptide
KASQNVGTNVA

SEQ ID NO: 176 - protein, 7 aa, Artificial Sequence
  Description of Artificial Sequence Synthetic peptide
SASYRYS

SEQ ID NO: 177 - protein, 9 aa, Artificial Sequence
  Description of Artificial Sequence Synthetic peptide
QQYNNYPLT

SEQ ID NO: 178 - protein, 123 aa, Artificial Sequence
  Description of Artificial Sequence Synthetic polypeptide
EVKLVESGGGLVQPGGSLSLSCAASGFTFTDYYMNWVRQPPGKALEWLALIRNKANGYTTEYNASVKGRFTISRDNSQNILYLQMNALRAEDSATYYCARDSDGYYEYYFDYWGQGTTLTVSS

SEQ ID NO: 179 - protein, 107 aa, Artificial Sequence
  Description of Artificial Sequence Synthetic polypeptide
DIQMTQSPSSLSASLGERVSLTCRASQEISDYLSWLQQKPDGTIKRLIFAASTLDSGVPKRFSGSRSGSDFSLSISSLESEDFADYYCLQYASYPYTFGSGTKLEIK

SEQ ID NO: 180 - protein, 5 aa, Artificial Sequence
  Description of Artificial Sequence Synthetic peptide
DYYMN

SEQ ID NO: 181 - protein, 19 aa, Artificial Sequence
  Description of Artificial Sequence Synthetic peptide
LIRNKANGYTTEYNASVKG

SEQ ID NO: 182 - protein, 12 aa, Artificial Sequence
  Description of Artificial Sequence Synthetic peptide
DSDGYYEYYFDY

SEQ ID NO: 183 - protein, 11 aa, Artificial Sequence
  Description of Artificial Sequence Synthetic peptide
RASQEISDYLS

SEQ ID NO: 184 - protein, 7 aa, Artificial Sequence
  Description of Artificial Sequence Synthetic peptide
AASTLDS

SEQ ID NO: 185 - protein, 9 aa, Artificial Sequence
  Description of Artificial Sequence Synthetic peptide
LQYASYPYT

SEQ ID NO: 186 - protein, 118 aa, Artificial Sequence
  Description of Artificial Sequence Synthetic polypeptide
QVQLQESGPGLVKPSETLSLTCTVSGGSISSYYWSWIRQPPGKGLEWIGYVYYSGTTNYNPSLKSRVTISVDTSKNQFSLKLSSVTAADTAVYYCASIAVTGFYFDYWGQGTLVTVSS

SEQ ID NO: 187 - protein, 108 aa, Artificial Sequence
  Description of Artificial Sequence Synthetic polypeptide
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGGGTKLEIK

SEQ ID NO: 188 - protein, 5 aa, Artificial Sequence
  Description of Artificial Sequence Synthetic peptide
SYYWS

SEQ ID NO: 189 - protein, 16 aa, Artificial Sequence
  Description of Artificial Sequence Synthetic peptide
YVYYSGTTNYNPSLKS

SEQ ID NO: 190 - protein, 10 aa, Artificial Sequence
  Description of Artificial Sequence Synthetic peptide
IAVTGFYFDY

SEQ ID NO: 191 - protein, 12 aa, Artificial Sequence
  Description of Artificial Sequence Synthetic peptide
RASQRVNNNYLA

SEQ ID NO: 192 - protein, 7 aa, Artificial Sequence
  Description of Artificial Sequence Synthetic peptide
GASSRAT

SEQ ID NO: 193 - protein, 9 aa, Artificial Sequence
  Description of Artificial Sequence Synthetic peptide
QQYDRSPLT

SEQ ID NO: 194 - protein, 118 aa, Artificial Sequence
  Description of Artificial Sequence Synthetic polypeptide
QVQLQESGPGLVKPSETLSLTCTVSGASISSFYWSWIRQPPGKGLEWIGYIYYSGTTNYNPSLKSRVTISVDTSKNQFSLKLSSVTAADTAVYYCARIAVAGFFFDYWGQGTLVTVSS

SEQ ID NO: 195 - protein, 108 aa, Artificial Sequence
  Description of Artificial Sequence Synthetic polypeptide
EIVLTQSPGTLSLSPGESATLSCRASQSVNKNYLAWYQQKPGQAPRLLIYGASSRATGIPDRFSGSGSGTDFTLTISRLEPEDFAVYYCQQYDRSPLTFGGGTRLEIK

SEQ ID NO: 196 - protein, 5 aa, Artificial Sequence
  Description of Artificial Sequence Synthetic peptide
SFYWS

SEQ ID NO: 197 - protein, 16 aa, Artificial Sequence
  Description of Artificial Sequence Synthetic peptide
YIYYSGTTNYNPSLKS

SEQ ID NO: 198 - protein, 10 aa, Artificial Sequence
  Description of Artificial Sequence Synthetic peptide
IAVAGFFFDY

SEQ ID NO: 199 - protein, 12 aa, Artificial Sequence
  Description of Artificial Sequence Synthetic peptide
RASQSVNKNYLA

SEQ ID NO: 200 - protein, 7 aa, Artificial Sequence
  Description of Artificial Sequence Synthetic peptide
GASSRAT

SEQ ID NO: 201 - protein, 9 aa, Artificial Sequence
  Description of Artificial Sequence Synthetic peptide
QQYDRSPLT

SEQ ID NO: 202 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 203 - protein, 120 aa, Artificial Sequence
  Description of Artificial Sequence Synthetic polypeptide
QVQLQQSGAEVKKPGASVKVSCEASGYTFPSYVLHWVKQAPGQGLEWIGYINPYNDGTQYNEKFKGKATLTRDTSINTAYMELSRLRSDDTAVYYCARGFGGSYGFAYWGQGTLVTVSSA

SEQ ID NO: 204 - protein, 7 aa, Artificial Sequence
  Description of Artificial Sequence Synthetic peptide
GYTFPSY

SEQ ID NO: 205 - protein, 6 aa, Artificial Sequence
  Description of Artificial Sequence Synthetic peptide
NPYNDG

SEQ ID NO: 206 - protein, 10 aa, Artificial Sequence
  Description of Artificial Sequence Synthetic peptide
GFGGSYGFAY

SEQ ID NO: 207 - protein, 109 aa, Artificial Sequence
  Description of Artificial Sequence Synthetic polypeptide
DIQLTQSPSSLSASVGDRVTMTCSASSSVSSSYLYWYQQKPGKAPKLWIYSTSNLASGVPARFSGSGSGTDFTLTISSLQPEDSASYFCHQWNRYPYTFGGGTRLEIKR

SEQ ID NO: 208 - protein, 9 aa, Artificial Sequence
  Description of Artificial Sequence Synthetic peptide
SSVSSSYLY

SEQ ID NO: 209 - protein, 7 aa, Artificial Sequence
  Description of Artificial Sequence Synthetic peptide
STSNLAS

SEQ ID NO: 210 - protein, 9 aa, Artificial Sequence
  Description of Artificial Sequence Synthetic peptide
HQWNRYPYT

SEQ ID NO: 211 - protein, 118 aa, Artificial Sequence
  Description of Artificial Sequence Synthetic polypeptide
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSSA

SEQ ID NO: 212 - protein, 5 aa, Artificial Sequence
  Description of Artificial Sequence Synthetic peptide
NYWMN

SEQ ID NO: 213 - protein, 16 aa, Artificial Sequence
  Description of Artificial Sequence Synthetic peptide
EIRLKSNNYTTHYAES

SEQ ID NO: 214 - protein, 6 aa, Artificial Sequence
  Description of Artificial Sequence Synthetic peptide
HYYFDY

SEQ ID NO: 215 - protein, 113 aa, Artificial Sequence
  Description of Artificial Sequence Synthetic polypeptide
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKR

SEQ ID NO: 216 - protein, 16 aa, Artificial Sequence
  Description of Artificial Sequence Synthetic peptide
RSSKSLLHSNGITYFF

SEQ ID NO: 217 - protein, 7 aa, Artificial Sequence
  Description of Artificial Sequence Synthetic peptide
QMSNLAS

SEQ ID NO: 218 - protein, 9 aa, Artificial Sequence
  Description of Artificial Sequence Synthetic peptide
AQNLELPPT

SEQ ID NO: 219 - protein, 1255 aa, Unknown
  Description of Unknown Mucin 1 sequence
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 220 - protein, 122 aa, Artificial Sequence
  Description of Artificial Sequence Synthetic polypeptide
QVQLQQSGPGLVKPSQTLSLTCAISGDSLSSTSAAWNWIRQSPSGGLEWLGRTYYRSKWYNDYAVSVKSRITINPDTSKNQFSLQLNSVTPEDTSVYYCARDRGYYNGVDVWGQGTMVTVSS

SEQ ID NO: 221 - protein, 7 aa, Artificial Sequence
  Description of Artificial Sequence Synthetic peptide
STSAAWN

SEQ ID NO: 222 - protein, 18 aa, Artificial Sequence
  Description of Artificial Sequence Synthetic peptide
RTYYRSKWYNDYAVSVKS

SEQ ID NO: 223 - protein, 10 aa, Artificial Sequence
  Description of Artificial Sequence Synthetic peptide
DRGYYNGVDV

SEQ ID NO: 224 - protein, 106 aa, Artificial Sequence
  Description of Artificial Sequence Synthetic polypeptide
SYELTQPPSVSVSPGQTASITCSGDKLGDKYASWYQQRPGQSPLLVIYQDNKRPSGIPQRFSGSNSGNTATLTISGTQAMDEADYFCQAWDSGTFVFGTGTKVTIL

SEQ ID NO: 225 - protein, 11 aa, Artificial Sequence
  Description of Artificial Sequence Synthetic peptide
SGDKLGDKYAS

SEQ ID NO: 226 - protein, 7 aa, Artificial Sequence
  Description of Artificial Sequence Synthetic peptide
QDNKRPS

SEQ ID NO: 227 - protein, 9 aa, Artificial Sequence
  Description of Artificial Sequence Synthetic peptide
QAWDSGTFV

SEQ ID NO: 228 - protein, 120 aa, Artificial Sequence
  Description of Artificial Sequence Synthetic polypeptide
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARAPGHYYYGMDVWGQGTTVTVSS

SEQ ID NO: 229 - protein, 5 aa, Artificial Sequence
  Description of Artificial Sequence Synthetic peptide
SYYMH

SEQ ID NO: 230 - protein, 16 aa, Artificial Sequence
  Description of Artificial Sequence Synthetic peptide
IINPSGGSTSYAQKFQ

SEQ ID NO: 231 - protein, 11 aa, Artificial Sequence
  Description of Artificial Sequence Synthetic peptide
APGHYYYGMDV

SEQ ID NO: 232 - protein, 108 aa, Artificial Sequence
  Description of Artificial Sequence Synthetic polypeptide
SYELTQPPSVSVSPGQTASITCSGDKLEDKYASWYQLKPGHSPVLVIYQDSKRPSGIPERFSGSNSGNTATLTISGTQAMDEADYYCRAWDSNTGDVVFGGGTKLTVL

SEQ ID NO: 233 - protein, 11 aa, Artificial Sequence
  Description of Artificial Sequence Synthetic peptide
SGDKLEDKYAS

SEQ ID NO: 234 - protein, 7 aa, Artificial Sequence
  Description of Artificial Sequence Synthetic peptide
QDSKRPS

SEQ ID NO: 235 - protein, 11 aa, Artificial Sequence
  Description of Artificial Sequence Synthetic peptide
RAWDSNTGDVV

SEQ ID NO: 236 - protein, 1896 aa, Unknown
  Description of Unknown Plexin-A1 sequence
MPLPPRSLQVLLLLLLLLLLLPGMWAEAGLPRAGGGSQPPFRTFSASDWGLTHLVVHEQTGEVYVGAVNRIYKLSGNLTLLRAHVTGPVEDNEKCYPPPSVQSCPHGLGSTDNVNKLLLLDYAANRLLACGSASQGICQFLRLDDLFKLGEPHHRKEHYLSSVQEAGSMAGVLIAGPPGQGQAKLFVGTPIDGKSEYFPTLSSRRLMANEEDADMFGFVYQDEFVSSQLKIPSDTLSKFPAFDIYYVYSFRSEQFVYYLTLQLDTQLTSPDAAGEHFFTSKIVRLCVDDPKFYSYVEFPIGCEQAGVEYRLVQDAYLSRPGRALAHQLGLAEDEDVLFTVFAQGQKNRVKPPKESALCLFTLRAIKEKIKERIQSCYRGEGKLSLPWLLNKELGCINSPLQIDDDFCGQDFNQPLGGTVTIEGTPLFVDKDDGLTAVAAYDYRGRTVVFAGTRSGRIRKILVDLSNPGGRPALAYESVVAQEGSPILRDLVLSPNHQYLYAMTEKQVTRVPVESCVQYTSCELCLGSRDPHCGWCVLHSICSRRDACERADEPQRFAADLLQCVQLTVQPRNVSVTMSQVPLVLQAWNVPDLSAGVNCSFEDFTESESVLEDGRIHCRSPSAREVAPITRGQGDQRVVKLYLKSKETGKKFASVDFVFYNCSVHQSCLSCVNGSFPCHWCKYRHVCTHNVADCAFLEGRVNVSEDCPQILPSTQIYVPVGVVKPITLAARNLPQPQSGQRGYECLFHIPGSPARVTALRFNSSSLQCQNSSYSYEGNDVSDLPVNLSVVWNGNFVIDNPQNIQAHLYKCPALRESCGLCLKADPRFECGWCVAERRCSLRHHCAADTPASWMHARHGSSRCTDPKILKLSPETGPRQGGTRLTITGENLGLRFEDVRLGVRVGKVLCSPVESEYISAEQIVCEIGDASSVRAHDALVEVCVRDCSPHYRALSPKRFTFVTPTFYRVSPSRGPLSGGTWIGIEGSHLNAGSDVAVSVGGRPCSFSWRNSREIRCLTPPGQSPGSAPIIININRAQLTNPEVKYNYTEDPTILRIDPEWSINSGGTLLTVTGTNLATVREPRIRAKYGGIERENGCLVYNDTTMVCRAPSVANPVRSPPELGERPDELGFVMDNVRSLLVLNSTSFLYYPDPVLEPLSPTGLLELKPSSPLILKGRNLLPPAPGNSRLNYTVLIGSTPCTLTVSETQLLCEAPNLTGQHKVTVRAGGFEFSPGTLQVYSDSLLTLPAIVGIGGGGGLLLLVIVAVLIAYKRKSRDADRTLKRLQLQMDNLESRVALECKEAFAELQTDIHELTNDLDGAGIPFLDYRTYAMRVLFPGIEDHPVLKEMEVQANVEKSLTLFGQLLTKKHFLLTFIRTLEAQRSFSMRDRGNVASLIMTALQGEMEYATGVLKQLLSDLIEKNLESKNHPKLLLRRTESVAEKMLTNWFTFLLYKFLKECAGEPLFMLYCAIKQQMEKGPIDAITGEARYSLSEDKLIRQQIDYKTLTLNCVNPENENAPEVPVKGLDCDTVTQAKEKLLDAAYKGVPYSQRPKAADMDLEWRQGRMARIILQDEDVTTKIDNDWKRLNTLAHYQVTDGSSVALVPKQTSAYNISNSSTFTKSLSRYESMLRTASSPDSLRSRTPMITPDLESGTKLWHLVKNHDHLDQREGDRGSKMVSEIYLTRLLATKGTLQKFVDDLFETIFSTAHRGSALPLAIKYMFDFLDEQADKHQIHDADVRHTWKSNCLPLRFWVNVIKNPQFVFDIHKNSITDACLSVVAQTFMDSCSTSEHKLGKDSPSNKLLYAKDIPNYKSWVERYYADIAKMPAISDQDMSAYLAEQSRLHLSQFNSMSALHEIYSYITKYKDEILAALEKDEQARRQRLRSKLEQVVDTMALSS

SEQ ID NO: 237 - protein, 120 aa, Artificial Sequence
  Description of Artificial Sequence Synthetic polypeptide
EVQLVESGGGLVQPGGSLRLSCAASGFTFSSYVMSWVRQAPGKGLEWVATISSGGSYTYYPDSVKGRFTISRDNAKNTLYLQMNSLRAEDTAVYYCARRGDSMITTDYWGQGTLVTVSSA

SEQ ID NO: 238 - protein, 7 aa, Artificial Sequence
  Description of Artificial Sequence Synthetic peptide
GFTFSSY

SEQ ID NO: 239 - protein, 6 aa, Artificial Sequence
  Description of Artificial Sequence Synthetic peptide
SSGGSY

SEQ ID NO: 240 - protein, 10 aa, Artificial Sequence
  Description of Artificial Sequence Synthetic peptide
RGDSMITTDY

SEQ ID NO: 241 - protein, 107 aa, Artificial Sequence
  Description of Artificial Sequence Synthetic polypeptide
DIQMTQSPSSLSASVGDRVTITCKASQDVGTAVAWYQQKPGKAPKLLIYWASTRHTGVPSRFSGSGSGTDFTLTISSLQPEDFATYYCQQYSSYRTFGQGTKVEIKR

SEQ ID NO: 242 - protein, 8 aa, Artificial Sequence
  Description of Artificial Sequence Synthetic peptide
QDVGTAVA

SEQ ID NO: 243 - protein, 7 aa, Artificial Sequence
  Description of Artificial Sequence Synthetic peptide
WASTRHT

SEQ ID NO: 244 - protein, 8 aa, Artificial Sequence
  Description of Artificial Sequence Synthetic peptide
QQYSSYRT

SEQ ID NO: 245 - protein, 123 aa, Artificial Sequence
  Description of Artificial Sequence Synthetic polypeptide
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSA

SEQ ID NO: 246 - protein, 9 aa, Artificial Sequence
  Description of Artificial Sequence Synthetic peptide
GGSISSGDY

SEQ ID NO: 247 - protein, 5 aa, Artificial Sequence
  Description of Artificial Sequence Synthetic peptide
HNSGT

SEQ ID NO: 248 - protein, 12 aa, Artificial Sequence
  Description of Artificial Sequence Synthetic peptide
DRGGDYYYGMDV

SEQ ID NO: 249 - protein, 109 aa, Artificial Sequence
  Description of Artificial Sequence Synthetic polypeptide
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIKR

SEQ ID NO: 250 - protein, 9 aa, Artificial Sequence
  Description of Artificial Sequence Synthetic peptide
QGISRSYLA

SEQ ID NO: 251 - protein, 7 aa, Artificial Sequence
  Description of Artificial Sequence Synthetic peptide
GASSRAT

SEQ ID NO: 252 - protein, 9 aa, Artificial Sequence
  Description of Artificial Sequence Synthetic peptide
QQFGSSPWT

SEQ ID NO: 253 - protein, 122 aa, Artificial Sequence
  Description of Artificial Sequence Synthetic polypeptide
EVQLVQSGGGVERPGGSLRLSCAASGFTFDDYAMSWVRQAPGKGLEWVSGINWQGGSTGYADSVKGRVTISRDNAKNSLYLQMNSLRAEDTAVYYCAKILGAGRGWYFDYWGKGTTVTVSSA

SEQ ID NO: 254 - protein, 7 aa, Artificial Sequence
  Description of Artificial Sequence Synthetic peptide
GFTFDDY

SEQ ID NO: 255 - protein, 6 aa, Artificial Sequence
  Description of Artificial Sequence Synthetic peptide
NWQGGS

SEQ ID NO: 256 - protein, 12 aa, Artificial Sequence
  Description of Artificial Sequence Synthetic peptide
ILGAGRGWYFDY

SEQ ID NO: 257 - protein, 108 aa, Artificial Sequence
  Description of Artificial Sequence Synthetic polypeptide
SELTQDPAVSVALGQTVRITCSGDSLRSYYASWYQQKPGQAPVLVIYGANNRPSGIPDRFSGSSSGNTASLTITGAQAEDEADYYCNSADSSGNHVVFGGGTKLTVLG

SEQ ID NO: 258 - protein, 8 aa, Artificial Sequence
  Description of Artificial Sequence Synthetic peptide
SLRSYYAS

SEQ ID NO: 259 - protein, 7 aa, Artificial Sequence
  Description of Artificial Sequence Synthetic peptide
GANNRPS

SEQ ID NO: 260 - protein, 11 aa, Artificial Sequence
  Description of Artificial Sequence Synthetic peptide
NSADSSGNHVV

SEQ ID NO: 261 - protein, 440 aa, Unknown
  Description of Unknown TNFRSF10B sequence
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 262 - protein, 124 aa, Artificial Sequence
  Description of Artificial Sequence Synthetic polypeptide
EVQLVESGGGLVQPGGSLRLSCAASGYSITSDYAWNWVRQAPGKGLEWVGYISNSGSTSYNPSLKSRFTISRDNSKNTLYLQMNSLRAEDTAVYYCARERNYDYDDYYYAMDYWGQGTLVTVSS

SEQ ID NO: 263 - protein, 8 aa, Artificial Sequence
  Description of Artificial Sequence Synthetic peptide
GYSITSDY

SEQ ID NO: 264 - protein, 5 aa, Artificial Sequence
  Description of Artificial Sequence Synthetic peptide
SNSGS

SEQ ID NO: 265 - protein, 15 aa, Artificial Sequence
  Description of Artificial Sequence Synthetic peptide
ERNYDYDDYYYAMDY

SEQ ID NO: 266 - protein, 114 aa, Artificial Sequence
  Description of Artificial Sequence Synthetic polypeptide
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKR

SEQ ID NO: 267 - protein, 14 aa, Artificial Sequence
  Description of Artificial Sequence Synthetic peptide
QSLLYRSNQKNYLA

SEQ ID NO: 268 - protein, 7 aa, Artificial Sequence
  Description of Artificial Sequence Synthetic peptide
WASTRES

SEQ ID NO: 269 - protein, 9 aa, Artificial Sequence
  Description of Artificial Sequence Synthetic peptide
QQYYNYPRT

SEQ ID NO: 270 - protein, 124 aa, Artificial Sequence
  Description of Artificial Sequence Synthetic polypeptide
EVQLVESGGGLVQPGGSLRLSCAVSGYSITSDYAWNWVRQAPGKGLEWVGYISNSGSTSYNPSLKSRFTISRDNSKNTLYLQMNSLRAEDTAVYYCARERNYDYDDYYYAMDYWGQGTLVTVSS

SEQ ID NO: 271 - protein, 5 aa, Artificial Sequence
  Description of Artificial Sequence Synthetic peptide
DYAWN

SEQ ID NO: 272 - protein, 16 aa, Artificial Sequence
  Description of Artificial Sequence Synthetic peptide
YISNSGSTSYNPSLKS

SEQ ID NO: 273 - protein, 15 aa, Artificial Sequence
  Description of Artificial Sequence Synthetic peptide
ERNYDYDDYYYAMDY

SEQ ID NO: 274 - protein, 113 aa, Artificial Sequence
  Description of Artificial Sequence Synthetic polypeptide
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIK

SEQ ID NO: 275 - protein, 17 aa, Artificial Sequence
  Description of Artificial Sequence Synthetic peptide
KSSQSLLYRSNQKNYLA

SEQ ID NO: 276 - protein, 7 aa, Artificial Sequence
  Description of Artificial Sequence Synthetic peptide
WASTRES

SEQ ID NO: 277 - protein, 9 aa, Artificial Sequence
  Description of Artificial Sequence Synthetic peptide
QQYYNYPRT

SEQ ID NO: 278 - protein, 339 aa, Unknown
  Description of Unknown STEAP1 sequence
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQL

SEQ ID NO: 279 - protein, 120 aa, Artificial Sequence
  Description of Artificial Sequence Synthetic polypeptide
EVQLVESGGGLVQPGGSLRLSCAASGFTFNSYGMSWVRQAPGKGLEWVATISSGGSYKYYVDSVKGRFTISRDNAKNSLYLQMNSLRAEDTAVYYCARHPDYDGVWFAYWGQGTLVTVSS

SEQ ID NO: 280 - protein, 5 aa, Artificial Sequence
  Description of Artificial Sequence Synthetic peptide
SYGMS

SEQ ID NO: 281 - protein, 17 aa, Artificial Sequence
  Description of Artificial Sequence Synthetic peptide
TISSGGSYKYYVDSVKG

SEQ ID NO: 282 - protein, 11 aa, Artificial Sequence
  Description of Artificial Sequence Synthetic peptide
HPDYDGVWFAY

SEQ ID NO: 283 - protein, 106 aa, Artificial Sequence
  Description of Artificial Sequence Synthetic polypeptide
EIVLTQSPATLSLSPGERATMSCSVSSSVFYLHWYQQKPGQAPRLWIYDTSKLASGIPARFSGSGSGTDFTLTISSLEPEDFAVYYCQQWNSNPPTFGGGTKVEIK

SEQ ID NO: 284 - protein, 10 aa, Artificial Sequence
  Description of Artificial Sequence Synthetic peptide
SVSSSVFYLH

SEQ ID NO: 285 - protein, 7 aa, Artificial Sequence
  Description of Artificial Sequence Synthetic peptide
DTSKLAS

SEQ ID NO: 286 - protein, 9 aa, Artificial Sequence
  Description of Artificial Sequence Synthetic peptide
QQWNSNPPT

SEQ ID NO: 287 - protein, 106 aa, Artificial Sequence
  Description of Artificial Sequence Synthetic polypeptide
DIQMTQSPSSLSASVGDRVTITCSVSSSVFYVHWYQQKPGKAPKLLIYDTSKLASGVPSRFSGSGSGTDFTFTISSLQPEDIATYYCQQWNSNPPTFGGGTKVEIK

SEQ ID NO: 288 - protein, 10 aa, Artificial Sequence
  Description of Artificial Sequence Synthetic peptide
SVSSSVFYVH

SEQ ID NO: 289 - protein, 7 aa, Artificial Sequence
  Description of Artificial Sequence Synthetic peptide
DTSKLAS

SEQ ID NO: 290 - protein, 9 aa, Artificial Sequence
  Description of Artificial Sequence Synthetic peptide
QQWNSNPPT

SEQ ID NO: 291 - protein, 836 aa, Unknown
  Description of Unknown CDCP1 sequence
MAGLNCGVSIALLGVLLLGAARLPRGAEAFEIALPRESNITVLIKLGTPTLLAKPCYIVISKRHITMLSIKSGERIVFTFSCQSPENHFVIEIQKNIDCMSGPCPFGEVQLQPSTSLLPTLNRTFIWDVKAHKSIGLELQFSIPRLRQIGPGESCPDGVTHSISGRIDATVVRIGTFCSNGTVSRIKMQEGVKMALHLPWFHPRNVSGFSIANRSSIKRLCIIESVFEGEGSATLMSANYPEGFPEDELMTWQFVVPAHLRASVSFLNFNLSNCERKEERVEYYIPGSTTNPEVFKLEDKQPGNMAGNFNLSLQGCDQDAQSPGILRLQFQVLVQHPQNESNKIYVVDLSNERAMSLTIEPRPVKQSRKFVPGCFVCLESRTCSSNLTLTSGSKHKISFLCDDLTRLWMNVEKTISCTDHRYCQRKSYSLQVPSDILHLPVELHDFSWKLLVPKDRLSLVLVPAQKLQQHTHEKPCNTSFSYLVASAIPSQDLYFGSFCPGGSIKQIQVKQNISVTLRTFAPSFQQEASRQGLTVSFIPYFKEEGVFTVTPDTKSKVYLRTPNWDRGLPSLTSVSWNISVPRDQVACLTFFKERSGVVCQTGRAFMIIQEQRTRAEEIFSLDEDVLPKPSFHHHSFWVNISNCSPTSGKQLDLLFSVTLTPRTVDLTVILIAAVGGGVLLLSALGLIICCVKKKKKKTNKGPAVGIYNDNINTEMPRQPKKFQKGRKDNDSHVYAVIEDTMVYGHLLQDSSGSFLQPEVDTYRPFQGTMGVCPPSPPTICSRAPTAKLATEEPPPRSPPESESEPYTFSHPNNGDVSSKDTDIPLLNTQEPMEPAE

SEQ ID NO: 292 - protein, 120 aa, Artificial Sequence
  Description of Artificial Sequence Synthetic polypeptide
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYFYALDYWGQGTSVTVSSA

SEQ ID NO: 293 - protein, 5 aa, Artificial Sequence
  Description of Artificial Sequence Synthetic peptide
DYAVH

SEQ ID NO: 294 - protein, 16 aa, Artificial Sequence
  Description of Artificial Sequence Synthetic peptide
VISTYNDYTYNNQDFK

SEQ ID NO: 295 - protein, 10 aa, Artificial Sequence
  Description of Artificial Sequence Synthetic peptide
GNSYFYALDY

SEQ ID NO: 296 - protein, 112 aa, Artificial Sequence
  Description of Artificial Sequence Synthetic polypeptide
EIVLTQSPATLSLSPGERATLSCRASESVDSYGKSFMHWYQQKPGQAPRLLIYRASNLESGIPARFSGSGSGTDFTLTISSLEPEDFAVYYCQQSNEDPWTFGGGTKLEIKR

SEQ ID NO: 297 - protein, 15 aa, Artificial Sequence
  Description of Artificial Sequence Synthetic peptide
RASESVDSYGKSFMH

SEQ ID NO: 298 - protein, 7 aa, Artificial Sequence
  Description of Artificial Sequence Synthetic peptide
RASNLES

SEQ ID NO: 299 - protein, 9 aa, Artificial Sequence
  Description of Artificial Sequence Synthetic peptide
QQSNEDPWT

SEQ ID NO: 300 - protein, 1070 aa, Unknown
  Description of Unknown PTK7 sequence
MGAARGSPARPRRLPLLSVLLLPLLGGTQTAIVFIKQPSSQDALQGRRALLRCEVEAPGPVHVYWLLDGAPVQDTERRFAQGSSLSFAAVDRLQDSGTFQCVARDDVTGEEARSANASFNIKWIEAGPVVLKHPASEAEIQPQTQVTLRCHIDGHPRPTYQWFRDGTPLSDGQSNHTVSSKERNLTLRPAGPEHSGLYSCCAHSAFGQACSSQNFTLSIADESFARVVLAPQDVVVARYEEAMFHCQFSAQPPPSLQWLFEDETPITNRSRPPHLRRATVFANGSLLLTQVRPRNAGIYRCIGQGQRGPPIILEATLHLAEIEDMPLFEPRVFTAGSEERVTCLPPKGLPEPSVWWEHAGVRLPTHGRVYQKGHELVLANIAESDAGVYTCHAANLAGQRRQDVNITVATVPSWLKKPQDSQLEEGKPGYLDCLTQATPKPTVVWYRNQMLISEDSRFEVFKNGTLRINSVEVYDGTWYRCMSSTPAGSIEAQARVQVLEKLKFTPPPQPQQCMEFDKEATVPCSATGREKPTIKWERADGSSLPEWVTDNAGTLHFARVTRDDAGNYTCIASNGPQGQIRAHVQLTVAVFITFKVEPERTTVYQGHTALLQCEAQGDPKPLIQWKGKDRILDPTKLGPRMHIFQNGSLVIHDVAPEDSGRYTCIAGNSCNIKHTEAPLYVVDKPVPEESEGPGSPPPYKMIQTIGLSVGAAVAYIIAVLGLMFYCKKRCKAKRLQKQPEGEEPEMECLNGGPLQNGQPSAEIQEEVALTSLGSGPAATNKRHSTSDKMHFPRSSLQPITTLGKSEFGEVFLAKAQGLEEGVAETLVLVKSLQSKDEQQQLDFRRELEMFGKLNHANVVRLLGLCREAEPHYMVLEYVDLGDLKQFLRISKSKDEKLKSQPLSTKQKVALCTQVALGMEHLSNNRFVHKDLAARNCLVSAQRQVKVSALGLSKDVYNSEYYHFRQAWVPLRWMSPEAILEGDFSTKSDVWAFGVLMWEVFTHGEMPHGGQADDEVLADLQAGKARLPQPEGCPSKLYRLMQRCWALSPKDRPSFSEIASALGDSTVDSKP

SEQ ID NO: 301 - protein, 121 aa, Artificial Sequence
  Description of Artificial Sequence Synthetic polypeptide
EVQLVESGGGLVQPGGSLRLSCAASGFSLSGSWIHWVRQAPGKGLEWVGWINPYRGYAYYADSVKGRFTISADTSKNTAYLQMNSLRAEDTAVYYCAREYSGWGGSSVGYAMDYWGQGTLV

SEQ ID NO: 302 - protein, 7 aa, Artificial Sequence
  Description of Artificial Sequence Synthetic peptide
GFSLSGS

SEQ ID NO: 303 - protein, 6 aa, Artificial Sequence
  Description of Artificial Sequence Synthetic peptide
NPYRGY

SEQ ID NO: 304 - protein, 16 aa, Artificial Sequence
  Description of Artificial Sequence Synthetic peptide
EYSGWGGSSVGYAMDY

SEQ ID NO: 305 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKR

SEQ ID NO: 306 - protein, 8 aa, Artificial Sequence
  Description of Artificial Sequence Synthetic peptide
QDVSTAVA

SEQ ID NO: 307 - protein, 7 aa, Artificial Sequence
  Description of Artificial Sequence Synthetic peptide
SASFLYS

SEQ ID NO: 308 - protein, 9 aa, Artificial Sequence
  Description of Artificial Sequence Synthetic peptide
QQSYTTPPT

SEQ ID NO: 309 - protein, 121 aa, Artificial Sequence
  Description of Artificial Sequence Synthetic polypeptide
QVQLQQSGAELMKPGASVKMSCKAAGYTFSSYWIEWVRQRPGHGLEWIGEIFPGSDSTNYNEKFNDRATFTADTSSNTAYMQLSSLTSEDSAVYYCARPLYYGSSAWFAYWGQGTLVTVSA

SEQ ID NO: 310 - protein, 7 aa, Artificial Sequence
  Description of Artificial Sequence Synthetic peptide
GYTFSSY

SEQ ID NO: 311 - protein, 6 aa, Artificial Sequence
  Description of Artificial Sequence Synthetic peptide
FPGSDS

SEQ ID NO: 312 - protein, 12 aa, Artificial Sequence
  Description of Artificial Sequence Synthetic peptide
PLYYGSSAWFAY

SEQ ID NO: 313 - protein, 107 aa, Artificial Sequence
  Description of Artificial Sequence Synthetic polypeptide
DIQMTQSPASLSASVGETVTITCRASENIYSYLTWYQQKQRKSPQLLVYNAKTLAEGVPSRFSGSGSGTQFSLKINSLQPEDFGTYYCQHHYATPWTFGGGTKVEIK

SEQ ID NO: 314 - protein, 8 aa, Artificial Sequence
  Description of Artificial Sequence Synthetic peptide
ENIYSYLT

SEQ ID NO: 315 - protein, 7 aa, Artificial Sequence
  Description of Artificial Sequence Synthetic peptide
NAKTLAE

SEQ ID NO: 316 - protein, 9 aa, Artificial Sequence
  Description of Artificial Sequence Synthetic peptide
QHHYATPWT

SEQ ID NO: 317 - protein, 894 aa, Unknown
  Description of Unknown AXL sequence
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSNDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 318 - protein, 119 aa, Artificial Sequence [sequence omitted]

SEQ ID NO: 319 - protein, 7 aa, Artificial Sequence
  Description of Artificial Sequence Synthetic peptide
GFTFSHY

SEQ ID NO: 320 - protein, 6 aa, Artificial Sequence
  Description of Artificial Sequence Synthetic peptide
SSSGGW

SEQ ID NO: 321 - protein, 10 aa, Artificial Sequence
  Description of Artificial Sequence Synthetic peptide
GLKMATIFDY

SEQ ID NO: 322 - protein, 111 aa, Artificial Sequence
  Description of Artificial Sequence Synthetic polypeptide
QSALTQPASVSGSPGQSRRISCTGTSSDVGSYNVVSWYQQHPGKAPKLIIYEVSQRPSGVSNRFSGSKSGNTASLTISGLQTEDEADYYCCSYAGSSIFVIFGGGTKVTVL

SEQ ID NO: 323 - protein, 11 aa, Artificial Sequence
  Description of Artificial Sequence Synthetic peptide
SSDVGSYNVVS

SEQ ID NO: 324 - protein, 7 aa, Artificial Sequence
  Description of Artificial Sequence Synthetic peptide
EVSQRPS

SEQ ID NO: 325 - protein, 11 aa, Artificial Sequence
  Description of Artificial Sequence Synthetic peptide
CSYAGSSIFVI

SEQ ID NO: 326 - protein, 118 aa, Artificial Sequence
  Description of Artificial Sequence Synthetic polypeptide
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSA

SEQ ID NO: 327 - protein, 7 aa, Artificial Sequence
  Description of Artificial Sequence Synthetic peptide
GGSFSGY

SEQ ID NO: 328 - protein, 5 aa, Artificial Sequence
  Description of Artificial Sequence Synthetic peptide
NHSGS

SEQ ID NO: 329 - protein, 9 aa, Artificial Sequence
  Description of Artificial Sequence Synthetic peptide
DKWTWYFDL

SEQ ID NO: 330 - protein, 114 aa, Artificial Sequence
  Description of Artificial Sequence Synthetic polypeptide
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKR

SEQ ID NO: 331 - protein, 14 aa, Artificial Sequence
  Description of Artificial Sequence Synthetic peptide
QSVLYSSSNRNYLA

SEQ ID NO: 332 - protein, 7 aa, Artificial Sequence
  Description of Artificial Sequence Synthetic peptide
WASTRES

SEQ ID NO: 333 - protein, 9 aa, Artificial Sequence
  Description of Artificial Sequence Synthetic peptide
QQYYSTPRT

SEQ ID NO: 334 - protein, 122 aa, Artificial Sequence
  Description of Artificial Sequence Synthetic polypeptide
EVQLVESGGGLVQPGGSLRLSCAASGFTLSGDWIHWVRQAPGKGLEWVGEISAAGGYTDYADSVKGRFTISADTSKNTAYLQMNSLRAEDTAVYYCARESRVSFEAAMDYWGQGTLVTVSSA

SEQ ID NO: 335 - protein, 7 aa, Artificial Sequence
  Description of Artificial Sequence Synthetic peptide
GFTLSGD

SEQ ID NO: 336 - protein, 6 aa, Artificial Sequence
  Description of Artificial Sequence Synthetic peptide
SAAGGY

SEQ ID NO: 337 - protein, 12 aa, Artificial Sequence
  Description of Artificial Sequence Synthetic peptide
ESRVSFEAAMDY

SEQ ID NO: 338 - protein, 108 aa, Artificial Sequence
  Description of Artificial Sequence Synthetic polypeptide
DIQMTQSPSSLSASVGDRVTITCRASQNIATDVAWYQQKPGKAPKLLIYSASFLYSGVPSRFSGSGSGTDFTLTISSLQPEDFATYYCQQSEPEPYTFGQGTKVEIKR

SEQ ID NO: 339 - protein, 8 aa, Artificial Sequence
  Description of Artificial Sequence Synthetic peptide
QNIATDVA

SEQ ID NO: 340 - protein, 7 aa, Artificial Sequence
  Description of Artificial Sequence Synthetic peptide
SASFLYS

SEQ ID NO: 341 - protein, 9 aa, Artificial Sequence
  Description of Artificial Sequence Synthetic peptide
QQSEPEPYT

SEQ ID NO: 342 - protein, 1342 aa, Unknown
  Description of Unknown ERBB-3 sequence
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 343 - protein, 109 aa, Artificial Sequence
  Description of Artificial Sequence Synthetic polypeptide
EVQLVESGGGLVQPGGSLRLSCAASGYTFTSYWMQWVRQAPGKGLEWIGTIYPGDGDTSYAQKFKGRATLSTDKSKNTAYLQMNSLRAEDTAVYYCARWGYAYDIDNWG

SEQ ID NO: 344 - protein, 10 aa, Artificial Sequence
  Description of Artificial Sequence Synthetic peptide
GYTFTSYWMQ

SEQ ID NO: 345 - protein, 16 aa, Artificial Sequence
  Description of Artificial Sequence Synthetic peptide
TIYPGDGDTSYAQKFK

SEQ ID NO: 346 - protein, 9 aa, Artificial Sequence
  Description of Artificial Sequence Synthetic peptide
WGYAYDIDN

SEQ ID NO: 347 - protein, 112 aa, Artificial Sequence
  Description of Artificial Sequence Synthetic polypeptide
DIQMTQSPSSLSASVGDRVTITCKSSQSLLDSDGKTYLNWLQQKPGKAPKRLIYLVSKLDSGVPSRFSGSGSGTDFTLTISSLQPEDFATYYCWQGTHFPYTFGQGTKVEIK

SEQ ID NO: 348 - protein, 16 aa, Artificial Sequence
  Description of Artificial Sequence Synthetic peptide
KSSQSLLDSDGKTYLN

SEQ ID NO: 349 - protein, 7 aa, Artificial Sequence
  Description of Artificial Sequence Synthetic peptide
LVSKLDS

SEQ ID NO: 350 - protein, 9 aa, Artificial Sequence
  Description of Artificial Sequence Synthetic peptide
WQGTHFPYT

SEQ ID NO: 351 - protein, 118 aa, Artificial Sequence
  Description of Artificial Sequence Synthetic polypeptide
QVQLQQPGAALVKPGASVKLSCKASGYTFISYWMLWVKQRPGRGLEWIGRIDPDSGGTKYNEKFKSKATLTVDKSSSTAYMQLSSLTSEDSAVYYCAREGDYAWFAYWGQGTLVPVSA

SEQ ID NO: 352 - protein, 8 aa, Artificial Sequence
  Description of Artificial Sequence Synthetic peptide
GYTFTSYW

SEQ ID NO: 353 - protein, 8 aa, Artificial Sequence
  Description of Artificial Sequence Synthetic peptide
IDPDSGGT

SEQ ID NO: 354 - protein, 11 aa, Artificial Sequence
  Description of Artificial Sequence Synthetic peptide
AREGDYAWFAY

SEQ ID NO: 355 - protein, 112 aa, Artificial Sequence
  Description of Artificial Sequence Synthetic polypeptide
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 356 - protein, 11 aa, Artificial Sequence
  Description of Artificial Sequence Synthetic peptide
QSIVHSNGNTY

SEQ ID NO: 357 - protein, 3 aa, Artificial Sequence
  Description of Artificial Sequence Synthetic peptide
KVS

SEQ ID NO: 358 - protein, 9 aa, Artificial Sequence
  Description of Artificial Sequence Synthetic peptide
FQGSHVPWT

SEQ ID NO: 359 - protein, 442 aa, Unknown
  Description of Unknown EDNRB sequence
MQPPPSLCGRALVALVLACGLSRIWGEERGFPPDRATPLLQTAEIMTPPTKTLWPKGSNASLARSLAPAEVPKGDRTAGSPPRTISPPPCQGPIEIKETFKYINTVVSCLVFVLGIIGNSTLLRIIYKNKCMRNGPNILIASLALGDLLHIVIDIPINVYKLLAEDWPFGAEMCKLVPFIQKASVGITVLSLCALSIDRYRAVASWSRIKGIGVPKWTAVEIVLIWVVSVVLAVPEAIGFDIITMDYKGSYLRICLLHPVQKTAFMQFYKTAKDWWLFSFYFCLPLAITAFFYTLMTCEMLRKKSGMQIALNDHLKQRREVAKTVFCLVLVFALCWLPLHLSRILKLTLYNQNDPNRCELLSFLLVLDYIGINMASLNSCINPIALYLVSKRFKNCFKSCLCCWCQSFEEKQSLEEKQSCLKFKANDHGYDNFRSSNKYSSS

SEQ ID NO: 360 - protein, 123 aa, Artificial Sequence
  Description of Artificial Sequence Synthetic polypeptide
EVQLQQSGAELVRPGALVKLSCKTSGFNIKDYFLHWVRQRPDQGLEWIGWINPDNGNTVYDPKFQGTASLTADTSSNTVYLQLSGLTSEDTAVYFCTRRDYTYEKAALDYWGQGASVIVFSAA

SEQ ID NO: 361 - protein, 7 aa, Artificial Sequence
  Description of Artificial Sequence Synthetic peptide
GFNIKDY

SEQ ID NO: 362 - protein, 7 aa, Artificial Sequence
  Description of Artificial Sequence Synthetic peptide
INPDNGN

SEQ ID NO: 363 - protein, 12 aa, Artificial Sequence
  Description of Artificial Sequence Synthetic peptide
RDYTYEKAALDY

SEQ ID NO: 364 - protein, 107 aa, Artificial Sequence
  Description of Artificial Sequence Synthetic polypeptide
AIQMSQSPASLSASVGETVTITCRASGNIYNYLAWYQQKQGKSPHLLVYDAKTLADGVPSRFSGSGSGTQYSLKISSLQTEDSGNYYCQHFWSLPFTFGSGTKLEIK

SEQ ID NO: 365 - protein, 11 aa, Artificial Sequence
  Description of Artificial Sequence Synthetic peptide
RASGNIYNYLA

SEQ ID NO: 366 - protein, 7 aa, Artificial Sequence
  Description of Artificial Sequence Synthetic peptide
DAKTLAD

SEQ ID NO: 367 - protein, 9 aa, Artificial Sequence
  Description of Artificial Sequence Synthetic peptide
QHFWSLPFT

SEQ ID NO: 368 - protein, 119 aa, Artificial Sequence
  Description of Artificial Sequence Synthetic polypeptide
QVQLVQSGSELKKPGASVKISCKASGYTFTSYAMNWVRQAPGQGLESMGWINTNTGNPTYAQGFTGRFVFSMDTSVSTAYLQISSLKAEDTAIYYCAPRYSSSWYLDYWGQGTLVTVSS

SEQ ID NO: 369 - protein, 15 aa, Artificial Sequence
  Description of Artificial Sequence Synthetic peptide
SCKASGYTFTSYAMN

SEQ ID NO: 370 - protein, 17 aa, Artificial Sequence
  Description of Artificial Sequence Synthetic peptide
WINTNTGNPTYAQGFTG

SEQ ID NO: 371 - protein, 10 aa, Artificial Sequence
  Description of Artificial Sequence Synthetic peptide
RYSSSWYLDY

SEQ ID NO: 372 - protein, 108 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWLMYTFGQGTKLEIK

SEQ ID NO: 373 - protein, 11 aa, Artificial Sequence
  Description of Artificial Sequence Synthetic peptide
RASQSVSSYLA

SEQ ID NO: 374 - protein, 7 aa, Artificial Sequence
  Description of Artificial Sequence Synthetic peptide
DASNRAT

SEQ ID NO: 375 - protein, 10 aa, Artificial Sequence
  Description of Artificial Sequence Synthetic peptide
QQRSNWLMYT

SEQ ID NO: 376 - protein, 119 aa, Artificial Sequence
  Description of Artificial Sequence Synthetic polypeptide
QVQLVQSGSELKKPGASVKISCKASGYTFTSYAMNWVRQAPGQGLECMGWINTNTGNPTYAQGFTGRFVFSMDTSVSTAYLQISSLKAEDTAIYYCAPRYSSSWYLDYWGQGTLVTVSS

SEQ ID NO: 377 - protein, 15 aa, Artificial Sequence
  Description of Artificial Sequence Synthetic peptide
SCKASGYTFTSYAMN

SEQ ID NO: 378 - protein, 17 aa, Artificial Sequence
  Description of Artificial Sequence Synthetic peptide
WINTNTGNPTYAQGFTG

SEQ ID NO: 379 - protein, 10 aa, Artificial Sequence
  Description of Artificial Sequence Synthetic peptide
RYSSSWYLDY

SEQ ID NO: 380 - protein, 108 aa, Artificial Sequence; identical to SEQ ID NO: 372
  Description of Artificial Sequence Synthetic polypeptide

SEQ ID NO: 381 - protein, 11 aa, Artificial Sequence
  Description of Artificial Sequence Synthetic peptide
RASQSVSSYLA

SEQ ID NO: 382 - protein, 7 aa, Artificial Sequence
  Description of Artificial Sequence Synthetic peptide
DASNRAT

SEQ ID NO: 383 - protein, 10 aa, Artificial Sequence
  Description of Artificial Sequence Synthetic peptide
QQRSNWLMYT

SEQ ID NO: 384 - protein, 537 aa, Unknown
  Description of Unknown TYRP1 sequence
MSAPKLLSLGCIFFPLLLFQQARAQFPRQCATVEALRSGMCCPDLSPVSGPGTDRCGSSSGRGRCEAVTADSRPHSPQYPHDGRDDREVWPLRFFNRTCHCNGNFSGHNCGTCRPGWRGAACDQRVLIVRRNLLDLSKEEKNHFVRALDMAKRTTHPLFVIATRRSEEILGPDGNTPQFENISIYNYFVWTHYYSVKKTFLGVGQESFGEVDFSHEGPAFLTWHRYHLLRLEKDMQEMLQEPSFSLPYWNFATGKNVCDICTDDLMGSRSNFDSTLISPNSVFSQWRVVCDSLEDYDTLGTLCNSTEDGPIRRNPAGNVARPMVQRLPEPQDVAQCLEVGLFDTPPFYSNSTNSFRNTVEGYSDPTGKYDPAVRSLHNLAHLFLNGTGGQTHLSPNDPIFVLLHTFTDAVFDEWLRRYNADISTFPLENAPIGHNRQYNMVPFWPPVTNTEMFVTAPDNLGYTYEIQWPSREFSVPEIIAIAVVGALLLVALIFGTASYLIRARRSMDEANQPLLTDQYQCYAEEYEKLQNPNQSVV

SEQ ID NO: 385 - protein, 119 aa, Artificial Sequence
  Description of Artificial Sequence Synthetic polypeptide
QVQLLESGGGLVQPGGSLRLSCAASGFTFSDYALHWVRQAPGKGLEWVSVISYQGGFIYYADSVKGRFTISRDNSKNTLYLQMNSLRAEDTAVYYCARSPGLTSYHDSWGQGTLVTVSS

SEQ ID NO: 386 - protein, 5 aa, Artificial Sequence
  Description of Artificial Sequence Synthetic peptide
DYALH

SEQ ID NO: 387 - protein, 17 aa, Artificial Sequence
  Description of Artificial Sequence Synthetic peptide
VISYQGGFIYYADSVKG

SEQ ID NO: 388 - protein, 10 aa, Artificial Sequence
  Description of Artificial Sequence Synthetic peptide
SPGLTSYHDS

SEQ ID NO: 389 - protein, 110 aa, Artificial Sequence
  Description of Artificial Sequence Synthetic polypeptide
QSVLTQPPSVSGAPGQRVTISCSGSSSNTGSNYVSWYQQLPGTAPKLLIHSTSHRPSGVPDRFSGSKSGTSASLAITGLQAEDSADYYCQSWDVESERVVFGGGTKLTVL

SEQ ID NO: 390 - protein, 13 aa, Artificial Sequence
  Description of Artificial Sequence Synthetic peptide
SGSSSNIGSNYVS

SEQ ID NO: 391 - protein, 7 aa, Artificial Sequence
  Description of Artificial Sequence Synthetic peptide
STSHRPS

SEQ ID NO: 392 - protein, 11 aa, Artificial Sequence
  Description of Artificial Sequence Synthetic peptide
QSWDYESERVV

SEQ ID NO: 393 - protein, 128 aa, Artificial Sequence
  Description of Artificial Sequence Synthetic polypeptide
QVQLVQSGAEVKKPGAVKVSCKVSGYTLTELSMHWVRQAPGKGLEWMGGFDPEDFKYHTHQKFQGRVTMTEDTSTDTAYMELSSLRSEDTAVYYCALVWGTQGKGVRGWDYYYGMDVWGQGTTVTVSS

SEQ ID NO: 394 - protein, 5 aa, Artificial Sequence
  Description of Artificial Sequence Synthetic peptide
ELSMH

SEQ ID NO: 395 - protein, 17 aa, Artificial Sequence
  Description of Artificial Sequence Synthetic peptide
GFDPEDFKYHTHQKFQG

SEQ ID NO: 396 - protein, 20 aa, Artificial Sequence
  Description of Artificial Sequence Synthetic peptide
VWGTQGKGVRGWDYYYGMDV

SEQ ID NO: 397 - protein, 111 aa, Artificial Sequence
  Description of Artificial Sequence Synthetic polypeptide
QSVVTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGWVFGGGTKLTVL

SEQ ID NO: 398 - protein, 14 aa, Artificial Sequence
  Description of Artificial Sequence Synthetic peptide
TGSSSNIGAGYDVH

SEQ ID NO: 399 - protein, 7 aa, Artificial Sequence
  Description of Artificial Sequence Synthetic peptide
GNSNRPS

SEQ ID NO: 400 - protein, 11 aa, Artificial Sequence
  Description of Artificial Sequence Synthetic peptide
QSYDSSLSGWV

SEQ ID NO: 401 - protein, 273 aa, Unknown
  Description of Unknown OLR1 isoform 1 sequence
MTFDDLKIQTVKDQPDEKSNGKKAKGLQFLYSPWWCLAAATLGVLCLGLVVTIMVLGMQLSQVSDLLTQEQANLTHQKKKLEGQISARQQAEEASQESENELKEMIETLARKLNEKSKEQMELHHQNLNLQETLKRVANCSAPCPQDWIWHGENCYLFSSGSFNWEKSQEKCLSLDAKLLKINSTADLDFIQQAISYSSFPFWMGLSRRNPSYPWLWEDGSPLMPHLFRVRGAVSQTYPSGTCAYIQRGAVYAENCILAAFSICQKKANLRAQ

SEQ ID NO: 402 - protein, 181 aa, Unknown
  Description of Unknown OLR1 isoform 2 sequence
MTFDDLKIQTVKDQPDEKSNGKKAKGLQFLYSPWWCLAAATLGVLCLGLVVTIMVLGMQLSQVSDLLTQEQANLTHQKKKLEGQISARQQAEEASQESENELKEMIETLARKLNEKSKEQMELHHQNLNLQETLKRVANCSGLHPASNFLFQFSILDGAVSEEPQLPMALGGRFSFDAPLI

SEQ ID NO: 403 - protein, 189 aa, Unknown
  Description of Unknown OLR1 isoform 3 sequence
MTFDDLKIQTVKDQPDEKSNGKKAKGLQFLYSPWWCLAAATLGVLCLGLVVTIMVLGMQLSQVSDLLTQEQANLTHQKKKLEGQISARQQAEEASQESENELKEMIETLARKLNEKSKEQMELHHQNLNLQETLKRVANCSAPCPQDWIWHGENCYLFSSGSFNWEKSQEKCLSLDAKLLKINSTADLI

SEQ ID NO: 404 - protein, 909 aa, Unknown
  Description of Unknown ADAM12 sequence
MAARPLPVSPARALLLALAGALLAPCEARGVSLWNQGRADEVVSASVGSGDLWIPVKSFDSKNHPEVLNIRLQRESKELIINLERNEGLIASSFTETHYLQDGTDVSLARNYTVILGHCYYHGHVRGYSDSAVSLSTCSGLRGLIVFENESYVLEPMKSATNRYKLFPAKKLKSVRGSCGSHHNTPNLAAKNVFPPPSQTWARRHKRETLKATKYVELVIVADNREFQRQGKDLEKVKQRLIEIANHVDKFYRPLNIRIVLVGVEVWNDMDKCSVSQDPFTSLHEFLDWRKMKLLPRKSHDNAQLVSGVYFQGTTIGMAPIMSMCTADQSGGIVMDHSDNPLGAAVTLAHELGHNFGMNHDTLDRGCSCQMAVEKGGCIMNASTGYPFPMVFSSCSRKDLETSLEKGMGVCLFNLPEVRESFGGQKCGNRFVEEGEECDCGEPEECMNRCCNATTCTLKPDAVCAHGLCCEDCQLKPAGTACRDSSNSCDLPEFCTGASPHCPANVYLHDGHSCQDVDGYCYNGICQTHEQQCVTLWGPGAKPAPGICFERVNSAGDPYGNCGKVSKSSFAKCEMRDAKCGKIQCQGGASRPVIGTNAVSIETNIPLQQGGRILCRGTHVYLGDDMPDPGLVLAGTKCADGKICLNRQCQNISVFGVHECAMQCHGRGVCNNRKNCHCEAHWAPPFCDKFGFGGSTDSGPIRQADNQGLTIGILVTILCLLAAGFVVYLKRKTLIRLLFTNKKTTIEKLRCVRPSRPPRGFQPCQAHLGHLGKGLMRKPPDSYPPKDNPRRLLQCQNVDISRPLNGLNVPQPQSTQRVLPPLHRAPRAPSVPARPLPAKPALRQAQGTCKPNPPQKPLPADPLARTTRLTHALARTPGQWETGLRLAPLRPAPQYPHQVPRSTHTAYIK

SEQ ID NO: 405 - protein, 119 aa, Artificial Sequence
  Description of Artificial Sequence Synthetic polypeptide
EVQLQQSGPELVKTGASVKISCKASGYSFTSYYMHWVKQSHGKSLEWIGEINPYNGGASYNQKIKGRATFTVDTSSRTAYMQFNSLTSEDSAVYYCARSIYGHSVLDYWGQGTSVSVSS

SEQ ID NO: 406 - protein, 7 aa, Artificial Sequence
  Description of Artificial Sequence Synthetic peptide
GYSFTSY

SEQ ID NO: 407 - protein, 6 aa, Artificial Sequence
  Description of Artificial Sequence Synthetic peptide
NPYNGG

SEQ ID NO: 408 - protein, 10 aa, Artificial Sequence
  Description of Artificial Sequence Synthetic peptide
SIYGHSVLDY

SEQ ID NO: 409 - protein, 113 aa, Artificial Sequence
  Description of Artificial Sequence Synthetic polypeptide
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPLTFGAGTKLELKR

SEQ ID NO: 410 - protein, 13 aa, Artificial Sequence
  Description of Artificial Sequence Synthetic peptide
QSLLDSDGKTYLN

SEQ ID NO: 411 - protein, 7 aa, Artificial Sequence
  Description of Artificial Sequence Synthetic peptide
LVSKLDS

SEQ ID NO: 412 - protein, 9 aa, Artificial Sequence
  Description of Artificial Sequence Synthetic peptide
WQGTHFPLT

SEQ ID NO: 413 - protein, 227 aa, Artificial Sequence
  Description of Artificial Sequence Synthetic polypeptide
QVQLQQSGPGLVKPSQTLSLTCAISGDSVSSNSAAWNWIRQSPSRGLEWLGRTYYRSKWYNDYAVSVKSRIIINPDTSKNQFSLQLNSVTPEDTAVYYCARDPGGPLDDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 414 - protein, 9 aa, Artificial Sequence
  Description of Artificial Sequence Synthetic peptide
MQALQTPFT

SEQ ID NO: 415 - protein, 18 aa, Artificial Sequence
  Description of Artificial Sequence Synthetic peptide
RTYYRSKWYNDYAVSVKS

SEQ ID NO: 416 - protein, 12 aa, Artificial Sequence
  Description of Artificial Sequence Synthetic peptide
DPGGPLDDSFDI

SEQ ID NO: 417 - protein, 221 aa, Artificial Sequence
  Description of Artificial Sequence Synthetic polypeptide
LDVVMTQSPLSLPVTPGEPASISCRSSQSLLRSNGYNYLDWYLQKPGQSPQLLIYLGSIRASGVPDRFSGSGSGTDFTLRISRVEAEDVGVYYCMQALQTPFTFGQGTKLEIKRTVAAPSVFIFPPSDEQLKSGTASVVCLLNNFYPREAKVQWKVDNALQSGNSQESVTEQDSICDSTYSLSSTLTLSKADYEKHKVYACEVTHQGLSSPVTKSFNRGEC

SEQ ID NO: 418 - protein, 16 aa, Artificial Sequence
  Description of Artificial Sequence Synthetic peptide
RSSQSLLRSNGYNYLD

SEQ ID NO: 419 - protein, 7 aa, Artificial Sequence
  Description of Artificial Sequence Synthetic peptide
LGSIRAS

SEQ ID NO: 420 - protein, 9 aa, Artificial Sequence
  Description of Artificial Sequence Synthetic peptide
MQALQTPFT

SEQ ID NO: 421 - protein, 335 aa, Unknown
  Description of Unknown PLAUR sequence
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 422 - protein, 117 aa, Artificial Sequence
  Description of Artificial Sequence Synthetic polypeptide
EVQLVESGGGLVQPGGSLRLSCAASGFSFSDYYMYWVRQAPGKGLEWVSYITNGGITYYPDSVKGRFTISRDNSKNTLYLQMNSLRAEDTAVYYCTSPLRGAWFAYWGQGTLVTVSS

SEQ ID NO: 423 - protein, 10 aa, Artificial Sequence
  Description of Artificial Sequence Synthetic peptide
GFSFSDYYMY

SEQ ID NO: 424 - protein, 16 aa, Artificial Sequence
  Description of Artificial Sequence Synthetic peptide
YITNGGITYYPDSVKG

SEQ ID NO: 425 - protein, 11 aa, Artificial Sequence
  Description of Artificial Sequence Synthetic peptide
TSPLRGAWFAY

SEQ ID NO: 426 - protein, 112 aa, Artificial Sequence
  Description of Artificial Sequence Synthetic polypeptide
DVVMTQSPLSLPVTLGQPASISCRSSQSIVHSNGNTYLEWYQQRPGQSPRLLIYKVSNRFSGVPDRFSGSGSGTDFTLKISRVEAEDVGVYYCFQGSHVPLTFGQGTKLEIK

SEQ ID NO: 427 - protein, 13 aa, Artificial Sequence
  Description of Artificial Sequence Synthetic peptide
QSIVHSNGNTYLE

SEQ ID NO: 428 - protein, 7 aa, Artificial Sequence
  Description of Artificial Sequence Synthetic peptide
KVSNRFS

SEQ ID NO: 429 - protein, 9 aa, Artificial Sequence
  Description of Artificial Sequence Synthetic peptide
FQGSHVPLT

SEQ ID NO: 430 - protein, 451 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAMYYCARHGYSATRQGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 431 - protein, 12 aa, Artificial Sequence
  Description of Artificial Sequence Synthetic peptide
FTFSSYAMSWVR

SEQ ID NO: 432 - protein, 16 aa, Artificial Sequence
  Description of Artificial Sequence Synthetic peptide
VSAISGSGGSTYYADS

SEQ ID NO: 433 - protein, 18 aa, Artificial Sequence
  Description of Artificial Sequence Synthetic peptide
CARHGYSATRQGLDYWGQ

SEQ ID NO: 434 - protein, 215 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYSSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 435 - protein, 12 aa, Artificial Sequence
  Description of Artificial Sequence Synthetic peptide
RASQSVSSSYLA

SEQ ID NO: 436 - protein, 7 aa, Artificial Sequence
  Description of Artificial Sequence Synthetic peptide
GASSRAT

SEQ ID NO: 437 - protein, 13 aa, Artificial Sequence
  Description of Artificial Sequence Synthetic peptide
CQQYSSSPLTFGQ

SEQ ID NO: 438 - protein, 374 aa, Unknown
  Description of Unknown CCR6 sequence
MSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTM

SEQ ID NO: 439 - protein, 122 aa, Artificial Sequence
  Description of Artificial Sequence Synthetic polypeptide
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATAQPFHWGDDAFDIWGQGTMVTVSS

SEQ ID NO: 440 - protein, 5 aa, Artificial Sequence
  Description of Artificial Sequence Synthetic peptide
ELSMH

SEQ ID NO: 441 - protein, 17 aa, Artificial Sequence
  Description of Artificial Sequence Synthetic peptide
GFDPEDGETIYAQKFQG

SEQ ID NO: 442 - protein, 13 aa, Artificial Sequence
  Description of Artificial Sequence Synthetic peptide
AQPFHWGDDAFDI

SEQ ID NO: 443 - protein, 111 aa, Artificial Sequence
  Description of Artificial Sequence Synthetic polypeptide
QSVLTQPPSASGTPGQRVTISCSGSSSNIGSNTVNWYQQPPGTAPKLLIYSNNQRPSGVPDRFSGSKSGTSASLAISGLQSEDEADYYCAAWDDSLNGPVFGGGTKLTVLG

SEQ ID NO: 444 - protein, 13 aa, Artificial Sequence
  Description of Artificial Sequence Synthetic peptide
SGSSSNIGSNTVN

SEQ ID NO: 445 - protein, 7 aa, Artificial Sequence
  Description of Artificial Sequence Synthetic peptide
SNNQRPS

SEQ ID NO: 446 - protein, 11 aa, Artificial Sequence
  Description of Artificial Sequence Synthetic peptide
AAWDDSLNGPV

SEQ ID NO: 447 - protein, 126 aa, Artificial Sequence
  Description of Artificial Sequence Synthetic polypeptide
QVQLQQSGAEVKKPGSSVKVSCKASGYTFTGYYMHWVRQAPGQGLEWMGGIIPIFGTANYAQKFQGRVTITADKSTSTAYMELSSLRSEDTAVYYCATAPMVCSSTSCYLRGFDYWGQGTLVTVSS

SEQ ID NO: 448 - protein, 8 aa, Artificial Sequence
  Description of Artificial Sequence Synthetic peptide
GYTFTGYY

SEQ ID NO: 449 - protein, 8 aa, Artificial Sequence
  Description of Artificial Sequence Synthetic peptide
IIPIFGTA

SEQ ID NO: 450 - protein, 19 aa, Artificial Sequence
  Description of Artificial Sequence Synthetic peptide
ATAPMVCSSTSCYLRGFDY

SEQ ID NO: 451 - protein, 112 aa, Artificial Sequence
  Description of Artificial Sequence Synthetic polypeptide
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPITFGQGTRLEIK

SEQ ID NO: 452 - protein, 11 aa, Artificial Sequence
  Description of Artificial Sequence Synthetic peptide
QSLLHSNGYNY

SEQ ID NO: 453 - protein, 3 aa, Artificial Sequence
  Description of Artificial Sequence Synthetic peptide
LGS

SEQ ID NO: 454 - protein, 7 aa, Artificial Sequence
  Description of Artificial Sequence Synthetic peptide
MQALQTP

SEQ ID NO: 455 - protein, 986 aa, Unknown
  Description of Unknown EPHA4 sequence
MAGIFYFALFSCLFGICDAVTGSRVYPANEVTLLDSRSVQGELGWIASPLEGGWEEVSIMDEKNTPIRTYQVCNVMEPSQNNWLRTDWITREGAQRVYIEIKFTLRDCNSLPGVMGTCKETFNLYYYESDNDKERFIRENQFVKIDTIAADESFTQVDIGDRIMKLNTEIRDVGPLSKKGFYLAFQDVGACIALVSVRVFYKKCPLTVRNLAQFPDTITGADTSSLVEVRGSCVNNSEEKDVPKMYCGADGEWLVPIGNCLCNAGHEERSGECQACKIGYYKALSTDATCAKCPPHSYSVWEGATSCTCDRGFFRADNDAASMPCTRPPSAPLNLISNVNETSVNLEWSSPQNTGGRQDISYNVVCKKCGAGDPSKCRPCGSGVHYTPQQNGLKTTKVSITDLLAHTNYTFEIWAVNGVSKYNPNPDQSVSVTVTTNQAAPSSIALVQAKEVTRYSVALAWLEPDRPNGVILEYEVKYYEKDQNERSYRIVRTAARNTDIKGLNPLTSYVFHVRARTAAGYGDFSEPLEVTTNTVPSRIIGDGANSTVLLVSVSGSVVLVVILIAAFVISRRRSKYSKAKQEADEEKHLNQGVRTYVDPFTYEDPNQAVREFAKEIDASCIKIEKVIGVGEFGEVCSGRLKVPGKREICVAIKTLKAGYTDKQRRDFLSEASIMGQFDHPNIIHLEGVVTKCKPVMIITEYMENGSLDAFLRKNDGRFTVIQLVGMLRGIGSGMKYLSDMSYVHRDLAARNILVNSNLVCKVSDFGMSRVLEDDPEAAYTTRGGKIPIRWTAPEAIAYRKFTSASDVWSYGIVMWEVMSYGERPYWDMSNQDVIKAIEEGYRLPPPMDCPIALHQLMLDCWQKERSDRPKFGQIVNMLDKLIRNPNSLKRTGTESSRPNTALLDPSSPEFSAVVSVGDWLQAIKMDRYKDNFTAAGYTTLEAVVHVNQEDLARIGITAITHQNKILSSVQAMRTQMQQMHGRMVPV

SEQ ID NO: 456 - protein, 131 aa, Artificial Sequence
  Description of Artificial Sequence Synthetic polypeptide
MGWSCIILFLVATATGVHSNIVLTQSPASLAVSLGQRATISCRASESVDSYGNSFMHWYQQKPGQPPKLLIYLASNLESGVPARFSGSGSRTDFTLTIDPVEADDVATYYCQQNNGDPYTFGGGTKLEIIR

SEQ ID NO: 457 - protein, 14 aa, Artificial Sequence
  Description of Artificial Sequence Synthetic peptide
RASESVDSYGNSFM

SEQ ID NO: 458 - protein, 7 aa, Artificial Sequence
  Description of Artificial Sequence Synthetic peptide
LASNLES

SEQ ID NO: 459 - protein, 9 aa, Artificial Sequence
  Description of Artificial Sequence Synthetic peptide
QQNNGDPYT

SEQ ID NO: 460 - protein, 140 aa, Artificial Sequence
  Description of Artificial Sequence Synthetic polypeptide
MGWSCIILFLVATATGVHSEVQLVESGGGLMQPKGSLKLSCAASGFTFKTYALNWVRQAPGTGLEWVARIRSKSNNYTTYYADSVKDRFTISRDDSQNMLYLQMNNLKTEDTAMYYCVRPQSGTSFAYWGQGTLVTVSAA

SEQ ID NO: 461 - protein, 10 aa, Artificial Sequence
  Description of Artificial Sequence Synthetic peptide
GFTFKTYALN

SEQ ID NO: 462 - protein, 18 aa, Artificial Sequence
  Description of Artificial Sequence Synthetic peptide
RIRSKSNNYTTYYADSVK

SEQ ID NO: 463 - protein, 9 aa, Artificial Sequence
  Description of Artificial Sequence Synthetic peptide
PQSGTSFAY

SEQ ID NO: 464 - protein, 5 aa, Artificial Sequence
  Description of Artificial Sequence Synthetic peptide
RYTMG

SEQ ID NO: 465 - protein, 15 aa, Artificial Sequence
  Description of Artificial Sequence Synthetic peptide
IINSGATYYASWAKG

SEQ ID NO: 466 - protein, 3 aa, Artificial Sequence
  Description of Artificial Sequence Synthetic peptide
GDA

SEQ ID NO: 467 - protein, 11 aa, Artificial Sequence
  Description of Artificial Sequence Synthetic peptide
QASQSIGSNLA

SEQ ID NO: 468 - protein, 7 aa, Artificial Sequence
  Description of Artificial Sequence Synthetic peptide
KASKLAS

SEQ ID NO: 469 - protein, 13 aa, Artificial Sequence
  Description of Artificial Sequence Synthetic peptide
QCSYTAIGNYGHV

SEQ ID NO: 470 - protein, 375 aa, Unknown
  Description of Unknown CD14 sequence
MERASCLLLLLLPLVHVSATTPEPCELDDEDFRCVCNFSEPQPDWSEAFQCVSAVEVEIHAGGLNLEPFLKRVDADADPRQYADTVKALRVRRLTVGAAQVPAQLLVGALRVLAYSRLKELTLEDLKITGTMPPLPLEATGLALSSLRLRNVSWATGRSWLAELQQWLKPGLKVLSIAQAHSPAFSCEQVRAFPALTSLDLSDNPGLGERGLMAALCPHKFPAIQNLALRNTGMETPTGVCAALAAAGVQPHSLDLSHNSLRATVNPSAPRCMWSSALNSLNLSFAGLEQVPKGLPAKLRVLDLSCNRLNRAPQPDELPEVDNLTLDGNPFLVPGTALPHEGSMNSGVVPACARSTLSVGVSGTLVLLQGARGFA

SEQ ID NO: 471 - protein, 116 aa, Artificial Sequence
  Description of Artificial Sequence Synthetic polypeptide
QVQLQESGPGLVKPSETLSLTCTVSGYSITSDYAWNWIRQFPGKKLEWMGSIYYSGSTYYNPSLKSRVTISVDTSKNQFSLKLSSVTAADTATYYCVSGTYYFDYWGQGTTLTVSS

SEQ ID NO: 472 - protein, 8 aa, Artificial Sequence
  Description of Artificial Sequence Synthetic peptide
GYSITSDY

SEQ ID NO: 473 - protein, 3 aa, Artificial Sequence
  Description of Artificial Sequence Synthetic peptide
YSG

SEQ ID NO: 474 - protein, 12 aa, Artificial Sequence
  Description of Artificial Sequence Synthetic peptide
CVSGTYYFDYWG

SEQ ID NO: 475 - protein, 109 aa, Artificial Sequence
  Description of Artificial Sequence Synthetic polypeptide
DIVMTQSPSSLSASVGDRVTITCRASQSVSSDVAWFQQKPGKSPKPLIYYASNRYSGVPSRFSGSGSGTDFTLTISSLQAEDFAVYFCGQDYTSPRTFGGGTKLEIKRA

SEQ ID NO: 476 - protein, 9 aa, Artificial Sequence
  Description of Artificial Sequence Synthetic peptide
ASQSVSSDV

SEQ ID NO: 477 - protein, 3 aa, Artificial Sequence
  Description of Artificial Sequence Synthetic peptide
YAS

SEQ ID NO: 478 - protein, 8 aa, Artificial Sequence
  Description of Artificial Sequence Synthetic peptide
QDYTSPRT

SEQ ID NO: 479 - protein, 116 aa, Artificial Sequence
  Description of Artificial Sequence Synthetic polypeptide
DVQLQESGPGLVKPSQSLSLTCTVTGYSITSDYAWNWIRQFPGNKLEWMGYITYSGITNYNPSLKSQISITRDTSKNQFFLQLNSVTTEDTATYYCVSGTYYFDYWGQGTTLTVSS

SEQ ID NO: 480 - protein, 8 aa, Artificial Sequence
  Description of Artificial Sequence Synthetic peptide
GYSITSDY

SEQ ID NO: 481 - protein, 3 aa, Artificial Sequence
  Description of Artificial Sequence Synthetic peptide
YSG

SEQ ID NO: 482 - protein, 7 aa, Artificial Sequence
  Description of Artificial Sequence Synthetic peptide
YYFDYWG

SEQ ID NO: 483 - protein, 108 aa, Artificial Sequence
  Description of Artificial Sequence Synthetic polypeptide
SVVMTQTPKSLLISIGDRVTITCKASQSVSSDVAWFQQKPGQSPKPLIYYASNRYTGVPDRFTGSGYGTDFTFTISSVQAEDLAVYFCGQDYTSPRTFGGGTKLEIKR

SEQ ID NO: 484 - protein, 9 aa, Artificial Sequence
  Description of Artificial Sequence Synthetic peptide
ASQSVSSDV

SEQ ID NO: 485 - protein, 3 aa, Artificial Sequence
  Description of Artificial Sequence Synthetic peptide
YAS

SEQ ID NO: 486 - protein, 8 aa, Artificial Sequence
  Description of Artificial Sequence Synthetic peptide
QDYTSPRT

SEQ ID NO: 487 - protein, 1156 aa, Unknown
  Description of Unknown CD163 sequence
MSKLRMVLLEDSGSADFRRHFVNLSPFTITVVLLLSACFVTSSLGGTDKELRLVDGENKCSGRVEVKVQEEWGTVCNNGWSMEAVSVICNQLGCPTAIKAPGWANSSAGSGRIWMDHVSCRGNESALWDCKHDGWGKHSNCTHQQDAGVTCSDGSNLEMRLTRGGNMCSGRIEIKFQGRWGTVCDDNFNIDHASVICRQLECGSAVSFSGSSNFGEGSGPIWFDDLICNGNESALWNCKHQGWGKHNCDHAEDAGVICSKGADLSLRLVDGVTECSGRLEVRFQGEWGTICDDGWDSYDAAVACKQLGCPTAVTAIGRVNASKGFGHIWLDSVSCQGHEPAIWQCKHHEWGKHYCNHNEDAGVTCSDGSDLELRLRGGGSRCAGTVEVEIQRLLGKVCDRGWGLKEADVVCRQLGCGSALKTSYQVYSKIQATNTWLFLSSCNGNETSLWDCKNWQWGGLTCDHYEEAKITCSAHREPRLVGGDIPCSGRVEVKHGDTWGSICDSDFSLEAASVLCRELQCGTVVSILGGAHFGEGNGQIWAEEFQCEGHESHLSLCPVAPRPEGTCSHSRDVGVVCSRYTEIRLVNGKTPCEGRVELKTLGAWGSLCNSHWDIEDAHVLCQQLKCGVALSTPGGARFGKGNGQIWRHMFHCTGTEQHMGDCPVTALGASLCPSEQVASVICSGNQSQTLSSCNSSSLGPTRPTIPEESAVACIESGQLRLVNGGGRCAGRVEIYHEGSWGTICDDSWDLSDAHVVCRQLGCGEAINATGSAHFGEGTGPIWLDEMKCNGKESRIWQCHSHGWGQQNCRHKEDAGVICSEFMSLRLTSEASREACAGRLEVFYNGAWGTVGKSSMSETTVGVVCRQLGCADKGKINPASLDKAMSIPMWVDNVQCPKGPDTLWQCPSSPWEKRLASPSEETWITCDNKIRLQEGPTSCSGRVEIWHGGSWGTVCDDSWDLDDAQVVCQQLGCGPALKAFKEAEFGQGTGPIWLNEVKCKGNESSLWDCPARRWGHSECGHKEDAAVNCTDISVQKTPQKATTGRSSRQSSFIAVGILGVVLLAIFVALFFLTKKRRQRQRLAVSSRGENLVHQIQYREMNSCLNADDLDLMNSSENSHESADFSAAELISVSKFLPISGMEKEAILSHTEKENGNL

SEQ ID NO: 488 - protein, 118 aa, Artificial Sequence
  Description of Artificial Sequence Synthetic polypeptide
EVQLLESGGGLVQPGGSLRLSCAASGFTFSLYWMGWVRQAPGKGLEWVSSISSSGGVTPYADSVKGRFTISRDNSKNTLYLQMNSLRAEDTAVYYCAMLGELGWFDPWGQGTLVTVSS

SEQ ID NO: 489 - protein, 5 aa, Artificial Sequence
  Description of Artificial Sequence Synthetic peptide
LYWMG

SEQ ID NO: 490 - protein, 17 aa, Artificial Sequence
  Description of Artificial Sequence Synthetic peptide
SISSSGGVTPYADSVKG

SEQ ID NO: 491 - protein, 9 aa, Artificial Sequence
  Description of Artificial Sequence Synthetic peptide
LGELGWFDP

SEQ ID NO: 492 - protein, 107 aa, Artificial Sequence
  Description of Artificial Sequence Synthetic polypeptide
DIQMTQSPSALSASVGDRVTITCRASQGISSYLNWYQQKPGKAPKLLIYYASNLQNGIPSRFSGSGSGTDFTLTISSLQPEDFATYHCQQSYSTPLTFGGGTNVEIR

SEQ ID NO: 493 - protein, 11 aa, Artificial Sequence
  Description of Artificial Sequence Synthetic peptide
RASQGISSYLN

SEQ ID NO: 494 - protein, 6 aa, Artificial Sequence
  Description of Artificial Sequence Synthetic peptide
ASNLQN

SEQ ID NO: 495 - protein, 9 aa, Artificial Sequence
  Description of Artificial Sequence Synthetic peptide
QQSYSTPLT

SEQ ID NO: 496 - protein, 836 aa, Unknown
  Description of Unknown CSF3R sequence
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 497 - protein, 116 aa, Artificial Sequence
  Description of Artificial Sequence Synthetic polypeptide
DVQLQESGPGLVKPSQSLSLTCTVTGYSITGGFAWNWIRQFPGNTLEWMGYIGYGGSTSYNPSLNSRISITRDTSKNHFFLQFNSVTTDDSATYYCARGDYLFAYWGQGTLVTVSA

SEQ ID NO: 498 - protein, 6 aa, Artificial Sequence
  Description of Artificial Sequence Synthetic peptide
GGFAWN

SEQ ID NO: 499 - protein, 16 aa, Artificial Sequence
  Description of Artificial Sequence Synthetic peptide
YIGYGGSTSYNPSLNS

SEQ ID NO: 500 - protein, 7 aa, Artificial Sequence
  Description of Artificial Sequence Synthetic peptide
GDYLFAY

SEQ ID NO: 501 - protein, 107 aa, Artificial Sequence
  Description of Artificial Sequence Synthetic polypeptide
DIVMTQSHKFMSTSVGDRVSITCKASQDVNTAVAWYQQKPGQSPKLLIYSASYRYTGVPDRFTGSGSGTDFTFTISSVQAEDLAVYYCQQHYSTPRTFGGGTKLEIK

SEQ ID NO: 502 - protein, 11 aa, Artificial Sequence
  Description of Artificial Sequence Synthetic peptide
KASQDVNTAVA

SEQ ID NO: 503 - protein, 7 aa, Artificial Sequence
  Description of Artificial Sequence Synthetic peptide
SASYRYT

SEQ ID NO: 504 - protein, 9 aa, Artificial Sequence
  Description of Artificial Sequence Synthetic peptide
QQHYSTPRT

SEQ ID NO: 505 - protein, 119 aa, Artificial Sequence
  Description of Artificial Sequence Synthetic polypeptide
EVKLVESGGALVKPGGSLKLSCAASGFTFSNYAMSWVRQTPEKRLEWVATINNGGSYTYYSDSVKGRFAISRDNAKNTLYLQMSNLRSEDTALYYCVRRDYGTSDFDYWGQGTTLTVSS

SEQ ID NO: 506 - protein, 9 aa, Artificial Sequence
  Description of Artificial Sequence Synthetic peptide
FTFSNYAMS

SEQ ID NO: 507 - protein, 14 aa, Artificial Sequence
  Description of Artificial Sequence Synthetic peptide
VATINNGGSYTYYS

SEQ ID NO: 508 - protein, 12 aa, Artificial Sequence
  Description of Artificial Sequence Synthetic peptide
VRRDYGTSDFDY

SEQ ID NO: 509 - protein, 107 aa, Artificial Sequence
  Description of Artificial Sequence Synthetic polypeptide
DIQMTQSPSSLSASLGERVSLTCRASQEISGYLGWLQQKPDGTIKRLIFSTSTLDSGVPKRFSGSRSGSDYSLTISSLESEDFADYYCLQYASYPPTFGGGTKLEIK

SEQ ID NO: 510 - protein, 11 aa, Artificial Sequence
  Description of Artificial Sequence Synthetic peptide
RASQEISGYLG

SEQ ID NO: 511 - protein, 7 aa, Artificial Sequence
  Description of Artificial Sequence Synthetic peptide
STSTLDS

SEQ ID NO: 512 - protein, 9 aa, Artificial Sequence
  Description of Artificial Sequence Synthetic peptide
LQYASYPPT

SEQ ID NO: 513 - protein, 479 aa, Unknown
  Description of Unknown Siglec-9 sequence
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGVVGGAGATALVFLSFCVIFVVVRSCRKKSARPAAGVGDTGIEDANAVRGSASQILNHFIGFPTFLGLGFEFLLNLRDLCCHPDSEFYVYHFSHFRLIKNIAGEIVWSLEGKILWLLDVSDFFHWFFLICVG

SEQ ID NO: 514 - protein, 121 aa, Artificial Sequence
  Description of Artificial Sequence Synthetic polypeptide
VQLVESGGGLVQPGGSLRLSCAASGFNIKDTYIHWVRQAPGKGLEWVARIDPANDKTRYADSVKGRFTISADTSKNTAYLQMNSLRAEDTAVYYCSSEGHYGYDGYAMDYWGQGTLVTVSS

SEQ ID NO: 515 - protein, 6 aa, Artificial Sequence
  Description of Artificial Sequence Synthetic peptide
GFNIKD

SEQ ID NO: 516 - protein, 6 aa, Artificial Sequence
  Description of Artificial Sequence Synthetic peptide
DPANDK

SEQ ID NO: 517 - protein, 8 aa, Artificial Sequence
  Description of Artificial Sequence Synthetic peptide
HYGYDGYA

SEQ ID NO: 518 - protein, 112 aa, Artificial Sequence
  Description of Artificial Sequence Synthetic polypeptide
DIVMSQSPDSLAVSLGERVTLNCKSSQLLYSSNQKNYLAWYQQKPGQSPKLLIYWASARESGVPDRFSGSGSGTDFTLTISSVQAEDVAVYYCQQYYSYPLTFGAGTKLELK

SEQ ID NO: 519 - protein, 8 aa, Artificial Sequence
  Description of Artificial Sequence Synthetic peptide
YSSNQKNY

SEQ ID NO: 520 - protein, 3 aa, Artificial Sequence
  Description of Artificial Sequence Synthetic peptide
WAS

SEQ ID NO: 521 - protein, 6 aa, Artificial Sequence
  Description of Artificial Sequence Synthetic peptide
YYSYPL

SEQ ID NO: 522 - protein, 1153 aa, Unknown
  Description of Unknown ITGAM sequence
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGQRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 523 - protein, 121 aa, Artificial Sequence
  Description of Artificial Sequence Synthetic polypeptide
DVKLVESGEGLVKPGGSLKLSCAASGFTFSRNAMSWVRQTPEKRLEWVAYISSGSDYIYYADTVKGRFTVSRDNARNTLYLQMTSLRSEDTAMYFCTRFSYGYGKNAPDYWGQGTSVTVSS

SEQ ID NO: 524 - protein, 5 aa, Artificial Sequence
  Description of Artificial Sequence Synthetic peptide
RNAMS

SEQ ID NO: 525 - protein, 16 aa, Artificial Sequence
  Description of Artificial Sequence Synthetic peptide
YISSGSDYIYYADTVK

SEQ ID NO: 526 - protein, 12 aa, Artificial Sequence
  Description of Artificial Sequence Synthetic peptide
FSYGYGKNAPDY

SEQ ID NO: 527 - protein, 106 aa, Artificial Sequence
  Description of Artificial Sequence Synthetic polypeptide
DIQMTQSPSSLSASLGGKVTITCKASQDINKYIAWYQHKPGQGPRLLIHYTSSLQPGIPSRFSGSGSGRDYSFSISNLEPEDIATYYCLQYDYTMTFGGGTKLEIR

SEQ ID NO: 528 - protein, 11 aa, Artificial Sequence
  Description of Artificial Sequence Synthetic peptide
KASQDINKYIA

SEQ ID NO: 529 - protein, 7 aa, Artificial Sequence
  Description of Artificial Sequence Synthetic peptide
YTSSLQP

SEQ ID NO: 530 - protein, 8 aa, Artificial Sequence
  Description of Artificial Sequence Synthetic peptide
LQYDYTMT

SEQ ID NO: 531 - protein, 119 aa, Artificial Sequence
  Description of Artificial Sequence Synthetic polypeptide
QVQLKQSGPGLVQPSQSLSITCTVSGFSLNNYGVHWVRQPPGKGLEWLGVIWSAGTTVYNAAFISRLSISKDDSKSQVFFKMNSLQAGDTAIYYCAKDGSRYYTAMDYWGQGTSVTVSS

SEQ ID NO: 532 - protein, 5 aa, Artificial Sequence
  Description of Artificial Sequence Synthetic peptide
NYGVH

SEQ ID NO: 533 - protein, 16 aa, Artificial Sequence
  Description of Artificial Sequence Synthetic peptide
VIWSAGTTVYNAAFIS

SEQ ID NO: 534 - protein, 11 aa, Artificial Sequence
  Description of Artificial Sequence Synthetic peptide
DGSRYYTAMDY

SEQ ID NO: 535 - protein, 112 aa, Artificial Sequence
  Description of Artificial Sequence Synthetic polypeptide
DVVMTQTPRSLPVSLGDQASISCRSRQSLIHSNGITFLHWYLQKAGQSPKLLIYKVSNRFSGVPDRFSGSGSGTDFTLRISRVEAEDLGVYFCSQGTHVPPTFGGGTKLEIK

SEQ ID NO: 536 - protein, 16 aa, Artificial Sequence
  Description of Artificial Sequence Synthetic peptide
RSRQSLIHSNGITFLH

SEQ ID NO: 537 - protein, 7 aa, Artificial Sequence
  Description of Artificial Sequence Synthetic peptide
KVSNRFS

SEQ ID NO: 538 - protein, 9 aa, Artificial Sequence
  Description of Artificial Sequence Synthetic peptide
SQGTHVPPT

SEQ ID NO: 539 - protein, 355 aa, Unknown
  Description of Unknown CCR1 sequence
METPNTTEDYDTTTEFDYGDATPCQKVNERAFGAQLLPPLYSLVFVIGLVGNILVVLVLVQYKRLKNMTSIYLLNLAISDLLFLFTLPFWIDYKLKDDWVFGDAMCKILSGFYYTGLYSEIFFIILLTIDRYLAIVHAVFALRARTVTFGVITSIIIWALAILASMPGLYFSKTQWEFTHHTCSLHFPHESLREWKLFQALKLNLFGLVLPLLVMIICYTGIIKILLRRPNEKKSKAVRLIFVIMIIFFLFWTPYNLTILISVFQDFLFTHECEQSRHLDLAVQVTEVIAYTHCCVNPVIYAFVGERFRKYLRQLFHRRVAVHLVKWLPFLSVDRLERVSSTSPSTGEHELSAGF

SEQ ID NO: 540 - protein, 305 aa, Unknown
  Description of Unknown LRRC25 sequence
MGGTLAWTLLLPLLLRESDSLEPSCTVSSADVDWNAEFSATCLNFSGLSLSLPHNQSLRASNVILLDLSGNGLRELPVTFFAHLQKLEVLNVLRNPLSRVDGALAARCDLDLQADCNCALESWHDIRRDNCSGQKPLLCWDTTSSQHNLSAFLEVSCAPGLASATIGAVVVSGCLLLGLAIAGPVLAWRLWRCRVARSRELNKPWAAQDGPKPGLGLQPRYGSRSAPKPQVAVPSCPSTPDYENMFVGQPAAEHQWDEQGAHPSEDNDFYINYKDIDLASQPVYCNLQSLGQAPMDEEEYVIPGH

SEQ ID NO: 541 - protein, 342 aa, Unknown
  Description of Unknown PTAFR sequence
MEPHDSSHMDSEFRYTLFPIVYSIIFVLGVIANGYVLWVFARLYPCKKFNEIKIFMVNLTMADMLFLITLPLWIVYYQNQGNWILPKFLCNVAGCLFFINTYCSVAFLGVITYNRFQAVTRPIKTAQANTRKRGISLSLVIWVAIVGAASYFLILDSTNTVPDSAGSGNVTRCFEHYEKGSVPVLIIHIFIVFSFFLVFLIILFCNLVIIRTLLMQPVQQQRNAEVKRRALWMVCTVLAVFIICFVPHHVVQLPWTLAELGFQDSKFHQAINDAHQVTLCLLSTNCVLDPVIYCFLTKKFRKHLTEKFYSMRSSRKCSRATTDTVTEVVVPFNQIPGNSLKN

SEQ ID NO: 542 - protein, 398 aa, Unknown
  Description of Unknown SIRPB1 isoform 1 sequence
MPVPASWPHLPSPFLLMTLLLGRLTGVAGEDELQVIQPEKSVSVAAGESATLRCAMTSLIPVGPIMWFRGAGAGRELIYNQKEGHFPRVTTVSELTKRNNLDFSISISNITPADAGTYYCVKFRKGSPDDVEFKSGAGTELSVRAKPSAPVVSGPAVRATPEHTVSFTCESHGFSPRDITLKWFKNGNELSDFQTNVDPAGDSVSYSIHSTARVVLTRGDVHSQVICEIAHITLQGDPLRGTANLSEAIRVPPTLEVTQQPMRAENQANVTCQVSNFYPRGLQLTWLENGNVSRTETASTLIENKDGTYNWMSWLLVNTCAHRDDVVLTCQVEHDGQQAVSKSYALEISAHQKEHGSDITHEAALAPTAPLLVALLLGPKLLLVVGVSAIYICWKQKA

SEQ ID NO: 543 - protein, 398 aa, Unknown
  Description of Unknown SIRPB1 isoform 3 sequence
MPVPASWPHLPSPFLLMTLLLGRLTGVAGEEELQVIQPDKSISVAAGESATLHCTVTSLIPVGPIQWFRGAGPGRELIYNQKEGHFPRVTTVSDLTKRNNMDFSIRISNITPADAGTYYCVKFRKGSPDHVEFKSGAGTELSVRAKPSAPVVSGPAARATPQHTVSFTCESHGFSPRDITLKWFKNGNELSDFQTNVDPAGDSVSYSIHSTAKVVLTREDVHSQVICEVAHVTLQGDPLRGTANLSETIRVPPTLEVTQQPVRAENQVNVTCQVRKFYPQRLQLTWLENGNVSRTETASTLTENKDGTYNWMSWLLVNVSAHRDDVKLTCQVEHDGQPAVSKSHDLKVSAHPKEQGSNTAPGPALASAAPLLIAFLLGPKVLLVVGVSVIYVYWKQKA

SEQ ID NO: 544 - protein, 167 aa, Artificial Sequence
  Description of Artificial Sequence Synthetic polypeptide
MSSPQSLKTLILTMGWSWIFLFLLSGTAGVHSQVQLQQSGPELVNPGASVKLSCKASGFTFTTYGINWVKQGPGQGLEWIGWIYPRDGSTNFNENFKDKAALTVDTSSSTAYMELHSLTSEDSAVYFCARLTGGTFLDYWGQGTTLTVSSAKTTPPSVYPLAPGSAA

SEQ ID NO: 545 - protein, 8 aa, Artificial Sequence
  Description of Artificial Sequence Synthetic peptide
GFTFTTYG

SEQ ID NO: 546 - protein, 8 aa, Artificial Sequence
  Description of Artificial Sequence Synthetic peptide
IYPRDGST

SEQ ID NO: 547 - protein, 11 aa, Artificial Sequence
  Description of Artificial Sequence Synthetic peptide
ARLTGGTFLDY

SEQ ID NO: 548 - protein, 147 aa, Artificial Sequence
  Description of Artificial Sequence Synthetic polypeptide
MESDTLLLWVLLLWVPGSTGDIVLTQSPASLAVSLGQRATISCRASESVEYYGTSLMQWYQQKPGQPPKLLIFGASNVESGVPVRFSGSGSGTDFSLNIHPVEEDDIVMYFCQQSRKLPWTFGGGTKLEIKRADAAPTVSIFPPSSE

SEQ ID NO: 549 - protein, 10 aa, Artificial Sequence
  Description of Artificial Sequence Synthetic peptide
ESVEYYGTSL

SEQ ID NO: 550 - protein, 3 aa, Artificial Sequence
  Description of Artificial Sequence Synthetic peptide
GAS

SEQ ID NO: 551 - protein, 9 aa, Artificial Sequence
  Description of Artificial Sequence Synthetic peptide
QQSRKLPWT

SEQ ID NO: 552 - protein, 119 aa, Artificial Sequence
  Description of Artificial Sequence Synthetic polypeptide
QVQLVQSGSELKKPGASVKLSCKASGFTFTTYGINWVRQAPGQGLEWIGWIYPRDGSTNFNENFKDRATITVDTSASTAYMELSSLRSEDTAVYFCARLTGGTFLDYWGQGTTVTVSSA

SEQ ID NO: 553 - protein, 7 aa, Artificial Sequence
  Description of Artificial Sequence Synthetic peptide
GFTFTTY

SEQ ID NO: 554 - protein, 6 aa, Artificial Sequence
  Description of Artificial Sequence Synthetic peptide
YPRDGS

SEQ ID NO: 555 - protein, 9 aa, Artificial Sequence
  Description of Artificial Sequence Synthetic peptide
LTGGTFLDY

SEQ ID NO: 556 - protein, 112 aa, Artificial Sequence
  Description of Artificial Sequence Synthetic polypeptide
DIVLTQSPATLSLSPGERATLSCRASESVEYYGTSLMQWYQQKPGQPPKLLIFGASNVESGVPDRFSGSGSGTDFTLKISRVEAEDVGMYFCQQSRKLPWTFGGGTKVEIKR

SEQ ID NO: 557 - protein, 12 aa, Artificial Sequence
  Description of Artificial Sequence Synthetic peptide
ESVEYYGTSLMQ

SEQ ID NO: 558 - protein, 7 aa, Artificial Sequence
  Description of Artificial Sequence Synthetic peptide
GASNVES

SEQ ID NO: 559 - protein, 9 aa, Artificial Sequence
  Description of Artificial Sequence Synthetic peptide
QQSRKLPWT

SEQ ID NO: 560 - protein, 784 aa, Unknown
  Description of Unknown TLR2 sequence
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 561 - protein, 121 aa, Artificial Sequence
  Description of Artificial Sequence Synthetic polypeptide
QVTLKESGPGILQPSQTLSLTCSFSGFSLTTYNIGVGWIRQPSGKGLEWLAHIWWNDNIYYNTVLKSRLTFSKDTSNNQVFLKIASVDIADTATYYCIRMAEGRYDAMDYWGQGTSVTVSS

SEQ ID NO: 562 - protein, 7 aa, Artificial Sequence
  Description of Artificial Sequence Synthetic peptide
TYNIGVG

SEQ ID NO: 563 - protein, 16 aa, Artificial Sequence
  Description of Artificial Sequence Synthetic peptide
HIWWNDNIYYNTVLKS

SEQ ID NO: 564 - protein, 11 aa, Artificial Sequence
  Description of Artificial Sequence Synthetic peptide
MAEGRYDAMDY

SEQ ID NO: 565 - protein, 108 aa, Artificial Sequence
  Description of Artificial Sequence Synthetic polypeptide
AIQMTQSTSSLSASLGDRVTINCRASQDITNYLNWYQQKPDGTVRLLIYYTSKLHSGAPSRFSGRGSGTDYSLTISNLEQEDIATYFCQQGNTFPWTFGGGTKLEIKR

SEQ ID NO: 566 - protein, 11 aa, Artificial Sequence
  Description of Artificial Sequence Synthetic peptide
RASQDITNYLN

SEQ ID NO: 567 - protein, 7 aa, Artificial Sequence
  Description of Artificial Sequence Synthetic peptide
YTSKLHS

SEQ ID NO: 568 - protein, 9 aa, Artificial Sequence
  Description of Artificial Sequence Synthetic peptide
QQGNTFPWT

SEQ ID NO: 569 - protein, 119 aa, Artificial Sequence
  Description of Artificial Sequence Synthetic polypeptide
EVQLVESGGGLVQPGGSLRLSCAASGFTFDTYAMHWVRQAPGKGLEWVAGISWNSGNIGYADSVKGRFTISRDNSKNTLYLQMNSLRAEDTAVYYCAKDWDNWNLFDYWGQGTLVTVSS

SEQ ID NO: 570 - protein, 8 aa, Artificial Sequence
  Description of Artificial Sequence Synthetic peptide
GFTFDTYA

SEQ ID NO: 571 - protein, 16 aa, Artificial Sequence
  Description of Artificial Sequence Synthetic peptide
ISWNSGNIGYADSVKG

SEQ ID NO: 572 - protein, 10 aa, Artificial Sequence
  Description of Artificial Sequence Synthetic peptide
DWDNWNLFDY

SEQ ID NO: 573 - protein, 108 aa, Artificial Sequence
  Description of Artificial Sequence Synthetic polypeptide
DIQMTQSPSSLSASVGDRVTITCRASQSISSWLAWYQQKPGKAPKLLIYKASSLESGVPSRFSGSGSGTDFTLTISSLQPEDFATYYCQQYSSYSWTFGQGTKVEIKR

SEQ ID NO: 574 - protein, 11 aa, Artificial Sequence
  Description of Artificial Sequence Synthetic peptide
RASQSISSWLA

SEQ ID NO: 575 - protein, 7 aa, Artificial Sequence
  Description of Artificial Sequence Synthetic peptide
KASSLES

SEQ ID NO: 576 - protein, 9 aa, Artificial Sequence
  Description of Artificial Sequence Synthetic peptide
QQYSSYSWT

SEQ ID NO: 577 - protein, 839 aa, Unknown
  Description of Unknown TLR4 sequence
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 578 - protein, 201 aa, Unknown
  Description of Unknown CD300LB sequence
MWLPPALLLLSLSGCFSIQGPESVRAPEQGSLTVQCHYKQGWETYIKWWCRGVRWDTCKILIETRGSEQGEKSDRVSIKDNQKDRTFTVTMEGLRRDDADVYWCGIERRGPDLGTQVKVIVDPEGAASTTASSPTNSNMAVFIGSHKRNHYMLLVFVKVPILLILVTAILWLKGSQRVPEEPGEQPIYMNFSEPLTKDMAT

SEQ ID NO: 579 - protein, 1024 aa, Unknown
  Description of Unknown ATP1A3 isoform 2 sequence
MGGWEEERNRRATDKKDDKDSPKKNKGKERRDLDDLKKEVAMTEHKMSVEEVCRKYNTDCVQGLTHSKAQEILARDGPNALTPPPTTPEWVKFCRQLFGGFSILLWIGAILCFLAYGIQAGTEDDPSGDNLYLGIVLAAVVIITGCFSYYQEAKSSKIMESFKNMVPQQALVIREGEKMQVNAEEVVVGDLVEIKGGDRVPADLRIISAHGCKVDNSSLTGESEPQTRSPDCTHDNPLETRNITFFSTNCVEGTARGVVVATGDRTVMGRIATLASGLEVGKTPIAIEIEHFIQLITGVAVFLGVSFFILSLILGYTWLEAVIFLIGIIVANVPEGLLATVTVCLTLTAKRMARKNCLVKNLEAVETLGSTSTICSDKTGTLTQNRMTVAHMWFDNQIHEADTTEDQSGTSFDKSSHTWVALSHIAGLCNRAVFKGGQDNIPVLKRDVAGDASESALLKCIELSSGSVKLMRERNKKVAEIPFNSTNKYQLSIHETEDPNDNRYLLVMKGAPERILDRCSTILLQGKEQPLDEEMKEAFQNAYLELGGLGERVLGFCHYYLPEEQFPKGFAFDCDDVNFTTDNLCFVGLMSMIDPPRAAVPDAVGKCRSAGIKVIMVTGDHPITAKAIAKGVGIISEGNETVEDIAARLNIPVSQVNPRDAKACVIHGTDLKDFTSEQIDEILQNHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLLFIMANIPLPLGTITILCIDLGTDMVPAISLAYEAAESDIMKRQPRNPRTDKLVNERLISMAYGQIGMIQALGGFFSYFVILAENGFLPGNLVGIRLNWDDRTVNDLEDSYGQQWTYEQRKVVEFTCHTAFFVSIVVVQWADLIICKTRRNSVFQQGMKNKILIFGLFEETALAAFLSYCPGMDVALRMYPLKPSWWFCAFPYSFLIFVYDEIRKLILRRNPGGWVEKETYY

SEQ ID NO: 580 - protein, 1026 aa, Unknown
  Description of Unknown ATP1A3 isoform 3 sequence
MGSGGSDSYRIATSQDKKDDKDSPKKNKGKERRDLDDLKKEVAMTEHKMSVEEVCRKYNTDCVQGLTHSKAQEILARDGPNALTPPPTTPEWVKFCRQLFGGFSILLWIGAILCFLAYGIQAGTEDDPSGDNLYLGIVLAAVVIITGCFSYYQEAKSSKIMESFKNMVPQQALVIREGEKMQVNAEEVVVGDLVEIKGGDRVPADLRIISAHGCKVDNSSLTGESEPQTRSPDCTHDNPLETRNITFFSTNCVEGTARGVVVATGDRTVMGRIATLASGLEVGKTPIAIEIEHFIQLITGVAVFLGVSFFILSLILGYTWLEAVIFLIGIIVANVPEGLLATVTVCLTLTAKRMARKNCLVKNLEAVETLGSTSTICSDKTGTLTQNRMTVAHMWFDNQIHEADTTEDQSGTSFDKSSHTWVALSHIAGLCNRAVFKGGQDNIPVLKRDVAGDASESALLKCIELSSGSVKLMRERNKKVAEIPFNSTNKYQLSIHETEDPNDNRYLLVMKGAPERILDRCSTILLQGKEQPLDEEMKEAFQNAYLELGGLGERVLGFCHYYLPEEQFPKGFAFDCDDVNFTTDNLCFVGLMSMIDPPRAAVPDAVGKCRSAGIKVIMVTGDHPITAKAIAKGVGIISEGNETVEDIAARLNIPVSQVNPRDAKACVIHGTDLKDFTSEQIDEILQNHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLLFIMANIPLPLGTITILCIDLGTDMVPAISLAYEAAESDIMKRQPRNPRTDKLVNERLISMAYGQIGMIQALGGFFSYFVILAENGFLPGNLVGIRLNWDDRTVNDLEDSYGQQWTYEQRKVVEFTCHTAFFVSIVVVQWADLIICKTRRNSVFQQGMKNKILIFGLFEETALAAFLSYCPGMDVALRMYPLKPSWWFCAFPYSFLIFVYDEIRKLILRRNPGGWVEKETYY

SEQ ID NO: 581 - protein, 141 aa, Artificial Sequence
  Description of Artificial Sequence Synthetic polypeptide
MEWSGVFIFLLSVTAGVHSEVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSS

SEQ ID NO: 582 - protein, 10 aa, Artificial Sequence
  Description of Artificial Sequence Synthetic peptide
GYTFSNYWIG

SEQ ID NO: 583 - protein, 17 aa, Artificial Sequence
  Description of Artificial Sequence Synthetic peptide
DIYPGGNYIRNNEKFKD

SEQ ID NO: 584 - protein, 13 aa, Artificial Sequence
  Description of Artificial Sequence Synthetic peptide
SFGSNYVFAWFTY

SEQ ID NO: 585 - protein, 131 aa, Artificial Sequence
  Description of Artificial Sequence Synthetic polypeptide
MKLPVRLLVLMFWIPASSSDIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIK

SEQ ID NO: 586 - protein, 16 aa, Artificial Sequence
  Description of Artificial Sequence Synthetic peptide
RSSQRLLSSYGHTYLH

SEQ ID NO: 587 - protein, 7 aa, Artificial Sequence
  Description of Artificial Sequence Synthetic peptide
EVSNRFS

SEQ ID NO: 588 - protein, 9 aa, Artificial Sequence
  Description of Artificial Sequence Synthetic peptide
SQSTHVPLT

SEQ ID NO: 589 - protein, 141 aa, Artificial Sequence
  Description of Artificial Sequence Synthetic polypeptide
MEWSGVFIFLLSVTAGVHSQVQLVQSGPDVKKPGTSMKMSCKTSGYTFSNYWIGWVRQAPGQGLEWIGDIYPGGNYIRNNEKFKDKTTLTADTSTSTAYMQLGSLRSEDTAVYYCGSSFGSNYVFAWFTYWGQGTLVTVSS

SEQ ID NO: 590 - protein, 10 aa, Artificial Sequence
  Description of Artificial Sequence Synthetic peptide
GYTFSNYWIG

SEQ ID NO: 591 - protein, 17 aa, Artificial Sequence
  Description of Artificial Sequence Synthetic peptide
DIYPGGNYIRNNEKFKD

SEQ ID NO: 592 - protein, 13 aa, Artificial Sequence
  Description of Artificial Sequence Synthetic peptide
SFGSNYVFAWFTY

SEQ ID NO: 593 - protein, 131 aa, Artificial Sequence; identical to SEQ ID NO: 585
  Description of Artificial Sequence Synthetic polypeptide

SEQ ID NO: 594 - protein, 16 aa, Artificial Sequence
  Description of Artificial Sequence Synthetic peptide
RSSQRLLSSYGHTYLH

SEQ ID NO: 595 - protein, 7 aa, Artificial Sequence
  Description of Artificial Sequence Synthetic peptide
EVSNRFS

SEQ ID NO: 596 - protein, 9 aa, Artificial Sequence
  Description of Artificial Sequence Synthetic peptide
SQSTHVPLT

SEQ ID NO: 597 - protein, 352 aa, Unknown [sequence omitted]

SEQ ID NO: 598 - protein, 126 aa, Artificial Sequence
  Description of Artificial Sequence Synthetic polypeptide
GVQLVESGGGLVQPGGSLRLSCAASGFTISRNDMHWVRQATGKGLEWVSAIGTGGDTYYPGSVKGRFTISRENAKNSLYLQMNSLRAGDTAVYYCARGHMTTFGGFIVIGNGMDVWGQGTTVTVSS

SEQ ID NO: 599 - protein, 7 aa, Artificial Sequence
  Description of Artificial Sequence Synthetic peptide
GFTISRN

SEQ ID NO: 600 - protein, 5 aa, Artificial Sequence
  Description of Artificial Sequence Synthetic peptide
GTGGD

SEQ ID NO: 601 - protein, 18 aa, Artificial Sequence
  Description of Artificial Sequence Synthetic peptide
GHMTTFGGFIVIGNGMDV

SEQ ID NO: 602 - protein, 108 aa, Artificial Sequence
  Description of Artificial Sequence Synthetic polypeptide
EIVMTQSPATLSVSPGERATLSCRASQSVRSNLAWYQQKPGQAPRLLIYGASTRATGIPARFSGSGSGTEFTLTISSLQSEDFAVYSCQQYNNWPWTFGQGTKVEIKR

SEQ ID NO: 603 - protein, 8 aa, Artificial Sequence
  Description of Artificial Sequence Synthetic peptide
QSVRSNLA

SEQ ID NO: 604 - protein, 7 aa, Artificial Sequence
  Description of Artificial Sequence Synthetic peptide
GASTRAT

SEQ ID NO: 605 - protein, 9 aa, Artificial Sequence
  Description of Artificial Sequence Synthetic peptide
QQYNNWPWT

SEQ ID NO: 606 - protein, 118 aa, Artificial Sequence
  Description of Artificial Sequence Synthetic polypeptide
QVQLQQWGAGLLKPSETLSLTCAVYGGSFSDYFWTWIRQPPGKGLEWIGEINHSGTTNYNPSLKSRVTISADTSKNQFSRLSSVTAADTAVYYCARLSSWSNWAFEYWGQGTLVTVSS

SEQ ID NO: 607 - protein, 5 aa, Artificial Sequence
  Description of Artificial Sequence Synthetic peptide
DYFWT

SEQ ID NO: 608 - protein, 16 aa, Artificial Sequence
  Description of Artificial Sequence Synthetic peptide
EINHSGTTNYNPSLKS

SEQ ID NO: 609 - protein, 11 aa, Artificial Sequence
  Description of Artificial Sequence Synthetic peptide
LSSWSNWAFEY

SEQ ID NO: 610 - protein, 108 aa, Artificial Sequence
  Description of Artificial Sequence Synthetic polypeptide
EIVLTQFPGTLSLSPGERATLSCRASQSVSSTYLAWYQQKPGQAPRVLIYGASRRATGIPDRFSGSGSGTDFTLTISRLEPEDFAVYYCQQYGSSPLTFGGGTKVEIK

SEQ ID NO: 611 - protein, 12 aa, Artificial Sequence
  Description of Artificial Sequence Synthetic peptide
RASQSVSSTYLA

SEQ ID NO: 612 - protein, 7 aa, Artificial Sequence
  Description of Artificial Sequence Synthetic peptide
GASRRAT

SEQ ID NO: 613 - protein, 9 aa, Artificial Sequence
  Description of Artificial Sequence Synthetic peptide
QQYGSSPLT

SEQ ID NO: 614 - protein, 282 aa, Unknown
  Description of Unknown B7-H4 sequence
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 615 - protein, 117 aa, Artificial Sequence
  Description of Artificial Sequence Synthetic polypeptide
QVQLQQSGAELVKPGASVKLSCKASGYTFTSHDINWVRQRPELGLEWIGWIFPGDGSTKFNEKFKGKATLTTDKSSSTAYIQLSRLTSEDSAVYFCARNSFYSMDYWGQGTSVTVSS

SEQ ID NO: 616 - protein, 8 aa, Artificial Sequence
  Description of Artificial Sequence Synthetic peptide
GYTFTSHD

SEQ ID NO: 617 - protein, 8 aa, Artificial Sequence
  Description of Artificial Sequence Synthetic peptide
IFPGDGST

SEQ ID NO: 618 - protein, 10 aa, Artificial Sequence
  Description of Artificial Sequence Synthetic peptide
ARNSFYSMDY

SEQ ID NO: 619 - protein, 112 aa, Artificial Sequence
  Description of Artificial Sequence Synthetic polypeptide
DIVMSQSPSSLAVSAGEKVTMSCKSSQSLLNSRTRKNQLAWYQQKPGQSPKLLIYWAFIRESGVPDRFTGSGSGTDFTLTISSVQAEDLAVYYCKQSYNLRTFGGGIKLEIK

SEQ ID NO: 620 - protein, 12 aa, Artificial Sequence
  Description of Artificial Sequence Synthetic peptide
QSLLNSRTRKNQ

SEQ ID NO: 621 - protein, 3 aa, Artificial Sequence
  Description of Artificial Sequence Synthetic peptide
WAF

SEQ ID NO: 622 - protein, 8 aa, Artificial Sequence
  Description of Artificial Sequence Synthetic peptide
KQSYNLRT

SEQ ID NO: 623 - protein, 118 aa, Artificial Sequence
  Description of Artificial Sequence Synthetic polypeptide
EVQLLQSGPELEKPGASVKISCKASGYSFTGYNMNWVKQSNGKSLEWIGNIDPYYDYTSYNLKFKDKATLTVDKSSSTAYMQLKSLTSEDSAVYYCATSTMITPFDYWGQGTTLTVSS

SEQ ID NO: 624 - protein, 10 aa, Artificial Sequence
  Description of Artificial Sequence Synthetic peptide
GYSFTGYNMN

SEQ ID NO: 625 - protein, 17 aa, Artificial Sequence
  Description of Artificial Sequence Synthetic peptide
NIDPYYDYTSYNLKFKD

SEQ ID NO: 626 - protein, 9 aa, Artificial Sequence
  Description of Artificial Sequence Synthetic peptide
STMITPFDY

SEQ ID NO: 627 - protein, 112 aa, Artificial Sequence
  Description of Artificial Sequence Synthetic polypeptide
DVLMTQTPLSLPVSLGDQASISCRSSQSIVHSNGNTYLEWYLQKPGQSPKLLIYKVSNRFSGVPDRFSGSGSGTDFTLKINRVEAEDLGVYYCFQGSHVPWTFGGGTKLEIK

SEQ ID NO: 628 - protein, 16 aa, Artificial Sequence
  Description of Artificial Sequence Synthetic peptide
RSSQSIVHSNGNTYLE

SEQ ID NO: 629 - protein, 7 aa, Artificial Sequence
  Description of Artificial Sequence Synthetic peptide
KVSNRFS

SEQ ID NO: 630 - protein, 9 aa, Artificial Sequence
  Description of Artificial Sequence Synthetic peptide
FQGSHVPWT

SEQ ID NO: 631 - protein, 311 aa, Unknown
  Description of Unknown VISTA sequence
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 632 - protein, 246 aa, Unknown
  Description of Unknown ULBP-6 sequence
MAAAAIPALLLCLPLLFLLFGWSRARRDDPHSLCYDITVIPKFRPGPRWCAVQGQVDEKTFLHYDCGNKTVTPVSPLGKKLNVTMAWKAQNPVLREVVDILTEQLLDIQLENYTPKEPLTLQARMSCEQKAEGHSSGSWQFSIDGQTFLLFDSEKRMWTTVHPGARKMKEKWENDKDVAMSFHYISMGDCIGWLEDFLMGMDSTLEPSAGAPLAMSSGTTQLRATATTLILCCLLIILPCFILPGI

SEQ ID NO: 633 - protein, 20 aa, Unknown
  Description of Unknown Mucin 1 sequence
HGVTSAPDTRPAPGSTAPPA

SEQ ID NO: 634 - protein, 5 aa, Artificial Sequence
  Description of Artificial Sequence Synthetic peptide
GYYWS

SEQ ID NO: 635 - protein, 9 aa, Artificial Sequence
  Description of Artificial Sequence Synthetic peptide
ARGPWSFDP

SEQ ID NO: 636 - protein, 5 aa, Artificial Sequence
  Description of Artificial Sequence Synthetic peptide
SYAIS

SEQ ID NO: 637 - protein, 16 aa, Artificial Sequence
  Description of Artificial Sequence Synthetic peptide
GDSSIRHAYYYYGMDV

SEQ ID NO: 638 - protein, 7 aa, Artificial Sequence
  Description of Artificial Sequence Synthetic peptide
SSSYYWG

SEQ ID NO: 639 - protein, 11 aa, Artificial Sequence
  Description of Artificial Sequence Synthetic peptide
GSDRFHPYFDY

SEQ ID NO: 640 - protein, 5 aa, Artificial Sequence
  Description of Artificial Sequence Synthetic peptide
SYYMH

SEQ ID NO: 641 - protein, 17 aa, Artificial Sequence
  Description of Artificial Sequence Synthetic peptide
GAPNYGDTTHDYYYMDV

SEQ ID NO: 642 - protein, 5 aa, Artificial Sequence
  Description of Artificial Sequence Synthetic peptide
GYYMH

SEQ ID NO: 643 - protein, 15 aa, Artificial Sequence
  Description of Artificial Sequence Synthetic peptide
DTGEYYDTDDHGMDV

SEQ ID NO: 644 - protein, 5 aa, Artificial Sequence
  Description of Artificial Sequence Synthetic peptide
SYAMS

SEQ ID NO: 645 - protein, 12 aa, Artificial Sequence
  Description of Artificial Sequence Synthetic peptide
DGGYYDSGAGDY

SEQ ID NO: 646 - protein, 5 aa, Artificial Sequence
  Description of Artificial Sequence Synthetic peptide
SYSMN

SEQ ID NO: 647 - protein, 13 aa, Artificial Sequence
  Description of Artificial Sequence Synthetic peptide
GAPMGAAAGWFDP

SEQ ID NO: 648 - protein, 5 aa, Artificial Sequence
  Description of Artificial Sequence Synthetic peptide
SYYMH

SEQ ID NO: 649 - protein, 16 aa, Artificial Sequence
  Description of Artificial Sequence Synthetic peptide
EGAGFAYGMDYYYMDV

SEQ ID NO: 650 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSS

SEQ ID NO: 651 - protein, 15 aa, Artificial Sequence
  Description of Artificial Sequence Synthetic peptide
ARGAPIGAAAGWFDP

SEQ ID NO: 652 - protein, 13 aa, Artificial Sequence
  Description of Artificial Sequence Synthetic peptide
GAPIGAAAGWFDP

SEQ ID NO: 653 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPQGAAAGWFDPWGQGTLVTVSS

SEQ ID NO: 654 - protein, 15 aa, Artificial Sequence
  Description of Artificial Sequence Synthetic peptide
ARGAPQGAAAGWFDP

SEQ ID NO: 655 - protein, 13 aa, Artificial Sequence
  Description of Artificial Sequence Synthetic peptide
GAPQGAAAGWFDP

SEQ ID NO: 656 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPLGAAAGWFDPWGQGTLVTVSS

SEQ ID NO: 657 - protein, 15 aa, Artificial Sequence
  Description of Artificial Sequence Synthetic peptide
ARGAPLGAAAGWFDP

SEQ ID NO: 658 - protein, 13 aa, Artificial Sequence
  Description of Artificial Sequence Synthetic peptide
GAPLGAAAGWFDP

SEQ ID NO: 659 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPFGAAAGWFDPWGQGTLVTVSS

SEQ ID NO: 660 - protein, 15 aa, Artificial Sequence
  Description of Artificial Sequence Synthetic peptide
ARGAPFGAAAGWFDP

SEQ ID NO: 661 - protein, 13 aa, Artificial Sequence
  Description of Artificial Sequence Synthetic peptide
GAPFGAAAGWFDP

SEQ ID NO: 662 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPVGAAAGWFDPWGQGTLVTVSS

SEQ ID NO: 663 - protein, 15 aa, Artificial Sequence
  Description of Artificial Sequence Synthetic peptide
ARGAPVGAAAGWFDP

SEQ ID NO: 664 - protein, 13 aa, Artificial Sequence
  Description of Artificial Sequence Synthetic peptide
GAPVGAAAGWFDP

SEQ ID NO: 665 - protein, 122 aa, Artificial Sequence
  Description of Artificial Sequence Synthetic polypeptide
  MOD_RES (102)..(102): M, L, I, V, Q, or F
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPXGAAAGWFDPWGQGTLVTVSS

SEQ ID NO: 666 - protein, 15 aa, Artificial Sequence
  Description of Artificial Sequence Synthetic peptide
  MOD_RES (6)..(6): M, L, I, V, Q, or F
ARGAPXGAAAGWFDP

SEQ ID NO: 667 - protein, 13 aa, Artificial Sequence
  Description of Artificial Sequence Synthetic peptide
  MOD_RES (4)..(4): M, L, I, V, Q, or F
GAPXGAAAGWFDP

SEQ ID NO: 668 - protein, 120 aa, Artificial Sequence
  Description of Artificial Sequence Synthetic polypeptide
QVQLVQSGAEVKKPGASVKVSCKASGFNIKDDYIHWVRQAPGQGLEWMGWIDSENGDTEYASKFQGRVTITADTSANTAYMELSSLRSEDTAVYYCATSSYYSYDLFVYWGQGTLVTVSS

SEQ ID NO: 669 - protein, 111 aa, Artificial Sequence
  Description of Artificial Sequence Synthetic polypeptide
DVQMTQSPSSLSASVGDRVTITCKSSQSLLHSNGKTYLNWLQQKPGQAPKLLLYLVSKLESGVPSRFSGSGSGTDYTLTISSLQPEDFATYYCLQTTHLYTFGQGTKLEIK

SEQ ID NO: 670 - protein, 11 aa, Artificial Sequence
  Description of Artificial Sequence Synthetic peptide
SSYYSYDLFVY

SEQ ID NO: 671 - protein, 120 aa, Artificial Sequence
  Description of Artificial Sequence Synthetic polypeptide
QVQLVQSGAEVKKPGASVKVSCKASGFNIKDDYIHWVRQAPGQGLEWMGWIDSENGDTEYASKFQGRVTITADTSANTAYMELSSLRSEDTAVYYCATSEYYSYDLFVYWGQGTLVTVSS

SEQ ID NO: 672 - protein, 11 aa, Artificial Sequence
  Description of Artificial Sequence Synthetic peptide
SEYYSYDLFVY

SEQ ID NO: 673 - protein, 120 aa, Artificial Sequence
  Description of Artificial Sequence Synthetic polypeptide
QVQLVQSGAEVKKPGASVKVSCKASGFNIKDDYIHWVRQAPGQGLEWMGWIDSENGDTEYASKFQGRVTITADTSANTAYMELSSLRSEDTAVYYCATSSYWSYDLLVYWGQGTLVTVSS

SEQ ID NO: 674 - protein, 11 aa, Artificial Sequence
  Description of Artificial Sequence Synthetic peptide
SSYWSYDLLVY

SEQ ID NO: 675 - protein, 120 aa, Artificial Sequence
  Description of Artificial Sequence Synthetic polypeptide
QVQLVQSGAEVKKPGASVKVSCKASGFNIKDDYIHWVRQAPGQGLEWMGWIDSENGDTEYASKFQGRVTITADTSANTAYMELSSLRSEDTAVYYCATSSYWSYDLFVYWGQGTLVTVSS

SEQ ID NO: 676 - protein, 11 aa, Artificial Sequence
  Description of Artificial Sequence Synthetic peptide
SSYWSYDLFVY

SEQ ID NO: 677 - protein, 120 aa, Artificial Sequence
  Description of Artificial Sequence Synthetic polypeptide
QVQLVQSGAEVKKPGASVKVSCKASGFNIKDDYIHWVRQAPGQGLEWMGWIDSENGDTEYASKFQGRVTITADTSANTAYMELSSLRSEDTAVYYCATSTYWSYDLFVYWGQGTLVTVSS

SEQ ID NO: 678 - protein, 11 aa, Artificial Sequence
  Description of Artificial Sequence Synthetic peptide
STYWSYDLFVY

SEQ ID NO: 679 - protein, 120 aa, Artificial Sequence
  Description of Artificial Sequence Synthetic Polypeptide
  MOD_RES (100)..(100): S, T, or E
  MOD_RES (102)..(102): Y or W
  MOD_RES (107)..(107): F or L
QVQLVQSGAEVKKPGASVKVSCKASGFNIKDDYIHWVRQAPGQGLEWMGWIDSENGDTEYASKFQGRVTITADTSANTAYMELSSLRSEDTAVYYCATSXYXSYDLXVYWGQGTLVTVSS

SEQ ID NO: 680 - protein, 11 aa, Artificial Sequence
  Description of Artificial Sequence Synthetic Polypeptide
  MOD_RES (2)..(2): S, T, or E
  MOD_RES (4)..(4): Y or W
  MOD_RES (9)..(9): F or L
SXYXSYDLXVY

(19) United States
(12) Patent Application Publication
(10) Pub. No.: US 2021/0221894 A1
(43) Pub. Date: Jul. 22, 2021

(54) PROTEINS BINDING NKG2D, CD16 AND AN ANTIGEN ASSOCIATED WITH TUMORS, MDSCS AND/OR TAMS

(71) Applicant: Dragonfly Therapeutics, Inc., Waltham, MA (US)

(72) Inventors: Mitchell Bigelow, Cambridge, MA (US); Gregory P. Chang, Medford, MA (US); Ann F. Cheung, Lincoln, MA (US); Jean-Marie Cuillerot, Somerville, MA (US); Jinyan Du, Waltham, MA (US); Asya Grinberg, Lexington, MA (US); William Haney, Wayland, MA (US); Steven O'Neil, Wayland, MA (US); Nicolai Wagtmann, Concord, MA (US); Bradley M. Lunde, Lebanon, NH (US); Bianka Prinz, Lebanon, NH (US)

(21) Appl. No.: 17/045,016

(22) PCT Filed: Apr. 3, 2019
(86) PCT No.: PCT/US2019/025567

§ 371 Date: Oct. 2, 2020

Related U.S. Application Data
(60) Provisional application No. 62/672,299, filed on May 16, 2018.
(60) Provisional application No. 62/667,844, filed on May 7, 2018.
(60) Provisional application No. 62/663,607, filed on Apr. 27, 2018.
(60) Provisional application No. 62/651,951, filed on Apr. 3, 2018.

(51) Int. Cl. C07K 16/28; A61P 35/00

(52) U.S. Cl. CPC C07K 16/2851; C07K 16/28; C07K 16/283; A61P 35/00; C07K 2317/31; C07K 2317/524; C07K 2317/92; C07K 2317/565; C07K 2317/73; C07K 2317/76; C07K 2317/569; C07K 2317/24; C07K 2317/33

(57) ABSTRACT

Multi-specific binding proteins that bind the NKG2D receptor, CD 16, and a tumor-associated antigen on tumor cells, or an antigen on myeloid-derived suppressor cells or tumor-associated macrophages are described, as well as pharmaceutical compositions and therapeutic methods useful for the treatment of cancer.

### CROSS-REFERENCE TO RELATED APPLICATIONS

This application claims the benefit of and priority to U.S. Provisional patent Application No. 62/651,951, filed Apr. 3, 2018, the disclosure of which is hereby incorporated by reference in its entirety for all purposes; U.S. Provisional Patent Application No. 62/667,844, filed May 7, 2018; U.S. Provisional Patent Application No. 62/672,299, filed May 16, 2018; and U.S. Provisional Patent Application No. 62/663,607, filed Apr. 27, 2018, the disclosure of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Apr. 2, 2019, is named DFY-053WO_SL.txt and is 541,768 bytes in size.

### FIELD OF THE INVENTION

The invention relates to multi-specific binding proteins that bind to the NKG2D receptor, CD16, and a tumor-associated antigen on tumor cells or an antigen on myeloid-derived suppressor cells (MDSCs) or tumor-associated macrophages (TAMs), as well as pharmaceutical compositions and therapeutic methods useful for the treatment of cancer.

### BACKGROUND

Cancer continues to be a significant health problem despite the substantial research efforts and scientific advances reported in the literature for treating this disease. Some of the most frequently diagnosed cancers include prostate cancer, breast cancer, and lung cancer. Prostate cancer is the most common form of cancer in men. Breast cancer remains a leading cause of death in women. Current treatment options for these cancers are not effective for all patients and/or can have substantial adverse side effects. Other types of cancers also remain challenging to treat using existing therapeutic options.

Cancer immunotherapies are desirable because they are highly specific and can facilitate destruction of cancer cells using the patient's own immune system. Fusion proteins such as bi-specific T-cell engagers are cancer immunotherapies described in the literature that bind to tumor cells and T-cells to facilitate destruction of tumor cells. Antibodies that bind to certain tumor-associated antigens and to certain immune cells have been described in the literature. See, e.g., WO 2016/134371 and WO 2015/095412.

Natural killer (NK) cells are a component of the innate immune system and make up approximately 15% of circulating lymphocytes. NK cells infiltrate virtually all tissues and were originally characterized by their ability to kill tumor cells effectively without the need for prior sensitization. Activated NK cells kill target cells by means similar to cytotoxic T cells—i.e., via cytolytic granules that contain perforin and granzymes as well as via death receptor pathways. Activated NK cells also secrete inflammatory cytokines such as IFNγ and chemokines that promote the recruitment of other leukocytes to the target tissue.

NK cells respond to signals through a variety of activating and inhibitory receptors on their surface. For example, when NK cells encounter healthy self-cells, their activity is inhibited through activation of the killer-cell immunoglobulin-like receptors (KIRs). Alternatively, when NK cells encounter foreign cells or cancer cells, they are activated via their activating receptors (e.g., NKG2D, natural cytotoxicity receptors (NCRs), DNAX accessory molecule 1 (DNAM1)). NK cells are also activated by the constant region of some immunoglobulins through CD16 receptors on their surface. The overall sensitivity of NK cells to activation depends on the sum of stimulatory and inhibitory signals.

Delta like canonical Notch ligand 3 (DLL3) belongs to the delta protein ligand family, and acts as a ligand in the notch signaling pathway. DLL3 has been associated with a variety of neuroendocrine cancers. It is expressed on the surface of tumor cells in about 85% of patients with small-cell lung cancer and large-cell neuroendocrine cancer, but not in healthy tissues. It is also implicated in glioblastoma, Ewing Sarcoma and other cancers with neuroendocrine phenotype. DLL3 binds to Notch receptors and promotes the proliferation and inhibits the apoptosis of cancer cells.

Mucin 1 (MUC1) is a transmembrane mucin family protein having highly conserved 20 amino acid repeats (HGVTSAPDTRPAPGSTAPPA (SEQ ID NO:633)) decorated with a dense O-linked glycosylation pattern. MUC1 lines the apical surface of epithelial cells in the lungs, stomach, intestines, eyes and several other organs, and provides a protective barrier for the epithelial cells. MUC1 is normally expressed at a basal level in human epithelial cells, but is over-expressed in cancers, including gastric cancer, colorectal cancer, pancreatic cancer, breast cancer, endometrial cancer, lung cancer, bladder cancer, cervical cancer, head and neck cancer, ovarian cancer, renal cell cancer, and multiple myeloma. MUC1 is often shed from cells; in this case, a small extracellular domain (MUC1-C) remains on the cell surface following cleavage of the N-terminal ectodomain Furthermore, MUC1 in cancer cells is aberrantly glycosylated. For example, MUC1 is highly expressed in an underglycosylated form in multiple tumor types of epithelial origin, including over 90% of breast cancers.

Plexins are the signal-transducing elements of semaphorins, which are a large family of evolutionarily conserved molecules implicated in axon guidance, organogenesis, angiogenesis, immune responses, and oncogenesis. In particular, the type A plexins (Plexin-A1, Plexin-A2, Plexin-A3, and Plexin-A4), together with ligand-binding neuropilins, are the signaling moiety of the receptor complex for class 3 semaphorins. Moreover, the type A plexins are also the primary receptors for class 6 transmembrane semaphorins (e.g., Semaphorin-6A and Semaphorin-6D) that do not bind neuropilins. Type A plexins modulate the affinity of the receptor complex for specific semaphorins, and the cytoplasmic domain of type A plexins is required for the activation of down-stream signaling events in the cytoplasm. It has been identified that semaphorins and their receptor plexin-A1 are over-expressed in many cancers, including head and neck cancer, gastric cancer, pancreatic cancer, prostate cancer, and glioma.

Tumor Necrosis Factor (TNF) is a pro-inflammatory cytokine involved in the progression and development of cancer. Tumor necrosis factor receptor superfamily member 10B (TNFRSF10B) is a cell surface receptor of the TNF-receptor superfamily that binds TRAIL and mediates apoptosis. It is over-expressed in many types of cancers, such as liver cancer, pancreatic cancer, stomach cancer, renal cancer, breast cancer, ovarian cancer, endometrial cancer, and melanoma.

Six-transmembrane epithelial antigen of prostate member 1 (STEAP1) is a metalloreductase involved in a wide range of biologic processes, such as molecular trafficking in the endocytic and exocytic pathways, and control of cell proliferation and apoptosis. It is over-expressed in several types of human cancers, such as prostate cancer, bladder cancer, colon cancer, pancreatic cancer, ovarian cancer, testicular cancer, breast cancer, cervical cancer and Ewing sarcoma.

CUB domain-containing protein 1 (CDCP1) is a type I integral membrane glycoprotein that directly interacts with proteins involved in both cell-cell and cell-extracellular matrix adhesion, thereby playing a role in cell motility and adhesion. Increased CDCP1 expression has been found in various types of cancers, including colon cancer, lung cancer, gastric cancer, breast cancer, pancreatic cancer, head and neck cancer, bladder cancer, ovarian cancer, endometrial cancer, and skin cancer.

Tyrosine-protein kinase-like 7 (PTK7), also known as colon carcinoma kinase 4 (CCK4), is a member of the receptor protein tyrosine kinase family. PTK7 plays a role in vertebrate tissue morphogenesis, by regulating the canonical and non-canonical Wnt pathways, and orientation of cells in a tissue plane. Expression of PTK7 is upregulated in lung cancer, head and neck cancer, stomach cancer, prostate cancer, testicular cancer, endometrial cancer, breast cancer, melanoma, skin cancer, and leukemia.

AXL receptor tyrosine kinase (AXL), a cell surface receptor tyrosine kinase, transduces signals from the extracellular matrix into the cytoplasm by binding growth factors, and is involved in stimulation of cell proliferation and survival. AXL is over-expressed in many human cancers, including breast cancer, lung cancer, colon cancer, prostate cancer, renal cancer, esophageal cancer, liver cancer, pancreatic cancer, Kaposi's sarcoma, acute myeloid leukemia, glioma, and mesothelioma. AXL oncogenic signaling promotes cancer cell survival, proliferation, migration, and invasion.

Receptor tyrosine-protein kinase ERBB-3 (ERBB-3), also known as HER3, is a member of the epidermal growth factor receptor (EGFR/ERBB) family of receptor tyrosine kinases. It forms heterodimers with other EGF receptor family members and heterodimerization leads to the activation of pathways involved in cell proliferation or differentiation. Over-expression of ERBB-3 has been reported in numerous cancers, including prostate cancer, bladder cancer, and breast cancer, ovarian cancer, colon cancer, pancreatic cancer, stomach cancer, oral cavity cancer, head and neck cancer, lung cancer, and melanoma.

Endothelin receptor type B (EDNRB) is a G protein-coupled receptor which activates a phosphatidylinositol-calcium second messenger system. Its ligand, endothelin, consists of a family of three potent vasoactive peptides: endothelin-1, endothelin-2, and endothelin-3. Tumors over-express EDNRB and the endothelins. The interaction between EDNRB and the endothelins induces tumor growth and metastasis by promoting tumor cell survival and proliferation, angiogenesis, and tissue remodeling. Exemplary tumors include melanoma, uveal melanoma, and glioma.

Tyrosinase related protein-1 (TYRP1) belongs to a family of Cu⁺⁺/Zn⁺⁺metalloenzymes, which are expressed in melanocytes where they play key roles in promoting melanogenesis. The mature form of TYRP1, also called gp75, is a 75 kDa transmembrane glycoprotein produced within the endoplasmic reticulum (ER) and transported through the Golgi to specialized organelles called melanosomes. There is growing evidence indicating an important role of Tyrp1 in melanoma progression.

Oxidized low-density lipoprotein receptor 1 (OLR1) is the main receptor for oxidized low-density lipoprotein on endothelial cells, macrophages, smooth muscle cells, and other cell types. OLR1 binds, internalizes and degrades oxidized low-density lipoprotein. Over-expression of OLR1 has been associated with gastric cancer, colorectal cancer, pancreatic cancer, prostate cancer, breast cancer, and endometrial cancer.

ADAM12 is a member of the “a disintegrin and metalloprotease” (ADAM) protein family. Members of this family are membrane-anchored proteins structurally related to snake venom disintegrins, and have been implicated in a variety of biological processes involving cell-cell and cell-matrix interactions, including fertilization, muscle development, and neurogenesis. ADAM12 has two alternatively-spliced gene products: a shorter secreted form and a longer membrane-bound form. The shorter form is found to stimulate myogenesis. Numerous studies have demonstrated the importance of ADAM12 in cancer, and ADAM12 is markedly upregulated in a variety of human cancers, including prostate cancer, breast cancer, basal cell carcinoma, squamous cell carcinoma, melanoma, pancreatic cancer, bladder cancer, colorectal cancer, lung cancer, liver cancer, esophageal cancer, Non-Hodgkin's lymphoma, ovarian cancer, and uterine cancer.

The urokinase receptor, also known as urokinase plasminogen activator receptor, CD87 or PLAUR, is a multidomain glycoprotein tethered to the cell membrane with a glycosylphosphotidylinositol (GPI) anchor. PLAUR is a part of the plasminogen activation system, which in the healthy body is involved in tissue reorganization events such as mammary gland involution and wound healing. Elevated levels of PLAUR is detected in various cancer types (for example, breast cancer, colorectal cancer, non-small cell lung cancer, and oral cancer), and is closely associated with poor prognosis of cancers. Binding of uPA to PLAUR triggers the conversion of plasminogen to plasmin and the subsequent activation of metalloproteinases. These events confer tumor cells with the ability to degrade the components of the surrounding extracellular matrix, thus contributing to tumor cell invasion and metastasis. uPA-PLAR interaction also elicits signals that stimulate cell proliferation/survival and the expression of tumor-promoting genes, thus assisting tumor development. In addition to its interaction with uPA, PLAUR also interacts with vitronectin and this interaction promotes cancer metastasis by activating Rac and stimulating cell migration. Although underlying mechanisms are yet to be fully elucidated, PLAUR has been shown to facilitate epithelial-mesenchymal transition (EMT) and induce cancer stem cell-like properties in breast cancer cells.

C-C motif chemokine receptor 6 (CCR6) is a member of the beta chemokine receptor family, which is a seven transmembrane protein similar to G protein-coupled receptors. This receptor is preferentially expressed by immature dendritic cells and memory T cells, and the ligand of this receptor is macrophage inflammatory protein 3 alpha (MIP-3 alpha). CCR6 has been shown to be important for B-lineage maturation and antigen-driven B-cell differentiation, and it may regulate the migration and recruitment of dentritic and T cells during inflammatory and immunological responses. In addition, expression of CCR6 was found to be upregulated in many cancer types, for example, colorectal cancer, breast cancer, cervical cancer, liver cancer, lung cancer, and cutaneous T-cell lymphoma, and to contribute to the proliferation and migration of the cancers.

Ephrin type-A receptor 4 (EPHA4) belongs to the ephrin receptor subfamily of protein-tyrosine kinases. EPHA4 relays a direct cell-cell contact-mediated bidirectional signaling pathway. EPHA4 signaling mainly affects cell shape and motility by regulating cytoskeletal organization and cellular adhesion. EPHA4 signaling also influences cell proliferation and cell-fate. The genes for Ephrin receptors and ephrins have been recognized to be differentially expressed in various human tumors including melanoma, glioma, prostate cancer, breast cancer, small cell lung cancer, endometrial cancer, esophageal cancer, gastric cancer, and colorectal cancer. Abnormal EPHA4 expression can be correlated with altered tumor behavior such as increased invasiveness or increased metastatic potential and, consequently, poor patient outcome.

Myeloid-derived suppressor cells (MDSCs) represent a heterogeneous population of immature myeloid cells consisting of precursors for granulocytes, macrophages or dendritic cells (DCs) that accumulate during chronic inflammation and tumor progression. It has been shown that established tumors are able to produce multiple factors that impair myelopoiesis favoring the formation of MDSCs, trafficking of MDSCs to the tumor site and activation of MDSCs. Numerous recent studies have demonstrated that after the generation and migration to the tumor site, MDSCs significantly upregulate their immunosuppressive functions inhibiting the anti-tumor functions of T cells and NK cells. Moreover, MDSCs directly stimulate tumor development by promoting neovascularization, and tumor cell invasion by creating a pre-metastatic environment.

There are two different types of MDSCs identified in humans: polymorphonuclear MDSCs (PMN-MDSCs), and monocytic MDSCs (M-MDSCs). In the tumor, M-MDSCs are more prominent, and rapidly differentiate to tumor-associated macrophages (TAMs) (see Kumar et al. (2016) Trends Immunol.; 37(3): 208-220.). In addition to being differentiated from MDSCs, TAMs can be tissue resident. Alternatively, peripheral blood monocytes can be recruited locally to the tissue and differentiate into TAMS in response to various chemokines and growth factors produced by stromal and tumor cells in the tumor microenvironment. TAMs play an important role in connecting inflammation with cancer. They can promote proliferation, invasion, and metastasis of tumor cells, stimulate tumor angiogenesis, and inhibit antitumor immune response mediated by T cells (Yang et al. (2017) Journal of Hematology & Oncology; 10:58.).

A variety of antigens may be expressed on MDSCs and/or TAMs, which are present in a tumor microenvironment, including CD14, CD163, colony stimulating factor 3 receptor (CSF3R), sialic acid-binding Ig-like lectin 9 (Siglec-9), integrin alpha M (ITGAM), V-domain Ig suppressor of T cell activation (VISTA), B7-H4 (also known as V-Set Domain Containing T Cell Activation Inhibitor 1; VTCN1), C-C chemokine receptor type 1 (CCR1), leucine rich repeat containing 25 (LRRC25), platelet activating factor receptor (PTAFR), signal regulatory protein beta 1 (SIRPB1), Toll-like receptor 2 (TLR2), Toll-like receptor 4 (TLR4), CD300 molecule like family member b (CD300LB), ATPase Na⁺/K⁺ transporting subunit alpha 3 (ATP1A3), and C-C chemokine receptor type 5 (CCR5).

The present invention provides certain advantages to improve treatments for the above-mentioned cancers.

### SUMMARY

The invention provides multi-specific binding proteins that bind to the NKG2D receptor and CD16 receptor on natural killer cells, and a tumor-associated antigen selected from DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, and EPHA4. The invention also provides multi-specific binding proteins that bind to the NKG2D receptor and CD16 receptor on natural killer cells, and an antigen selected from CD14, CD163, CSF3R, Siglec-9, ITGAM, VISTA, B7-H4 (VTCN1), CCR1, LRRC25, PTAFR, SIRPB1, TLR2, TLR4, CD300LB, ATP1A3, and CCR5. Such proteins can engage more than one kind of NK-activating receptor, and may block the binding of natural ligands to NKG2D. In certain embodiments, the proteins can agonize NK cells in humans, and in other species such as rodents and cynomolgus monkeys. Various aspects and embodiments of the invention are described in further detail below.

Accordingly, in certain embodiments the invention provides a protein that incorporates a first antigen-binding site that binds NKG2D; a second antigen-binding site that binds a tumor-associated antigen selected from DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, and EPHA4; and an antibody fragment crystallizable (Fc) domain, a portion thereof sufficient to bind CD16, or a third antigen-binding site that binds CD16.

In certain embodiments, the present invention provides multi-specific binding proteins that bind to a tumor-associated antigen selected from DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, and EPHA4 on a cancer cell, and to the NKG2D receptor and CD16 receptor on natural killer cells, in which the NKG2D-binding site includes a heavy chain variable domain at least 90% identical to an amino acid sequence selected from: SEQ ID NO:1, SEQ ID NO:41, SEQ ID NO:49, SEQ ID NO: 57, SEQ ID NO:59, SEQ ID NO:61, SEQ ID NO:69, SEQ ID NO:77, SEQ ID NO:85, SEQ ID NO:650, SEQ ID NO:653, SEQ ID NO:656, SEQ ID NO:659, SEQ ID NO:662, SEQ ID NO:665, and SEQ ID NO:93.

In certain other embodiments, the invention provides a protein that incorporates a first antigen-binding site that binds NKG2D; a second antigen-binding site that binds an antigen selected from CD14, CD163, CSF3R, Siglec-9, ITGAM, VISTA, B7-H4 (VTCN1), CCR1, LRRC25, PTAFR, SIRPB1, TLR2, TLR4, CD300LB, ATP1A3, and CCR5; and an antibody Fc domain, a portion thereof sufficient to bind CD16, or a third antigen-binding site that binds CD16.

In certain other embodiments, the invention provides a protein that incorporates a first antigen-binding site that binds NKG2D; a second antigen-binding site that binds an antigen selected from CD14, CD163, CSF3R, Siglec-9, ITGAM, VISTA, B7-H4 (VTCN1), CCR1, LRRC25, PTAFR, SIRPB1, TLR2, TLR4, CD300LB, ATP1A3, and CCR5; an antibody Fc domain, or a portion thereof, sufficient to bind CD16, or a third antigen-binding site that binds CD16; and a fourth antigen-binding site that binds to a tumor-associated antigen, which includes any antigen that is associated with cancer, such as, but not limited to, a protein, glycoprotein, ganglioside, carbohydrate, or lipid. Such antigen can be expressed on malignant cells or in the tumor microenvironment such as on tumor-associated blood vessels, extracellular matrix, mesenchymal stroma, or immune infiltrates.

The antigen-binding sites may each incorporate an antibody heavy chain variable domain and an antibody light chain variable domain (e.g., arranged as in an antibody, or fused together to from an scFv), or one or more of the antigen-binding sites may be a single domain antibody, such as a V_{H}H antibody like a camelid antibody or a V_{NAR} antibody like those found in cartilaginous fish.

The first antigen-binding site, which binds to NKG2D, in some embodiments, can incorporate a heavy chain variable domain related to SEQ ID NO:1, such as by having an amino acid sequence at least 90% (e.g., 90%, 91%, 92%, 93%, 94%, 95%, 96%, 97%, 98%, 99%, or 100%) identical to SEQ ID NO:1, and/or incorporating amino acid sequences identical to the CDR1 (SEQ ID NO:105), CDR2 (SEQ ID NO:106), and CDR3 (SEQ ID NO:107 or SEQ ID NO:635) sequences of SEQ ID NO:1. The heavy chain variable domain related to SEQ ID NO:1 can be coupled a variety of light chain variable domains to form a NKG2D binding site. For example, the first antigen-binding site that incorporates a heavy chain variable domain related to SEQ ID NO:1 can further incorporate a light chain variable domain selected from any one of the sequences related to SEQ ID NOs:2, 4, 6, 8, 10, 12, 14, 16, 18, 20, 22, 24, 26, 28, 30, 32, 34, 36, and 40. For example, the first antigen-binding site incorporates a heavy chain variable domain with amino acid sequences at least 90% (e.g., 90%, 91%, 92%, 93%, 94%, 95%, 96%, 97%, 98%, 99%, or 100%) identical to SEQ ID NO:1 and a light chain variable domain with amino acid sequences at least 90% (e.g., 90%, 91%, 92%, 93%, 94%, 95%, 96%, 97%, 98%, 99%, or 100%) identical to any one of the sequences selected from SEQ ID NOs:2, 4, 6, 8, 10, 12, 14, 16, 18, 20, 22, 24, 26, 28, 30, 32, 34, 36, and 40.

Alternatively, in certain embodiments the first antigen-binding site can incorporate a heavy chain variable domain related to SEQ ID NO:41 and a light chain variable domain related to SEQ ID NO:42. For example, the heavy chain variable domain of the first antigen binding site can be at least 90% (e.g., 90%, 91%, 92%, 93%, 94%, 95%, 96%, 97%, 98%, 99%, or 100%) identical to SEQ ID NO:41, and/or incorporate amino acid sequences identical to the CDR1 (SEQ ID NO:43 or SEQ ID NO:636), CDR2 (SEQ ID NO:44), and CDR3 (SEQ ID NO:45 or SEQ ID NO:637) sequences of SEQ ID NO:41. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:42, and/or incorporate amino acid sequences identical to the CDR1 (SEQ ID NO:46), CDR2 (SEQ ID NO:47), and CDR3 (SEQ ID NO:48) sequences of SEQ ID NO:42.

In certain embodiments, the first antigen-binding site can incorporate a heavy chain variable domain related to SEQ ID NO:49 and a light chain variable domain related to SEQ ID NO:50. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:49, and/or incorporate amino acid sequences identical to the CDR1 (SEQ ID NO:51 or SEQ ID NO:638), CDR2 (SEQ ID NO:52), and CDR3 (SEQ ID NO:53 or SEQ ID NO:639) sequences of SEQ ID NO:49. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:50, and/or incorporate amino acid sequences identical to the CDR1 (SEQ ID NO:54), CDR2 (SEQ ID NO:55), and CDR3 (SEQ ID NO:56) sequences of SEQ ID NO:50.

Alternatively, the first antigen-binding site can incorporate a heavy chain variable domain related to SEQ ID NO:57 and a light chain variable domain related to SEQ ID NO:58, such as by having amino acid sequences at least 90% (e.g., 90%, 91%, 92%, 93%, 94%, 95%, 96%, 97%, 98%, 99%, or 100%) identical to SEQ ID NO:57 and at least 90% (e.g., 90%, 91%, 92%, 93%, 94%, 95%, 96%, 97%, 98%, 99%, or 100%) identical to SEQ ID NO:58 respectively.

In another embodiment, the first antigen-binding site can incorporate a heavy chain variable domain related to SEQ ID NO:59 and a light chain variable domain related to SEQ ID NO:60, such as by having amino acid sequences at least 90% (e.g., 90%, 91%, 92%, 93%, 94%, 95%, 96%, 97%, 98%, 99%, or 100%) identical to SEQ ID NO:59 and at least 90% (e.g., 90%, 91%, 92%, 93%, 94%, 95%, 96%, 97%, 98%, 99%, or 100%) identical to SEQ ID NO:60 respectively. For example, the heavy chain variable domain of the first antigen binding site can be at least 90% (e.g., 90%, 91%, 92%, 93%, 94%, 95%, 96%, 97%, 98%, 99%, or 100%) identical to SEQ ID NO:59, and/or incorporate amino acid sequences identical to the CDR1 (SEQ ID NO:108), CDR2 (SEQ ID NO:109), and CDR3 (SEQ ID NO:110) sequences of SEQ ID NO:59. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:60, and/or incorporate amino acid sequences identical to the CDR1 (SEQ ID NO:111), CDR2 (SEQ ID NO:112), and CDR3 (SEQ ID NO:113) sequences of SEQ ID NO:60.

In some embodiments, the first antigen-binding site can incorporate a heavy chain variable domain related to SEQ ID NO:101 and a light chain variable domain related to SEQ ID NO:102, such as by having amino acid sequences at least 90% (e.g., 90%, 91%, 92%, 93%, 94%, 95%, 96%, 97%, 98%, 99%, or 100%) identical to SEQ ID NO:101 and at least 90% (e.g., 90%, 91%, 92%, 93%, 94%, 95%, 96%, 97%, 98%, 99%, or 100%) identical to SEQ ID NO:102 respectively. In some embodiments, the first antigen-binding site can incorporate a heavy chain variable domain related to SEQ ID NO:103 and a light chain variable domain related to SEQ ID NO:104, such as by having amino acid sequences at least 90% (e.g., 90%, 91%, 92%, 93%, 94%, 95%, 96%, 97%, 98%, 99%, or 100%) identical to SEQ ID NO:103 and at least 90% (e.g., 90%, 91%, 92%, 93%, 94%, 95%, 96%, 97%, 98%, 99%, or 100%) identical to SEQ ID NO:104 respectively.

The first antigen-binding site, which binds to NKG2D, in some embodiments, can incorporate a heavy chain variable domain related to SEQ ID NO:61 and a light chain variable domain related to SEQ ID NO:62. For example, the heavy chain variable domain of the first antigen binding site can be at least 90% (e.g., 90%, 91%, 92%, 93%, 94%, 95%, 96%, 97%, 98%, 99%, or 100%) identical to SEQ ID NO:61, and/or incorporate amino acid sequences identical to the CDR1 (SEQ ID NO:63 or SEQ ID NO:640), CDR2 (SEQ ID NO:64), and CDR3 (SEQ ID NO:65 or SEQ ID NO:641) sequences of SEQ ID NO:61. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:62, and/or incorporate amino acid sequences identical to the CDR1 (SEQ ID NO:66), CDR2 (SEQ ID NO:67), and CDR3 (SEQ ID NO:68) sequences of SEQ ID NO:62. In some embodiments, the first antigen-binding site can incorporate a heavy chain variable domain related to SEQ ID NO:69 and a light chain variable domain related to SEQ ID NO:70. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:69, and/or incorporate amino acid sequences identical to the CDR1 (SEQ ID NO:71 or SEQ ID NO:642), CDR2 (SEQ ID NO:72), and CDR3 (SEQ ID NO:73 or SEQ ID NO:643) sequences of SEQ ID NO:69. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:70, and/or incorporate amino acid sequences identical to the CDR1 (SEQ ID NO:74), CDR2 (SEQ ID NO:75), and CDR3 (SEQ ID NO:76) sequences of SEQ ID NO:70.

In some embodiments, the first antigen-binding site can incorporate a heavy chain variable domain related to SEQ ID NO:77 and a light chain variable domain related to SEQ ID NO:78. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:77, and/or incorporate amino acid sequences identical to the CDR1 (SEQ ID NO:79 or SEQ ID NO:644), CDR2 (SEQ ID NO:80), and CDR3 (SEQ ID NO:81 or SEQ ID NO:645) sequences of SEQ ID NO:77. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:78, and/or incorporate amino acid sequences identical to the CDR1 (SEQ ID NO:82), CDR2 (SEQ ID NO:83), and CDR3 (SEQ ID NO:84) sequences of SEQ ID NO:78.

In some embodiments, the first antigen-binding site can incorporate a heavy chain variable domain related to SEQ ID NO:85 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:85, and/or incorporate amino acid sequences identical to the CDR1 (SEQ ID NO:87 or SEQ ID NO:646), CDR2 (SEQ ID NO:88), and CDR3 (SEQ ID NO:89 or SEQ ID NO:647) sequences of SEQ ID NO:85. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:650 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:650, and/or incorporate amino acid sequences identical to the CDR1 (SEQ ID NO:87 or SEQ ID NO:646), CDR2 (SEQ ID NO:88), and CDR3 (SEQ ID NO:651 or SEQ ID NO:652) sequences of SEQ ID NO:650. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:653 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:653, and/or incorporate amino acid sequences identical to the CDR1 (SEQ ID NO:87 or SEQ ID NO:646), CDR2 (SEQ ID NO:88), and CDR3 (SEQ ID NO:654 or SEQ ID NO:655) sequences of SEQ ID NO:653. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:656 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:656, and/or incorporate amino acid sequences identical to the CDR1 (SEQ ID NO:87 or SEQ ID NO:646), CDR2 (SEQ ID NO:88), and CDR3 (SEQ ID NO:657 or SEQ ID NO:658) sequences of SEQ ID NO:656. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:659 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:659, and/or incorporate amino acid sequences identical to the CDR1 (SEQ ID NO:87 or SEQ ID NO:646), CDR2 (SEQ ID NO:88), and CDR3 (SEQ ID NO:660 or SEQ ID NO:661) sequences of SEQ ID NO:659. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:662 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:662, and/or incorporate amino acid sequences identical to the CDR1 (SEQ ID NO:87 or SEQ ID NO:646), CDR2 (SEQ ID NO:88), and CDR3 (SEQ ID NO:663 or SEQ ID NO:664) sequences of SEQ ID NO:662. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:665 and a light chain variable domain related to SEQ ID NO:86. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:665, and/or incorporate amino acid sequences identical to the CDR1 (SEQ ID NO:87 or SEQ ID NO:646), CDR2 (SEQ ID NO:88), and CDR3 (SEQ ID NO:666 or SEQ ID NO:667) sequences of SEQ ID NO:665. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:90), CDR2 (SEQ ID NO:91), and CDR3 (SEQ ID NO:92) sequences of SEQ ID NO:86.

In some embodiments, the first antigen-binding site can incorporate a heavy chain variable domain related to SEQ ID NO:93 and a light chain variable domain related to SEQ ID NO:94. For example, the heavy chain variable domain of the first antigen-binding site can be at least 90% (e.g., 90%, 91%, 92%, 93%, 94%, 95%, 96%, 97%, 98%, 99%, or 100%) identical to SEQ ID NO:93, and/or incorporate amino acid sequences identical to the CDR1 (SEQ ID NO:95 or SEQ ID NO:648), CDR2 (SEQ ID NO:96), and CDR3 (SEQ ID NO:97 or SEQ ID NO:649) sequences of SEQ ID NO:93. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:94, and/or incorporate amino acid sequences identical to the CDR1 (SEQ ID NO:98), CDR2 (SEQ ID NO:99), and CDR3 (SEQ ID NO:100) sequences of SEQ ID NO:94.

In certain embodiments, the second antigen-binding site can bind DLL3 and can optionally incorporate a heavy chain variable domain related to SEQ ID NO:114 and a light chain variable domain related to SEQ ID NO:115. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:114, and/or incorporate amino acid sequences identical to the CDR1 (SEQ ID NO:116), CDR2 (SEQ ID NO:117), and CDR3 (SEQ ID NO:118) sequences of SEQ ID NO:114. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:115 and/or incorporate amino acid sequences identical to the CDR1 (SEQ ID NO:119), CDR2 (SEQ ID NO:120), and CDR3 (SEQ ID NO:121) sequences of SEQ ID NO:115.

Alternatively, the second antigen-binding site binding to DLL3 can incorporate a heavy chain variable domain related to SEQ ID NO:122 and a light chain variable domain related to SEQ ID NO:123. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:122, and/or incorporate amino acid sequences identical to the CDR1 (SEQ ID NO:124), CDR2 (SEQ ID NO:125), and CDR3 (SEQ ID NO:126) sequences of SEQ ID NO:122. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:123, and/or incorporate amino acid sequences identical to the CDR1 (SEQ ID NO:127), CDR2 (SEQ ID NO:128), and CDR3 (SEQ ID NO:129) sequences of SEQ ID NO:123.

In some embodiments, the second antigen-binding site binding to DLL3 can incorporate a heavy chain variable domain related to SEQ ID NO:679, 668, 671, 673, 675, 677, or 130 and a light chain variable domain related to SEQ ID NO:669 or 131. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:679, 668, 671, 673, 675, 677, or 130, and/or incorporate amino acid sequences identical to the CDR1 (SEQ ID NO:132), CDR2 (SEQ ID NO:133), and CDR3 (SEQ ID NO: 670, 672, 674, 676, 678, 680, or 134) sequences of SEQ ID NO:679, 668, 671, 673, 675, 677, or 130. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:669 or 131, and/or incorporate amino acid sequences identical to the CDR1, CDR2, and CDR3 sequences of SEQ ID NO:669 or 131.

In some embodiments, the second antigen-binding site binding to DLL3 can incorporate a heavy chain variable domain related to SEQ ID NO:138 and a light chain variable domain related to SEQ ID NO:139. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:138, and/or incorporate amino acid sequences identical to the CDR1 (SEQ ID NO:140), CDR2 (SEQ ID NO:141), and CDR3 (SEQ ID NO:142) sequences of SEQ ID NO:138. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:139, and/or incorporate amino acid sequences identical to the CDR1 (SEQ ID NO:143), CDR2 (SEQ ID NO:144), and CDR3 (SEQ ID NO:145) sequences of SEQ ID NO:139.

In some embodiments, the second antigen-binding site binding to DLL3 can incorporate a heavy chain variable domain related to SEQ ID NO:146 and a light chain variable domain related to SEQ ID NO:147. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:146, and/or incorporate amino acid sequences identical to the CDR1 (SEQ ID NO:148), CDR2 (SEQ ID NO:149), and CDR3 (SEQ ID NO:150) sequences of SEQ ID NO:146. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:147, and/or incorporate amino acid sequences identical to the CDR1 (SEQ ID NO:151), CDR2 (SEQ ID NO:152), and CDR3 (SEQ ID NO:153) sequences of SEQ ID NO:147.

In some embodiments, the second antigen-binding site binding to DLL3 can incorporate a heavy chain variable domain related to SEQ ID NO:154 and a light chain variable domain related to SEQ ID NO:155. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:154, and/or incorporate amino acid sequences identical to the CDR1 (SEQ ID NO:156), CDR2 (SEQ ID NO:157), and CDR3 (SEQ ID NO:158) sequences of SEQ ID NO:154. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:155, and/or incorporate amino acid sequences identical to the CDR1 (SEQ ID NO:159), CDR2 (SEQ ID NO:160), and CDR3 (SEQ ID NO:161) sequences of SEQ ID NO:155.

In some embodiments, the second antigen-binding site binding to DLL3 can incorporate a heavy chain variable domain related to SEQ ID NO:162 and a light chain variable domain related to SEQ ID NO:163. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:162, and/or incorporate amino acid sequences identical to the CDR1 (SEQ ID NO:164), CDR2 (SEQ ID NO:165), and CDR3 (SEQ ID NO:166) sequences of SEQ ID NO:162. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:163, and/or incorporate amino acid sequences identical to the CDR1 (SEQ ID NO:167), CDR2 (SEQ ID NO:168), and CDR3 (SEQ ID NO:169) sequences of SEQ ID NO:163.

In some embodiments, the second antigen-binding site binding to DLL3 can incorporate a heavy chain variable domain related to SEQ ID NO:170 and a light chain variable domain related to SEQ ID NO:171. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:170, and/or incorporate amino acid sequences identical to the CDR1 (SEQ ID NO:172), CDR2 (SEQ ID NO:173), and CDR3 (SEQ ID NO:174) sequences of SEQ ID NO:170. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:171, and/or incorporate amino acid sequences identical to the CDR1 (SEQ ID NO:175), CDR2 (SEQ ID NO:176), and CDR3 (SEQ ID NO:177) sequences of SEQ ID NO:171.

In some embodiments, the second antigen-binding site binding to DLL3 can incorporate a heavy chain variable domain related to SEQ ID NO:178 and a light chain variable domain related to SEQ ID NO:179. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:178, and/or incorporate amino acid sequences identical to the CDR1 (SEQ ID NO:180), CDR2 (SEQ ID NO:181), and CDR3 (SEQ ID NO:182) sequences of SEQ ID NO:178. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:179, and/or incorporate amino acid sequences identical to the CDR1 (SEQ ID NO:183), CDR2 (SEQ ID NO:184), and CDR3 (SEQ ID NO:185) sequences of SEQ ID NO:179.

In some embodiments, the second antigen-binding site binding to DLL3 can incorporate a heavy chain variable domain related to SEQ ID NO:186 and a light chain variable domain related to SEQ ID NO:187. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:186, and/or incorporate amino acid sequences identical to the CDR1 (SEQ ID NO:188), CDR2 (SEQ ID NO:189), and CDR3 (SEQ ID NO:190) sequences of SEQ ID NO:186. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:187, and/or incorporate amino acid sequences identical to the CDR1 (SEQ ID NO:191), CDR2 (SEQ ID NO:192), and CDR3 (SEQ ID NO:193) sequences of SEQ ID NO:187.

In some embodiments, the second antigen-binding site binding to DLL3 can incorporate a heavy chain variable domain related to SEQ ID NO:194 and a light chain variable domain related to SEQ ID NO:195. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:194, and/or incorporate amino acid sequences identical to the CDR1 (SEQ ID NO:196), CDR2 (SEQ ID NO:197), and CDR3 (SEQ ID NO:198) sequences of SEQ ID NO:194. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:195, and/or incorporate amino acid sequences identical to the CDR1 (SEQ ID NO:199), CDR2 (SEQ ID NO:200), and CDR3 (SEQ ID NO:201) sequences of SEQ ID NO:195.

In certain embodiments, the second antigen-binding site can bind MUC1 (or MUC1-C) and can optionally incorporate a heavy chain variable domain related to SEQ ID NO:203 and a light chain variable domain related to SEQ ID NO:207. For example, the heavy chain variable domain of the second antigen-binding site can be at least 90% (e.g., 90%, 91%, 92%, 93%, 94%, 95%, 96%, 97%, 98%, 99%, or 100%) identical to 203, and/or incorporate amino acid sequences identical to the CDR1 (SEQ ID NO:204), CDR2 (SEQ ID NO:205), and CDR3 (SEQ ID NO:206) sequences of SEQ ID NO:203. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:207, and/or incorporate amino acid sequences identical to the CDR1 (SEQ ID NO:208), CDR2 (SEQ ID NO:209), and CDR3 (SEQ ID NO:210) sequences of SEQ ID NO:207.

Alternatively, the second antigen-binding site binding to MUC1 (or MUC1-C) can incorporate a heavy chain variable domain related to SEQ ID NO:211 and a light chain variable domain related to SEQ ID NO:215. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:211, and/or incorporate amino acid sequences identical to the CDR1 (SEQ ID NO:212), CDR2 (SEQ ID NO:213), and CDR3 (SEQ ID NO:214) sequences of SEQ ID NO:211. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:215, and/or incorporate amino acid sequences identical to the CDR1 (SEQ ID NO:216), CDR2 (SEQ ID NO:217), and CDR3 (SEQ ID NO:218) sequences of SEQ ID NO:215.

The certain embodiments, the second antigen-binding site can bind to Plexin-A1 and can optionally incorporate a heavy chain variable domain related to SEQ ID NO:220 and a light chain variable domain related to SEQ ID NO:224. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:220, and/or incorporate amino acid sequences identical to the CDR1 (SEQ ID NO:221), CDR2 (SEQ ID NO:222), and CDR3 (SEQ ID NO:223) sequences of SEQ ID NO:220. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:224, and/or incorporate amino acid sequences identical to the CDR1 (SEQ ID NO:225), CDR2 (SEQ ID NO:226), and CDR3 (SEQ ID NO:227) sequences of SEQ ID NO:224. Alternatively, the second antigen-binding site binding to Plexin-A1 can optionally incorporate a heavy chain variable domain related to SEQ ID NO:228 and a light chain variable domain related to SEQ ID NO:232. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:228, and/or incorporate amino acid sequences identical to the CDR1 (SEQ ID NO:229), CDR2 (SEQ ID NO:230), and CDR3 (SEQ ID NO:231) sequences of SEQ ID NO:228. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:232, and/or incorporate amino acid sequences identical to the CDR1 (SEQ ID NO:233), CDR2 (SEQ ID NO:234), and CDR3 (SEQ ID NO:235) sequences of SEQ ID NO:232.

In certain embodiments, the second antigen-binding site can bind to TNFRSF10B and can optionally incorporate a heavy chain variable domain related to SEQ ID NO:237 and a light chain variable domain related to SEQ ID NO:241. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:237, and/or incorporate amino acid sequences identical to the CDR1 (SEQ ID NO:238), CDR2 (SEQ ID NO:239), and CDR3 (SEQ ID NO:240) sequences of SEQ ID NO:237. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:241, and/or incorporate amino acid sequences identical to the CDR1 (SEQ ID NO:242), CDR2 (SEQ ID NO:243), and CDR3 (SEQ ID NO:244) sequences of SEQ ID NO:241. Alternatively, the second antigen-binding site binding to TNFRSF10B can optionally incorporate a heavy chain variable domain related to SEQ ID NO:245 and a light chain variable domain related to SEQ ID NO:249. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:245, and/or incorporate amino acid sequences identical to the CDR1 (SEQ ID NO:246), CDR2 (SEQ ID NO:247), and CDR3 (SEQ ID NO:248) sequences of SEQ ID NO:248. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:249, and/or incorporate amino acid sequences identical to the CDR1 (SEQ ID NO:250), CDR2 (SEQ ID NO:251), and CDR3 (SEQ ID NO:252) sequences of SEQ ID NO:249. Alternatively, the second antigen-binding site binding to TNFRSF10B can optionally incorporate a heavy chain variable domain related to SEQ ID NO:253 and a light chain variable domain related to SEQ ID NO:257. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:253, and/or incorporate amino acid sequences identical to the CDR1 (SEQ ID NO:254), CDR2 (SEQ ID NO:255), and CDR3 (SEQ ID NO:256) sequences of SEQ ID NO:253. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:257, and/or incorporate amino acid sequences identical to the CDR1 (SEQ ID NO:258), CDR2 (SEQ ID NO:259), and CDR3 (SEQ ID NO:260) sequences of SEQ ID NO:257.

In certain embodiments, the second antigen-binding site can bind to STEAP1 and can optionally incorporate a heavy chain variable domain related to SEQ ID NO:262 and a light chain variable domain related to SEQ ID NO:266. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:262, and/or incorporate amino acid sequences identical to the CDR1 (SEQ ID NO:263), CDR2 (SEQ ID NO:264), and CDR3 (SEQ ID NO:265) sequences of SEQ ID NO:262. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:266, and/or incorporate amino acid sequences identical to the CDR1 (SEQ ID NO:267), CDR2 (SEQ ID NO:268), and CDR3 (SEQ ID NO:269) sequences of SEQ ID NO:266. Alternatively, the second antigen-binding site binding to STEAP1 can optionally incorporate a heavy chain variable domain related to SEQ ID NO:270 and a light chain variable domain related to SEQ ID NO:274. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:270, and/or incorporate amino acid sequences identical to the CDR1 (SEQ ID NO:271), CDR2 (SEQ ID NO:272), and CDR3 (SEQ ID NO:273) sequences of SEQ ID NO:270. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:274, and/or incorporate amino acid sequences identical to the CDR1 (SEQ ID NO:275), CDR2 (SEQ ID NO:276), and CDR3 (SEQ ID NO:277) sequences of SEQ ID NO:274.

In certain embodiments, the second antigen-binding site can bind to CDCP1 and can optionally incorporate a heavy chain variable domain related to SEQ ID NO:279 and a light chain variable domain related to SEQ ID NO:283 or SEQ ID NO:287. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:279, and/or incorporate amino acid sequences identical to the CDR1 (SEQ ID NO:280), CDR2 (SEQ ID NO:281), and CDR3 (SEQ ID NO:282) sequences of SEQ ID NO:279. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:283, and/or incorporate amino acid sequences identical to the CDR1 (SEQ ID NO:284), CDR2 (SEQ ID NO:285), and CDR3 (SEQ ID NO:286) sequences of SEQ ID NO:283. Alternatively, the light chain variable domain of the second antigen-binding site can be at least 90% (e.g., 90%, 91%, 92%, 93%, 94%, 95%, 96%, 97%, 98%, 99%, or 100%) identical to SEQ ID NO:287, and/or incorporate amino acid sequences identical to the CDR1 (SEQ ID NO:288), CDR2 (SEQ ID NO:289), and CDR3 (SEQ ID NO:290) sequences of SEQ ID NO:287.

In certain embodiments, the second antigen-binding site can bind to to PTK7 and can optionally incorporate a heavy chain variable domain related to SEQ ID NO:292 and a light chain variable domain related to SEQ ID NO:296. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:292, and/or incorporate amino acid sequences identical to the CDR1 (SEQ ID NO:293), CDR2 (SEQ ID NO:294), and CDR3 (SEQ ID NO:295) sequences of SEQ ID NO:292. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:296, and/or incorporate amino acid sequences identical to the CDR1 (SEQ ID NO:297), CDR2 (SEQ ID NO:298), and CDR3 (SEQ ID NO:299) sequences of SEQ ID NO:296.

The second antigen-binding site binding to AXL can optionally incorporate a heavy chain variable domain related to SEQ ID NO:301 and a light chain variable domain related to SEQ ID NO:305. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:301, and/or incorporate amino acid sequences identical to the CDR1 (SEQ ID NO:302), CDR2 (SEQ ID NO:303), and CDR3 (SEQ ID NO:304) sequences of SEQ ID NO:301. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:305, and/or incorporate amino acid sequences identical to the CDR1 (SEQ ID NO:306), CDR2 (SEQ ID NO:307), and CDR3 (SEQ ID NO:308) sequences of SEQ ID NO:305. Alternatively, the second antigen-binding site binding to AXL can optionally incorporate a heavy chain variable domain related to SEQ ID NO:309 and a light chain variable domain related to SEQ ID NO:313. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:309, and/or incorporate amino acid sequences identical to the CDR1 (SEQ ID NO:310), CDR2 (SEQ ID NO:311), and CDR3 (SEQ ID NO:312) sequences of SEQ ID NO:309. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:313, and/or incorporate amino acid sequences identical to the CDR1 (SEQ ID NO:314), CDR2 (SEQ ID NO:315), and CDR3 (SEQ ID NO:316) sequences of SEQ ID NO:313.

In certain embodiments, the second antigen-binding site can bind to ERBB-3 and can optionally incorporate a heavy chain variable domain related to SEQ ID NO:318 and a light chain variable domain related to SEQ ID NO:322. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:318, and/or incorporate amino acid sequences identical to the CDR1 (SEQ ID NO:319), CDR2 (SEQ ID NO:320), and CDR3 (SEQ ID NO:321) sequences of SEQ ID NO:318. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:322, and/or incorporate amino acid sequences identical to the CDR1 (SEQ ID NO:323), CDR2 (SEQ ID NO:324), and CDR3 (SEQ ID NO:325) sequences of SEQ ID NO:322. Alternatively, the second antigen-binding site binding to ERBB-3 can optionally incorporate a heavy chain variable domain related to SEQ ID NO:326 and a light chain variable domain related to SEQ ID NO:330. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:326, and/or incorporate amino acid sequences identical to the CDR1 (SEQ ID NO:327), CDR2 (SEQ ID NO:328), and CDR3 (SEQ ID NO:329) sequences of SEQ ID NO:326. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:330, and/or incorporate amino acid sequences identical to the CDR1 (SEQ ID NO:331), CDR2 (SEQ ID NO:332), and CDR3 (SEQ ID NO:333) sequences of SEQ ID NO:330. Alternatively, the second antigen-binding site binding to ERBB-3 can optionally incorporate a heavy chain variable domain related to SEQ ID NO:334 and a light chain variable domain related to SEQ ID NO:338. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:334, and/or incorporate amino acid sequences identical to the CDR1 (SEQ ID NO:335), CDR2 (SEQ ID NO:336), and CDR3 (SEQ ID NO:337) sequences of SEQ ID NO:334. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:338, and/or incorporate amino acid sequences identical to the CDR1 (SEQ ID NO:339), CDR2 (SEQ ID NO:340), and CDR3 (SEQ ID NO:341) sequences of SEQ ID NO:338.

In certain embodiments, the second antigen-binding site can bind to EDNRB and can optionally incorporate a heavy chain variable domain related to SEQ ID NO:343 and a light chain variable domain related to SEQ ID NO:347. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:343, and/or incorporate amino acid sequences identical to the CDR1 (SEQ ID NO:344), CDR2 (SEQ ID NO:345), and CDR3 (SEQ ID NO:346) sequences of SEQ ID NO:343. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:347, and/or incorporate amino acid sequences identical to the CDR1 (SEQ ID NO:348), CDR2 (SEQ ID NO:349), and CDR3 (SEQ ID NO:350) sequences of SEQ ID NO:347. Alternatively, the second antigen-binding site binding to EDNRB can optionally incorporate a heavy chain variable domain related to SEQ ID NO:351 and a light chain variable domain related to SEQ ID NO:355. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:351, and/or incorporate amino acid sequences identical to the CDR1 (SEQ ID NO:352), CDR2 (SEQ ID NO:353), and CDR3 (SEQ ID NO:354) sequences of SEQ ID NO:351. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:355, and/or incorporate amino acid sequences identical to the CDR1 (SEQ ID NO:356), CDR2 (SEQ ID NO:357), and CDR3 (SEQ ID NO:358) sequences of SEQ ID NO:355.

In certain embodiments, the second antigen-binding site can bind to TYRP1 and can optionally incorporate a heavy chain variable domain related to SEQ ID NO:360 and a light chain variable domain related to SEQ ID NO:364. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:360, and/or incorporate amino acid sequences identical to the CDR1 (SEQ ID NO:361), CDR2 (SEQ ID NO:362), and CDR3 (SEQ ID NO:363) sequences of SEQ ID NO:360. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:364, and/or incorporate amino acid sequences identical to the CDR1 (SEQ ID NO:365), CDR2 (SEQ ID NO:366), and CDR3 (SEQ ID NO:367) sequences of SEQ ID NO:364. Alternatively, the second antigen-binding site binding to TYRP1 can optionally incorporate a heavy chain variable domain related to SEQ ID NO:368 and a light chain variable domain related to SEQ ID NO:372. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:368, and/or incorporate amino acid sequences identical to the CDR1 (SEQ ID NO:369), CDR2 (SEQ ID NO:370), and CDR3 (SEQ ID NO:371) sequences of SEQ ID NO:368. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:372, and/or incorporate amino acid sequences identical to the CDR1 (SEQ ID NO:373), CDR2 (SEQ ID NO:374), and CDR3 (SEQ ID NO:375) sequences of SEQ ID NO:372. Alternatively, the second antigen-binding site binding to TYRP1 can optionally incorporate a heavy chain variable domain related to SEQ ID NO:376 and a light chain variable domain related to SEQ ID NO:380. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:376, and/or incorporate amino acid sequences identical to the CDR1 (SEQ ID NO:377), CDR2 (SEQ ID NO:378), and CDR3 (SEQ ID NO:379) sequences of SEQ ID NO:376. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:380, and/or incorporate amino acid sequences identical to the CDR1 (SEQ ID NO:381), CDR2 (SEQ ID NO:382), and CDR3 (SEQ ID NO:383) sequences of SEQ ID NO:380.

In certain embodiments, the second antigen-binding site can bind to OLR1 and can optionally incorporate a heavy chain variable domain related to SEQ ID NO:385 and a light chain variable domain related to SEQ ID NO:389. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:385, and/or incorporate amino acid sequences identical to the CDR1 (SEQ ID NO:386), CDR2 (SEQ ID NO:387), and CDR3 (SEQ ID NO:388) sequences of SEQ ID NO:385. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:389, and/or incorporate amino acid sequences identical to the CDR1 (SEQ ID NO:390), CDR2 (SEQ ID NO:391), and CDR3 (SEQ ID NO:392) sequences of SEQ ID NO:389. Alternatively, the second antigen-binding site binding to OLR1 can optionally incorporate a heavy chain variable domain related to SEQ ID NO:393 and a light chain variable domain related to SEQ ID NO:397. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:393, and/or incorporate amino acid sequences identical to the CDR1 (SEQ ID NO:394), CDR2 (SEQ ID NO:395), and CDR3 (SEQ ID NO:396) sequences of SEQ ID NO:393. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:397, and/or incorporate amino acid sequences identical to the CDR1 (SEQ ID NO:398), CDR2 (SEQ ID NO:399), and CDR3 (SEQ ID NO:400) sequences of SEQ ID NO:397.

In certain embodiments, the second antigen-binding site can bind to PLAUR and can optionall incorporate a heavy chain variable domain related to SEQ ID NO:405 and a light chain variable domain related to SEQ ID NO:409. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:405, and/or incorporate amino acid sequences identical to the CDR1 (SEQ ID NO:406), CDR2 (SEQ ID NO:407), and CDR3 (SEQ ID NO:408) sequences of SEQ ID NO:405. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:409, and/or incorporate amino acid sequences identical to the CDR1 (SEQ ID NO:410), CDR2 (SEQ ID NO:411), and CDR3 (SEQ ID NO:412) sequences of SEQ ID NO:409.

Alternatively, the second antigen-binding site binding to PLAUR can optionally incorporate a heavy chain variable domain related to SEQ ID NO:413 and a light chain variable domain related to SEQ ID NO:417. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:413, and/or incorporate amino acid sequences identical to the CDR1 (SEQ ID NO:414), CDR2 (SEQ ID NO:415), and CDR3 (SEQ ID NO:416) sequences of SEQ ID NO:413. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:417, and/or incorporate amino acid sequences identical to the CDR1 (SEQ ID NO:418), CDR2 (SEQ ID NO:419), and CDR3 (SEQ ID NO:420) sequences of SEQ ID NO:417.

In certain embodiments, the second antigen-binding site can bind to CCR6 and can optionally incorporate a heavy chain variable domain related to SEQ ID NO:422 and a light chain variable domain related to SEQ ID NO:426. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:422, and/or incorporate amino acid sequences identical to the CDR1 (SEQ ID NO:423), CDR2 (SEQ ID NO:424), and CDR3 (SEQ ID NO:425) sequences of SEQ ID NO:422. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:426, and/or incorporate amino acid sequences identical to the CDR1 (SEQ ID NO:427), CDR2 (SEQ ID NO:428), and CDR3 (SEQ ID NO:429) sequences of SEQ ID NO:426.

Alternatively, the second antigen-binding site binding to CCR6 can optionally incorporate a heavy chain variable domain related to SEQ ID NO:430 and a light chain variable domain related to SEQ ID NO:434. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:430, and/or incorporate amino acid sequences identical to the CDR1 (SEQ ID NO:431), CDR2 (SEQ ID NO:432), and CDR3 (SEQ ID NO:433) sequences of SEQ ID NO:430. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:434, and/or incorporate amino acid sequences identical to the CDR1 (SEQ ID NO:435), CDR2 (SEQ ID NO:436), and CDR3 (SEQ ID NO:437) sequences of SEQ ID NO:434.

In certain embodiments, the second antigen-binding site can bind to EPII A4 and can optionally incorporate a heavy chain variable domain related to SEQ ID NO:439 and a light chain variable domain related to SEQ ID NO:443. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:439, and/or incorporate amino acid sequences identical to the CDR1 (SEQ ID NO:440), CDR2 (SEQ ID NO:441), and CDR3 (SEQ ID NO:442) sequences of SEQ ID NO:439. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:443, and/or incorporate amino acid sequences identical to the CDR1 (SEQ ID NO:444), CDR2 (SEQ ID NO:445), and CDR3 (SEQ ID NO:446) sequences of SEQ ID NO:443.

Alternatively, the second antigen-binding site binding to EPH A4 can optionally incorporate a heavy chain variable domain related to SEQ ID NO:447 and a light chain variable domain related to SEQ ID NO:451. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:447, and/or incorporate amino acid sequences identical to the CDR1 (SEQ ID NO:448), CDR2 (SEQ ID NO:449), and CDR3 (SEQ ID NO:450) sequences of SEQ ID NO:447. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:451, and/or incorporate amino acid sequences identical to the CDR1 (SEQ ID NO:452), CDR2 (SEQ ID NO:453), and CDR3 (SEQ ID NO:454) sequences of SEQ ID NO:451.

In certain embodiments, the second antigen-binding site can bind to CD14 and can optionally incorporate a heavy chain variable domain related to SEQ ID NO:456 and a light chain variable domain related to SEQ ID NO:460. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:456, and/or incorporate amino acid sequences identical to the CDR1 (SEQ ID NO:457), CDR2 (SEQ ID NO:458), and CDR3 (SEQ ID NO:459) sequences of SEQ ID NO:456. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:460, and/or incorporate amino acid sequences identical to the CDR1 (SEQ ID NO:461), CDR2 (SEQ ID NO:462), and CDR3 (SEQ ID NO:463) sequences of SEQ ID NO:460.

Alternatively, the second antigen-binding site binding to CD14 can incorporate a heavy chain variable domain incorporating amino acid sequences identical to the CDR1 (SEQ ID NO:464), CDR2 (SEQ ID NO:465), and CDR3 (SEQ ID NO:466) sequences; and a light chain variable domain incorporating amino acid sequences identical to the CDR1 (SEQ ID NO:467), CDR2 (SEQ ID NO:468), and CDR3 (SEQ ID NO:469) sequences.

The second antigen-binding site binding to CD163 can optionally incorporate a heavy chain variable domain related to SEQ ID NO:471 and a light chain variable domain related to SEQ ID NO:475. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:471, and/or incorporate amino acid sequences identical to the CDR1 (SEQ ID NO:472), CDR2 (SEQ ID NO:473), and CDR3 (SEQ ID NO:474) sequences of SEQ ID NO:471. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:475, and/or incorporate amino acid sequences identical to the CDR1 (SEQ ID NO:476), CDR2 (SEQ ID NO:477), and CDR3 (SEQ ID NO:478) sequences of SEQ ID NO:475.

Alternatively, the second antigen-binding site binding to CD163 can optionally incorporate a heavy chain variable domain related to SEQ ID NO:479 and a light chain variable domain related to SEQ ID NO:483. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:479, and/or incorporate amino acid sequences identical to the CDR1 (SEQ ID NO:480), CDR2 (SEQ ID NO:481), and CDR3 (SEQ ID NO:482) sequences of SEQ ID NO:479. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:483, and/or incorporate amino acid sequences identical to the CDR1 (SEQ ID NO:484), CDR2 (SEQ ID NO:485), and CDR3 (SEQ ID NO:486) sequences of SEQ ID NO:483.

The second antigen-binding site binding to CSF3R can optionally incorporate a heavy chain variable domain related to SEQ ID NO:488 and a light chain variable domain related to SEQ ID NO:492. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:488, and/or incorporate amino acid sequences identical to the CDR1 (SEQ ID NO:489), CDR2 (SEQ ID NO:490), and CDR3 (SEQ ID NO:491) sequences of SEQ ID NO:488. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:492, and/or incorporate amino acid sequences identical to the CDR1 (SEQ ID NO:493), CDR2 (SEQ ID NO:494), and CDR3 (SEQ ID NO:495) sequences of SEQ ID NO:492.

The second antigen-binding site binding to Siglec-9 can optionally incorporate a heavy chain variable domain related to SEQ ID NO:497 and a light chain variable domain related to SEQ ID NO:501. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:497, and/or incorporate amino acid sequences identical to the CDR1 (SEQ ID NO:498), CDR2 (SEQ ID NO:499), and CDR3 (SEQ ID NO:500) sequences of SEQ ID NO:497. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:501, and/or incorporate amino acid sequences identical to the CDR1 (SEQ ID NO:502), CDR2 (SEQ ID NO:503), and CDR3 (SEQ ID NO:504) sequences of SEQ ID NO:501.

Alternatively, the second antigen-binding site binding to Siglec-9 can optionally incorporate a heavy chain variable domain related to SEQ ID NO:505 and a light chain variable domain related to SEQ ID NO:509. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:505, and/or incorporate amino acid sequences identical to the CDR1 (SEQ ID NO:506), CDR2 (SEQ ID NO:507), and CDR3 (SEQ ID NO:508) sequences of SEQ ID NO:505. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:509, and/or incorporate amino acid sequences identical to the CDR1 (SEQ ID NO:510), CDR2 (SEQ ID NO:511), and CDR3 (SEQ ID NO:512) sequences of SEQ ID NO:509.

The second antigen-binding site binding to ITGAM can optionally incorporate a heavy chain variable domain related to SEQ ID NO:514 and a light chain variable domain related to SEQ ID NO:518. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:514, and/or incorporate amino acid sequences identical to the CDR1 (SEQ ID NO:515), CDR2 (SEQ ID NO:516), and CDR3 (SEQ ID NO:517) sequences of SEQ ID NO:514. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:518, and/or incorporate amino acid sequences identical to the CDR1 (SEQ ID NO:519), CDR2 (SEQ ID NO:520), and CDR3 (SEQ ID NO:521) sequences of SEQ ID NO:518.

The second antigen-binding site binding to CCR1 can optionally incorporate a heavy chain variable domain related to SEQ ID NO:523 and a light chain variable domain related to SEQ ID NO:527. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:523, and/or incorporate amino acid sequences identical to the CDR1 (SEQ ID NO:524), CDR2 (SEQ ID NO:525), and CDR3 (SEQ ID NO:526) sequences of SEQ ID NO:523. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:527, and/or incorporate amino acid sequences identical to the CDR1 (SEQ ID NO:528), CDR2 (SEQ ID NO:529), and CDR3 (SEQ ID NO:530) sequences of SEQ ID NO:527.

Alternatively, the second antigen-binding site binding to CCR1 can optionally incorporate a heavy chain variable domain related to SEQ ID NO:531 and a light chain variable domain related to SEQ ID NO:535. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:531, and/or incorporate amino acid sequences identical to the CDR1 (SEQ ID NO:532), CDR2 (SEQ ID NO:533), and CDR3 (SEQ ID NO:534) sequences of SEQ ID NO:531. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:535, and/or incorporate amino acid sequences identical to the CDR1 (SEQ ID NO:536), CDR2 (SEQ ID NO:537), and CDR3 (SEQ ID NO:538) sequences of SEQ ID NO:535.

The second antigen-binding site binding to TLR2 can optionally incorporate a heavy chain variable domain related to SEQ ID NO:544 and a light chain variable domain related to SEQ ID NO:548. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:544, and/or incorporate amino acid sequences identical to the CDR1 (SEQ ID NO:545), CDR2 (SEQ ID NO:546), and CDR3 (SEQ ID NO:547) sequences of SEQ ID NO:544. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:548, and/or incorporate amino acid sequences identical to the CDR1 (SEQ ID NO:549), CDR2 (SEQ ID NO:550), and CDR3 (SEQ ID NO:551) sequences of SEQ ID NO:548.

Alternatively, the second antigen-binding site binding to TLR2 can optionally incorporate a heavy chain variable domain related to SEQ ID NO:552 and a light chain variable domain related to SEQ ID NO:556. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:552, and/or incorporate amino acid sequences identical to the CDR1 (SEQ ID NO:553), CDR2 (SEQ ID NO:554), and CDR3 (SEQ ID NO:555) sequences of SEQ ID NO:552. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:556, and/or incorporate amino acid sequences identical to the CDR1 (SEQ ID NO:557), CDR2 (SEQ ID NO:558), and CDR3 (SEQ ID NO:559) sequences of SEQ ID NO:556.

The second antigen-binding site binding to TLR4 can optionally incorporate a heavy chain variable domain related to SEQ ID NO:561 and a light chain variable domain related to SEQ ID NO:565. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:561, and/or incorporate amino acid sequences identical to the CDR1 (SEQ ID NO:562), CDR2 (SEQ ID NO:563), and CDR3 (SEQ ID NO:564) sequences of SEQ ID NO:561. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:565, and/or incorporate amino acid sequences identical to the CDR1 (SEQ ID NO:566), CDR2 (SEQ ID NO:567), and CDR3 (SEQ ID NO:568) sequences of SEQ ID NO:565.

Alternatively, the second antigen-binding site binding to TLR4 can optionally incorporate a heavy chain variable domain related to SEQ ID NO:569 and a light chain variable domain related to SEQ ID NO:573. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:569, and/or incorporate amino acid sequences identical to the CDR1 (SEQ ID NO:570), CDR2 (SEQ ID NO:571), and CDR3 (SEQ ID NO:572) sequences of SEQ ID NO:569. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:573, and/or incorporate amino acid sequences identical to the CDR1 (SEQ ID NO:574), CDR2 (SEQ ID NO:575), and CDR3 (SEQ ID NO:576) sequences of SEQ ID NO:573.

The second antigen-binding site binding to CCR5 can optionally incorporate a heavy chain variable domain related to SEQ ID NO:581 and a light chain variable domain related to SEQ ID NO:585. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:581, and/or incorporate amino acid sequences identical to the CDR1 (SEQ ID NO:582), CDR2 (SEQ ID NO:583), and CDR3 (SEQ ID NO:584) sequences of SEQ ID NO:581. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:585, and/or incorporate amino acid sequences identical to the CDR1 (SEQ ID NO:586), CDR2 (SEQ ID NO:587), and CDR3 (SEQ ID NO:588) sequences of SEQ ID NO:585.

Alternatively, the second antigen-binding site binding to CCR5 can optionally incorporate a heavy chain variable domain related to SEQ ID NO:589 and a light chain variable domain related to SEQ ID NO:593. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:589, and/or incorporate amino acid sequences identical to the CDR1 (SEQ ID NO:590), CDR2 (SEQ ID NO:591), and CDR3 (SEQ ID NO:592) sequences of SEQ ID NO:589. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:593, and/or incorporate amino acid sequences identical to the CDR1 (SEQ ID NO:594), CDR2 (SEQ ID NO:595), and CDR3 (SEQ ID NO:596) sequences of SEQ ID NO:593.

The second antigen-binding site binding to B7-H4 (VTCN1) can optionally incorporate a heavy chain variable domain related to SEQ ID NO:598 and a light chain variable domain related to SEQ ID NO:602. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:598, and/or incorporate amino acid sequences identical to the CDR1 (SEQ ID NO:599), CDR2 (SEQ ID NO:600), and CDR3 (SEQ ID NO:601) sequences of SEQ ID NO:598. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:602, and/or incorporate amino acid sequences identical to the CDR1 (SEQ ID NO:603), CDR2 (SEQ ID NO:604), and CDR3 (SEQ ID NO:605) sequences of SEQ ID NO:602.

Alternatively, the second antigen-binding site binding to B7-H4 (VTCN1) can optionally incorporate a heavy chain variable domain related to SEQ ID NO:606 and a light chain variable domain related to SEQ ID NO:610. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:606, and/or incorporate amino acid sequences identical to the CDR1 (SEQ ID NO:607), CDR2 (SEQ ID NO:608), and CDR3 (SEQ ID NO:609) sequences of SEQ ID NO:606. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:610, and/or incorporate amino acid sequences identical to the CDR1 (SEQ ID NO:611), CDR2 (SEQ ID NO:612), and CDR3 (SEQ ID NO:613) sequences of SEQ ID NO:610.

The second antigen-binding site binding to VISTA can optionally incorporate a heavy chain variable domain related to SEQ ID NO:615 and a light chain variable domain related to SEQ ID NO:619. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:615, and/or incorporate amino acid sequences identical to the CDR1 (SEQ ID NO:616), CDR2 (SEQ ID NO:617), and CDR3 (SEQ ID NO:618) sequences of SEQ ID NO:615. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:619, and/or incorporate amino acid sequences identical to the CDR1 (SEQ ID NO:620), CDR2 (SEQ ID NO:621), and CDR3 (SEQ ID NO:622) sequences of SEQ ID NO:619.

Alternatively, the second antigen-binding site binding to VISTA can optionally incorporate a heavy chain variable domain related to SEQ ID NO:623 and a light chain variable domain related to SEQ ID NO:627. For example, the heavy chain variable domain of the second antigen-binding site can be at least 90% (e.g., 90%, 91%, 92%, 93%, 94%, 95%, 96%, 97%, 98%, 99%, or 100%) identical to SEQ ID NO:623, and/or incorporate amino acid sequences identical to the CDR1 (SEQ ID NO:624), CDR2 (SEQ ID NO:625), and CDR3 (SEQ ID NO:626) sequences of SEQ ID NO:623. Similarly, the light chain variable domain of the second antigen-binding site can be at least 90% (e.g., 90%, 91%, 92%, 93%, 94%, 95%, 96%, 97%, 98%, 99%, or 100%) identical to SEQ ID NO:627, and/or incorporate amino acid sequences identical to the CDR1 (SEQ ID NO:628), CDR2 (SEQ ID NO:629), and CDR3 (SEQ ID NO:630) sequences of SEQ ID NO:627.

In some embodiments, the second antigen binding site incorporates a light chain variable domain having an amino acid sequence identical to the amino acid sequence of the light chain variable domain present in the first antigen binding site.

In some embodiments, the protein incorporates a portion of an antibody Fc domain sufficient to bind CD16, wherein the antibody Fc domain comprises hinge and CH2 domains, and/or amino acid sequences at least 90% identical to amino acid sequence 234-332 of a human IgG antibody.

In certain embodiments, the protein further incorporates a fourth antigen-binding site that binds to a tumor-associated antigen, which includes any antigen that is associated with cancer. For example, the fourth antigen-binding site may bind to human epidermal growth factor receptor 2 (HER2), CD20, CD33, B-cell maturation antigen (BCMA), prostate-specific membrane (PSMA), DLL3, ganglioside GD2 (GD2), CD123, anoctamin-1 (Ano1), mesothelin, carbonic anhydrase IX (CAIX), tumor-associated calcium signal transducer 2 (TROP2), carcinoembryonic antigen (CEA), claudin-18.2, receptor tyrosine kinase-like orphan receptor 1 (ROR1), trophoblast glycoprotein (5T4), glycoprotein non-metastatic melanoma protein B (GPNMB), folate receptor-alpha (FR-alpha), pregnancy-associated plasma protein A (PAPP-A), CD37, epithelial cell adhesion molecule (EpCAM), CD2, CD19, CD30, CD38, CD40, CD52, CD70, CD79b, fms-like tyrosine kinase 3 (FLT3), glypican 3 (GPC3), B7 homolog 6 (B7H6), C-C chemokine receptor type 4 (CCR4), C-X-C motif chemokine receptor 4 (CXCR4), receptor tyrosine kinase-like orphan receptor 2 (ROR2), CD133, HLA class I histocompatibility antigen, alpha chain E (HLA-E), epidermal growth factor receptor (EGFR/ERBB1), insulin-like growth factor 1-receptor (IGF1R), human epidermal growth factor receptor 3 (HER3)/ERBB-3, human epidermal growth factor receptor 4 (HER4)/ERBB-4, MUC1, tyrosine protein kinase MET (cMET), signaling lymphocytic activation molecule F7 (SLAMF7), prostate stem cell antigen (PSCA), MHC class I polypeptide-related sequence A (MICA), MHC class I polypeptide-related sequence B (MICB), TNF-related apoptosis inducing ligand receptor 1 (TRAILR1), TNF-related apoptosis inducing ligand receptor 2 (TRAILR2), melanoma associated antigen 3 (MAGE-A3), B-lymphocyte activation antigen B7.1 (B7.1), B-lymphocyte activation antigen B7.2 (B7.2), cytotoxic T-lymphocyte associated protein 4 (CTLA4), programmed cell death protein 1 (PD1), programmed cell death 1 ligand 1 (PD-L1), or CD25 antigen expressed on cancer cells.

Formulations containing one of these proteins; cells containing one or more nucleic acids expressing these proteins, and methods of enhancing tumor cell death using these proteins are also provided.

Another aspect of the invention provides a method of treating cancer in a patient. The method comprises administering to a patient in need thereof a therapeutically effective amount of the multi-specific binding protein described herein. Exemplary cancers for treatment using the DLL3-targeting multi-specific binding proteins include, for example, small cell lung cancer, large cell neuroendocrine carcinoma, glioblastoma, Ewing sarcoma, and cancers with neuroendocrine phenotype. Cancers to be treated using MUC1 (or MUC1-C)-targeting multi-specific binding proteins include, for example, gastric cancer, colorectal cancer, pancreatic cancer, breast cancer, endometrial cancer, lung cancer, bladder cancer, cervical cancer, head and neck cancer, ovarian cancer, renal cell cancer, and multiple myeloma. Cancers to be treated using Plexin-A1-targeting multi-specific binding proteins include, for example, head and neck cancer, gastric cancer, pancreatic cancer, prostate cancer, and glioma. Cancers to be treated using TNFRSF10B-targeting multi-specific binding proteins include, for example, liver cancer, pancreatic cancer, stomach cancer, renal cancer, breast cancer, ovarian cancer, endometrial cancer, and melanoma. Cancers to be treated using STEAP1-targeting multi-specific binding proteins include, for example, prostate cancer, bladder cancer, colon cancer, pancreatic cancer, ovarian cancer, testicular cancer, breast cancer, cervical cancer, and Ewing sarcoma. Cancers to be treated using CDCP1-targeting multi-specific binding proteins include, for example, colon cancer, lung cancer, gastric cancer, breast cancer, pancreatic carcer, head and neck cancer, bladder cancer, ovarian cancer, endometrial cancer, and skin cancer. Cancers to be treated using PTK7-targeting multi-specific binding proteins include, for example, lung cancer, head and neck cancer, stomach cancer, prostate cancer, testicular cancer, endometrial cancer, breast cancer, melanoma, skin cancer, and leukemia. Cancers to be treated using Axl-targeting multi-specific binding proteins include, for example, breast cancer, lung cancer, colon cancer, prostate cancer, renal cancer, esophageal cancer, liver cancer, pancreatic cancer, Kaposi's sarcoma, acute myeloid leukemia, glioma, and mesothelioma. Cancers to be treated using ERBB-3-targeting multi-specific binding proteins include, for example, prostate cancer, bladder cancer, breast cancer, ovarian cancer, colon cancer, pancreatic cancer, stomach cancer, oral cavity cancer, head and neck cancer, lung cancer, and melanoma. Cancers to be treated using EDNRB-targeting multi-specific binding proteins include, for example, melanoma, uveal melanoma, and glioma. Cancers to be treated using TYRP1-targeting multi-specific binding proteins include, for example, melanoma. Cancers to be treated using OLR1-targeting multi-specific binding proteins include, for example, gastric cancer, colorectal cancer, pancreatic cancer, prostate cancer, breast cancer, and endometrial cancer. Cancers to be treated using ADAM12-targeting multi-specific binding proteins include any cancers that express ADAM12, for example, prostate cancer, breast cancer, basal cell carcinoma, squamous cell carcinoma, melanoma, pancreatic cancer, bladder cancer, colorectal cancer, lung cancer, liver cancer, esophageal cancer, non-Hodgkin's lymphoma, ovarian cancer, and uterine cancer. Cancers to be treated using PLAUR-targeting multi-specific binding proteins include any cancers that express PLAUR, for example, breast cancer, colorectal cancer, non-small cell lung cancer, multiple myeloma, and oral cancer. Cancers to be treated using CCR6-targeting multi-specific binding proteins include any cancers that express CCR6, for example, colorectal cancer, breast cancer, cervical cancer, liver cancer, lung cancer, prostate cancer, and cutaneous T-cell lymphoma. Cancers to be treated using EPHA4-targeting multi-specific binding proteins include any cancers that express EPHA4, for example, melanoma, glioma, prostate cancer, breast cancer, small cell lung cancer, endometrial cancer, esophageal cancer, gastric cancer, and colorectal cancer.

In certain embodiments, the invention provides a method of treating cancer in a patient by targeting MDSCs and/or TAMs present in the tumor environment, and the method comprises administering to a patient in need thereof a therapeutically effective amount of the multi-specific binding proteins described herein. In some embodiments, the multi-specific binding proteins target the MDSCs and/or TAMs in a tumor environment. In some embodiments, the multi-specific binding proteins target cancer cells as well as the MDSCs and/or TAMs in the same tumor environment. Exemplary cancers to be treated may be hematological malignancies such as acute myeloid leukemia, myelodysplastic and/or myeloproliferative neoplasms, acute lymphoblastic leukemia, B-cell lymphoma, chronic neutrophilic leukemia, chronic lymphocytic leukemia, and chronic myeloid leukemia. Alternatively, the cancers to be treated may be solid tumors such as bladder cancer, colon cancer, prostate cancer, breast cancer, glioblastoma, hepatocellular carcinoma, head and neck cancer, lung cancer, ovarian cancer, pancreatic cancer, kidney cancer and melanoma.

### BRIEF DESCRIPTION OF THE DRAWINGS

FIG. 1 is a representation of a heterodimeric multi-specific binding protein. Each arm can represent either an NKG2D-binding domain, or a binding domain to a tumor-associated antigen, MDSC-associated antigen, or TAM-associated antigen. The multi-specfic binding protein further comprises an Fc domain or a portion thereof that binds to CD16. In some embodiments, the NKG2D-binding and antigen-binding domains can share a common light chain.

FIG. 2 is a representation of a heterodimeric multi-specific binding protein. Either the NKG2D binding domain, or the binding domain to a tumor-associated antigen, MDSC-associated antigen, or TAM-associated antigen can take an scFv format (right arm).

FIG. 3 is a line graph showing the binding affinity of NKG2D-binding domains (listed as clones) to human recombinant NKG2D in an ELISA assay.

FIG. 4 is a line graph showing the binding affinity of NKG2D-binding domains (listed as clones) to cynomolgus recombinant NKG2D in an ELISA assay.

FIG. 5 is a line graph showing the binding affinity of NKG2D-binding domains (listed as clones) to mouse recombinant NKG2D in an ELISA assay.

FIG. 6 is a bar graph showing the binding of NKG2D-binding domains (listed as clones) to EL4 cells expressing human NKG2D, measured by flow cytometry as mean fluorescence intensity (MFI) fold-over-background (FOB).

FIG. 7 is a bar graph showing the binding of NKG2D-binding domains (listed as clones) to EL4 cells expressing mouse NKG2D, measured by flow cytometry as mean fluorescence intensity (MFI) fold-over-background (FOB).

FIG. 8 is a line graph showing the binding affinity of NKG2D-binding domains (listed as clones) for recombinant human NKG2D-Fc in a competitive binding assay with NKG2D's natural ligand, ULBP-6.

FIG. 9 is a line graph showing the binding affinity of NKG2D-binding domains (listed as clones) for recombinant human NKG2D-Fc in a competitive binding assay with NKG2D's natural ligand, MICA.

FIG. 10 is a line graph showing the binding affinity of NKG2D-binding domains (listed as clones) for recombinant mouse NKG2D-Fc in a competitive binding assay with NKG2D's natural ligand, Rae-1 delta.

FIG. 11 is a bar graph showing activation of cells expressing human NKG2D-CD3 zeta fusion proteins by NKG2D-binding domains (listed as clones) as measured by flow cytometry and quantified as the percentage of TNFα-positive cells.

FIG. 12 is a bar graph showing activation of cells expressing mouse NKG2D-CD3 zeta fusions proteins by NKG2D-binding domains (listed as clones) as measured by flow cytometry and quantified as the percentage of TNFα-positive cells.

FIG. 13 is a bar graph showing activation of human NK cells by NKG2D-binding domains (listed as clones) as measured by flow cytometry and quantified as the percentage of IFNγ⁺/CD107a⁺ cells.

FIG. 14 is a bar graph showing activation of human NK cells by NKG2D-binding domains (listed as clones) as measured by flow cytometry and quantified as the percentage of IFNγ⁺/CD107a⁺ cells.

FIG. 15 is a bar graph showing activation of mouse NK cells by NKG2D-binding domains (listed as clones) as measured by flow cytometry and quantified as the percentage of IFNγ⁺/CD107a⁺ cells.

FIG. 16 is a bar graph showing activation of mouse NK cells by NKG2D-binding domains (listed as clones) as measured by flow cytometry and quantified as the percentage of IFNγ⁺/CD107a⁺ cells.

FIG. 17 is a bar graph showing the cytotoxic effect of NKG2D-binding domains (listed as clones) on THP-1 tumor cells as measured using a Perkin Elmer DELFIA® Cytotoxicity kit assay.

FIG. 18 is a bar graph showing the melting temperature of NKG2D-binding domains (listed as clones) measured by differential scanning fluorimetry.

FIGS. 19A-19C are bar graphs showing synergistic activation of NK cells by CD16 and NKG2D binding as measured by flow cytometry and quantified as the percentage of positive cells for NK activation markers. FIG. 19A shows the percentage of CD107a⁺ cells 4 hours post-treatment with plate-bound anti-CD16 monoclonal antibody alone, anti-NKG2D antibody alone, or anti-CD16 antibody in combination with anti-NKG2D antibody. FIG. 19B shows the percentage of IFNγ⁺ cells 4 hours post-treatment with plate-bound anti-CD16 monoclonal antibody alone, anti-NKG2D antibody alone, or anti-CD16 antibody in combination with anti-NKG2D antibody. FIG. 19C shows the percentage of CD107a⁺/IFNγ⁺ cells 4 hours post-treatment with plate-bound anti-CD16 monoclonal antibody alone, anti-NKG2D antibody alone, or anti-CD16 antibody in combination with anti-NKG2D antibody. Graphs indicate the mean (n=2)±SD. Data are representative of five independent experiments using five different healthy donors.

FIG. 20 are line graphs showing binding kinetics of murine anti-DLL3 antibodies to the extracellular domain (ECD) of DLL3 obtained by surface plasmon resonance (SPR) analysis at 37° C. Antibodies have affinities from <0.011 up to 8.44 nM. Stemcentrx antibody was used as a control.

FIG. 21A are line graphs showing binding kinetics of a murine anti-DLL3 antibody, that includes the 5E7 clone, to the different domains of DLL3 obtained by SPR analysis at 37° C. FIG. 21B is an illustration of different domains of DLL3, including an N-terminal domain (N-term), a DSL domain, EGF domains, and a C-terminal domains (C). “PM” indicates the plasma membrane.

FIG. 22A is a time-response curve showing the epitope binning of an anti-DLL3 antibody corresponding to the 9E6 clone with the Stemcentrx anti-DLL3 antibody measured by SPR analysis at 25° C. FIG. 22B is a time-response curve showing the epitope binning of an anti-DLL3 antibody corresponding to the 2F7 clone with the Stemcentrx anti-DLL3 antibody measured by SPR analysis at 25° C. FIG. 22C is a time-response curve showing the epitope binning of an anti-DLL3 antibody corresponding to the 5E7 clone with the Stemcentrx anti-DLL3 antibody measured by SPR analysis at 25° C. Anti-DLL3 antibodies were captured in a uniform orientation on the anti-mouse Fc Biacore chip, followed by injection of DLL3 ECD, followed by injection of the Stemcentrx antibody.

FIG. 23 is a bar graph showing melting temperatures of different anti-DLL3 antibodies as measured by differential scanning fluorimetry. All antibodies demonstrate melting temperatures above 70° C.

FIG. 24 is a line graph showing dose-dependent binding of anti-DLL3 antibodies to human DLL3. Antibody for DLL3 (R&D Systems, Minneapolis, Minn.; Cat #MAB4215) was used as a positive control.

FIGS. 25A-25B are line graphs showing limited cross-reactive binding of anti-DLL3 antibodies to recombinant DLL1 and DLL4. FIG. 25A shows binding of anti-DLL3 antibodies to human DLL1. Antibody for DLL1 (BioLegend, San Diego, Calif.; Cat #MHD1-314) was used as a positive control. FIG. 25B shows binding of anti-DLL3 antibodies to human DLL4. Antibody for DLL4 (BioLegend, San Diego, Calif.; Cat #MHD4-46) was used as a positive control.

FIG. 26A is a flow cytometry histogram profile showing the binding of anti-DLL3 antibodies (2 μg/mL) to DLL3 expressed on NCI-H82 cells. FIG. 26B is a line graph showing a dose-response profile of anti-DLL3 antibody binding to DLL3 on NCI-H82 cells.

FIG. 27A is a line graph showing antibody internalization by SHP-77 cells expressing DLL3 after 1-3 hours of incubation with anti-DLL3 antibodies. FIG. 27B is a line graph showing antibody internalization by DMS-79 cells expressing DLL3 after 1-3 hours of incubation with anti-DLL3 antibodies.

FIG. 28A is a line graph showing the dose-response curves of an anti-DLL3 multi-specific binding protein and an anti-DLL3 monoclonal antibody binding to the full-length extracellular domain of DLL3 (DLL3-D1-6). FIG. 28B is a line graph showing the dose-response curves of anti-DLL3 multi-specific binding protein and anti-DLL3 monoclonal antibody binding to a truncated form of the DLL3 extracellular domain (DLL3-1-2).

FIG. 29A is a line graph showing activation of purified human NK cells co-cultured with RPMI-8226 cells expressing DLL3-D1-6, as measured by flow cytometry and quantified as the percentage of IFNγ⁺/CD107a⁺ cells. FIG. 29B is a line graph showing activation of purified human NK cells co-cultured with RPMI-8226 cells expressing DLL3-D1-6, as measured by flow cytometry and quantified as the mean fluorescence intensity (MFI) of CD69⁺-labelled cells.

FIG. 30A is a line graph showing lysis of RPMI-8226 cells expressing DLL3-D1-2 by IL-2 activated peripheral blood mononuclear cells (PBMCs) in the presence of anti-DLL3 multi-specific binding protein or anti-DLL3 monoclonal antibody. FIG. 30B is a bar graph showing % killing of RPMI-8226 cells expressing DLL3-D1-6 by IL-2 activated human NK cells in the presence of anti-DLL3 multi-specific binding protein or anti-DLL3 monoclonal antibody.

FIG. 31 is a representative illustration of a multi-specific binding protein in a Triomab form.

FIG. 32 is a representative illustration of a multi-specific binding protein in a KiH Common Light Chain (LC) form.

FIG. 33 is a representative illustration of a multi-specific binding protein in a dual-variable domain immunoglobulin (DVD-Ig™) form.

FIG. 34 is representative illustration of a multi-specific binding protein in an Orthogonal Fab interface (Ortho-Fab) form.

FIG. 35 is a representative illustration of a multi-specific binding protein in a 2-in-1 Ig form.

FIG. 36 is a representative illustration of a multi-specific binding protein in an electrostatic-steering (ES) form.

FIG. 37 is a representative illustration of a multi-specific binding protein in a controlled Fab-Arm Exchange (cFAE) form.

FIG. 38 is representative illustration of a multi-specific binding protein in a strand-exchange engineered domain (SEED) body form.

FIG. 39 is a representative illustration of a multi-specific binding protein in a LuZ-Y form.

FIG. 40 is a representative illustration of a multi-specific binding protein in a Cov-X-Body form.

FIGS. 41A-41B are representative illustrations of a multi-specific binding protein in a κλ-Body form. FIG. 41A is an exemplary representative illustration of one form of a κλ-Body; FIG. 41B is an exemplary representative illustration of another κλ-Body.

FIG. 42 is a representative illustration of a multi-specific binding protein in a one-arm single chain (OAsc)-Fab form.

FIG. 43 is a representative illustration of a multi-specific binding protein in a DuetMab form.

FIG. 44 is a representative illustration of a multi-specific binding protein in a CrossmAb form.

FIG. 45 is a representative illustration of a multi-specific binding protein in a Fit-Ig form.

FIG. 46 is a series of sensograms generated from a Biacore analysis of DLL3-His binding to murine and humanized variants of the 5E7 antibody.

FIG. 47 is a graph showing the binding of humanized variants of the 5E7 antibody to RPMI-8226 cells transduced to express DLL3, compared to binding of a chimeric protein of murine 5E7 variable regions and human IgG1/Igκ constant regions to the same cells.

### DETAILED DESCRIPTION

The invention provides multi-specific binding proteins that bind a tumor-associated antigen selected from DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, and EPHA4 on a cancer cell, and the NKG2D receptor and CD16 receptor on natural killer cells to activate the natural killer cells. In certain embodiments, the multi-specific binding proteins further include an additional antigen-binding site that binds a tumor-associated antigen.

The invention also provides multi-specific binding proteins that bind the NKG2D receptor and CD16 receptor on natural killer cells, and an antigen selected from CD14, CD163, CSF3R, Siglec-9, ITGAM, VISTA, B7-H4, CCR1, LRRC25, PTAFR, SIRPB1, TLR2, TLR4, CD300LB, ATP1A3, and CCR5. In some embodiments, the multi-specific binding proteins further include an additional antigen-binding site that binds a tumor-associated antigen.

The invention additionally provides pharmaceutical compositions comprising such multi-specific binding proteins, and therapeutic methods using such multi-specific binding proteins and pharmaceutical compositions, for purposes such as treating cancer. Various aspects of the invention are set forth below in sections; however, aspects of the invention described in one particular section are not to be limited to any particular section.

To facilitate an understanding of the present invention, a number of terms and phrases are defined below.

The terms “a” and “an” as used herein mean “one or more” and include the plural unless the context is inappropriate.

As used herein, the term “antigen-binding site” refers to the part of the immunoglobulin molecule that participates in antigen binding. In human antibodies, the antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide.

The term “tumor associated antigen” as used herein means any antigen including but not limited to a protein, glycoprotein, ganglioside, carbohydrate, or lipid that is associated with cancer. Such antigen can be expressed on malignant cells or in the tumor microenvironment such as on tumor-associated blood vessels, extracellular matrix, mesenchymal stroma, or immune infiltrates.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Remington's Pharmaceutical Sciences, 15th Ed., Mack Publishing Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Exemplary bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Exemplary salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Proteins

The invention provides multi-specific binding proteins that bind a tumor-associated antigen selected from DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, and EPHA4 on cancer cells and the NKG2D receptor and CD16 receptor on natural killer cells to activate the natural killer cell. The multi-specific binding proteins are useful in the pharmaceutical compositions and therapeutic methods described herein. Binding of the multi-specific binding protein to the NKG2D receptor and CD16 receptor on a natural killer cell enhances the activity of the natural killer cell toward destruction of a cancer cell. Binding of the multi-specific binding protein to DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, and/or EPHA4 on a cancer cell brings the cancer cell into proximity to the natural killer cell, which facilitates direct and indirect destruction of the cancer cell by the natural killer cell.

The invention also provides multi-specific binding proteins that bind to the NKG2D receptor and CD16 receptor on natural killer cells, and an antigen selected from CD14, CD163, CSF3R, Siglec-9, ITGAM, VISTA, B7-H4, CCR1, LRRC25, PTAFR, SIRPB1, TLR2, TLR4, CD300LB, ATP1A3, and CCR5. In some embodiments, the multi-specific binding proteins further include an additional antigen-binding site that binds a tumor-associated antigen. The multi-specific binding proteins are useful in the pharmaceutical compositions and therapeutic methods described herein. Binding of the multi-specific binding proteins to the NKG2D receptor and CD16 receptor on a natural killer cell enhances the activity of the natural killer cell toward destruction of cells expressing CD14, CD163, CSF3R, Siglec-9, ITGAM, VISTA, B7-H4, CCR1, LRRC25, PTAFR, SIRPB1, TLR2, TLR4, CD300LB, ATP1A3, and/or CCR5 antigen. Binding of the multi-specific binding proteins to CD14, CD163, CSF3R, Siglec-9, ITGAM, VISTA, B7-H4, CCR1, LRRC25, PTAFR, SIRPB1, TLR2, TLR4, CD300LB, ATP1A3, and/or CCR5-expressing cells brings the cells into proximity with the natural killer cell, which facilitates direct and indirect destruction of the cells by the natural killer cell. In some embodiments, the cells are MDSCs. In some other embodiments, the cells are TAMs. Destruction of the MDSCs and/or TAMs by the natural killer cell may de-repress/enhance the immune response against tumor cells, which co-exist with the MDSCs and/or TAMs in a tumor microenvironment. In some embodiments, the multi-specific binding proteins that include an additional antigen-binding site for a tumor-associated antigen, enhances the activity of the natural killer cell toward destruction of cells expressing the tumor-associated antigen as well as the cells expressing CD14, CD163, CSF3R, Siglec-9, ITGAM, VISTA, B7-H4, CCR1, LRRC25, PTAFR, SIRPB1, TLR2, TLR4, CD300LB, ATP1A3, and/or CCR5. Further description of some exemplary multi-specific binding proteins is provided below.

The first component of the multi-specific binding proteins binds to NKG2D receptor-expressing cells, which can include but are not limited to NK cells, γδ T cells and CD8⁺ αβ T cells. Upon NKG2D binding, the multi-specific binding proteins may block natural ligands, such as ULBP6 and MICA, from binding to NKG2D and activating NKG2D receptors.

In certain embodiments, the second component of the multi-specific binding proteins binds to DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, or EPHA4-expressing cells. DLL3-expressing cells may be found for example in, but not limited to, small cell lung cancer, large cell neuroendocrine carcinoma, glioblastoma, Ewing sarcoma, and cancers with neuroendocrine phenotype. MUC1 (or MUC1-C)-expressing cells may be found for example in, but not limited to, gastric cancer, colorectal cancer, pancreatic cancer, breast cancer, endometrial cancer, lung cancer, bladder cancer, cervical cancer, head and neck cancer, ovarian cancer, renal cell cancer, and multiple myeloma. Plexin-A1-expressing cells may be found for example in, but not limited to, head and neck cancer, gastric cancer, pancreatic cancer, prostate cancer, and glioma. TNFRSF10B-expressing cells may be found for example in, but not limited to, liver cancer, pancreatic cancer, stomach cancer, renal cancer, breast cancer, ovarian cancer, endometrial cancer, and melanoma. STEAP1-expressing cells may be found for example in, but not limited to, prostate cancer, bladder cancer, colon cancer, pancreas cancer, ovarian cancer, testicular cancer, breast cancer, cervical cancer and Ewing sarcoma. CDCP1-expressing cells may be found for example in, but not limited to, colon cancer, lung cancer, gastric cancer, breast cancer, pancreatic cancer, head and neck cancer, bladder cancer, ovarian cancer, endometrial cancer, and skin cancer. PTK7-expressing cells may be found for example in, but not limited to, lung cancer, head and neck cancer, stomach cancer, prostate cancer, testicular cancer, endometrial cancer, breast cancer, melanoma, skin cancer, and leukemia. AXL-expressing cells may be found for example in, but not limited to, breast cancer, lung cancer, colon cancer, prostate cancer, renal cancer, esophageal cancer, liver cancer, pancreatic cancer, Kaposi's sarcoma, acute myeloid leukemia, glioma, and mesothelioma. ERBB-3-expressing cells may be found for example in, but not limited to, prostate cancer, bladder cancer, breast cancer, ovarian cancer, colon cancer, pancreatic cancer, stomach cancer, oral cavity cancer, head and neck cancer, lung cancer and melanoma. EDNRB-expressing cells may be found for example in, but not limited to, melanoma, uveal melanoma, and glioma. TYRP1-expressing cells may be found for example in, but not limited to, melanoma. OLR1-expressing cells may be found for example in, but not limited to, gastric cancer, colorectal cancer, pancreatic cancer, prostate cancer, breast cancer, and endometrial cancer. ADAM12-expressing cells may be found, for example in, but not limited to, prostate cancer, breast cancer, basal cell carcinoma, squamous cell carcinoma, melanoma, pancreatic cancer, bladder cancer, colorectal cancer, lung cancer, liver cancer, esophageal cancer, Non-Hodgkin's lymphoma, ovarian cancer, and uterine cancer. PLAUR-expressing cells may be found, for example in, but not limited to, breast cancer, colorectal cancer, non-small cell lung cancer, and oral cancer. CCR6-expressing cells may be found, for example in, but not limited to, colorectal cancer, breast cancer, cervical cancer, liver cancer, lung cancer, and cutaneous T-cell lymphoma. EPHA4-expressing cells may be found, for example in, but not limited to, melanoma, glioma, prostate cancer, breast cancer, small cell lung cancer, endometrial cancer, esophageal cancer, gastric cancer, and colorectal cancer.

In certain other embodiments, the second component of the multi-specific binding proteins binds to an antigen selected from CD14, CD163, CSF3R, Siglec-9, ITGAM, VISTA, B7-H4, CCR1, LRRC25, PTAFR, SIRPB1, TLR2, TLR4, CD300LB, ATP1A3, and CCR5. In some embodiments, one or more of CD14, CD163, CSF3R, Siglec-9, ITGAM, VISTA, B7-H4, CCR1, LRRC25, PTAFR, SIRPB1, TLR2, TLR4, CD300LB, ATP1A3, and CCR5 antigens are expressed by MDSCs and/or TAMs in the micro-microenvironment of a variety of hematological and solid tumors, such as acute myeloid leukemia, myelodysplastic and/or myeloproliferative neoplasms, acute lymphoblastic leukemia, B-cell lymphoma, chronic neutrophilic leukemia, chronic lymphocytic leukemia, chronic myeloid leukemia, as well as bladder cancer, colon cancer, prostate cancer, breast cancer, glioblastoma, hepatocellular carcinoma, head and neck cancer, lung cancer, ovarian cancer, pancreatic cancer, kidney cancer and melanoma.

In some embodiments, multi-specific binding proteins described herein further incorporate an additional antigen-binding site that binds to a tumor-associated antigen, which includes any antigen that is associated with cancer, such as but not limited to a protein, glycoprotein, ganglioside, carbohydrate, or lipid. Such antigens can be expressed on malignant cells or in the tumor microenvironment such as on tumor-associated blood vessels, extracellular matrix, mesenchymal stroma, or immune infiltrates. For example, the additional antigen-binding site can bind to HER2, CD20, CD33, BCMA, PSMA, DLL3, GD2, CD123, Ano1, Mesothelin, CAIX, TROP2, CEA, Claudin-18.2, ROR1, 5T4, GPNMB, FR-alpha, PAPP-A, CD37, EpCAM, CD2, CD19, CD30, CD38, CD40, CD52, CD70, CD79b, FLT3, GPC3, B7H6, CCR4, CXCR4, ROR2, CD133, HLA-E, EGFR/ERBB-1, IGF1R, HER3/ERBB-3, HER4/ERBB-4, MUC1, cMET, SLAMF7, PSCA, MICA, MICB, TRAILR1, TRAILR2, MAGE-A3, B7.1, B7.2, CTLA4, PD1, PD-L1, or CD25 antigen expressed on cancer cells. Accordingly, in some embodiments, binding of the multi-specific binding proteins to a tumor-associated antigen expressed on cancer cells brings the cells into proximity with the natural killer cell, which facilitates direct and indirect destruction of the cancer cells by the natural killer cell in addition to the destruction of MDSCs and/or TAMs by the natural killer cell.

The third component for the multi-specific binding proteins binds to cells expressing CD16, an Fc receptor on the surface of leukocytes including natural killer cells, macrophages, neutrophils, eosinophils, mast cells, and follicular dendritic cells.

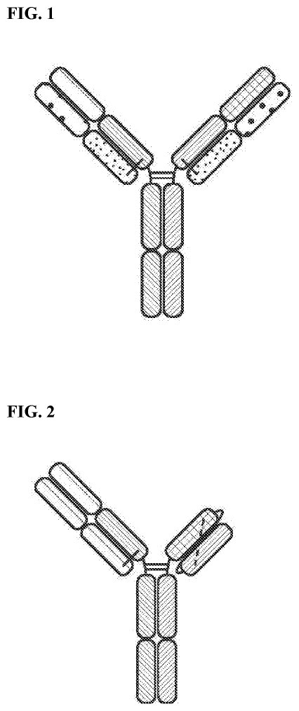

The multi-specific binding proteins described herein can take various formats. For example, one format is a heterodimeric, multi-specific antibody including a first immunoglobulin heavy chain, a first immunoglobulin light chain, a second immunoglobulin heavy chain and a second immunoglobulin light chain (FIG. 1). The first immunoglobulin heavy chain includes a first Fc (hinge-CH2-CH3) domain, a first heavy chain variable domain and optionally a first CH1 heavy chain domain. The first immunoglobulin light chain includes a first light chain variable domain and a first light chain constant domain. The first immunoglobulin light chain, together with the first immunoglobulin heavy chain, forms an antigen-binding site that binds NKG2D. The second immunoglobulin heavy chain comprises a second Fc (hinge-CH2-CH3) domain, a second heavy chain variable domain and optionally a second CH1 heavy chain domain. The second immunoglobulin light chain includes a second light chain variable domain and a second light chain constant domain. In certain embodiments, the second immunoglobulin light chain, together with the second immunoglobulin heavy chain, forms an antigen-binding site that binds a tumor-associated antigen selected from DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, and EPHA4. In certain other embodiments, the second immunoglobulin light chain, together with the second immunoglobulin heavy chain, forms an antigen-binding site that binds CD14, CD163, CSF3R, Siglec-9, ITGAM, VISTA, B7-H4, CCR1, LRRC25, PTAFR, SIRPB1, TLR2, TLR4, CD300LB, ATP1A3, or CCR5. The first Fc domain and second Fc domain together are able to bind to CD16 (FIG. 1). In some embodiments, the first immunoglobulin light chain is identical to the second immunoglobulin light chain.

Another exemplary format involves a heterodimeric, multi-specific antibody including a first immunoglobulin heavy chain, a second immunoglobulin heavy chain and an immunoglobulin light chain (FIG. 2). The first immunoglobulin heavy chain includes a first Fc (hinge-CH2-CH3) domain fused via either a linker or an antibody hinge to a single-chain variable fragment (scFv) composed of a heavy variable domain and light chain variable domain which pair and bind NKG2D; bind a tumor-associated antigen selected from DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, and EPHA4; or bind a MDSC-associated or TAM-associated antigen selected from CD14, CD163, CSF3R, Siglec-9, ITGAM, VISTA, B7-H4, CCR1, LRRC25, PTAFR, SIRPB1, TLR2, TLR4, CD300LB, ATP1A3, and CCR5. The second immunoglobulin heavy chain includes a second Fc (hinge-CH2-CH3) domain, a second heavy chain variable domain and optionally a CH1 heavy chain domain. The immunoglobulin light chain includes a light chain variable domain and a light chain constant domain. The second immunoglobulin heavy chain pairs with the immunoglobulin light chain and binds to NKG2D; binds a tumor-associated antigen selected from DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, and EPHA4; or binds a MDSC-associated or TAM-associated antigen selected from CD14, CD163, CSF3R, Siglec-9, ITGAM, VISTA, B7-H4, CCR1, LRRC25, PTAFR, SIRPB1, TLR2, TLR4, CD300LB, ATP1A3, and CCR5. The first Fc domain and the second Fc domain together are able to bind to CD16 (FIG. 2).

One or more additional binding motifs may be fused to the C-terminus of the constant region CH3 domain, optionally via a linker sequence. In certain embodiments, the antigen-binding site could be a single-chain or disulfide-stabilized variable region (scFv) or could form a tetravalent or trivalent molecule.

In some embodiments, the multi-specific binding protein is in a Triomab form, which is a trifunctional, bispecific antibody that maintains an IgG-like shape (e.g., the multi-specific binding protein represented in FIG. 31). This chimeric bispecific antibody comprises of two half antibodies, each with one light and one heavy chain, that originate from two parental antibodies. The Triomab form may be a heterodimer, comprising of ½ of a rat antibody and ½ of a mouse antibody.

In some embodiments, the multi-specific binding protein is in a KiH Common Light Chain (LC) form, which incorporates the knobs-into-holes (KiH) technology (e.g., the multi-specific binding protein represented in FIG. 32). The KiH Common LC form is a heterodimer comprising a Fab which binds to a first target, a Fab which binds to a second target, and an Fc domain stabilized by heterodimerization mutations. The two Fabs each comprise a heavy chain and light chain, wherein the heavy chain of each Fab differs from the other, and the light chain that pairs with each respective heavy chain is common to both Fabs.

The KiH technology involves engineering CH3 domains to create either a “knob” or a “hole” in each heavy chain to promote heterodimerization. Introduction of a “knob” in one CH3 domain (CH3A) comprises substitution of a small residue with a bulky one (e.g., T366W_{CH3A} in EU numbering). To accommodate the “knob,” a complementary “hole” surface is introduced on the other CH3 domain (CH3B) by replacing the closest neighboring residues to the knob with smaller ones (e.g., T366S/L368A/Y407V_{CH3B}). The “hole” mutation was optimized by structure-guided phage library screening (Atwell S., et al. (1997) J. Mol. Biol.; 270(1):26-35.). X-ray crystal structures of KiH Fc variants (Elliott J. M., et al. (2014) J. Mol. Biol.; 426(9):1947-57.; Mimoto F., et al. (2014) Mol. Immunol.; 58(1):132-8.) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

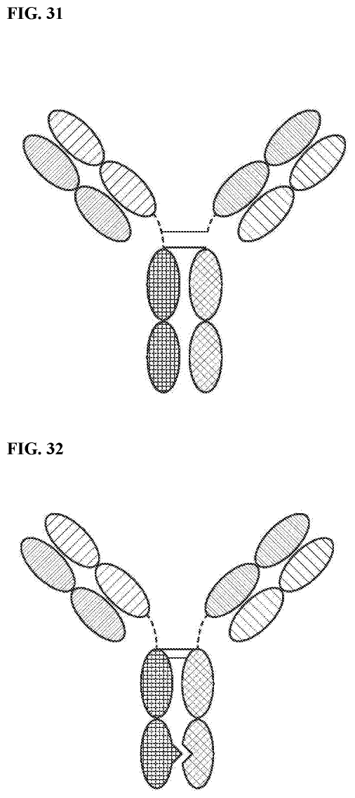

In some embodiments, the multi-specific binding protein is in a dual-variable domain immunoglobulin (DVD-Ig™) form, which is a tetravalent IgG-like structure comprising the target-binding domains of two monoclonal antibodies and flexible naturally occurring linkers (e.g., FIG. 33). The DVD-Ig™ form is homodimeric comprising a variable domain targeting antigen 2 fused to the N-terminus of a Fab variable domain targeting antigen 1. The representative multi-specific binding protein shown in FIG. 33 comprises an unmodified Fc.

In some embodiments, the multi-specific binding protein is in an Orthogonal Fab interface (Ortho-Fab) form (e.g., the multi-specific binding protein represented in FIG. 34). In the Ortho-Fab IgG approach (Lewis S. M., et al. (2014) Nat. Biotechnol.; 32(2):191-8.), structure-based regional design introduces complementary mutations at the LC and HC_{VH-CH1} interface in only one Fab, without any changes being made to the other Fab.

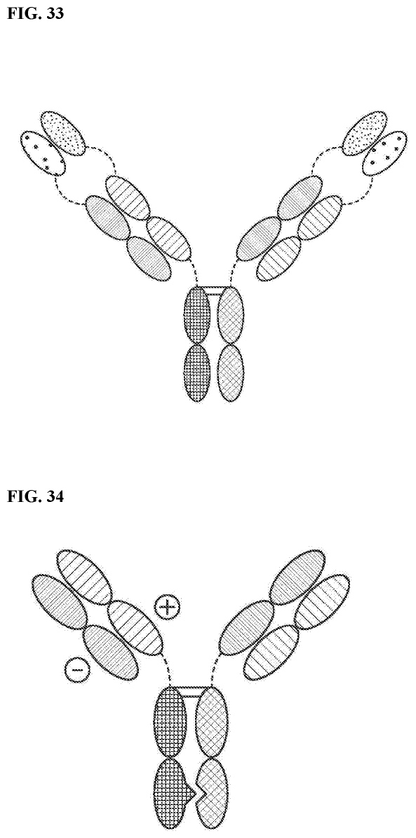

In some embodiments, the multi-specific binding protein is in a 2-in-1 Ig form (e.g., the multi-specific binding protein represented in FIG. 35).

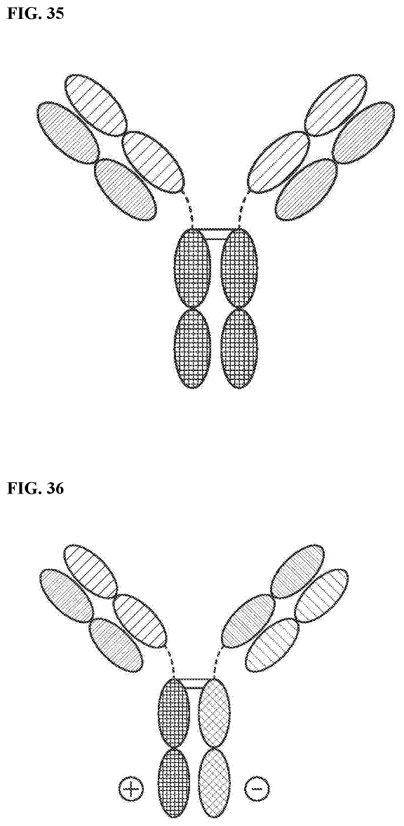

In some embodiments, the multi-specific binding protein is an electrostatic-steering (ES) form, which is a heterodimer comprising two different Fabs binding to targets 1 and target 2, and an Fc domain (e.g., the multi-specific binding protein represented in FIG. 36). Heterodimerization is ensured by electrostatic steering mutations in the Fc domain.

In some embodiments, the multi-specific binding protein is in a controlled Fab-Arm Exchange (cFAE) form (e.g., the multi-specific binding protein represented in FIG. 37). The cFAE form is a bispecific heterodimer comprising two different Fabs binding to targets 1 and 2, wherein a LC-HC pair (half-molecule) has been swapped with a LC-HC pair from another molecule. Heterodimerization is ensured by mutations in the Fc.

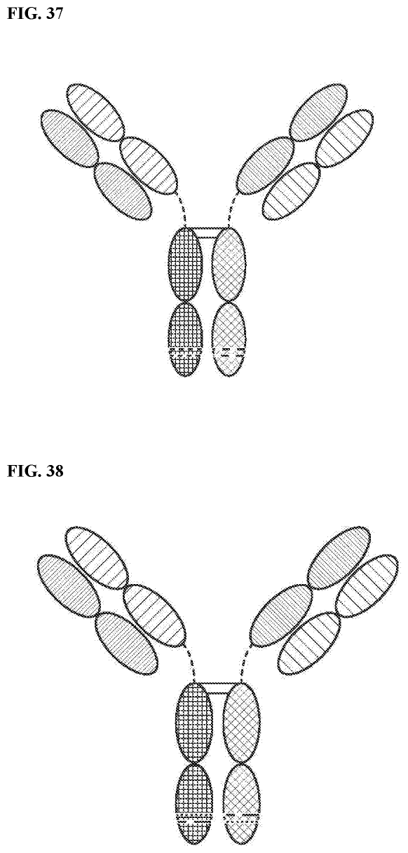

In some embodiments, the multi-specific binding protein is in a strand-exchange engineered domain (SEED) body form (e.g., the multi-specific binding protein represented in FIG. 38). The SEED platform was designed to generate asymmetric and bispecific antibody-like molecules in order to expand the therapeutic applications of natural antibodies. This protein engineering platform is based on exchanging structurally related sequences of immunoglobulin classes within the conserved CH3 domains (e.g., alternating segments of IgA and IgG CH3 domain sequences). The SEED design allows efficient generation of heterodimers, while disfavoring homodimerization of SEED CH3 domains. (Muda M. et al. (2011) Protein Eng. Des. Sel.; 24(5):447-54.).

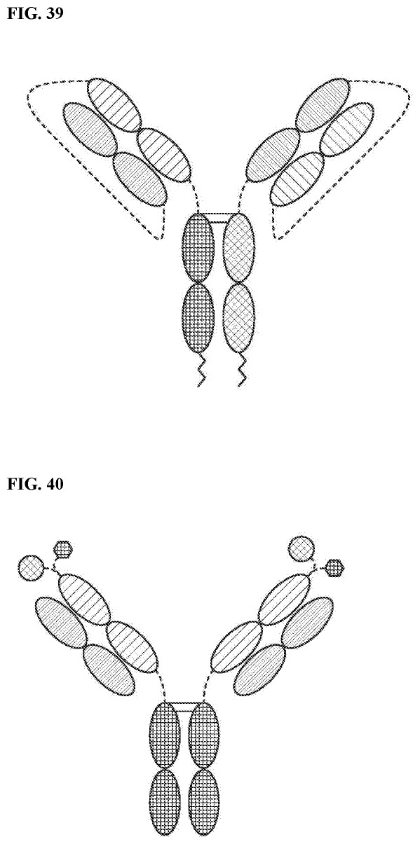

In some embodiments, the multi-specific binding protein is in a LuZ-Y form (e.g., the multi-specific binding protein represented in FIG. 39). The LuZ-Y form is a heterodimer comprising two different scFabs binding to targets 1 and 2, fused to an Fc domain. Heterodimerization is ensured through the introduction of leucine zipper motifs fused to the C-terminus of the Fc domain (Wranik B. J. et al. (2012) J. Biol. Chem.; 287:43331-9.).

In some embodiments, the multi-specific binding protein is in a Cov-X-Body form (e.g., the multi-specific binding protein represented in FIG. 40). Bispecific Cov-X-Bodies comprise a scaffold antibody having a pharmacophore peptide heterodimer covalently linked to each Fab arm, wherein one molecule of the peptide heterodimer binds to a first target and the other molecule of the peptide heterodimer binds to a second target, and wherein the two molecules are joined by an azetidinone linker. Whereas the pharmacophores are responsible for functional activities, the antibody scaffold imparts long half-life and Ig-like distribution. The pharmacophores can be chemically optimized or replaced with other pharmacophores to generate optimized or unique bispecific antibodies. (Doppalapudi V. R. et al. (2010) PNAS; 107(52):22611-22616.).

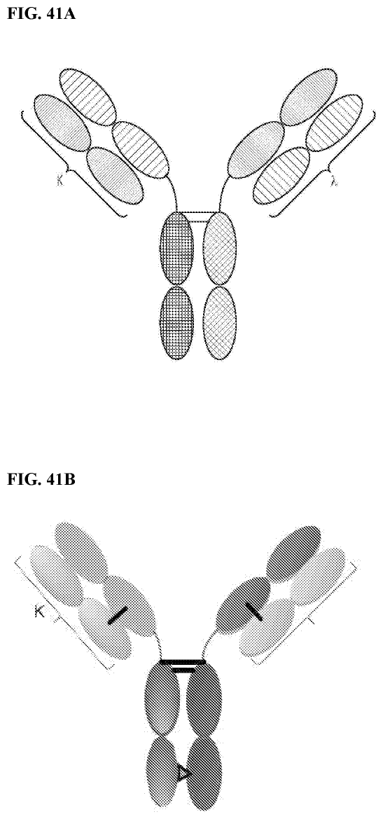

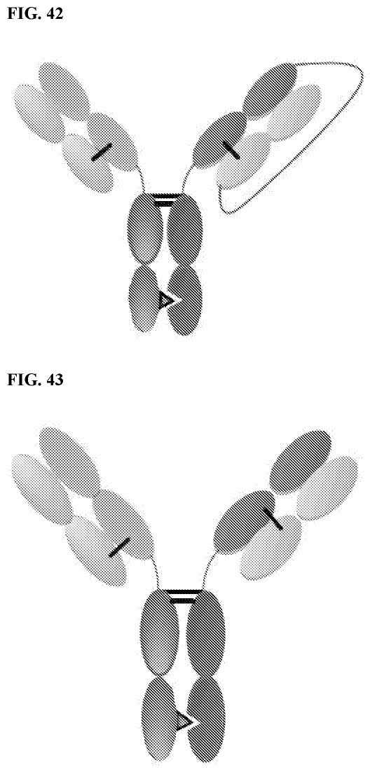

In some embodiments, the multi-specific binding protein is in a κλ-Body form, which is a heterodimer comprising two different Fabs fused to Fc domains stabilized by heterodimerization mutations (e.g., the multi-specific binding protein represented in FIG. 41). A first Fab binding target 1 comprises a kappa LC, and a second Fab binding target 2 comprises a lambda LC. FIG. 41A is an exemplary representation of one form of a κλ-Body; FIG. 41B is an exemplary representation of another κλ-Body.

In some embodiments, the multi-specific binding protein is in a one-arm single chain (OAsc)-Fab form (e.g., the multi-specific binding protein represented in FIG. 42). The OAsc-Fab form is a heterodimer that includes a Fab binding to target 1 and an scFab binding to target 2 fused to an Fc domain. Heterodimerization is ensured by mutations in the Fc domain.

In some embodiments, the multi-specific binding protein is in a DuetMab form (e.g., the multi-specific binding protein represented in FIG. 43). The DuetMab form is a heterodimercomprising two different Fabs binding to targets 1 and 2, and an Fc domain stabilized by heterodimerization mutations. The two different Fabs comprise different S-S bridges that ensure correct LC and HC pairing.

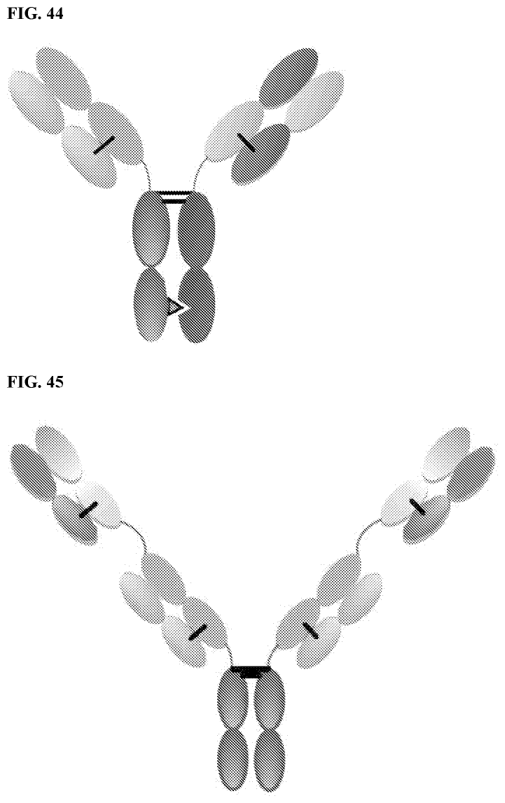

In some embodiments, the multi-specific binding protein is in a CrossmAb form (e.g., the multi-specific binding protein represented in FIG. 44). The CrossmAb form is a heterodimer comprising two different Fabs binding to targets 1 and 2, and an Fc domain stabilized by heterodimerization mutations. CL and CH1 domains and VH and VL domains are switched, e.g., CH1 is fused in-line with VL, while CL is fused in-line with VH.

In some embodiments, the multi-specific binding protein is in a Fit-Ig form (e.g., the multi-specific binding protein represented in FIG. 45). The Fit-Ig form is a homodimer comprising a Fab binding to target 2 fused to the N-terminus of the HC of a Fab that binds to target 1. The representative multi-specific binding protein of FIG. 45 comprises an unmodified Fcdomain.

Table 1 lists peptide sequences of heavy chain variable domains and light chain variable domains that, in combination, can bind to NKG2D. Unless indicated otherwise, the CDR sequences provided in Table 1 are determined under Kabat. The NKG2D binding domains can vary in their binding affinity to NKG2D, nevertheless, they all activate human NKG2D and NK cells.

TABLE 1

| | Heavy chain variable region | Light chain variable region |
|---|---|---|
| Clones | amino acid sequence | amino acid sequence |
| ADI- | QVQLQQWGAGLLKPSETLSLTCAV | DIQMTQSPSTLSASVGDRVTI |
| 27705 | YGGSFSGYYWSWIRQPPGKGLEWI | TCRASQSISSWLAWYQQKPG |
| | GEIDHSGSTNYNPSLKSRVTISVDTS | KAPKLLIYKASSLESGVPSRF |
| | KNQFSLKLSSVTAADTAVYYCARA | SGSGSGTEFTLTISSLQPDDF |
| | RGPWSFDPWGQGTLVTVSS | ATYYCQQYNSYPITFGGGTK |
| | (SEQ ID NO: 1) | VEIK |
| | CDR1 (SEQ ID NO: 105)- | (SEQ ID NO: 2) |
| | GSFSGYYWS (non-Kabat) or GYYWS | |
| | [SEQ ID NO: 634] | |
| | CDR2 (SEQ ID NO: 106)- | |
| | EIDHSGSTNYNPSLKS | |
| | CDR3 (SEQ ID NO: 107)- | |
| | ARARGPWSFDP (non-Kabat) or | |
| | ARGPWSFDP [SEQ ID NO: 635] | |
| ADI- | QVQLQQWGAGLLKPSETLSLTCAV | EIVLTQSPGTLSLSPGERATL |
| 27724 | YGGSFSGYYWSWIRQPPGKGLEWI | SCRASQSVSSSYLAWYQQKP |
| | GEIDHSGSTNYNPSLKSRVTISVDTS | GQAPRLLIYGASSRATGIPDR |
| | KNQFSLKLSSVTAADTAVYYCARA | FSGSGSGTDFTLTISRLEPEDF |
| | RGPWSFDPWGQGTLVTVSS | AVYYCQQYGSSPITFGGGTK |
| | (SEQ ID NO: 3) | VEIK |
| | | (SEQ ID NO: 4) |
| ADI- | QVQLQQWGAGLLKPSETLSLTCAV | DIQMTQSPSTLSASVGDRVTI |
| 27740 | YGGSFSGYYWSWIRQPPGKGLEWI | TCRASQSIGSWLAWYQQKP |
| (A40) | GEIDHSGSTNYNPSLKSRVTISVDTS | GKAPKLLIYKASSLESGVPSR |
| | KNQFSLKLSSVTAADTAVYYCARA | FSGSGSGTEFTLTISSLQPDDF |
| | RGPWSFDPWGQGTLVTVSS | ATYYCQQYHSFYTFGGGTK |
| | (SEQ ID NO: 5) | VEIK |
| | | (SEQ ID NO: 6) |
| ADI- | QVQLQQWGAGLLKPSETLSLTCAV | DIQMTQSPSTLSASVGDRVTI |
| 27741 | YGGSFSGYYWSWIRQPPGKGLEWI | TCRASQSIGSWLAWYQQKP |
| | GEIDHSGSTNYNPSLKSRVTISVDTS | GKAPKLLIYKASSLESGVPSR |
| | KNQFSLKLSSVTAADTAVYYCARA | FSGSGSGTEFTLTISSLQPDDF |
| | RGPWSFDPWGQGTLVTVSS | ATYYCQQSNSYYTFGGGTK |
| | (SEQ ID NO: 7) | VEIK |
| | | (SEQ ID NO: 8) |
| ADI- | QVQLQQWGAGLLKPSETLSLTCAV | DIQMTQSPSTLSASVGDRVTI |
| 27743 | YGGSFSGYYWSWIRQPPGKGLEWI | TCRASQSISSWLAWYQQKPG |
| | GEIDHSGSTNYNPSLKSRVTISVDTS | KAPKLLIYKASSLESGVPSRF |
| | KNQFSLKLSSVTAADTAVYYCARA | SGSGSGTEFTLTISSLQPDDF |
| | RGPWSFDPWGQGTLVTVSS | ATYYCQQYNSYPTFGGGTK |
| | (SEQ ID NO: 9) | VEIK |
| | | (SEQ ID NO: 10) |
| ADI- | QVQLQQWGAGLLKPSETLSLTCAV | ELQMTQSPSSLSASVGDRVTI |
| 28153 | YGGSFSGYYWSWIRQPPGKGLEWI | TCRTSQSISSYLNWYQQKPG |
| | GEIDHSGSTNYNPSLKSRVTISVDTS | QPPKLLIYWASTRESGVPDR |
| | KNQFSLKLSSVTAADTAVYYCARA | FSGSGSGTDFTLTISSLQPEDS |
| | RGPWGFDPWGQGTLVTVSS | ATYYCQQSYDIPYTFGQGTK |
| | (SEQ ID NO: 11) | LEIK |
| | | (SEQ ID NO: 12) |
| ADI- | QVQLQQWGAGLLKPSETLSLTCAV | DIQMTQSPSTLSASVGDRVTI |
| 28226 | YGGSFSGYYWSWIRQPPGKGLEWI | TCRASQSISSWLAWYQQKPG |
| (C26) | GEIDHSGSTNYNPSLKSRVTISVDTS | KAPKLLIYKASSLESGVPSRF |
| | KNQFSLKLSSVTAADTAVYYCARA | SGSGSGTEFTLTISSLQPDDF |
| | RGPWSFDPWGQGTLVTVSS | ATYYCQQYGSFPITFGGGTK |
| | (SEQ ID NO: 13) | VEIK |
| | | (SEQ ID NO: 14) |
| ADI- | QVQLQQWGAGLLKPSETLSLTCAV | DIQMTQSPSTLSASVGDRVTI |
| 28154 | YGGSFSGYYWSWIRQPPGKGLEWI | TCRASQSISSWLAWYQQKPG |
| | GEIDHSGSTNYNPSLKSRVTISVDTS | KAPKLLIYKASSLESGVPSRF |
| | KNQFSLKLSSVTAADTAVYYCARA | SGSGSGTDFTLTISSLQPDDF |
| | RGPWSFDPWGQGTLVTVSS | ATYYCQQSKEVPWTFGQGT |
| | (SEQ ID NO: 15) | KVEIK |
| | | (SEQ ID NO: 16) |
| ADI- | QVQLQQWGAGLLKPSETLSLTCAV | DIQMTQSPSTLSASVGDRVTI |
| 29399 | YGGSFSGYYWSWIRQPPGKGLEWI | TCRASQSISSWLAWYQQKPG |
| | GEIDHSGSTNYNPSLKSRVTISVDTS | KAPKLLIYKASSLESGVPSRF |
| | KNQFSLKLSSVTAADTAVYYCARA | SGSGSGTEFTLTISSLQPDDF |
| | RGPWSFDPWGQGTLVTVSS | ATYYCQQYNSFPTFGGGTK |
| | (SEQ ID NO: 17) | VEIK |
| | | (SEQ ID NO: 18) |
| ADI- | QVQLQQWGAGLLKPSETLSLTCAV | DIQMTQSPSTLSASVGDRVTI |
| 29401 | YGGSFSGYYWSWIRQPPGKGLEWI | TCRASQSIGSWLAWYQQKP |
| | GEIDHSGSTNYNPSLKSRVTISVDTS | GKAPKLLIYKASSLESGVPSR |
| | KNQFSLKLSSVTAADTAVYYCARA | FSGSGSGTEFTLTISSLQPDDF |
| | RGPWSFDPWGQGTLVTVSS | ATYYCQQYDIYPTFGGGTKV |
| | (SEQ ID NO: 19) | EIK |
| | | (SEQ ID NO: 20) |
| ADI- | QVQLQQWGAGLLKPSETLSLTCAV | DIQMTQSPSTLSASVGDRVTI |
| 29403 | YGGSFSGYYWSWIRQPPGKGLEWI | TCRASQSISSWLAWYQQKPG |
| | GEIDHSGSTNYNPSLKSRVTISVDTS | KAPKLLIYKASSLESGVPSRF |
| | KNQFSLKLSSVTAADTAVYYCARA | SGSGSGTEFTLTISSLQPDDF |
| | RGPWSFDPWGQGTLVTVSS | ATYYCQQYDSYPTFGGGTK |
| | (SEQ ID NO: 21) | VEIK |
| | | (SEQ ID NO: 22) |
| ADI- | QVQLQQWGAGLLKPSETLSLTCAV | DIQMTQSPSTLSASVGDRVTI |
| 29405 | YGGSFSGYYWSWIRQPPGKGLEWI | TCRASQSISSWLAWYQQKPG |
| | GEIDHSGSTNYNPSLKSRVTISVDTS | KAPKLLIYKASSLESGVPSRF |
| | KNQFSLKLSSVTAADTAVYYCARA | SGSGSGTEFTLTISSLQPDDF |
| | RGPWSFDPWGQGTLVTVSS | ATYYCQQYGSFPTFGGGTK |
| | (SEQ ID NO: 23) | VEIK |
| | | (SEQ ID NO: 24) |
| ADI- | QVQLQQWGAGLLKPSETLSLTCAV | DIQMTQSPSTLSASVGDRVTI |
| 29407 | YGGSFSGYYWSWIRQPPGKGLEWI | TCRASQSISSWLAWYQQKPG |
| | GEIDHSGSTNYNPSLKSRVTISVDTS | KAPKLLIYKASSLESGVPSRF |
| | KNQFSLKLSSVTAADTAVYYCARA | SGSGSGTEFTLTISSLQPDDF |
| | RGPWSFDPWGQGTLVTVSS | ATYYCQQYQSFPTFGGGTK |
| | (SEQ ID NO: 25) | VEIK |
| | | (SEQ ID NO: 26) |
| ADI- | QVQLQQWGAGLLKPSETLSLTCAV | DIQMTQSPSTLSASVGDRVTI |
| 29419 | YGGSFSGYYWSWIRQPPGKGLEWI | TCRASQSISSWLAWYQQKPG |
| | GEIDHSGSTNYNPSLKSRVTISVDTS | KAPKLLIYKASSLESGVPSRF |
| | KNQFSLKLSSVTAADTAVYYCARA | SGSGSGTEFTLTISSLQPDDF |
| | RGPWSFDPWGQGTLVTVSS | ATYYCQQYSSFSTFGGGTKV |
| | (SEQ ID NO: 27) | EIK |
| | | (SEQ ID NO: 28) |
| ADI- | QVQLQQWGAGLLKPSETLSLTCAV | DIQMTQSPSTLSASVGDRVTI |
| 29421 | YGGSFSGYYWSWIRQPPGKGLEWI | TCRASQSISSWLAWYQQKPG |
| | GEIDHSGSTNYNPSLKSRVTISVDTS | KAPKLLIYKASSLESGVPSRF |
| | KNQFSLKLSSVTAADTAVYYCARA | SGSGSGTEFTLTISSLQPDDF |
| | RGPWSFDPWGQGTLVTVSS | ATYYCQQYESYSTFGGGTK |
| | (SEQ ID NO: 29) | VEIK |
| | | (SEQ ID NO: 30) |
| ADI- | QVQLQQWGAGLLKPSETLSLTCAV | DIQMTQSPSTLSASVGDRVTI |
| 29424 | YGGSFSGYYWSWIRQPPGKGLEWI | TCRASQSISSWLAWYQQKPG |
| | GEIDHSGSTNYNPSLKSRVTISVDTS | KAPKLLIYKASSLESGVPSRF |
| | KNQFSLKLSSVTAADTAVYYCARA | SGSGSGTEFTLTISSLQPDDF |
| | RGPWSFDPWGQGTLVTVSS | ATYYCQQYDSFITFGGGTKV |
| | (SEQ ID NO: 31) | EIK |
| | | (SEQ ID NO: 32) |
| ADI- | QVQLQQWGAGLLKPSETLSLTCAV | DIQMTQSPSTLSASVGDRVTI |
| 29425 | YGGSFSGYYWSWIRQPPGKGLEWI | TCRASQSISSWLAWYQQKPG |
| | GEIDHSGSTNYNPSLKSRVTISVDTS | KAPKLLIYKASSLESGVPSRF |
| | KNQFSLKLSSVTAADTAVYYCARA | SGSGSGTEFTLTISSLQPDDF |
| | RGPWSFDPWGQGTLVTVSS | ATYYCQQYQSYPTFGGGTK |
| | (SEQ ID NO: 33) | VEIK |
| | | (SEQ ID NO: 34) |
| ADI- | QVQLQQWGAGLLKPSETLSLTCAV | DIQMTQSPSTLSASVGDRVTI |
| 29426 | YGGSFSGYYWSWIRQPPGKGLEWI | TCRASQSIGSWLAWYQQKP |
| | GEIDHSGSTNYNPSLKSRVTISVDTS | GKAPKLLIYKASSLESGVPSR |
| | KNQFSLKLSSVTAADTAVYYCARA | FSGSGSGTEFTLTISSLQPDDF |
| | RGPWSFDPWGQGTLVTVSS | ATYYCQQYHSFPTFGGGTK |
| | (SEQ ID NO: 35) | VEIK |
| | | (SEQ ID NO: 36) |
| ADI- | QVQLQQWGAGLLKPSETLSLTCAV | DIQMTQSPSTLSASVGDRVTI |
| 29429 | YGGSFSGYYWSWIRQPPGKGLEWI | TCRASQSIGSWLAWYQQKP |
| | GEIDHSGSTNYNPSLKSRVTISVDTS | GKAPKLLIYKASSLESGVPSR |
| | KNQFSLKLSSVTAADTAVYYCARA | FSGSGSGTEFTLTISSLQPDDF |
| | RGPWSFDPWGQGTLVTVSS | ATYYCQQYELYSYTFGGGT |
| | (SEQ ID NO: 37) | KVEIK |
| | | (SEQ ID NO: 38) |
| ADI- | QVQLQQWGAGLLKPSETLSLTCAV | DIQMTQSPSTLSASVGDRVTI |
| 29447 | YGGSFSGYYWSWIRQPPGKGLEWI | TCRASQSISSWLAWYQQKPG |
| (F47) | GEIDHSGSTNYNPSLKSRVTISVDTS | KAPKLLIYKASSLESGVPSRF |
| | KNQFSLKLSSVTAADTAVYYCARA | SGSGSGTEFTLTISSLQPDDF |
| | RGPWSFDPWGQGTLVTVSS | ATYYCQQYDTFITFGGGTKV |
| | (SEQ ID NO: 39) | EIK |
| | | (SEQ ID NO: 40) |
| ADI- | QVQLVQSGAEVKKPGSSVKVSCKA | DIVMTQSPDSLAVSLGERATI |
| 27727 | SGGTFSSYAISWVRQAPGQGLEWM | NCKSSQSVLYSSNNKNYLA |
| | GGIIPIFGTANYAQKFQGRVTITADE | WYQQKPGQPPKLLIYWAST |
| | STSTAYMELSSLRSEDTAVYYCAR | RESGVPDRFSGSGSGTDFTLT |
| | GDSSIRHAYYYYGMDVWGQGTTV | ISSLQAEDVAVYYCQQYYST |
| | TVSS | PITFGGGTKVEIK |
| | (SEQ ID NO: 41) | (SEQ ID NO: 42) |
| | CDR1 (SEQ ID NO: 43)- | CDR1 (SEQ ID NO: 46)- |
| | GTFSSYAIS (non-Kabat) or SYAIS | KSSQSVLYSSNNKNYLA |
| | [SEQ ID NO: 636] | CDR2 (SEQ ID NO: 47)- |
| | CDR2 (SEQ ID NO: 44)- | WASTRES |
| | GIIPIFGTANYAQKFQG | CDR3 (SEQ ID NO: 48)- |
| | CDR3 (SEQ ID NO: 45)- | QQYYSTPIT |
| | ARGDSSIRHAYYYYGMDV (non- | |
| | Kabat) or GDSSIRHAYYYYGMDV | |
| | [SEQ ID NO: 637] | |
| ADI- | QLQLQESGPGLVKPSETLSLTCTVS | EIVLTQSPATLSLSPGERATL |
| 29443 | GGSISSSSYYWGWIRQPPGKGLEWI | SCRASQSVSRYLAWYQQKP |
| (F43) | GSIYYSGSTYYNPSLKSRVTISVDTS | GQAPRLLIYDASNRATGIPA |
| | KNQFSLKLSSVTAADTAVYYCARG | RFSGSGSGTDFTLTISSLEPED |
| | SDRFHPYFDYWGQGTLVTVSS | FAVYYCQQFDTWPPTFGGG |
| | (SEQ ID NO: 49) | TKVEIK |
| | CDR1 (SEQ ID NO: 51)- | (SEQ ID NO: 50) |
| | GSISSSSYYWG (non-Kabat) or | CDR1 (SEQ ID NO: 54)- |
| | SSSYYWG [SEQ ID NO: 638] | RASQSVSRYLA |
| | CDR2 (SEQ ID NO: 52)- | CDR2 (SEQ ID NO: 55)- |
| | SIYYSGSTYYNPSLKS | DASNRAT |
| | CDR3 (SEQ ID NO: 53)- | CDR3 (SEQ ID NO: 56)- |
| | ARGSDRFHPYPDY (non-Kabat) or | QQFDTWPPT |
| | GSDRFHPYFDY [SEQ ID NO: 639] | |
| ADI- | QVQLQQWGAGLLKPSETLSLTCAV | DIQMTQSPSTLSASVGDRVTI |
| 29404 | YGGSFSGYYWSWIRQPPGKGLEWI | TCRASQSISSWLAWYQQKPG |
| (F04) | GEIDHSGSTNYNPSLKSRVTISVDTS | KAPKLLIYKASSLESGVPSRF |
| | KNQFSLKLSSVTAADTAVYYCARA | SGSGSGTEFTLTISSLQPDDF |
| | RGPWSFDPWGQGTLVTVSS | ATYYCEQYDSYPTFGGGTK |
| | (SEQ ID NO: 57) | VEIK |
| | | (SEQ ID NO: 58) |
| ADI- | QVQLVQSGAEVKKPGSSVKVSCKA | DIVMTQSPDSLAVSLGERATI |
| 28200 | SGGTFSSYAISWVRQAPGQGLEWM | NCESSQSLLNSGNQKNYLT |
| | GGIIPIFGTANYAQKFQGRVTITADE | WYQQKPGQPPKPLIYWAST |
| | STSTAYMELSSLRSEDTAVYYCAR | RESGVPDRFSGSGSGTDFTLT |
| | RGRKASGSFYYYYGMDVWGQGTT | ISSLQAEDVAVYYCQNDYSY |
| | VTVSS | PYTFGQGTKLEIK |
| | (SEQ ID NO: 59) | (SEQ ID NO: 60) |
| | CDR1 (SEQ ID NO: 108)- | CDR1 (SEQ ID NO: 111)- |
| | GTFSSYAIS | ESSQSLLNSGNQKNYLT |
| | CDR2 (SEQ ID NO: 109)- | CDR2 (SEQ ID NO: 112)- |
| | GIIPIFGTANYAQKFQG | WASTRES |
| | CDR3 (SEQ ID NO: 110)- | CDR3 (SEQ ID NO: 113)- |
| | ARRGRKASGSFYYYYGMDV | QNDYSYPYT |
| ADI-29379 | QVQLVQSGAEVKKPGASVKVSCK | EIVMTQSPATLSVSPGERATL |
| (E79) | ASGYTFTSYYMHWVRQAPGQGLE | SCRASQSVSSNLAWYQQKP |
| | WMGIINPSGGSTSYAQKFQGRVTM | GQAPRLLIYGASTRATGIPAR |
| | TRDTSTSTVYMELSSLRSEDTAVYY | FSGSGSGTEFTLTISSLQSEDF |
| | CARGAPNYGDTTHDYYYMDVWG | AVYYCQQYDDWPFTFGGGT |
| | KGTTVTVSS | KVEIK |
| | (SEQ ID NO: 61) | (SEQ ID NO: 62) |
| | CDR1 (SEQ ID NO: 63)- | CDR1 (SEQ ID NO: 66)- |
| | YTFTSYYMH (non-Kabat) or SYYMH | RASQSVSSNLA |
| | [SEQ ID NO: 640] | CDR2 (SEQ ID NO: 67)- |
| | CDR2 (SEQ ID NO: 64)- | GASTRAT |
| | IINPSGGSTSYAQKFQG | CDR3 (SEQ ID NO: 68)- |
| | CDR3 (SEQ ID NO: 65)- | QQYDDWPFT |
| | ARGAPNYGDTTHDYYYMDV (non- | |
| | Kabat) or GAPNYGDTTHDYYYMDV | |
| | [SEQ ID NO: 641] | |
| ADI- | QVQLVQSGAEVKKPGASVKVSCK | EIVLTQSPGTLSLSPGERATL |
| 29463 | ASGYTFTGYYMHWVRQAPGQGLE | SCRASQSVSSNLAWYQQKP |
| (F63) | WMGWINPNSGGTNYAQKFQGRVT | GQAPRLLIYGASTRATGIPAR |
| | MTRDTSISTAYMELSRLRSDDTAV | FSGSGSGTEFTLTISSLQSEDF |
| | YYCARDTGEYYDTDDHGMDVWG | AVYYCQQDDYWPPTFGGGT |
| | QGTTVTVSS | KVEIK |
| | (SEQ ID NO: 69) | (SEQ ID NO: 70) |
| | CDR1 (SEQ ID NO: 71)- | CDR1 (SEQ ID NO: 74)- |
| | YTFTGYYMH (non-Kabat) or | RASQSVSSNLA |
| | GYYMH [SEQ ID NO: 642] | CDR2 (SEQ ID NO: 75)- |
| | CDR2 (SEQ ID NO: 72)- | GASTRAT |
| | WINPNSGGTNYAQKFQG | CDR3 (SEQ ID NO: 76)- |
| | CDR3 (SEQ ID NO: 73)- | QQDDYWPPT |
| | ARDTGEYYDTDDHGMDV (non- | |
| | Kabat) or DTGEYYDTDDHGMDV | |
| | [SEQ ID NO: 643] | |
| ADI- | EVQLLESGGGLVQPGGSLRLSCAAS | DIQMTQSPSSVSASVGDRVTI |
| 27744 | GFTFSSYAMSWVRQAPGKGLEWV | TCRASQGIDSWLAWYQQKP |
| (A44) | SAISGSGGSTYYADSVKGRFTISRD | GKAPKLLIYAASSLQSGVPS |
| | NSKNTLYLQMNSLRAEDTAVYYC | RFSGSGSGTDFTLTISSLQPE |
| | AKDGGYYDSGAGDYWGQGTLVTV | DFATYYCQQGVSYPRTFGG |
| | SS | GTKVEIK |
| | (SEQ ID NO: 77) | (SEQ ID NO: 78) |
| | CDR1 (SEQ ID NO: 79)- | CDR1 (SEQ ID NO: 82)- |
| | FTFSSYAMS (non-Kabat) or SYAMS | RASQGIDSWLA |
| | [SEQ ID NO: 644] | CDR2 (SEQ ID NO: 83)- |
| | CDR2 (SEQ ID NO: 80)- | AASSLQS |
| | AISGSGGSTYYADSVKG | CDR3 (SEQ ID NO: 84)- |
| | CDR3 (SEQ ID NO: 81)- | QQGVSYPRT |
| | AKDGGYYDSGAGDY (non-Kabat) or | |
| | DGGYYDSGAGDY | |
| | [SEQ ID NO: 645] | |
| ADI- | EVQLVESGGGLVKPGGSLRLSCAA | DIQMTQSPSSVSASVGDRVTI |
| 27749 | SGFTFSSYSMNWVRQAPGKGLEW | TCRASQGISSWLAWYQQKP |
| (A49) | VSSISSSSSYIYYADSVKGRFTISRD | GKAPKLLIYAASSLQSGVPS |
| | NAKNSLYLQMNSLRAEDTAVYYC | RFSGSGSGTDFTLTISSLQPE |
| | ARGAPMGAAAGWFDPWGQGTLVT | DFATYYCQQGVSFPRTFGGG |
| | VSS | TKVEIK |
| | (SEQ ID NO: 85) | (SEQ ID NO: 86) |
| | CDR1 (SEQ ID NO: 87)- | CDR1 (SEQ ID NO: 90)- |
| | FTFSSYSMN or SYSMN [SEQ ID | RASQGISSWLA |
| | NO: 646] | CDR2 (SEQ ID NO: 91)- |
| | CDR2 (SEQ ID NO: 88)- | AASSLQS |
| | SISSSSSYIYYADSVKG | CDR3 (SEQ ID NO: 92)- |
| | CDR3 (SEQ ID NO: 89)- | QQGVSFPRT |
| | ARGAPMGAAAGWFDP (non-Kabat) | |
| | or GAPMGAAAGWFDP [SEQ ID | |
| | NO: 647] | |
| ADI- | QVQLVQSGAEVKKPGASVKVSCK | EIVLTQSPATLSLSPGERATL |
| 29378 | ASGYTFTSYYMHWVRQAPGQGLE | SCRASQSVSSYLAWYQQKP |
| (E78) | WMGIINPSGGSTSYAQKFQGRVTM | GQAPRLLIYDASNRATGIPA |
| | TRDTSTSTVYMELSSLRSEDTAVYY | RFSGSGSGTDFTLTISSLEPED |
| | CAREGAGFAYGMDYYYMDVWGK | FAVYYCQQSDNWPFTFGGG |
| | GTTVTVSS | TKVEIK |
| | (SEQ ID NO: 93) | (SEQ ID NO: 94) |
| | CDR1 (SEQ ID NO: 95)- | CDR1 (SEQ ID NO: 98)- |
| | YTFTSYYMH (non-Kabat) or SYYMH | RASQSVSSYLA |
| | [SEQ ID NO: 648] | CDR2 (SEQ ID NO: 99)- |
| | CDR2 (SEQ ID NO: 96)- | DASNRAT |
| | IINPSGGSTSYAQKFQG | CDR3 (SEQ ID NO: 100)- |
| | CDR3 (SEQ ID NO: 97)- | QQSDNWPFT |
| | AREGAGFAYGMDYYYMDV (non- | |
| | Kabat) or EGAGFAYGMDYYYMDV | |
| | [SEQ ID NO: 649] | |
| A49MI | EVQLVESGGGLVKPGGSLRLSCAA | DIQMTQSPSSVSASVGDRVTI |
| | SGFTFSSYSMNWVRQAPGKGLEW | TCRASQGISSWLAWYQQKP |
| | VSSISSSSSYIYYADSVKGRFTISRD | GKAPKLLIYAASSLQSGVPS |
| | NAKNSLYLQMNSLRAEDTAVYYC | RFSGSGSGTDFTLTISSLQPE |
| | ARGAPIGAAAGWFDPWGQGTLVT | DFATYYCQQGVSFPRTFGGG |
| | VSS [SEQ ID NO: 650] | TKVEIK |
| | CDR1: FTFSSYSMN [SEQ ID NO: 87] | (SEQ ID NO: 86) |
| | non kabat or SYSMN [SEQ ID NO: 646] | CDR1 (SEQ ID NO: 90)- |
| | CDR2: SISSSSSYIYYADSVKG | RASQGISSWLA |
| | [SEQ ID NO: 88] | CDR2 (SEQ ID NO: 91)- |
| | CDR3: (non-Kabat) | AASSLQS |
| | ARGAPIGAAAGWFDP [SEQ ID | CDR3 (SEQ ID NO: 92)- |
| | NO: 651] or GAPIGAAAGWFDP | QQGVSFPRT |
| | [SEQ ID NO: 652] | |
| A49MQ | EVQLVESGGGLVKPGGSLRLSCAA | DIQMTQSPSSVSASVGDRVTI |
| | SGFTFSSYSMNWVRQAPGKGLEW | TCRASQGISSWLAWYQQKP |
| | VSSISSSSSYIYYADSVKGRFTISRD | GKAPKLLIYAASSLQSGVPS |
| | NAKNSLYLQMNSLRAEDTAVYYC | RFSGSGSGTDFTLTISSLQPE |
| | ARGAPQGAAGWFDPWGQGTLVT | DFATYYCQQGVSFPRTFGGG |
| | VSS | TKVEIK |
| | [SEQ ID NO: 6531 | (SEQ ID NO: 86) |
| | CDR1: FTFSSYSMN [SEQ ID NO: 87] | CDR1 (SEQ ID NO: 90)- |
| | non kabat or SYSMN [SEQ ID NO:646] | RASQGISSWLA |
| | CDR2: SISSSSSYIYYADSVKG | CDR2 (SEQ ID NO: 91)- |
| | [SEQ ID NO: 88] | AASSLQS |
| | CDR3 (non-Kabat) [SEQ ID NO: 654]- | CDR3 (SEQ ID NO: 92)- |
| | ARGAPQGAAAGWFDP or CDR3 | QQGVSFPRT |
| | [SEQ ID NO: 655]- | |
| | GAPQGAAAGWFDP | |
| A49ML | EVQLVESGGGLVKPGGSLRLSCAA | DIQMTQSPSSVSASVGDRVTI |
| | SGFTFSSYSMNWVRQAPGKGLEW | TCRASQGISSWLAWYQQKP |
| | VSSISSSSSYIYYADSVKGRFTISRD | GKAPKLLIYAASSLQSGVPS |
| | NAKNSLYLQMNSLRAEDTAVYYC | RFSGSGSGTDFTLTISSLQPE |
| | ARGAPLGAAAGWFDPWGQGTLVT | DFATYYCQQGVSFPRTFGGG |
| | VSS | TKVEIK |
| | [SEQ ID NO: 656] | (SEQ ID NO: 86) |
| | CDR1: FTFSSYSMN [SEQ ID NO: 87] | CDR1 (SEQ ID NO: 90)- |
| | non kabat or SYSMN [SEQ ID NO: 646] | RASQGISSWLA |
| | CDR2: SISSSSSYIYYADSVKG | CDR2 (SEQ ID NO: 91)- |
| | [SEQ ID NO: 88] | AASSLQS |
| | CDR3 (non-Kabat) [SEQ ID NO: 657]- | CDR3 (SEQ ID NO: 92)- |
| | ARGAPLGAAAGWFDP or CDR3 | QQGVSFPRT |
| | [SEQ ID NO: 658]- | |
| | GAPLGAAAGWFDP | |
| A49MF | EVQLVESGGGLVKPGGSLRLSCAA | DIQMTQSPSSVSASVGDRVTI |
| | SGFTFSSYSMNWVRQAPGKGLEW | TCRASQGISSWLAWYQQKP |
| | VSSISSSSSYIYYADSVKGRFTISRD | GKAPKLLIYAASSLQSGVPS |
| | NAKNSLYLQMNSLRAEDTAVYYC | RFSGSGSGTDFTLTISSLQPE |
| | ARGAPFGAAAGWFDPWGQGTLVT | DFATYYCQQGVSFPRTFGGG |
| | VSS | TKVEIK |
| | [SEQ ID NO: 659] | (SEQ ID NO: 86) |
| | CDR1: FTFSSYSMN [SEQ ID NO: 87] | CDR1 (SEQ ID NO: 90)- |
| | non kabat or SYSMN [SEQ ID NO: 646] | RASQGISSWLA |
| | CDR2: SISSSSSYIYYADSVKG | CDR2 (SEQ ID NO: 91)- |
| | [SEQ ID NO: 88] | AASSLQS |
| | CDR3 (non-Kabat) [SEQ ID NO: 660]- | CDR3 (SEQ ID NO: 92)- |
| | ARGAPFGAAAGWFDP or CDR3 | QQGVSFPRT |
| | [SEQ ID NO: 661]- | |
| | GAPFGAAAGWFDP | |
| A49MV | EVQLVESGGGLVKPGGSLRLSCAA | DIQMTQSPSSVSASVGDRVTI |
| | SGFTFSSYSMNWVRQAPGKGLEW | TCRASQGISSWLAWYQQKP |
| | VSSISSSSSYIYYADSVKGRFTISRD | GKAPKLLIYAASSLQSGVPS |
| | NAKNSLYLQMNSLRAEDTAVYYC | RFSGSGSGTDFTLTISSLQPE |
| | ARGAPVGAAAGWFDPWGQGTLVT | DFATYYCQQGVSFPRTFGGG |
| | VSS | TKVEIK |
| | [SEQ ID NO: 662] | (SEQ ID NO: 86) |
| | CDR1: FTFSSYSMN [SEQ ID NO: 87] | CDR1 (SEQ ID NO: 90)- |
| | non kabat or SYSMN [SEQ ID NO: 646] | RASQGISSWLA |
| | CDR2: SISSSSSYIYYADSVKG | CDR2 (SEQ ID NO: 91)- |
| | [SEQ ID NO: 88] | AASSLQS |
| | CDR3 (non-Kabat) [SEQ ID NO: 663]- | CDR3 (SEQ ID NO: 92)- |
| | ARGAPVGAAAGWFDP or CDR3 | QQGVSFPRT |
| | [SEQ ID NO: 664]- | |
| | GAPVGAAAGWFDP | |
| A49- | EVQLVESGGGLVKPGGSLRLSCAA | DIQMTQSPSSVSASVGDRVTI |
| consensus | SGFTFSSYSMNWVRQAPGKGLEW | TCRASQGISSWLAWYQQKP |
| | VSSISSSSSYIYYADSVKGRFTISRD | GKAPKLLIYAASSLQSGVPS |
| | NAKNSLYLQMNSLRAEDTAVYYC | RFSGSGSGTDFTLTISSLQPE |
| | ARGAPXGAAAGWFDPWGQGTLVT | DFATYYCQQGVSFPRTFGGG |
| | TKVEIK | (SEQ ID NO: 86) |
| | VSS, wherein X is M, L, I, V, Q, or F | CDR1 (SEQ ID NO: 90)- |
| | [SEQ ID NO: 665] | RASQGISSWLA |
| | CDR1: FTFSSYSMN [SEQ ID NO: 87] | CDR2 (SEQ ID NO: 91)- |
| | non kabat or SYSMN [SEQ ID NO: 646] | AASSLQS |
| | CDR2: SISSSSSYIYYADSVKG | CDR3 (SEQ ID NO: 92)- |
| | [SEQ ID NO: 88] | QQGVSFPRT |
| | CDR3 (non-Kabat) [SEQ ID NO: 666]- | |
| | ARGAPXGAAAGWFDP or CDR3 | |
| | [SEQ ID NO: 667]- | |
| | GAPXGAAAGWFDP, wherein X is M, | |
| | L, I, V, Q, or F | |

Alternatively, a heavy chain variable domain defined by SEQ ID NO:101 can be paired with a light chain variable domain defined by SEQ ID NO:102 to form an antigen-binding site that can bind to NKG2D, as illustrated in U.S. Pat. No. 9,273,136.

SEQ ID NO: 101
QVQLVESGGGLVKPGGSLRLSCAASGFTFSSYGMHWVRQAPGKGLEWVA
FIRYDGSNKYYADSVKGRFTISRDNSKNTLYLQMNSLRAEDTAVYYCAK
DRGLGDGTYFDYWGQGTTVTVSS
SEQ ID NO: 102
QSALTQPASVSGSPGQSITISCSGSSSNIGNNAVNWYQQLPGKAPKLLI
YYDDLLPSGVSDRFSGSKSGTSAFLAISGLQSEDEADYYCAAWDDSLNG
PVFGGGTKLTVL

Alternatively, a heavy chain variable domain defined by SEQ ID NO:103 can be paired with a light chain variable domain defined by SEQ ID NO:104 to form an antigen-binding site that can bind to NKG2D, as illustrated in U.S. Pat. No. 7,879,985.

SEQ ID NO: 103
QVHLQESGPGLVKPSETLSLTCTVSDDSISSYYWSWIRQPPGKGLEWIG
HISYSGSANYNPSLKSRVTISVDTSKNQFSLKLSSVTAADTAVYYCANW
DDAFNIWGQGTMVTVSS
SEQ ID NO: 104
EIVLTQSPGTLSLSPGERATLSCRASQSVSSSYLAWYQQKPGQAPRLLI
YGASSRATGIPDRFSGSGSGTDFTLTISRLEPEDFAVYYCQQYGSSPWT
FGQGTKVEIK

In certain embodiments, the present disclosure provides multi-specific binding proteins that bind to the NKG2D receptor and CD16 receptor on natural killer cells, and the tumor-associated antigen DLL3. Table 2 lists peptide sequences of heavy chain variable domains and light chain variable domains that, in combination, can bind to DLL3.

TABLE 2

| | Heavy chain variable | Light chain variable |
|---|---|---|
| Clones | domain peptide sequence | domain peptide sequence |
| 2F7 | QVQLQQSGAELMKPGASVKLS | NIMMTQSPSSLAVSAGEKVTMS |
| | CKATGYTFTGYWIDWIKQRPG | CKSSQSVLYSSNQKNYLAWYQ |
| | HGLEWVGEILPGSDNINYNEKF | QKPGQSPRLLIYWASTRASGVP |
| | RGKATFTADTSSNTAYIQLSSL | DRFTGSGSGTDFTLTITNIQPED |
| | TTEDSAIYFCARCGTGPWFTY | LAVYYCHQFLSSTWTFGGGTK |
| | WGQGTLVTVSA | LEIK |
| | [SEQ ID NO: 114] | [SEQ ID NO: 115] |
| | CDR1: GYWID [SEQ ID NO: 116] | CDR1: KSSQSVLYSSNQKNYLA |
| | CDR2: EILPGSDNINYNEKFRG | [SEQ ID NO: 119] |
| | [SEQ ID NO: 117] | CDR2: WASTRAS [SEQ ID |
| | CDR3: CGTGPWFTY [SEQ ID | NO: 120] |
| | NO: 118] | CDR3: HQFLSSTWT [SEQ ID |
| | | NO: 121] |
| 9E6 | QLQLVQSGPELMRPGETVKISC | DIVMTQSPSSLSVSAGEKVTMS |
| or | KASGYTFTTYGMNWVKQAPG | CKSSQSLLNSGNQKNYLAWYQ |
| 10F5 | KGLKWVGWINTYSGVPTYAD | QKPGQPPKLLIYGASTRESGVP |
| | DFKGRFAFSLESSASTAFLQINN | DRFTGSGSGTDFTLTISSVQAED |
| | LKDEDTATYFCARFGNYGFDC | LAVYYCQNDHIYPYTFGGGTK |
| | WGQGTTLTVSS | LEIK |
| | [SEQ ID NO: 122] | [SEQ ID NO: 123] |
| | CDR1: TYGMN [SEQ ID NO: 124] | CDR1: KSSQSLLNSGNQKNYLA |
| | CDR2: WINTYSGVPTYADDFKG | [SEQ ID NO: 127] |
| | [SEQ ID NO: 125] | CDR2: GASTRES [SEQ ID |
| | CDR3: FGNYGFDC [SEQ ID | NO: 128] |
| | NO: 126] | CDR3: QNDHIYPYT [SEQ ID |
| | | NO: 129] |
| 5E7 | EVQLQQSGAELVRPGASVKLS | DVLMTQTPLTLSVPIGQPASISC |
| | CTASGFNIKDDYIHWVKQWPE | KSSQSLLHSNGKTYLNWLLQRP |
| | QGLEWIGWIDSENGDTEYASK | GQSPKLLIYLVSKLESGVPDRFS |
| | FQGKATMTADTSSNTAYLQLS | GSGSGTDFTLKISRVEAEDLGV |
| | GLTSEDTAVYYCTTSSYYSYDL | YYCLQTTHLYTFGGGTKLEIK |
| | FVYWGQGTLVTVSA | [SEQ ID NO: 131] |
| | [SEQ ID NO: 130] | CDR1: KSSQSLLHSNGKTYLN |
| | CDR1: DDYIH [SEQ ID NO: 132] | [SEQ ID NO: 135] |
| | CDR2: WIDSENGDTEYASKFQG | CDR2: LVSKLES [SEQ ID |
| | [SEQ ID NO: 133] | NO: 136] |
| | CDR3: SSYYSYDLFVY [SEQ ID | CDR3: LQTTHLYT [SEQ ID |
| | NO: 134] | NO: 137] |
| 2H6 | QIQLVQSGPELKKPGETVKISC | DIVMTQSPSSLSVSAGEKVTMS |
| | KASGYTFTTYGVNWVKQAPG | CKSSQSLVNSGNQKNYLAWYQ |
| | KGLKWMGWINTYSGVPTYAD | QKPGQPPKLLISGASTRESGVPD |
| | DFKGRFAFSLETIATTAYLQINN | RFTGSGSGTDFTLTISSVQAEDL |
| | LKNEDTATYFCARFGNYGFDY | AVYYCQNDHNYPYTFGGGTKL |
| | WGQGTTLTVSS [SEQ ID | EIK [SEQ ID NO: 139] |
| | NO: 138] | CDR1: KSSQSLVNSGNQKNYLA |
| | CDR1: TYGVN [SEQ ID NO: 140] | [SEQ ID NO: 143] |
| | CDR2: WINTYSGVPTYADDFKG | CDR2: GASTRES [SEQ ID |
| | [SEQ ID NO: 141] | NO: 144] |
| | CDR3: FGNYGFDY [SEQ ID | CDR3: QNDHNYPYT [SEQ ID |
| | NO: 142] | NO: 145] |
| h5E7 | QVQLVQSGAEVKKPGASVKVS | DVQMTQSPSSLSASVGDRVTIT |
| | CKASGFNIKDDYIHWVRQAPG | CKSSQSLLHSNGKTYLNWLQQ |
| | QGLEWMGWIDSENGDTEYAS | KPGQAPKLLLYLVSKLESGVPS |
| | KFQGRVTITADTSANTAYMELS | RFSGSGSGTDYTLTISSLQPEDF |
| | SLRSEDTAVYYCATSSYYSYDL | ATYYCLQTTHLYTFGQGTKLEI |
| | FVYWGQGTLVTVSS [SEQ ID | K [SEQ ID NO: 669] |
| | NO: 668] | CDR1: KSSQSLLHSNGKTYLN |
| | CDR1: DDYIH [SEQ ID NO: 132] | [SEQ ID NO: 135] |
| | CDR2: WIDSENGDTEYASKFQG | CDR2: LVSKLES [SEQ ID |
| | [SEQ ID NO: 133] | NO: 136] |
| | CDR3: SSYYSYDLFVY [SEQ ID | CDR3: LQTTHLYT [SEQ ID |
| | NO: 670] | NO: 137] |
| h5E7-YD- | QVQLVQSGAEVKKPGASVKVS | DVQMTQSPSSLSASVGDRVTIT |
| C6 | CKASGFNIKDDYIHWVRQAPG | CKSSQSLLHSNGKTYLNWLQQ |
| | QGLEWMGWIDSENGDTEYAS | KPGQAPKLLLYLVSKLESGVPS |
| | KFQGRVTITADTSANTAYMELS | RFSGSGSGTDYTLTISSLQPEDF |
| | SLRSEDTAVYYCATSEYYSYDL | ATYYCLQTTHLYTFGQGTKLEI |
| | FVYWGQGTLVTVSS [SEQ ID | K [SEQ ID NO: 669] |
| | NO: 671] | CDR1: KSSQSLLHSNGKTYLN |
| | CDR1: DDYIH [SEQ ID NO: 132] | [SEQ ID NO: 135] |
| | CDR2: WIDSENGDTEYASKFQG | CDR2: LVSKLES [SEQ ID |
| | [SEQ ID NO: 133] | NO: 136] |
| | CDR3: SEYYSYDLFVY [SEQ ID | CDR3: LQTTHLYT [SEQ ID |
| | NO: 672] | NO: 137] |
| h5E7-YD- | QVQLVQSGAEVKKPGASVKVS | DVQMTQSPSSLSASVGDRVTIT |
| F3 | CKASGFNIKDDYIHWVRQAPG | CKSSQSLLHSNGKTYLNWLQQ |
| | QGLEWMGWIDSENGDTEYAS | KPGQAPKLLLYLVSKLESGVPS |
| | KFQGRVTITADTSANTAYMELS | RFSGSGSGTDYTLTISSLQPEDF |
| | SLRSEDTAVYYCATSSYWSYD | ATYYCLQTTHLYTFGQGTKLEI |
| | LLVYWGQGTLVTVSS [SEQ ID | K [SEQ ID NO: 669] |
| | NO: 673] | CDR1: KSSQSLLHSNGKTYLN |
| | CDR1: DDYIH [SEQ ID NO: 132] | [SEQ ID NO: 135] |
| | CDR2: WIDSENGDTEYASKFQG | CDR2: LVSKLES [SEQ ID |
| | [SEQ ID NO: 133] | NO: 136] |
| | CDR3: SSYWSYDLLVY [SEQ ID | CDR3: LQTTHLYT [SEQ ID |
| | NO: 674] | NO: 137] |
| h5E7-YD- | QVQLVQSGAEVKKPGASVKVS | DVQMTQSPSSLSASVGDRVTIT |
| A6 | CKASGFNIKDDYIHWVRQAPG | CKSSQSLLHSNGKTYLNWLQQ |
| | QGLEWMGWIDSENGDTEYAS | KPGQAPKLLLYLVSKLESGVPS |
| | KFQGRVTITADTSANTAYMELS | RFSGSGSGTDYTLTISSLQPEDF |
| | SLRSEDTAVYYCATSSYWSYD | ATYYCLQTTHLYTFGQGTKLEI |
| | LFVYWGQGTLVTVSS [SEQ ID | K [SEQ ID NO: 669] |
| | NO: 675] | CDR1: KS SQSLLHSNGKTYLN |
| | CDR1: DDYIH [SEQ ID NO: 132] | [SEQ ID NO: 135] |
| | CDR2: WIDSENGDTEYASKFQG | CDR2: LVSKLES [SEQ ID |
| | [SEQ ID NO: 133] | NO: 136] |
| | CDR3: SSYWSYDLFVY [SEQ ID | CDR3: LQTTHLYT [SEQ ID |
| | NO: 676] | NO: 137] |
| h5E7-YD- | QVQLVQSGAEVKKPGASVKVS | DVQMTQSPSSLSASVGDRVTIT |
| B5 | CKASGFNIKDDYIHWVRQAPG | CKSSQSLLHSNGKTYLNWLQQ |
| | QGLEWMGWIDSENGDTEYAS | KPGQAPKLLLYLVSKLESGVPS |
| | KFQGRVTITADTSANTAYMELS | RFSGSGSGTDYTLTISSLQPEDF |
| | SLRSEDTAVYYCATSTYWSYD | ATYYCLQTTHLYTFGQGTKLEI |
| | LFVYWGQGTLVTVSS [SEQ ID | K [SEQ ID NO: 669] |
| | NO: 677] | CDR1: KSSQSLLHSNGKTYLN |
| | CDR1: DDYIH [SEQ ID NO: 132] | [SEQ ID NO: 135] |
| | CDR2: WIDSENGDTEYASKFQG | CDR2: LVSKLES [SEQ ID |
| | [SEQ ID NO: 133] | NO: 136] |
| | CDR3: STYWSYDLFVY [SEQ ID | CDR3: LQTTHLYT [SEQ ID |
| | NO: 6781 | NO: 137] |
| h5E7 | QVQLVQSGAEVKKPGASVKVS | DVQMTQSPSSLSASVGDRVTIT |
| variants | CKASGFNIKDDYIHWVRQAPG | CKSSQSLLHSNGKTYLNWLQQ |
| consensus | QGLEWMGWIDSENGDTEYAS | KPGQAPKLLLYLVSKLESGVPS |
| | KFQGRVTITADTSANTAYMELS | RFSGSGSGTDYTLTISSLQPEDF |
| | SLRSEDTAVYYCATSX₁YX₂SY | ATYYCLQTTHLYTFGQGTKLEI |
| | DLX₃VYWGQGTLVTVSS, | K [SEQ ID NO: 669] |
| | wherein | CDR1: KSSQSLLHSNGKTYLN |
| | X₁ is S, T, or E; | [SEQ ID NO: 135] |
| | X₂ is Y or W; and | CDR2: LVSKLES [SEQ ID |
| | X₃ is F or L [SEQ ID NO: 679] | NO: 136] |
| | CDR1: DDYIH [SEQ ID NO: 132] | CDR3: LQTTHLYT [SEQ ID |
| | CDR2: WIDSENGDTEYASKFQG | NO: 137] |
| | [SEQ ID NO: 133] | |
| | CDR3: SX₁YX₂SYDLX₃VY, | |
| | wherein | |
| | X₁ is S, T, or E; | |
| | X₂ is Y or W; and | |
| | X₃ is F or L [SEQ ID NO: 680] | |

In certain embodiments, the DLL3 binding site binds DLL3 with a K_{D} of 0.001 nM-10 nM, e.g., 0.001 nM-9 nM, 0.001 nM-8 nM, 0.001 nM-7 nM, 0.001 nM-6 nM, 0.001 nM-5 nM, 0.001 nM-4 nM, 0.001 nM-3 nM, 0.001 nM-2 nM, 0.001 nM-1 nM, 0.001 nM-0.9 nM, 0.001 nM-0.8 nM, 0.001 nM-0.7 nM, 0.001 nM-0.6 nM, 0.001 nM 0.5 nM, 0.001 nM-0.4 nM, 0.001 nM-0.3 nM, 0.001 nM-0.2 nM, 0.001 nM-0.1 nM, 0.05 nM-10 nM, 0.1 nM-10 nM, 0.2 nM-10 nM, 0.3 nM-10 nM, 0.4 nM-10 nM, 0.5 nM-10 nM, 1 nM-10 nM, 2 nM-10 nM, 3 nM-10 nM, 4 nM-10 nM, 5 nM-10 nM, 6 nM-10 nM, 7 nM-10 nM, 8 nM-10 nM, or 9 nM-10 nM, as measured using standard binding assays, for example, surface plasmon resonance or bio-layer interferometry. In certain embodiments, the antibody binds to DLL3 with a K_{D} of <0.011 nM, about 0.203 nM, about 0.669 nM, about 0.184 nM, about 1.12 nM, about 1.92 nM, about 5.11 nM, about 6.1 nM, or about 8.44 nM, as measured using surface plasmon resonance.

Table 3 lists publicly available peptide sequences of heavy chain variable domains and light chain variable domains that, in combination, can bind to DLL3.

TABLE 3

| | Heavy chain variable | Light chain variable |
|---|---|---|
| Clones | domain amino acid sequence | domain amino acid sequence |
| Rovalpituz- | QVQLVQSGAEVKKPGASVK | EIVMTQSPATLSVSPGERATLSCK |
| umab | VSCKASGYTFTNYGMNWVR | ASQSVSNDVVWYQQKPGQAPRL |
| (U.S. Pat. | QAPGQGLEWMGWINTYTGE | LIYYASNRYTGIPARFSGSGSGTE |
| No. | PTYADDFKGRVTMTTDTSTS | FTLTISSLQSEDFAVYYCQQDYTS |
| 9,089,615) | TAYMELRSLRSDDTAVYYC | PWTFGQGTKLEIKR |
| | ARIGDSSPSDYWGQGTLVTV | (SEQ ID NO: 147) |
| | SS | CDR1 (SEQ ID NO: 151)- |
| | (SEQ ID NO: 146) | QSVSNDVV |
| | CDR1 (SEQ ID NO: 148)- | CDR2 (SEQ ID NO: 152)- |
| | GYTFTNY | YASNRYT |
| | CDR2 (SEQ ID NO: 149)- | CDR3 (SEQ ID NO: 153)- |
| | NTYTGE | QQDYTSPWT |
| | CDR3 (SEQ ID NO: 150)- | |
| | IGDSSPSDY | |
| Chugai | QVQLQQSGADLVRPGTSVK | DIKMTQSPSSMYASLGERVTITC |
| DL301 | VSCKASGYAFTNYLIEWIKQ | KASQDINSYLIWFQQKPGKSPKT |
| (U.S. Pat. | RPGQGLEWIGVMNPGSGGT | LIYRTNRLVDGVPSRFSGSGSGQ |
| No. | HYSEKFRGKATLTADKSSST | DYSLTISSLEYGDMGIYYCLQYD |
| 9,127,071) | AYMQLISLTSDDSAVYFCAR | EFPFTFGSGTKLEIK |
| | SDYDYVTYAMDYWGQGTS | (SEQ ID NO: 155) |
| | VTVSS | CDR1 (SEQ ID NO: 159)- |
| | (SEQ ID NO: 154) | KASQDINSYLI |
| | CDR1 (SEQ ID NO: 156)- | CDR2 (SEQ ID NO: 160)- |
| | NYLIE | RTNRLVD |
| | CDR2 (SEQ ID NO: 157)- | CDR3 (SEQ ID NO: 161)- |
| | VMNPGSGGTHYSEKFRG | LQYDEFPFT |
| | CDR3 (SEQ ID NO: 158)- | |
| | SDYDYVTYAMDY | |
| Chugai | EVQLQQSGPELVKPGASVK | DIVLTQSPASLAVSLGQRATISCR |
| DL306 | MSCRASGYTFTDYYMKWV | ASKSVSTSGYSYMHWYQQKPGQ |
| (U.S. Pat. | KQSHGKSLEWIGDINPNNGD | PPKLLIFLASNLESGVPARFSGSG |
| No. | TFYNQKFKGKATLTIDKSSS | SGTDFTLNIHPVEEEDAATYYCQ |
| 9,127,071) | TAYMQLNSLTSDDSAVYYC | HSRHLPWTFGGGTKLEIK |
| | ARDGNYAYFDYWGQGTTLT | (SEQ ID NO: 163) |
| | VSS | CDR1 (SEQ ID NO: 167)- |
| | (SEQ ID NO: 162) | RASKSVSTSGYSYMH |
| | CDR1 (SEQ ID NO: 164)- | CDR2 (SEQ ID NO: 168)- |
| | DYYMK | LASNLES |
| | CDR2 (SEQ ID NO: 165)- | CDR3 (SEQ ID NO: 169)- |
| | DINPNNGDTFYNQKFKG | QHSRHLPWT |
| | CDR3 (SEQ ID NO: 166)- | |
| | DGNYAYFDY | |
| Chugai | QVQLQQSGGDLMKPGASVK | DIVMTQSQKFMSTSVGDRVSVTC |
| DL309 | ISCKAAGYTFSNYYIEWVKQ | KASQNVGTNVAWYQQKPGQSP |
| (U.S. Pat. | RPGHGLEWIGEILPGSGSTTY | KALIYSASYRYSGVPDRFTGSGS |
| No. | NEKFKGKASFTADTSSNTAY | GTDFTLTISNVQSEDLAEYFCQQ |
| 9,127,071) | MQLSSLTSEDSAVYYCARW | YNNYPLTFGAGTKLELK |
| | GAREPGFPYWGQGTLVTVS | (SEQ ID NO: 171) |
| | A | CDR1 (SEQ ID NO: 175)- |
| | (SEQ ID NO: 170) | KASQNVGTNVA |
| | CDR1 (SEQ ID NO: 172)- | CDR2 (SEQ ID NO: 176)- |
| | NYYIE | SASYRYS |
| | CDR2 (SEQ ID NO: 173)- | CDR3 (SEQ ID NO: 177)- |
| | EILPGSGSTTYNEKFKG | QQYNNYPLT |
| | CDR3 (SEQ ID NO: 174)- | |
| | WGAREPGFPY | |
| Chugai | EVKLVESGGGLVQPGGSLSL | DIQMTQSPSSLSASLGERVSLTCR |
| DL312 | SCAASGFTFTDYYMNWVRQ | ASQEISDYLSWLQQKPDGTIKRLI |
| (U.S. Pat. | PPGKALEWLALIRNKANGYT | FAASTLDSGVPKRFSGSRSGSD |
| No. | TEYNASVKGRFTISRDNSQNI | FSLSISSLESEDFADYYCLQYASY |
| 9,127,071) | LYLQMNALRAEDSATYYCA | PYTFGSGTKLEIK |
| | RDSDGYYEYYFDYWGQGTT | (SEQ ID NO: 179) |
| | LTVSS | CDR1 (SEQ ID NO: 183)- |
| | (SEQ ID NO: 178) | RASQEISDYLS |
| | CDR1 (SEQ ID NO: 180)- | CDR2 (SEQ ID NO: 184)- |
| | DYYMN | AASTLDS |
| | CDR2 (SEQ ID NO: 181)- | CDR3 (SEQ ID NO: 185)- |
| | LIRNKANGYTTEYNASVKG | LQYASYPYT |
| | CDR3 (SEQ ID NO: 182)- | |
| | DSDGYYEYYFDY | |
| Amgen | QVQLQESGPGLVKPSETLSL | EIVLTQSPGTLSLSPGERVTLSCR |
| DLL3-4 | TCTVSGGSISSYYWSWIRQPP | ASQRVNNNYLAWYQQRPGQAP |
| (U.S. Patent | GKGLEWIGYVYYSGTTNYN | RLLIYGASSRATGIPDRFSGSGSG |
| Publication | PSLKSRVTISVDTSKNQFSLK | TDFTLTISRLEPEDFAVYYCQQY |
| No. | LSSVTAADTAVYYCASIAVT | DRSPLTFGGGTKLEIK |
| 20170037130) | GFYFDYWGQGTLVTVSS | (SEQ ID NO: 187) |
| | (SEQ ID NO: 186) | CDR1 (SEQ ID NO: 191)- |
| | CDR1(SEQ ID NO: 188)- | RASQRVNNNYLA |
| | SYYWS | CDR2 (SEQ ID NO: 192)- |
| | CDR2 (SEQ ID NO: 189)- | GASSRAT |
| | YVYYSGTTNYNPSLKS | CDR3 (SEQ ID NO: 193)- |
| | CDR3 (SEQ ID NO: 190)- | QQYDRSPLT |
| | IAVTGFYFDY | |
| Amgen | QVQLQESGPGLVKPSETLSL | EIVLTQSPGTLSLSPGESATLSCR |
| DLL3-9 | TCTVSGASISSFYWSWIRQPP | ASQSVNKNYLAWYQQKPGQAPR |
| (U.S. Patent | GKGLEWIGYIYYSGTTNYNP | LLIYGASSRATGIPDRFSGSGSGT |
| Publication | SLKSRVTISVDTSKNQFSLKL | DFTLTISRLEPEDFAVYYCQQYD |
| No. | SSVTAADTAVYYCARIAVA | RSPLTFGGGTRLEIK |
| 20170037130) | GFPFDYWGQGTLVTVSS | (SEQ ID NO: 195) |
| | (SEQ ID NO: 194) | CDR1 (SEQ ID NO: 199)- |
| | CDR1 (SEQ ID NO: 196)- | RASQSVNKNYLA |
| | SFYWS | CDR2 (SEQ ID NO: 200)- |
| | CDR2 (SEQ ID NO: 197)- | GASSRAT |
| | YIYYSGTTNYNPSLKS | CDR3 (SEQ ID NO: 201)- |
| | CDR3 (SEQ ID NO: 198)- | QQYDRSPLT |
| | IAVAGFFFDY | |

Alternatively, novel antigen-binding sites that can bind to DLL3 can be identified by screening for binding to the amino acid sequence defined by SEQ ID NO:202.

SEQ ID NO: 202
MVSPRMSGLLSQTVILALIFLPQTRPAGVFELQIHSFGPGPGPGAPRSPC
SARLPCRLFFRVCLKPGLSEEAAESPCALGAALSARGPVYTEQPGAPAPD
LPLPDGLLQVPFRDAWPGTFSFIIETWREELGDQIGGPAWSLLARVAGRR
RLAAGGPWARDIQRAGAWELRFSYRARCEPPAVGTACTRLCRPRSAPSRC
GPGLRPCAPLEDECEAPLVCRAGCSPEHGFCEQPGECRCLEGWTGPLCTV
PVSTSSCLSPRGPSSATTGCLVPGPGPCDGNPCANGGSCSETPRSFECTC
PRGFYGLRCEVSGVTCADGPCFNGGLCVGGADPDSAYICHCPPGFQGSNC
EKRVDRCSLQPCRNGGLCLDLGHALRCRCRAGFAGPRCEHDLDDCAGRAC
ANGGTCVEGGGAHRCSCALGFGGRDCRERADPCAARPCAHGGRCYAHFSG
LVCACAPGYMGARCEFPVHPDGASALPAAPPGLRPGDPQRYLLPPALGLL
VAAGVAGAALLLVHVRRRGHSQDAGSRLLAGTPEPSVHALPDALNNLRTQ
EGSGDGPSSSVDWNRPEDVDPQGIYVISAPSIYAREVATPLFPPLHTGRA
GQRQHLLFPYPSSILSVK

In certain aspects, the present disclosure provides multi-specific binding proteins that bind to the NKG2D receptor and CD16 receptor on natural killer cells, and the tumor associated-antigen MUC1 (or MUC1-C). Table 4 lists some exemplary sequences of heavy chain variable domains and light chain variable domains that, in combination, can bind to MUC1 (or MUC1-C).

TABLE 4

| | Heavy chain variable domain | Light chain variable domain |
|---|---|---|
| Source | amino acid sequence | amino acid sequence |
| Immunomedics | QVQLQQSGAEVKKPGAS | DIQLTQSPSSLSASVGDRVT |
| Clivatuzumab | VKVSCEASGYTFPSYVLH | MTCSASSSVSSSYLYWYQQ |
| tetraxetan | WVKQAPGQGLEWIGYIN | KPGKAPKLWIYSTSNLASG |
| (U.S. Pat. No. | PYNDGTQYNEKFKGKAT | VPARFSGSGSGTDFTLTISSL |
| 9,452,228) | LTRDTSINTAYMELSRLR | QPEDSASYFCHQWNRYPYT |
| | SDDTAVYYCARGFGGSY | FGGGTRLEIKR |
| | GFAYWGQGTLVTVSSA | (SEQ ID NO: 207) |
| | (SEQ ID NO: 203) | CDR1(SEQ ID NO: 208) - |
| | CDR1(SEQ ID NO: 204) - | SSVSSSYLY |
| | GYTFPSY | CDR2 (SEQ ID NO: 209) - |
| | CDR2 (SEQ ID NO: 205) - | STSNLAS |
| | NPYNDG | CDR3 (SEQ ID NO: 210) - |
| | CDR3 (SEQ ID NO: 206) - | HQWNRYPYT |
| | GFGGSYGFAY | |
| Glycotope | EVQLVESGGGLVQPGGS | DIVMTQSPLSNPVTPGEPASI |
| Gatipotuzumab | MRLSCVASGFPFSNYWM | SCRSSKSLLHSNGITYFFWY |
| (U.S. Pat. No. | NWVRQAPGKGLEWVGEI | LQKPGQSPQLLIYQMSNLAS |
| 9,217,038) | RLKSNNYTTHYAESVKG | GVPDRFSGSGSGTDFTLRIS |
| | RFTISRDDSKNSLYLQMN | RVEAEDVGVYYCAQNLELP |
| | SLKTEDTAVYYCTRHYY | PTFGQGTKVEIKR (SEQ ID |
| | PDYWGQGTLVTVSSA | NO: 215) |
| | (SEQ ID NO: 211) | CDR1(SEQ ID NO: 216) - |
| | CDR1(SEQ ID NO: 212) - | RSSKSLLHSNGITYFF |
| | NYWMN | CDR2 (SEQ ID NO: 217) - |
| | CDR2 (SEQ ID NO: 213) - | QMSNLAS |
| | EIRLKSNNYTTHYAES | CDR3 (SEQ ID NO: 218) ?+0 |
| | CDR3 (SEQ ID NO: 214) - | AQNLELPPT |
| | HYYFDY | |

Alternatively, novel antigen-binding sites that can bind to MUC1 (or MUC1-C) can be identified by screening for binding to the amino acid sequence defined by SEQ ID NO:219.

SEQ ID NO: 219
MTPGTQSPFFLLLLLTVLTVVTGSGHASSTPGGEKETSATQRSSVPSSTE
KNAVSMTSSVLSSHSPGSGSSTTQGQDVTLAPATEPASGSAATWGQDVTS
VPVTRPALGSTTPPAHDVTSAPDNKPAPGSTAPPAHGVTSAPDTRPAPGS
TAPPAHGVTSAPDTRPAPGSTAPPAHGVTSAPDTRPAPGSTAPPAHGVTS
APDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTS
APDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTS
APDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTS
APDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTS
APDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTS
APDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTS
APDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTS
APDTRPAPGSTAPPAHGVTSAPDTRPAPGSTAPPAHGVTSAPDNRPALGS
TAPPVHNVTSASGSASGSASTLVHNGTSARATTTPASKSTPFSIPSHHSD
TPTTLASHSTKTDASSTHHSSVPPLTSSNHSTSPQLSTGVSFFFLSFHIS
NLQFNSSLEDPSTDYYQELQRDISEMFLQIYKQGGFLGLSNIKFRPGSVV
VQLTLAFREGTINVHDVETQFNQYKTEAASRYNLTISDVSVSDVPFPFSA
QSGAGVPGWGIALLVLVCVLVALAIVYLIALAVCQCRRKNYGQLDIFPAR
DTYHPMSEYPTYHTHGRYVPPSSTDRSPYEKVSAGNGGSSLSYTNPAVAA
TSANL

In certain aspects, the present disclosure provides multi-specific binding proteins that bind to the NKG2D receptor and CD16 receptor on natural killer cells, and the antigen Plexin-A1. Table 5 lists some exemplary peptide sequences of heavy chain variable domains and light chain variable domains that, in combination, can bind to Plexin-A1.

TABLE 5

| | Heavy chain variable domain | Light chain variable domain amino |
|---|---|---|
| Source | amino acid sequence | acid sequence |
| Chugai | QVQLQQSGPGLVKPSQTLSL | SYELTQPPSVSVSPGQTASITCS |
| (U.S. Pat. | TCAISGDSLSSTSAAWNWIR | GDKLGDKYASWYQQRPGQSPL |
| Publication | QSPSPSGGLEWLGRTYYRSKW | LVIYQDNKRPSGIPQRFSGSNSG |
| No. | YNDYAVSVKSRITINPDTSK | NTATLTISGTQAMDEADYFCQA |
| 20180193451) | NQFSLQLNSVTPEDTSVYYC | WDSGTFVFGTGTKVTIL (SEQ |
| | ARDRGYYNGVDVWGQGTM | ID NO: 224) |
| | VTVSS (SEQ ID NO: 220) | CDR1(SEQ ID NO: 225) - |
| | CDR1(SEQ ID NO: 221) - | SGDKLGDKYAS |
| | STSAAWN | CDR2 (SEQ ID NO: 226) - |
| | CDR2 (SEQ ID NO: 222) - | QDNKRPS |
| | RTYYRSKWYNDYAVSVKS | CDR3 (SEQ ID NO: 227) - |
| | CDR3 (SEQ ID NO: 223) - | QAWDSGTFV |
| | DRGYYNGVDV | |
| Chugai | QVQLVQSGAEVKKPGASVK | SYELTQPPSVSVSPGQTASITCS |
| (U.S. Pat. | VSCKASGYTFTSYYMHWVR | GDKLEDKYASWYQLKPGHSPV |
| Publication | AQPGQGLEWMGIINPSGGST | LVIYQDSKRPSGIPERFSGSNSG |
| No. | SYAQKFQGRVTMTRDTSTST | NTATLTISGTQAMDEADYYCRA |
| 20180193451) | VYMELSSLRSEDTAVYYCAR | WDSNTGDVVFGGGTKLTVL |
| | APGHYYYGMDVWGQGTTV | (SEQ ID NO: 232) |
| | TVSS (SEQ ID NO: 228) | CDR1(SEQ ID NO: 233) - |
| | CDR1(SEQ | ID NO: 229) - SGDKLEDKYAS |
| | SYYMH | CDR2 (SEQ ID NO: 234) - |
| | CDR2 (SEQ ID NO: 230) - | QDSKRPS |
| | IINPSGGSTSYAQKFQ | CDR3 (SEQ ID NO: 235) - |
| | CDR3 (SEQ ID NO: 231) - | RAWDSNTGDVV |
| | APGHYYYGMDV | |

Alternatively, novel antigen-binding sites that can bind to Plexin-A1 can be identified by screening for binding to the amino acid sequence defined by SEQ ID NO:236.

SEQ ID NO: 236
MPLPPRSLQVLLLLLLLLLLLPGMWAEAGLPRAGGGSQPPFRTFSASDWG
LTHLVVHEQTGEVYVGAVNRIYKLSGNLTLLRAHVTGPVEDNEKCYPPPS
VQSCPHGLGSTDNVNKLLLLDYAANRLLACGSASQGICQFLRLDDLFKLG
EPHHRKEHYLSSVQEAGSMAGVLIAGPPGQGQAKLFVGTPIDGKSEYFPT
LSSRRLMANEEDADMFGFVYQDEFVSSQLKIPSDTLSKFPAFDIYYVYSF
RSEQFVYYLTLQLDTQLTSPDAAGEHFFTSKIVRLCVDDPKFYSYVEFPI
GCEQAGVEYRLVQDAYLSRPGRALAHQLGLAEDEDVLFTVFAQGQKNRVK
PPKESALCLFTLRAIKEKIKERIQSCYRGEGKLSLPWLLNKELGCINSPL
QIDDDFCGQDFNQPLGGTVTIEGTPLFVDKDDGLTAVAAYDYRGRTVVFA
GTRSGRIRKILVDLSNPGGRPALAYESVVAQEGSPILRDLVLSPNHQYLY
AMTEKQVTRVPVESCVQYTSCELCLGSRDPHCGWCVLHSICSRRDACERA
DEPQRFAADLLQCVQLTVQPRNVSVTMSQVPLVLQAWNVPDLSAGVNCSF
EDFTESESVLEDGRIHCRSPSAREVAPITRGQGDQRVVKLYLKSKETGKK
FASVDFVFYNCSVHQSCLSCVNGSFPCHWCKYRHVCTHNVADCAFLEGRV
NVSEDCPQILPSTQIYVPVGVVKPITLAARNLPQPQSGQRGYECLFHIPG
SPARVTALRFNSSSLQCQNSSYSYEGNDVSDLPVNLSVVWNGNFVIDNPQ
NIQAHLYKCPALRESCGLCLKADPRPECGWCVAERRCSLRHHCAADTPAS
WMHARHGSSRCTDPKILKLSPETGPRQGGTRLTITGENLGLRFEDVRLGV
RVGKVLCSPVESEYISAEQIVCEIGDASSVRAHDALVEVCVRDCSPHYRA
LSPKRFTFVTPTFYRVSPSRGPLSGGTWIGIEGSHLNAGSDVAVSVGGRP
CSFSWRNSREIRCLTPPGQSPGSAPIIININRAQLTNPEVKYNYTEDPTI
LRIDPEWSINSGGTLLTVTGTNLATVREPRIRAKYGGIERENGCLVYNDT
TMVCRAPSVANPVRSPPELGERPDELGFVMDNVRSLLVLNSTSFLYYPDP
VLEPLSPTGLLELKPSSPLILKGRNLLPPAPGNSRLNYTVLIGSTPCTLT
VSETQLLCEAPNLTGQHKVTVRAGGFEFSPGTLQVYSDSLLTLPAIVGIG
GGGGLLLLVIVAVLIAYKRKSRDADRTLKRLQLQMDNLESRVALECKEAF
AELQTDIHELTNDLDGAGIPFLDYRTYAMRVLFPGIEDHPVLKEMEVQAN
VEKSLTLFGQLLTKKHFLLTFIRTLEAQRSFSMRDRGNVASLIMTALQGE
MEYATGVLKQLLSDLIEKNLESKNHPKLLLRRTESVAEKMLTNWFTFLLY
KFLKECAGEPLFMLYCAIKQQMEKGPIDAITGEARYSLSEDKLIRQQIDY
KTLTLNCVNPENENAPEVPVKGLDCDTVTQAKEKLLDAAYKGVPYSQRPK
AADMDLEWRQGRMARIILQDEDVTTKIDNDWKRLNTLAHYQVTDGSSVAL
VPKQTSAYNISNSSTFTKSLSRYESMLRTASSPDSLRSRTPMITPDLESG
TKLWHLVKNHDHLDQREGDRGSKMVSEIYLTRLLATKGTLQKFVDDLFET
IFSTAHRGSALPLAIKYMFDFLDEQADKHQIHDADVRHTWKSNCLPLRFW
VNVIKNPQFVFDIHKNSITDACLSVVAQTFMDSCSTSEHKLGKDSPSNKL
LYAKDIPNYKSWVERYYADIAKMPAISDQDMSAYLAEQSRLHLSQFNSMS
ALHEIYSYITKYKDEILAALEKDEQARRQRLRSKLEQVVDTMALSS

In certain aspects, the present disclosure provides multi-specific binding proteins that bind to the NKG2D receptor and CD16 receptor on natural killer cells, and the tumor-associated antigen TNFRSF10B. Table 6 lists some exemplary peptide sequences of heavy chain variable domains and light chain variable domains that, in combination, can bind to TNFRSF10B.

TABLE 6

| | Heavy chain variable domain | Light chain variable domain amino |
|---|---|---|
| Source | amino acid sequence | acid sequence |
| Tigatuzumab | EVQLVESGGGLVQPGGSLR | DIQMTQSPSSLSASVGDRVTIT |
| | LSCAASGFTFSSYVMSWVR | CKASQDVGTAVAWYQQKPG |
| | QAPGKGLEWVATISSGGSY | KAPKLLIYWASTRHTGVPSRF |
| | TYYPDSVKGRFTISRDNAK | SGSGSGTDFTLTISSLQPEDFA |
| | NTLYLQMNSLRAEDTAVY | TYYCQQYSSYRTFGQGTKVEI |
| | YCARRGDSMITTDYWGQG | KR (SEQ ID NO: 241) |
| | TLVTVSSA (SEQ ID NO: 237) | CDR1(SEQ ID NO: 242) - |
| | CDR1(SEQ ID NO: 238) - | QDVGTAVA |
| | GFTFSSY | CDR2 (SEQ ID NO: 243) - |
| | CDR2 (SEQ ID NO: 239) - | WASTRHT |
| | SSGGSY | CDR3 (SEQ ID NO: 244) - |
| | CDR3 (SEQ ID NO: 240) - | QQYSSYRT |
| | RGDSMITTDY | |
| Conatumumab | QVQLQESGPGLVKPSQTLSL | EIVLTQSPGTLSLSPGERATLS |
| | TCTVSGGSISSGDYFWSWIR | CRASQGISRSYLAWYQQKPGQ |
| | QLPGKGLEWIGHIHNSGTT | APSLLIYGASSRATGIPDRFSGS |
| | YYNPSLKSRVTISVDTSKKQ | GSGTDFTLTISRLEPEDFAVYY |
| | FSLRLSSVTAADTAVYYCA | CQQFGSSPWTFG |
| | RDRGGDYYYGMDVWGQG | QGTKVEIKR |
| | TTVTVSSA (SEQ ID NO: 245) | (SEQ ID NO: 249) |
| | CDR1(SEQ ID NO: 246) - | CDR1(SEQ ID NO: 250) - |
| | GGSISSGDY | QGISRSYLA |
| | CDR2 (SEQ ID NO: 247) - | CDR2 (SEQ ID NO: 251) - |
| | HNSGT | GASSRAT |
| | CDR3 (SEQ ID NO: 248) - | CDR3 (SEQ ID NO: 252) - |
| | DRGGDYYYGMDV | QQFGSSPWT |
| Drozitumab | EVQLVQSGGGVERPGGSLR | SELTQDPAVSVALGQTVRITCS |
| | LSCAASGFTFDDYAMSWVR | GDSLRSYYASWYQQKPGQAP |
| | QAPGKGLEWVSGINWQGG | VLVIYGANNRPSGIPDRFSGSS |
| | STGYADSVKGRVTISRDNA | SGNTASLTITGAQAEDEADYY |
| | KNSLYLQMNSLRAEDTAVY | CNSADSSGNHVVFGGGTKLT |
| | YCAKILGAGRGWYFDYWG | VLG |
| | KGTTVTVSSA (SEQ ID | (SEQ ID NO: 257) |
| | NO: 253) | CDR1(SEQ ID NO: 258) - |
| | CDR1(SEQ ID NO: 254) - | SLRSYYAS |
| | GFTFDDY | CDR2 (SEQ ID NO: 259) - |
| | CDR2 (SEQ ID NO: 255) - | GANNRPS |
| | NWQGGS | CDR3 (SEQ ID NO: 260) - |
| | CDR3 (SEQ ID NO: 256) - | NSADSSGNHVV |
| | ILGAGRGWYFDY | |

Alternatively, novel antigen-binding sites that can bind to TNFRSF10B can be identified by screening for binding to the amino acid sequence defined by SEQ ID NO:261.

SEQ ID NO: 261
MEQRGQNAPAASGARKRHGPGPREARGARPGPRVPKTLVLVVAAVLLLVS
AESALITQQDLAPQQRAAPQQKRSSPSEGLCPPGHHISEDGRDCISCKYG
QDYSTHWNDLLFCLRCTRCDSGEVELSPCTTTRNTVCQCEEGTFREEDSP
EMCRKCRTGCPRGMVKVGDCTPWSDIECVHKESGTKHSGEVPAVEETVTS
SPGTPASPCSLSGIIIGVTVAAVVLIVAVFVCKSLLWKKVLPYLKGICSG
GGGDPERVDRSSQRPGAEDNVLNEIVSILQPTQVPEQEMEVQEPAEPTGV
NMLSPGESEHLLEPAEAERSQRRRLLVPANEGDPTETLRQCFDDFADLVP
FDSWEPLMRKLGLMDNEIKVAKAEAAGHRDTLYTMLIKWVNKTGRDASVH
TLLDALETLGERLAKQKIEDHLLSSGKFMYLEGNADSAMS

In certain aspects, the present disclosure provides multi-specific binding proteins that bind to the NKG2D receptor and CD16 receptor on natural killer cells, and the tumor-associated antigen STEAP1. Table 7 lists some exemplary peptide sequences of heavy chain variable domains and light chain variable domains that, in combination, can bind to STEAP1.

TABLE 7

| | Heavy chain variable domain | Light chain variable domain |
|---|---|---|
| Source | amino acid sequence | amino acid sequence |
| Vandortuzumab | EVQLVESGGGLVQPGGSL | DIQMTQSPSSLSASVGDRVT |
| vedotin | RLSCAASGYSITSDYAWN | ITCKSSQSLLYRSNQKNYLA |
| | WVRQAPGKGLEWVGYISN | WYQQKPGKAPKLLIYWAST |
| | SGSTSYNPSLKSRFTISRDN | RESGVPSRFSGSGSGTDFTL |
| | SKNTLYLQMNSLRAEDTA | TISSLQPEDFATYYCQQYYN |
| | VYYCARERNYDYDDYYY | YPRTFGQGTKVEIKR |
| | AMDYWGQGTLVTVSS | (SEQ ID NO: 266) |
| | (SEQ ID NO: 262) | CDR1(SEQ ID NO: 267) - |
| | CDR1(SEQ ID NO: 263) - | QSLLYRSNQKNYLA |
| | GYSITSDY | CDR2 (SEQ ID NO: 268) - |
| | CDR2 (SEQ ID NO: 264) - | WASTRES |
| | SNSGS | CDR3 (SEQ ID NO: 269) - |
| | CDR3 (SEQ ID NO: 265) - | QQYYNYPRT |
| | ERNYDYDDYYYAMDY | |
| Roche | EVQLVESGGGLVQPGGSL | DIQMTQSPSSLSASVGDRVT |
| (U.S. Pat. | RLSCAVSGYSITSDYAWN | ITCKSSQSLLYRSNQKNYLA |
| Publication No. | WVRQAPGKGLEWVGYISN | WYQQKPGKAPKLLIYWAST |
| 20170096495) | SGSTSYNPSLKSRFTISRDN | RESGVPSRFSGSGSGTDFTL |
| | SKNTLYLQMNSLRAEDTA | TISSLQPEDFATYYCQQYYN |
| | VYYCARERNYDYDDYYY | YPRTFGQGTKVEIK |
| | AMDYWGQGTLVTVSS | (SEQ ID NO: 274) |
| | (SEQ ID NO: 270) | CDR1(SEQ ID NO: 275) - |
| | CDR1(SEQ ID NO: 271) - | KSSQSLLYRSNQKNYLA |
| | DYAWN | CDR2 (SEQ ID NO: 276) - |
| | CDR2 (SEQ ID NO: 272) - | WASTRES |
| | YISNSGSTSYNPSLKS | CDR3 (SEQ ID NO: 277) - |
| | CDR3 (SEQ ID NO: 273) - | QQYYNYPRT |
| | ERNYDYDDYYYAMDY | |

Alternatively, novel antigen-binding sites that can bind to STEAP1 can be identified by screening for binding to the amino acid sequence defined by SEQ ID NO:278.

SEQ ID NO: 278
MESRKDITNQEELWKMKPRRNLEEDDYLHKDTGETSMLKRPVLLHLHQTA
HADEFDCPSELQHTQELFPQWHLPIKIAAIIASLTFLYTLLREVIHPLAT
SHQQYFYKIPILVINKVLPMVSITLLALVYLPGVIAAIVQLHNGTKYKKF
PHWLDKWMLTRKQFGLLSFFFAVLHAIYSLSYPMRRSYRYKLLNWAYQQV
QQNKEDAWIEHDVWRMEIYVSLGIVGLAILALLAVTSIPSVSDSLTWREF
HYIQSKLGIVSLLLGTIHALIFAWNKWIDIKQFVWYTPPTFMIAVFLPIV
VLIFKSILFLPCLRKKILKIRHGWEDVTKINKTEICSQL

In certain aspects, the present disclosure provides multi-specific binding proteins that bind to the NKG2D receptor and CD16 receptor on natural killer cells, and the tumor-associated antigen CDCP1. Table 8 lists some exemplary peptide sequences of heavy chain variable domains and light chain variable domains that, in combination, can bind to CDCP1.

TABLE 8

| | Heavy chain variable domain | Light chain variable domain amino |
|---|---|---|
| Source | amino acid sequence | acid sequence |
| Roche | EVQLVESGGGLVQPGGSL | EIVLTQSPATLSLSPGERATMS |
| RG7287 | RLSCAASGFTFNSYGMSW | CSVSSSVFYLHWYQQKPGQAP |
| (U.S. Pat. No. | VRQAPGKGLEWVATISSG | RLWIYDTSKLASGIPARFSGSG |
| 9,346,886) | GSYKYYVDSVKGRFTISRD | SGTDFTLTISSLEPEDFAVYYC |
| | NAKNSLYLQMNSLRAEDT | QQWNSNPPTFGGGTKVEIK |
| | AVYYCARHPDYDGVWFA | (SEQ ID NO: 283) |
| | YWGQGTLVTVSS | CDR1(SEQ ID NO: 284) - |
| | (SEQ ID NO: 279) | SVSSSVFYLH |
| | CDR1(SEQ ID NO: 280) - | CDR2 (SEQ ID NO: 285) - |
| | SYGMS | DTSKLAS |
| | CDR2 (SEQ ID NO: 281) - | CDR3 (SEQ ID NO: 286) - |
| | TISSGGSYKYYVDSVKG | QQWNSNPPT |
| | CDR3 (SEQ ID NO: 282) - | |
| | HPDYDGVWFAY | |
| Roche | EVQLVESGGGLVQPGGSL | DIQMTQSPSSLSASVGDRVTIT |
| RG7287 | RLSCAASGFTFNSYGMSW | CSVSSSVFYVHWYQQKPGKA |
| (U.S. Pat. No. | VRQAPGKGLEWVATISSG | PKLLIYDTSKLASGVPSRFSGS |
| 9,346,886) | GSYKYYVDSVKGRFTISRD | GSGTDFTFTISSLQPEDIATYY |
| | NAKNSLYLQMNSLRAEDT | CQQWNSNPPTFGGGTKVEIK |
| | AVYYCARHPDYDGVWFA | (SEQ ID NO: 287) |
| | YWGQGTLVTVSS | CDR1(SEQ ID NO: 288) - |
| | (SEQ ID NO: 279) | SVSSSVFYVH |
| | CDR1(SEQ ID NO: 280) - | CDR2 (SEQ ID NO: 289) - |
| | SYGMS | DTSKLAS |
| | CDR2 (SEQ ID NO: 281) - | CDR3 (SEQ ID NO: 290) - |
| | TISSGGSYKYYVDSVKG | QQWNSNPPT |
| | CDR3 (SEQ ID NO: 282) - | |
| | HPDYDGVWFAY | |

Alternatively, novel antigen-binding sites that can bind to CDCP1 can be identified by screening for binding to the amino acid sequence defined by SEQ ID NO:291.

SEQ ID NO: 291
MAGLNCGVSIALLGVLLLGAARLPRGAEAFEIALPRESNITVLIKLGTPT
LLAKPCYIVISKRHITMLSIKSGERIVFTFSCQSPENHFVIEIQKNIDCM
SGPCPFGEVQLQPSTSLLPTLNRTFIWDVKAHKSIGLELQFSIPRLRQIG
PGESCPDGVTHSISGRIDATVVRIGTFCSNGTVSRIKMQEGVKMALHLPW
FHPRNVSGFSIANRSSIKRLCIIESVFEGEGSATLMSANYPEGFPEDELM
TWQFVVPAHLRASVSFLNFNLSNCERKEERVEYYIPGSTTNPEVFKLEDK
QPGNMAGNFNLSLQGCDQDAQSPGILRLQFQVLVQHPQNESNKIYVVDLS
NERAMSLTIEPRPVKQSRKFVPGCFVCLESRTCSSNLTLTSGSKHKISFL
CDDLTRLWMNVEKTISCTDHRYCQRKSYSLQVPSDILHLPVELHDFSWKL
LVPKDRLSLVLVPAQKLQQHTHEKPCNTSFSYLVASAIPSQDLYFGSFCP
GGSIKQIQVKQNISVTLRTFAPSFQQEASRQGLTVSFIPYFKEEGVFTVT
PDTKSKVYLRTPNWDRGLPSLTSVSWNISVPRDQVACLTFFKERSGVVCQ
TGRAFMIIQEQRTRAEEIFSLDEDVLPKPSFHHHSFWVNISNCSPTSGKQ
LDLLFSVTLTPRTVDLTVILIAAVGGGVLLLSALGLIICCVKKKKKKTNK
GPAVGIYNDNINTEMPRQPKKFQKGRKDNDSHVYAVIEDTMVYGHLLQDS
SGSFLQPEVDTYRPFQGTMGVCPPSPPTICSRAPTAKLATEEPPPRSPPE
SESEPYTFSHPNNGDVSSKDTDIPLLNTQEPMEPAE

In certain aspects, the present disclosure provides multi-specific binding proteins that bind to the NKG2D receptor and CD16 receptor on natural killer cells, and the tumor-associated antigen PTK7. Table 9 lists some exemplary peptide sequences of heavy chain variable domains and light chain variable domains that, in combination, can bind to PTK7.

TABLE 9

| | Heavy chain variable domain | Light chain variable domain |
|---|---|---|
| Source | amino acid sequence | amino acid sequence |
| Pfizer | QVQLVQSGPEVKKPGASV | EIVLTQSPATLSLSPGERATLS |
| Cofetuzumab | KVSCKASGYTFTDYAVHW | CRASESVDSYGKSFMHWYQ |
| (U.S. Pat. | VRQAPGKRLEWIGVISTYN | QKPGQAPRLLIYRASNLESGIP |
| Publication No. | DYTYNNQDFKGRVTMTRD | ARFSGSGSGTDFTLTISSLEPE |
| 20150315293) | TSASTAYMELSRLRSEDTA | DFAVYYCQQSNEDPWTFGGG |
| | VYYCARGNSYFYALDYW | TKLEIKR (SEQ ID NO: 296) |
| | GQGTSVTVSSA | CDR1(SEQ ID NO: 297) - |
| | (SEQ ID NO: 292) | RASESVDSYGKSFMH |
| | CDR1(SEQ ID NO: 293) - | CDR2 (SEQ ID NO: 298) - |
| | DYAVH | RASNLES |
| | CDR2 (SEQ ID NO: 294) - | CDR3 (SEQ ID NO: 299) - |
| | VISTYNDYTYNNQDFK | QQSNEDPWT |
| | CDR3 (SEQ ID NO: 295) - | |
| | GNSYFYALDY | |

Antigen-binding sites that can bind to PTK7 can also be identified, for example, by screening for binding to the amino acid sequence defined by SEQ ID NO:300.

SEQ ID NO: 300
MGAARGSPARPRRLPLLSVLLLPLLGGTQTAIVFIKQPSSQDALQGRRAL
LRCEVEAPGPVHVYWLLDGAPVQDTERRFAQGSSLSFAAVDRLQDSGTFQ
CVARDDVTGEEARSANASFNIKWIEAGPVVLKHPASEAEIQPQTQVTLRC
HIDGHPRPTYQWFRDGTPLSDGQSNHTVSSKERNLTLRPAGPEHSGLYSC
CAHSAFGQACSSQNFTLSIADESFARVVLAPQDVVVARYEEAMFHCQFSA
QPPPSLQWLFEDETPITNRSRPPHLRRATVFANGSLLLTQVRPRNAGIYR
CIGQGQRGPPIILEATLHLAEIEDMPLFEPRVFTAGSEERVTCLPPKGLP
EPSVWWEHAGVRLPTHGRVYQKGHELVLANIAESDAGVYTCHAANLAGQR
RQDVNITVATVPSWLKKPQDSQLEEGKPGYLDCLTQATPKPTVVWYRNQM
LISEDSRFEVFKNGTLRINSVEVYDGTWYRCMSSTPAGSIEAQARVQVLE
KLKFTPPPQPQQCMEFDKEATVPCSATGREKPTIKWERADGSSLPEWVTD
NAGTLHFARVTRDDAGNYTCIASNGPQGQIRAHVQLTVAVFITFKVEPER
TTVYQGHTALLQCEAQGDPKPLIQWKGKDRILDPTKLGPRMHIFQNGSLV
IHDVAPEDSGRYTCIAGNSCNIKHTEAPLYVVDKPVPEESEGPGSPPPYK
MIQTIGLSVGAAVAYIIAVLGLMFYCKKRCKAKRLQKQPEGEEPEMECLN
GGPLQNGQPSAEIQEEVALTSLGSGPAATNKRHSTSDKMHFPRSSLQPIT
TLGKSEFGEVFLAKAQGLEEGVAETLVLVKSLQSKDEQQQLDFRRELEMF
GKLNHANVVRLLGLCREAEPHYMVLEYVDLGDLKQFLRISKSKDEKLKSQ
PLSTKQKVALCTQVALGMEHLSNNRFVHKDLAARNCLVSAQRQVKVSALG
LSKDVYNSEYYHFRQAWVPLRWMSPEAILEGDFSTKSDVWAFGVLMWEVF
THGEMPHGGQADDEVLADLQAGKARLPQPEGCPSKLYRLMQRCWALSPKD
RPSFSEIASALGDSTVDSKP

In certain aspects, the present disclosure provides multi-specific binding proteins that bind to the NKG2D receptor and CD16 receptor on natural killer cells, and the tumor-associated antigen AXL. Table 10 lists some exemplary peptide sequences of heavy chain variable domains and light chain variable domains that, in combination, can bind to AXL.

TABLE 10

| | Heavy chain variable domain | Light chain variable domain amino |
|---|---|---|
| Source | amino acid sequence | acid sequence |
| Genentech | EVQLVESGGGLVQPGGSL | DIQMTQSPSSLSASVGDRVTIT |
| (U.S. Pat. No. | RLSCAASGFSLSGSWIHW | CRASQDVSTAVAWYQQKPGK |
| 8,853,369) | VRQAPGKGLEWVGWINP | APKLLIYSASFLYSGVPSRFSG |
| | YRGYAYYADSVKGRFTIS | SGSGTDFTLTISSLQPEDFATY |
| | ADTSKNTAYLQMNSLRAE | YCQQSYTTPPTFGQGTKVEIK |
| | DTAVYYCAREYSGWGGS | R (SEQ ID NO: 305) |
| | SVGYAMDYWGQGTLV | CDR1(SEQ ID NO: 306) - |
| | (SEQ ID NO: 301) | QDVSTAVA |
| | CDR1(SEQ ID NO: 302) - | CDR2 (SEQ ID NO: 307) - |
| | GFSLSGS | SASFLYS |
| | CDR2 (SEQ ID NO: 303) - | CDR3 (SEQ ID NO: 308) - |
| | NPYRGY | QQSYTTPPT |
| | CDR3 (SEQ ID NO: 304) - | |
| | EYSGWGGSSVGYAMDY | |
| Inserm | QVQLQQSGAELMKPGAS | DIQMTQSPASLSASVGETVTIT |
| (U.S. Pat. No. | VKMSCKAAGYTFSSYWIE | CRASENIYSYLTWYQQKQRKS |
| 9,249,228) | WVRQRPGHGLEWIGEIFP | PQLLVYNAKTLAEGVPSRFSG |
| | GSDSTNYNEKFNDRATFT | SGSGTQFSLKINSLQPEDFGTY |
| | ADTSSNTAYMQLSSLTSE | YCQHHYATPWTFGGGTKVEI |
| | DSAVYYCARPLYYGSSA | K (SEQ ID NO: 313) |
| | WFAYWGQGTLVTVSA | CDR1(SEQ ID NO: 314) - |
| | (SEQ ID NO: 309) | ENIYSYLT |
| | CDR1(SEQ ID NO: 310) - | CDR2 (SEQ ID NO: 315) - |
| | GYTFSSY | NAKTLAE |
| | CDR2 (SEQ ID NO: 311) - | CDR3 (SEQ ID NO: 316) - |
| | FPGSDS | QHHYATPWT |
| | CDR3 (SEQ ID NO: 312) - | |
| | PLYYGSSAWFAY | |

The antigen-binding sites that can bind to AXL can be identified, for example, by screening for binding to the amino acid sequence defined by SEQ ID NO:317.

SEQ ID NO: 317
MAWRCPRMGRVPLAWCLALCGWACMAPRGTQAEESPFVGNPGNITGARGL
TGTLRCQLQVQGEPPEVHWLRDGQILELADSTQTQVPLGEDEQDDWIVVS
QLRITSLQLSDTGQYQCLVFLGHQTFVSQPGYVGLEGLPYFLEEPEDRTV
AANTPFNLSCQAQGPPEPVDLLWLQDAVPLATAPGHGPQRSLHVPGLNKT
SSFSCEAHNAKGVTTSRTATITVLPQQPRNLHLVSRQPTELEVAWTPGLS
GIYPLTHCTLQAVLSNDGMGIQAGEPDPPEEPLTSQASVPPHQLRLGSLH
PHTPYHIRVACTSSQGPSSWTHWLPVETPEGVPLGPPENISATRNGSQAF
VHWQEPRAPLQGTLLGYRLAYQGQDTPEVLMDIGLRQEVTLELQGDGSVS
NLTVCVAAYTAAGDGPWSLPVPLEAWRPGQAQPVHQLVKEPSTPAFSWPW
WYVLLGAVVAAACVLILALFLVHRRKKETRYGEVFEPTVERGELVVRYRV
RKSYSRRTTEATLNSLGISEELKEKLRDVMVDRHKVALGKTLGEGEFGAV
MEGQLNQDDSILKVAVKTMKIAICTRSELEDFLSEAVCMKEFDHPNVMRL
IGVCFQGSERESFPAPVVILPFMKHGDLHSFLLYSRLGDQPVYLPTQMLV
KFMADIASGMEYLSTKRFIHRDLAARNCMLNENMSVCVADFGLSKKIYNG
DYYRQGRIAKMPVKWIAIESLADRVYTSKSDVWSFGVTMWEIATRGQTPY
PGVENSEIYDYLRQGNRLKQPADCLDGLYALMSRCWELNPQDRPSFTELR
EDLENTLKALPPAQEPDEILYVNMDEGGGYPEPPGAAGGADPPTQPDPKD
SCSCLTAAEVHPAGRYVLCPSTTPSPAQPADRGSPAAPGQEDGA

In certain aspects, the present disclosure provides multi-specific binding proteins that bind to the NKG2D receptor and CD16 receptor on natural killer cells, and the tumor-associated antigen ERBB-3. Table 11 lists some exemplary peptide sequences of heavy chain variable domains and light chain variable domains that, in combination, can bind to ERBB-3.

TABLE 11

| | Heavy chain | Light chain |
|---|---|---|
| | variable | variable |
| | domain | domain |
| | amino acid | amino acid |
| Source | sequence | sequence |
| Merrimack | EVQLLESGGGLVQP | QSALTQPASVSGS |
| Seribanlumab | GGSLRLSCAASGFT | PGQSRRISCTGTS |
| | FSHYVMAWVRQAPG | SDVGSYNVVSWY |
| | KGLEWVSSISSSG | QQHPGKAPKLIIY |
| | GWTLYADSVKGRF | EVSQRPSGVSNRF |
| | TISRDNSKNTLYL | SGSKSGNTASLTI |
| | QMNSLRAEDTAVY | SGLQTEDEADYYC |
| | YCTRGLKMATIFD | CSYAGSSIFVIFG |
| | YWGQGTLVTVSS | GGTKVTVL |
| | (SEQ ID | (SEQ ID |
| | NO: 318) | NO: 322) |
| | CDR1 | CDR1 |
| | (SEQ ID | (SEQ ID |
| | NO: 319- | NO: 323)- |
| | GFTFSHY | SSDVGSYNVVS |
| | CDR2 | CDR2 |
| | (SEQ ID | (SEQ ID |
| | NO: 320)- | NO: 324)- |
| | SSSGGW | EVSQRPS |
| | CDR3 | CDR3 |
| | (SEQ ID | (SEQ ID |
| | NO: 321)- | NO: 325)- |
| | GLKMATIFDY | CSYAGSSIFVI |
| Amgen | QVQLQQWGAGLLK | DIEMTQSPDSLAV |
| Patritumab | PSETLSLTCAVYG | SLGERATINCRSS |
| | GSFSGYYWSWIRQ | QSVLYSSSNRNYL |
| | PPGKGLEWIGEIN | AWYQQNPGQPPKL |
| | HSGSTNYNPSLKS | LIYWASTRESGVP |
| | RVTISVETSKNQF | DRFSGSGSGTDFT |
| | SLKLSSVTAADTA | LTISSLQAEDVAV |
| | VYYCARDKWTWYF | YYCQQYYSTPRTF |
| | DLWGRGTLVTVS | GQGTKVEIKR |
| | SA | (SEQ ID |
| | (SEQ ID | NO: 330) |
| | NO: 326) | CDR1 |
| | CDR1 | (SEQ ID |
| | (SEQ ID | NO: 331)- |
| | NO: 327)- | QSVLYSSSNRN |
| | GGSFSGY | YLA |
| | CDR2 | CDR2 |
| | (SEQ ID | (SEQ ID |
| | NO: 328)- | NO: 332)- |
| | NHSGS | WASTRES |
| | CDR3 | CDR3 |
| | (SEQ ID | (SEQ ID |
| | NO: 329)- | NO: 333)- |
| | DKWTWYFDL | QQYYSTPRT |
| Genentech | EVQLVESGGGLVQ | DIQMTQSPSSLSA |
| Duligotuzumab | PGGSLRLSCAASG | SVGDRVTITCRAS |
| | FTLSGDWIHWVRQ | QNIATDVAWYQQK |
| | APGKGLEWVGEIS | PGKAPKLLIYSAS |
| | AAGGYTDYADSVK | FLYSGVPSRFSGS |
| | GRFTISADTSKNT | GSGTDFTLTISSL |
| | AYLQMNSLRAEDT | QPEDFATYYCQQS |
| | AVYYCARESRVSF | EPEPYTFGQGTKV |
| | EAAMDYWGQGTLV | EIKR |
| | TVSSA | (SEQ ID |
| | (SEQ ID | NO: 338) |
| | NO: 334) | CDR 1 |
| | CDR1 | (SEQ ID |
| | (SEQ ID | NO: 339)- |
| | NO: 335)- | QNIATDVA |
| | GFTLSGD | CDR2 |
| | CDR2 | (SEQ ID |
| | (SEQ ID | NO: 340)- |
| | NO: 336)- | SASFLYS |
| | SAAGGY | CDR3 |
| | CDR3 | (SEQ ID |
| | (SEQ ID | NO: 341)- |
| | NO: 337)- | QQSEPEPYT |
| | ESRVSFEAAMDY | |

The antigen-binding sites that can bind to ERBB-3 can be identified, for example, by screening for binding to the amino acid sequence defined by SEQ ID NO:342.

SEQ ID NO: 342
MRANDALQVLGLLFSLARGSEVGNSQAVCPGTLNGLSVTGDAE
NQYQTLYKLYERCEVVMGNLEIVLTGHNADLSFLQWIREVTGY
VLVAMNEFSTLPLPNLRVVRGTQVYDGKFAIFVMLNYNTNSSH
ALRQLRLTQLTEILSGGVYIEKNDKLCHMDTIDWRDIVRDRDA
EIVVKDNGRSCPPCHEVCKGRCWGPGSEDCQTLTKTICAPQCN
GHCFGPNPNQCCHDECAGGCSGPQDTDCFACRHFNDSGACVPR
CPQPLVYNKLTFQLEPNPHTKYQYGGVCVASCPHNFVVDQTSC
VRACPPDKMEVDKNGLKMCEPCGGLCPKACEGTGSGSRFQTVD
SSNIDGFVNCTKILGNLDFLITGLNGDPWHKIPALDPEKLNVF
RTVREITGYLNIQSWPPHMHNFSVFSNLTTIGGRSLYNRGFSL
LIMKNLNVTSLGFRSLKEISAGRIYISANRQLCYHHSLNWTKV
LRGPTEERLDIKHNRPRRDCVAEGKVCDPLCSSGGCWGPGPGQ
CLSCRNYSRGGVCVTHCNFLNGEPREFAHEAECFSCHPECQPM
EGTATCNGSGSDTCAQCAHFRDGPHCVSSCPHGVLGAKGPIYK
YPDVQNECRPCHENCTQGCKGPELQDCLGQTLVLIGKTHLTMA
LTVIAGLVVIFMMLGGTFLYWRGRRIQNKRAMRRYLERGESIE
PLDPSEKANKVLARIFKETELRKLKVLGSGVFGTVHKGVWIPE
GESIKIPVCIKVIEDKSGRQSFQAVTDHMLAIGSLDHAHIVRL
LGLCPGSSLQLVTQYLPLGSLLDHVRQHRGALGPQLLLNWGVQ
IAKGMYYLEEHGMVHRNLAARNVLLKSPSQVQVADFGVADLLP
PDDKQLLYSEAKTPIKWMALESIHFGKYTHQSDVWSYGVTVWE
LMTFGAEPYAGLRLAEVPDLLEKGERLAQPQICTIDVYMVMVK
CWMIDENIRPTFKELANEFTRMARDPPRYLVIKRESGPGIAPG
PEPHGLTNKKLEEVELEPELDLDLDLEAEEDNLATTTLGSALS
LPVGTLNRPRGSQSLLSPSSGYMPMNQGNLGESCQESAVSGSS
ERCPRPVSLHPMPRGCLASESSEGHVTGSEAELQEKVSMCRSR
SRSRSPRPRGDSAYHSQRHSLLTPVTPLSPPGLEEEDVNGYVM
PDTHLKGTPSSREGTLSSVGLSSVLGTEEEDEDEEYEYMNRRR
RHSPPHPPRPSSLEELGYEYMDVGSDLSASLGSTQSCPLHPVP
IMPTAGTTPDEDYEYMNRQRDGGGPGGDYAAMGACPASEQGYE
EMRAFQGPGHQAPHVHYARLKTLRSLEATDSAFDNPDYWHSRL
FPKANAQRT

In certain aspects, the present disclosure provides multi-specific binding proteins that bind to the NKG2D receptor and CD16 receptor on natural killer cells, and the tumor-associated antigen EDNRB. Table 12 lists some exemplary peptide sequences of heavy chain variable domains and light chain variable domains that, in combination, can bind to EDNRB.

TABLE 12

| | Heavy chain | Light chain |
|---|---|---|
| | variable | variable |
| | domain | domain |
| | amino acid | amino acid |
| Source | sequence | sequence |
| Genentech | EVQLVESGGGLV | DIQMTQSPSSLS |
| RG7636 | QPGGSLRLSCAA | ASVGDRVTITCK |
| (U.S. Pat. No. | SGYTFTSYWMQW | SSQSLLDSDGKT |
| 9,464,141) | VRQAPGKGLEWI | YLNWLQQKPGKA |
| | GTIYPGDGDTSY | PKRLIYLVSKLD |
| | AQKFKGRATLST | SGVPSRFSGSGS |
| | DKSKNTAYLQMN | GTDFTLTISSLQ |
| | SLRAEDTAVYYC | PEDFATYYCWQG |
| | ARWGYAYDIDNW | THFPYTFGQGTK |
| | G | VEIK |
| | (SEQ ID | (SEQ ID |
| | NO: 343) | NO: 347) |
| | CDR1 | CDR1 |
| | (SEQ ID | (SEQ ID |
| | NO: 344)- | NO: 348)- |
| | GYTFTSYWMQ | KSSQSLLDSDGK |
| | CDR2 | TYLN |
| | (SEQ ID | CDR2 |
| | NO: 345)- | (SEQ ID |
| | TIYPGDGDTS | NO: 349)- |
| | YAQKFK | LVSKLDS |
| | CDR3 | CDR3 |
| | (SEQ ID | (SEQ ID |
| | NO: 346)- | NO: 350)- |
| | WGYAYDIDN | WQGTHFPYT |
| Rendomab-B49 | QVQLQQPGAALV | DVLMTQTPLSLPV |
| (PCT | KPGASVKLSCKA | SLGDQASISCRSS |
| Publication No. | SGYTFISYWMLW | QSIVHSNGNTYLE |
| WO2017220739 | VKQRPGRGLEWI | WYLQKPGQSPKLL |
| A1) | GRIDPDSGGTKY | IYKVSNRFSGVPD |
| | NEKFKSKATLTV | RFSGSGSGTDFTL |
| | DKSSSTAYMQLS | KISRVEAED |
| | SLTSEDSAVYY | LGVYYCFQGSH |
| | CAREGDYAWFA | VPWTFGGGT |
| | YWGQGTLVPVS | KLEIK |
| | A | (SEQ ID |
| | (SEQ ID | NO: 355) |
| | NO: 351) | CDR1 |
| | CDR1 | (SEQ ID |
| | (SEQ ID | NO: 356)- |
| | NO: 352)- | QSIVHSNGNTY |
| | GYTFTSYW | CDR2 |
| | CDR2 | (SEQ ID |
| | (SEQ ID | NO: 357)- |
| | NO: 353)- | KVS |
| | IDPDSGGT | CDR3 |
| | CDR3 | (SEQ ID |
| | (SEQ ID | NO: 358)- |
| | NO: 354)- | FQGSHVPWT |
| | AREGDYAWFAY | |

Alternatively, novel antigen-binding sites that can bind to EDNRB can be identified, for example, by screening for binding to the amino acid sequence defined by SEQ ID NO:359.

SEQ ID NO: 359
MQPPPSLCGRALVALVLACGLSRIWGEERGFPPDRATPLLQTAE
IMTPPTKTLWPKGSNASLARSLAPAEVPKGDRTAGSPPRTISPP
PCQGPIEIKETFKYINTVVSCLVFVLGUGNSTLLRIIYKNKCMR
NGPNILIASLALGDLLHIVIDIPINVYKLLAEDWPFGAEMCKLV
PFIQKASVGITVLSLCALSIDRYRAVASWSRIKGIGVPKWTAVE
IVLIWVVSVVLAVPEAIGFDIITMDYKGSYLRICLLHPVQKTAF
MQFYKTAKDWWLFSFYFCLPLAITAFFYTLMTCEMLRKKSGMQI
ALNDHLKQRREVAKTVFCLVLVFALCWLPLHLSRILKLTLYNQN
DPNRCELLSFLLVLDYIGINMASLNSCINPIALYLVSKRFKNCF
KSCLCCWCQSFEEKQSLEEKQSCLKFKANDHGYDNFRSSNKYSS
S

In certain aspects, the present disclosure provides multi-specific binding proteins that bind to the NKG2D receptor and CD16 receptor on natural killer cells, and the tumor-associated antigen TYRP1. Table 13 lists some exemplary peptide sequences of heavy chain variable domains and light chain variable domains that, in combination, can bind to TYRP1.

TABLE 13

| | Heavy chain | Light chain |
|---|---|---|
| | variable | variable |
| | domain | domain amino |
| | amino acid | acid |
| Source | sequence | sequence |
| CTA99 | EVQLQQSGAELVR | AIQMSQSPASL |
| | PGALVKLSCKTSG | SASVGETVTIT |
| | FNIKDYFLHWVRQ | CRASGNIYNYL |
| | RPDQGLEWIGWIN | AWYQQKQGKSP |
| | PDNGNTVYDPKFQ | HLLVYDAKTLA |
| | GTASLTADTSSNT | DGVPSRFSGSG |
| | VYLQLSGLTSEDT | SGTQYSLKISS |
| | AVYFCTRRDYTY | LQTEDSGNYYC |
| | EKAALDYWGQGA | QHFWSLPFTFG |
| | SVIVFSAA | SGTKLEIK |
| | (SEQ ID | (SEQ ID |
| | NO: 360) | NO: 364) |
| | CDR1 | CDR1 |
| | (SEQ ID | (SEQ ID |
| | NO: 361)- | NO: 365)- |
| | GFNIKDY | RASGNIYNYLA |
| | CDR2 | CDR2 |
| | (SEQ ID | (SEQ ID |
| | NO: 362)- | NO: 366)- |
| | INPDNGN | DAKTLAD |
| | CDR3 | CDR3 |
| | (SEQ ID | (SEQ ID |
| | NO: 363)- | NO: 367)- |
| | RDYTYEKAALDY | QHFWSLPFT |
| Imclone | QVQLVQSGSELK | EIVLTQSPATLS |
| Systems | KPGASVKISCKA | LSPGERATLSCR |
| Flanvotumab | SGYTFTSYAMNW | ASQSVSSYLAWY |
| (clone 20D7S) | VRQAPGQGLESM | QQKPGQAPRLLI |
| (U.S. Pat. No. | GWINTNTGNPTY | YDASNRATGIPA |
| 7,951,370) | AQGFTGRFVFSM | RFSGSGSGTDFT |
| | DTSVSTAYLQIS | LTISSLEPEDFA |
| | SLKAEDTAIYYC | VYYCQQRSNWLM |
| | APRYSSSWYLDY | YTFGQGTKLEIK |
| | WGQGTLVTVSS | (SEQ ID |
| | (SEQ ID | NO: 372) |
| | NO: 368) | CDR1 |
| | CDR1 | (SEQ ID |
| | (SEQ ID | NO: 373)- |
| | NO: 369)- | RASQSVSSYLA |
| | SCKASGYTFTS | CDR2 |
| | YAMN | (SEQ ID |
| | CDR2 | NO: 374)- |
| | (SEQ ID | DASNRAT |
| | NO: 370)- | CDR3 |
| | WINTNTGNPTY | (SEQ ID |
| | AQGFTG | NO: 375)- |
| | CDR3 | QQRSNWLMYT |
| | (SEQ ID | |
| | NO: 371)- | |
| | RYSSSWYLDY | |
| Imclone | QVQLVQSGSELK | EIVLTQSPATLSL |
| Systems | KPGASVKISCKA | SPGERATLSCRAS |
| Flanvotumab | SGYTFTSYAMNW | QSVSSYLAWYQQK |
| (clone 20D7) | VRQAPGQGLECM | PGQAPRLLIYDAS |
| (U.S. | GWINTN | NRATGIPARFSG |
| Pat No. | TGNPTYAQGF | SGSGTDFTLTI |
| 7,951,370) | TGRFVFSMDT | SSLEPEDFAVY |
| | SVSTAYLQIS | YCQQRSNWLMY |
| | SLKAEDTAIY | TFGQGTKLEIK |
| | YCAPRYSSSW | (SEQ ID |
| | YLDYWGQGTL | NO: 380) |
| | VTVSS | CDR1 |
| | (SEQ ID | (SEQ ID |
| | NO: 376) | NO: 381)- |
| | CDR1 | RASQSVSSYLA |
| | (SEQ ID | CDR2 |
| | NO: 377)- | (SEQ ID |
| | SCKASGYTFT | NO: 382)- |
| | SYAMN | DASNRAT |
| | CDR2 | CDR3 |
| | (SEQ ID | (SEQ ID |
| | NO: 378)- | NO: 383)- |
| | WINTNTGNPT | QQRSNWLMYT |
| | YAQGFTG | |
| | CDR3 | |
| | (SEQ ID | |
| | NO: 379)- | |
| | RYSSSWYLDY | |

Alternatively, novel antigen-binding sites that can bind to TYRP1 can be identified, for example, by screening for binding to the amino acid sequence defined by SEQ ID NO:384.

SEQ ID NO: 384
MSAPKLLSLGCIFFPLLLFQQARAQFPRQCATVEALRSGMCCPDLSPV
SGPGTDRCGSSSGRGRCEAVTADSRPHSPQYPHDGRDDREVWPLRFFN
RTCHCNGNFSGHNCGTCRPGWRGAACDQRVLIVRRNLLDLSKEEKNHF
VRALDMAKRTTHPLFVIATRRSEEILGPDGNTPQFENISIYNYFVWTH
YYSVKKTFLGVGQESFGEVDFSHEGPAFLTWHRYHLLRLEKDMQEMLQ
EPSFSLPYWNFATGKNVCDICTDDLMGSRSNFDSTLISPNSVFSQWRV
VCDSLEDYDTLGTLCNSTEDGPIRRNPAGNVARPMVQRLPEPQDVAQC
LEVGLFDTPPFYSNSTNSFRNTVEGYSDPTGKYDPAVRSLHNLAHLFL
NGTGGQTHLSPNDPIFVLLHTFTDAVFDEWLRRYNADISTFPLENAPI
GHNRQYNMVPFWPPVTNTEMFVTAPDNLGYTYEIQWPSREFSVPEIIA
IAVVGALLLVALIFGTASYLIRARRSMDEANQPLLTDQYQCYAEEYEK
LQNPNQSVV

In certain aspects, the present disclosure provides multi-specific binding proteins that bind to the NKG2D receptor and CD16 receptor on natural killer cells, and the tumor-associated antigen OLR1. Table 14 lists some exemplary peptide sequences of heavy chain variable domains and light chain variable domains that, in combination, can bind to OLR1.

TABLE 14

| | Heavy chain | Light chain |
|---|---|---|
| | variable | variable |
| | domain | domain amino |
| | amino acid | acid |
| Source | sequence | sequence |
| Novartis | QVQLLESGGGLV | QSVLTQPPSVSG |
| (U.S. Pat. No. | QPGGSLRLSCAA | APGQRVTISCSG |
| 9,562,101) | SGFTFSDYALHW | SSSNTGSNYVSW |
| | VRQAPGKGLEWV | YQQLPGTAPKLL |
| | SVISYQGGFIYY | IHSTSHRPSGVP |
| | ADSVKGRFTISR | DRFSGSKSGTSA |
| | DNSKNTLYLQMN | SLAITGLQAEDS |
| | SLRAEDTAVYYC | ADYYCQSWDVES |
| | ARSPGLTSYHDS | ERVVFGGGTKLT |
| | WGQGTLVTVSS | VL |
| | (SEQ ID | (SEQ ID |
| | NO: 385) | NO: 389) |
| | CDR1 | CDR1 |
| | (SEQ ID | (SEQ ID |
| | NO: 386)- | NO: 390)- |
| | DYALH | SGSSSNIGSNY |
| | CDR2 | VS |
| | (SEQ ID | CDR2 |
| | NO: 387)- | (SEQ ID |
| | VISYQGGFIYY | NO: 391)- |
| | ADSVKG | STSHRPS |
| | CDR3 | CDR3 |
| | (SEQ ID | (SEQ ID |
| | NO: 388)- | NO: 392)- |
| | SPGLTSYHDS | QSWDYESERVV |
| Medlmmune | QVQLVQSGAEVK | QSVVTQPPSVSG |
| (U.S. Pat. No. | KPGAVKVSCKVS | APGQRVTISCTG |
| 10,117,889) | GYTLTELSMHWV | SSSNIGAGYDVH |
| | RQAPGKGLEWMG | WYQQLPGTAPKL |
| | GFDPEDFKYHTH | LIYGNSNRPSGV |
| | QKFQGRVTMTED | PDRFSGSKSGTS |
| | TSTDTAYMELSS | ASLAITGLQAED |
| | LRSEDTAVYYCA | EADYYCQSYDSS |
| | LVWGTQGKGVRG | LSGWVFGGGTKL |
| | WDYYYGMDVWGQ | TVL |
| | GTTVTVSS | (SEQ ID |
| | (SEQ ID | NO: 397) |
| | NO: 393) | CDR1 |
| | CDR1 | (SEQ ID |
| | (SEQ ID | NO: 398)- |
| | NO: 394)- | TGSSSNIGAGY |
| | ELSMHCDR2 | DVHCDR2 |
| | (SEQ ID | (SEQ ID |
| | NO: 395)- | NO: 399)- |
| | GFDPEDFKYH | GNSNRPS |
| | THQKFQG | CDR3 |
| | CDR3 | (SEQ ID |
| | (SEQ ID | NO: 400)- |
| | NO: 396)- | QSYDSSLSGWV |
| | VWGTQGKGVR | |
| | GWDYYYGMDV | |

Alternatively, novel antigen-binding sites that can bind to OLR1 can be identified, for example, by screening for binding to the amino acid sequence defined by SEQ ID NO:401 (OLR1 isoform 1), SEQ ID NO:402 (OLR1 isoform 2), or SEQ ID NO:403 (OLR1 isoform 3).

SEQ ID NO: 401
MTFDDLKIQTVKDQPDEKSNGKKAKGLQFLYSPWWCLAAATLGVLCL
GLVVTIMVLGMQLSQVSDLLTQEQANLTHQKKKLEGQISARQQAEEA
SQESENELKEMIETLARKLNEKSKEQMELHHQNLNLQETLKRVANCS
APCPQDWIWHGENCYLFSSGSFNWEKSQEKCLSLDAKLLKINSTADL
DFIQQAISYSSFPFWMGLSRRNPSYPWLWEDGSPLMPHLFRVRGAVS
QTYPSGTCAYIQRGAVYAENCILAAFSICQKKANLRAQ
SEQ ID NO: 402
MTFDDLKIQTVKDQPDEKSNGKKAKGLQFLYSPWWCLAAATLGVLCL
GLVVTIMVLGMQLSQVSDLLTQEQANLTHQKKKLEGQISARQQAEEA
SQESENELKEMIETLARKLNEKSKEQMELHHQNLNLQETLKRVANCS
GLHPASNFLFQFSILDGAVSEEPQLPMALGGRFSFDAPLI
SEQ ID NO: 403
MTFDDLKIQTVKDQPDEKSNGKKAKGLQFLYSPWWCLAAATLGVLCL
GLVVTIMVLGMQLSQVSDLLTQEQANLTHQKKKLEGQISARQQAEEA
SQESENELKEMIETLARKLNEKSKEQMELHHQNLNLQETLKRVANCS
APCPQDWIWHGENCYLFSSGSFNWEKSQEKCLSLDAKLLKINSTADL
I

In certain embodiments, the present disclosure provides multi-specific binding proteins that bind to the NKG2D receptor and CD16 receptor on natural killer cells, and the tumor-associated antigen ADAM12. Exemplary monoclonal antibodies that bind to ADAM12 can be found in US Patent Publication No. 20160208016, and produced by the hybridoma cell lines 7B8 and 8F8.

Alternatively, novel antigen-binding sites that can bind to ADAM12 can be identified by screening for binding to the amino acid sequence defined by SEQ ID NO:404.

SEQ ID NO: 404
MAARPLPVSPARALLLALAGALLAPCEARGVSLWNQGRADEVVSA
SVGSGDLWIPVKSFDSKNHPEVLNIRLQRESKELIINLERNEGLI
ASSFTETHYLQDGTDVSLARNYTVILGHCYYHGHVRGYSDSAVSL
STCSGLRGLIVFENESYVLEPMKSATNRYKLFPAKKLKSVRGSCG
SHHNTPNLAAKNVFPPPSQTWARRHKRETLKATKYVELVIVADNR
EFQRQGKDLEKVKQRLIEIANHVDKFYRPLNIRIVLVGVEVWNDM
DKCSVSQDPFTSLHEFLDWRKMKLLPRKSHDNAQLVSGVYFQGTT
IGMAPIMSMCTADQSGGIVMDHSDNPLGAAVTLAHELGHNFGMNH
DTLDRGCSCQMAVEKGGCIMNASTGYPFPMVFSSCSRKDLETSLE
KGMGVCLFNLPEVRESFGGQKCGNRFVEEGEECDCGEPEECMNRC
CNATTCTLKPDAVCAHGLCCEDCQLKPAGTACRDSSNSCDLPEFC
TGASPHCPANVYLHDGHSCQDVDGYCYNGICQTHEQQCVTLWGPG
AKPAPGICFERVNSAGDPYGNCGKVSKSSFAKCEMRDAKCGKIQC
QGGASRPVIGTNAVSIETNIPLQQGGRILCRGTHVYLGDDMPDPG
LVLAGTKCADGKICLNRQCQNISVFGVHECAMQCHGRGVCNNRKN
CHCEAHWAPPFCDKFGFGGSTDSGPIRQADNQGLTIGILVTILCL
LAAGFVVYLKRKTLIRLLFTNKKTTIEKLRCVRPSRPPRGFQPCQ
AHLGHLGKGLMRKPPDSYPPKDNPRRLLQCQNVDISRPLNGLNVP
QPQSTQRVLPPLHRAPRAPSVPARPLPAKPALRQAQGTCKPNPPQ
KPLPADPLARTTRLTHALARTPGQWETGLRLAPLRPAPQYPHQVP
RSTHTAYIK

In certain embodiments, the present disclosure provides multi-specific binding proteins that bind to the NKG2D receptor and CD16 receptor on natural killer cells, and the tumor-associated antigen PLAUR. Table 15 lists some exemplary sequences of heavy chain variable domains and light chain variable domains that, in combination, can bind to PLAUR.

Table 15

| | Heavy chain | Light chain |
|---|---|---|
| | variable | variable |
| | domain | domain |
| | amino acid | amino acid |
| Source | sequence | sequence |
| Tactic | EVQLQQSGPEL | DVVMTQTPLTLS |
| Pharma | VKTGASVKISC | VTIGQPASISCK |
| ATN-658 | KASGYSFTSYY | SSQSLLDSDGKT |
| (U.S. | MHWVKQSHGKS | YLNWLLQRPGQS |
| Pat. No. | LEWIGEINPYN | PKRLIYLVSKLD |
| 8,105,602) | GGASYNQKIKG | SGVPDRFTGSGS |
| | RATFTVDTSSR | GTDFTLKISRVE |
| | TAYMQFNSLTS | AEDLGVYYCWQG |
| | EDSAVYYCARS | THFPLTFGAGTK |
| | IYGHSVLDYWGQ | LELKR |
| | GTSVSVSS | (SEQ ID |
| | (SEQ ID | NO: 409) |
| | NO: 405) | CDR1 |
| | CDR1 | (SEQ ID |
| | (SEQ ID | NO: 410)- |
| | NO: 406)- | QSLLDSDG |
| | GYSFTSY | KTYLN |
| | CDR2 | CDR2 |
| | (SEQ ID | (SEQ ID |
| | NO: 407)- | NO: 411)- |
| | NPYNGG | LVSKLDS |
| | CDR3 | CDR3 |
| | (SEQ ID | (SEQ ID |
| | NO: 408)- | NO: 412)- |
| | SIYGHSVLDY | WQGTHFPLT |
| University | QVQLQQSGPGL | LDVVMTQSPL |
| of | VKPSQTLSLTC | SLPVTPGEPA |
| California | AISGDSVSSNS | SISCRSSQSL |
| (U.S. Pat. No. | AAWNWIRQSPS | LRSNGYNYLD |
| 9,029,509) | RGLEWLGRTYY | WYLQKPGQSP |
| | RSKWYNDYAVS | QLLIYLGSIR |
| | VKSRIIINPDT | ASGVPDRFSG |
| | SKNQFSLQLNS | SGSGTDFTLR |
| | VTPEDTAVYYC | ISRVEAEDVG |
| | ARDPGGPLDDS | VYYCMQALQT |
| | FDIWGQGTMVT | PFTFGQGTKL |
| | VSSASTKGPSV | EIKRTVAAPS |
| | FPLAPSSKSTS | VFIFPPSDEQ |
| | GGTAALGCLVK | LKSGTASVVC |
| | DYFPEPVTVSW | LLNNFYPREA |
| | NSGALTSGVHT | KVQWKVDNAL |
| | FPAVLQSSGLY | QSGNSQESVT |
| | SLSSVVTVPSS | EQDSICDSTY |
| | SLGTQTYICNV | SLSSTLTLSK |
| | NHKPSNTKVDK | ADYEKHKVYA |
| | KVEPKSC | CEVTHQGLSS |
| | (SEQ ID | PVTKSFNRGE |
| | NO: 413) | C |
| | CDR1 | (SEQ ID |
| | (SEQ ID | NO: 4I7) |
| | NO: 414)- | CDR1 |
| | MQALQTPFT | (SEQ ID |
| | CDR2 | NO: 418)- |
| | (SEQ ID | RSSQSLLRSN |
| | NO: 415)- | GYNYLD |
| | RTYYRSKWYN | CDR2 |
| | DYAVSVKS | (SEQ ID |
| | CDR3 | NO: 419)- |
| | (SEQ ID | LGSIRAS |
| | NO: 416)- | CDR3 |
| | DPGGPLDDSFDI | (SEQ ID |
| | | NO: 420)- |
| | | MQALQTPFT |

Alternatively, novel antigen-binding sites that can bind to PLAUR can be identified by screening for binding to the amino acid sequence defined by SEQ ID NO:421.

SEQ ID NO: 421
MGHPPLLPLLLLLHTCVPASWGLRCMQCKTNGDCRVEECALGQDLCRTT
IVRLWEEGEELELVEKSCTHSEKTNRTLSYRTGLKITSLTEVVCGLDLC
NQGNSGRAVTYSRSRYLECISCGSSDMSCERGRHQSLQCRSPEEQCLDV
VTHWIQEGEEGRPKDDRHLRGCGYLPGCPGSNGFHNNDTFHFLKCCNTT
KCNEGPILELENLPQNGRQCYSCKGNSTHGCSSEETFLIDCRGPMNQCL
VATGTHEPKNQSYMVRGCATASMCQHAHLGDAFSMNHIDVSCCTKSGCN
HPDLDVQYRSGAAPQPGPAHLSLTITLLMTARLWGGTLLWT

In certain embodiments, the present disclosure provides multi-specific binding proteins that bind to the NKG2D receptor and CD16 receptor on natural killer cells, and the tumor-associated antigen CCR6. Table 16 lists some exemplary peptide sequences of heavy chain variable domains and light chain variable domains that, in combination, can bind to CCR6.

TABLE 16

| | Heavy chain | Light chain |
|---|---|---|
| | variable | variable |
| | domain | domain amino |
| | amino acid | acid |
| Source | sequence | sequence |
| Glenmark | EVQLVESGGG | DVVMTQSPLSL |
| (U.S. Pat. | LVQPGGSLRL | PVTLGQPASIS |
| Publication | SCAASGFSFS | CRSSQSIVHSN |
| No. | DYYMYWVRQA | GNTYLEWYQQ |
| 20180086836) | PGKGLEWVSY | RPGQSPRLLIY |
| | ITNGGITYYP | KVSNRFSGVPD |
| | DSVKGRFTIS | RFSGSGSGTDF |
| | RDNSKNTLYL | TLKISRVEAED |
| | QMNSLRAEDT | VGVYYCFQGSH |
| | AVYYCTSPLR | VPLTFGQGTKL |
| | GAWFAYWGQG | EIK |
| | TLVTVSS | (SEQ ID |
| | (SEQ ID | NO: 426) |
| | NO: 422) | CDR1 |
| | CDR1 | (SEQ ID |
| | (SEQ ID | NO: 427)- |
| | NO:423)- | QSIVHSNGNTY |
| | GFSFSDYYMY | LE |
| | CDR2 | CDR2 |
| | (SEQ ID | (SEQ ID |
| | NO: 424)- | NO: 428)- |
| | YITNGGITY | KVSNRFS |
| | YPDSVKG | CDR3 |
| | CDR3 | (SEQ ID |
| | (SEQ ID | NO: 429)- |
| | NO: 425)- | FQGSHVPLT |
| | TSPLRGAWFAY | |
| MSM Protein | EVQLLESGGGL | EIVLTQSPGTLSL |
| Technologies | VQPGGSLRLSC | SPGERATLSCRAS |
| (U.S. Pat. | AASGFTFSSYA | QSVSSSYLAWYQQ |
| Publication | MSWVRQAPGKG | KPGQAPRLLIYGA |
| No. | LEWVSAISGSG | SSRATGIPDRFSG |
| 20150337037) | GSTYYADSVKG | SGSGTDFTLTISR |
| | RFTISRDNSKNT | LEPEDFAV |
| | LYLQMNSLRAED | YYCQQYSS |
| | TAMYYCARHGY | SPLTFGQG |
| | SATRQGLDYWG | TKVEIKRT |
| | QGTLVTVSSAS | VAAPSVFI |
| | TKGPSVFPLAP | FPPSDEQL |
| | SSKSTSGGTAA | KSGTASVV |
| | LGCLVKDYFPE | CLLNNFYP |
| | PVTVSWNSGAL | REAKVQWK |
| | TSGVHTFPAVL | VDNALQSG |
| | QSSGLYSLSSV | NSQESVTE |
| | VTVPSSSLGTQ | QDSKDSTY |
| | TYICNVNHKPS | SLSSTLTL |
| | NTKVDKRVEPK | SKADYEKH |
| | SCDKTHTCPPC | KVYACEVT |
| | PAPELLGGPSV | HQGLSSPV |
| | FLFPPKPKDTL | TKSFNR |
| | MISRTPEVTCV | GEC |
| | VVDVSHEDPEV | (SEQ ID |
| | KFNWYVDGVEV | NO: 434) |
| | HNAKTKPREEQ | CDR1 |
| | YNSTYRVVSVL | (SEQ ID |
| | TVLHQDWLNGK | NO: 435)- |
| | EYKCKVSNKAL | RASQSVS |
| | PAPIEKTISKA | SSYLA |
| | KGQPREPQVYT | CDR2 |
| | LPPSREEMTKN | (SEQ ID |
| | QVSLTCLVKGF | NO: 436)- |
| | YPSDIAVEWES | GASSRAT |
| | NGQPENNYKTT | CDR3 |
| | PPVLDSDGSFF | (SEQ ID |
| | LYSKLTVDKSR | NO: 437)- |
| | WQQGNVFSCSV | CQQYSSSP |
| | MHEALHNHYTQ | LTFGQ |
| | KSLSLSPGK | |
| | (SEQ ID | |
| | NO :430) | |
| | CDR1 | |
| | (SEQ ID | |
| | NO: 431)- | |
| | FTFSSYAMSW | |
| | VR | |
| | CDR2 | |
| | (SEQ ID | |
| | NO: 432)- | |
| | VSAISGSGGST | |
| | YYADS | |
| | CDR3 | |
| | (SEQ ID | |
| | NO: 433)- | |
| | CARHGYSA | |
| | TRQGLDYWGQ | |

Alternatively, novel antigen-binding sites that can bind to CCR6 can be identified by screening for binding to the amino acid sequence defined by SEQ ID NO:438.

MSGESMNFSDVFDSSEDYFVSVNTSYYSVDSEMLLCSLQEVRQFSR
LFVPIAYSLICVFGLLGNILVVITFAFYKKARSMTDVYLLNMAIAD
ILFVLTLPFWAVSHATGAWVFSNATCKLLKGIYAINFNCGMLLLTC
ISMDRYIAIVQATKSFRLRSRTLPRSKIICLVVWGLSVIISSSTFV
FNQKYNTQGSDVCEPKYQTVSEPIRWKLLMLGLELLFGFFIPLMFM
IFCYTFIVKTLVQAQNSKRHKAIRVIIAVVLVFLACQIPHNMVLLV
TAANLGKMNRSCQSEKLIGYTKTVTEVLAFLHCCLNPVLYAFIGQK
FRNYFLKILKDLWCVRRKYKSSGFSCAGRYSENISRQTSETADNDN
ASSFTM

In certain embodiments, the present disclosure provides multi-specific binding proteins that bind to the NKG2D receptor and CD16 receptor on natural killer cells, and the tumor-associated antigen EPHA4. Table 17 lists some exemplary peptide sequences of heavy chain variable domains and light chain variable domains that, in combination, can bind to EPHA4.

TABLE 17

| | Heavy chain | Light chain |
|---|---|---|
| | variable | variable |
| | domain | domain amino |
| | amino acid | acid |
| Source | sequence | sequence |
| OncoTherapy | QVQLVQSGAEV | QSVLTQPPSAS |
| Science, | KKPGASVKVSC | GTPGQRVTISC |
| Inc. | KVSGYTLTELS | SGSSSNIGSNT |
| (U.S. | MHWVRQAPGKG | VNWYQQPPGTA |
| Pat. No. | LEWMGGFDPED | PKLLIYSNNQR |
| 8,003,098) | GETIYAQKFQG | PSGVPDRFSGS |
| | RVTMTEDTSTD | KSGTSASLAIS |
| | TAYMELSSLRS | GLQSEDEADYY |
| | EDTAVYYCATA | CAAWDDSLNGP |
| | QPFHWGDDAFD | VFGGGTKLTVL |
| | IWGQGTMVTVS | G |
| | S | (SEQ ID |
| | (SEQ ID | NO: 443) |
| | NO: 439) | CDR1 |
| | CDR1 | (SEQ ID |
| | (SEQ ID | NO: 444)- |
| | NO: 440)- | SGSSSNIGSN |
| | ELSMH | TVN |
| | CDR2 | CDR2 |
| | (SEQ ID | (SEQ ID |
| | NO: 441)- | NO: 445)- |
| | GFDPEDGET | SNNQRPS |
| | IYAQKFQG | CDR3 |
| | CDR3 | (SEQ ID |
| | (SEQ ID | NO:446)- |
| | NO: | AAWDDSLNGPV |
| | 442) - | |
| | AQPFHWGDD | |
| | AFDI | |
| HKUST | QVQLQQSGAEV | DVVMTQSPLSL |
| (U.S. Pat. | KKPGSSVKVSC | PVTPGEPASIS |
| Publication | KASGYTFTGYY | CRSSQSLLHSN |
| No. | MHWVRQAPGQG | GYNYLDWYLQK |
| 20170218075) | LEWMGGIIPIF | PGQSPQLLIYL |
| | GTANYAQKFQG | GSNRASGVPDR |
| | RVTITADKSTS | FSGSGSGTDFT |
| | TAYMELSSLRS | LKISRVEAEDV |
| | EDTAVYYCATA | GVYYCMQALQT |
| | PMVCSSTSCYL | PITFGQGTRLE |
| | RGFDYWGQGTL | IK |
| | VTVSS | (SEQ ID |
| | (SEQ ID | NO: 451) |
| | NO: 447) | CDR1 |
| | CDR1 | (SEQ ID |
| | (SEQ ID | NO: 452)- |
| | NO: 448)- | QSLLHSNGYNY |
| | GYTFTGYY | CDR2 |
| | CDR2 | (SEQ ID |
| | (SEQ ID | NO: 453)- |
| | NO: 449)- | LGS |
| | IIPIFGTA | CDR3 |
| | CDR3 | (SEQ ID |
| | (SEQ ID | NO: 454)- |
| | NO: 450)- | MQALQTP |
| | ATAPMVCS | |
| | STSCYLRG | |
| | FDY | |

Alternatively, novel antigen-binding sites that can bind to EPHA4 can be identified by screening for binding to the amino acid sequence defined by SEQ ID NO:455.

SEQ ID NO: 455
MAGIFYFALFSCLFGICDAVTGSRVYPANEVTLLDSRSVQGELGW
IASPLEGGWEEVSIMDEKNTPIRTYQVCNVMEPSQNNWLRTDWIT
REGAQRVYIEIKFTLRDCNSLPGVMGTCKETFNLYYYESDNDKER
FIRENQFVKIDTIAADESFTQVDIGDRIMKLNTEIRDVGPLSKKG
FYLAFQDVGACIALVSVRVFYKKCPLTVRNLAQFPDTITGADTSS
LVEVRGSCVNNSEEKDVPKMYCGADGEWLVPIGNCLCNAGHEERS
GECQACKIGYYKALSTDATCAKCPPHSYSVWEGATSCTCDRGFFR
ADNDAASMPCTRPPSAPLNLISNVNETSVNLEWSSPQNTGGRQDI
SYNVVCKKCGAGDPSKCRPCGSGVHYTPQQNGLKTTKVSITDLLA
HTNYTFEIWAVNGVSKYNPNPDQSVSVTVTTNQAAPSSIALVQAK
EVTRYSVALAWLEPDRPNGVILEYEVKYYEKDQNERSYRIVRTAA
RNTDIKGLNPLTSYVFHVRARTAAGYGDFSEPLEVTTNTVPSRII
GDGANSTVLLVSVSGSVVLVVILIAAFVISRRRSKYSKAKQEADE
EKHLNQGVRTYVDPFTYEDPNQAVREFAKEIDASCIKIEKVIGVG
EFGEVCSGRLKVPGKREICVAIKTLKAGYTDKQRRDFLSEASIMG
QFDHPNIIHLEGVVTKCKPVMHTEYMENGSLDAFLRKNDGRFTVI
QLVGMLRGIGSGMKYLSDMSYVHRDLAARNILVNSNLVCKVSDFG
MSRVLEDDPEAAYTTRGGKIPIRWTAPEAIAYRKFTSASDVWSYG
IVMWEVMSYGERPYWDMSNQDVIKAIEEGYRLPPPMDCPIALHQL
MLDCWQKERSDRPKFGQIVNMLDKLIRNPNSLKRTGTESSRPNTA
LLDPSSPEFSAVVSVGDWLQAIKMDRYKDNFTAAGYTTLEAVVHV
NQEDLARIGITAITHQNKILSSVQAMRTQMQQMHGRMVPV

In certain embodiments, the present disclosure provides multi-specific binding proteins that bind to the NKG2D receptor and CD16 receptor on natural killer cells, and the antigen CD14. Exemplary sequences of a heavy chain variable domain and a light chain variable domain that, in combination, bind to CD14 are listed in the US patent publication NO. 20170107294. In certain embodiments of the present invention, the heavy chain variable domain can be at least 95% identical to SEQ ID NO:456, and/or include amino acid sequences identical to the CDR1 (SEQ ID NO:457), CDR2 (SEQ ID NO:458), and CDR3 (SEQ ID NO:459) sequences of SEQ ID NO:456, and the light chain variable domain can be at least 95% identical to SEQ ID NO:460, and/or include amino acid sequences identical to the CDR1 (SEQ ID NO:461), CDR2 (SEQ ID NO:462), and CDR3 (SEQ ID NO:463) sequences of SEQ ID NO:460. Table 18 lists some exemplary peptide sequences of heavy chain variable domains and CDRs, and light chain variable domains and CDRs that, in combination, can bind to CD14.

TABLE 18

| | Heavy chain variable | Light chain variable |
|---|---|---|
| Source | domain amino acid sequence | domain amino acid sequence |
| Nordlandssykehuset | MGWSCIILFLVATATGVH | MGWSCIILFLVATATGVHSEVQ |
| Hf | SNIVLTQSPASLAVSLGQ | LVESGGGLMQPKGSLKLSCAA |
| (U.S. Patent | RATISCRASESVDSYGNS | SGFTFKTYALNWVRQAPGTGL |
| Publication No. | FMHWYQQKPGQPPKLLI | EWVARIRSKSNNYTTYYADSV |
| 20170107294) | YLASNLESGVPARFSGSG | KDRFTISRDDSQNMLYLQMNN |
| | SRTDFTLTIDPVEADDVA | LKTEDTAMYYCVRPQSGTSFA |
| | TYYCQQNNGDPYTFGGG | YWGQGTLVTVSAA (SEQ ID |
| | TKLEIIR (SEQ ID NO: 456) | NO: 460) |
| | CDR1 (SEQ ID NO: 457)- | CDR1 (SEQ ID NO: 461)- |
| | RASESVDSYGNSFM | GFTFKTYALN |
| | CDR2 (SEQ ID NO: 458)- | CDR2 (SEQ ID NO: 462)- |
| | LASNLES | RIRSKSNNYTTYYADSVK |
| | CDR3 (SEQ ID NO: 459)- | CDR3 (SEQ ID NO: 463)- |
| | QQNNGDPYT | PQSGTSFAY |
| Mochida Seiyaku | CDR1 (SEQ ID NO: 464)- | CDR1 (SEQ ID NO: 467)- |
| (U.S. Patent | RYTMG | QASQSIGSNLA |
| Publication No. | CDR2 (SEQ ID NO: 465)- | CDR2 (SEQ ID NO: 468)- |
| 20150239982) | IINSGATYYASWAKG | KASKLAS |
| | CDR3 (SEQ ID NO: 466)- | CDR3 (SEQ ID NO: 469)- |
| | GDA | QCSYTAIGNYGHV |

Alternatively, novel antigen-binding sites that bind to CD14 can be identified by screening for binding to the amino acid sequence defined by SEQ ID NO:470.

SEQ ID NO: 470
MERASCLLLLLLPLVHVSATTPEPCELDDEDFRCVCNFSEPQPDWSEAF
QCVSAVEVEIHAGGLNLEPFLKRVDADADPRQYADTVKALRVRRLTVGA
AQVPAQLLVGALRVLAYSRLKELTLEDLKITGTMPPLPLEATGLALSSL
RLRNVSWATGRSWLAELQQWLKPGLKVLSIAQAHSPAFSCEQVRAFPAL
TSLDLSDNPGLGERGLMAALCPHKFPAIQNLALRNTGMETPTGVCAALA
AAGVQPHSLDLSHNSLRATVNPSAPRCMWSSALNSLNLSFAGLEQVPKG
LPAKLRVLDLSCNRLNRAPQPDELPEVDNLTLDGNPFLVPGTALPHEGS
MNSGVVPACARSTLSVGVSGTLVLLQGARGFA

In certain embodiments, the present disclosure provides multi-specific binding proteins that bind to the NKG2D receptor and CD16 receptor on natural killer cells, and the antigen CD163. Table 19 lists some exemplary sequences of heavy chain variable domains and light chain variable domains that, in combination, can bind to CD163.

TABLE 19

| | Heavy chain variable | Light chain variable |
|---|---|---|
| Source | domain amino acid sequence | domain amino acid sequence |
| Cytoguide AS | QVQLQESGPGLVKPSETLS | DIVMTQSPSSLSASVGDRVTITCR |
| (U.S. Patent | LTCTVSGYSITSDYAWNW | ASQSVSSDVAWFQQKPGKSPKPL |
| Publication | IRFPGKKLEWMGSIYYS | IYYASNRYSGVPSRFSGSGSGTDF |
| No. | GSTYYNPSLKSRVTISVDT | TLTISSLQAEDFAVYFCGQDYTSP |
| 20170119790) | SKNQFSLKLSSVTAADTA | RTFGGGTKLEIKRA (SEQ ID |
| | TYYCVSGTYYFDYWGQG | NO: 475) |
| | TTLTVSS | CDR1 (SEQ ID NO: 476)- |
| | (SEQ ID NO: 471) | ASQSVSSDV |
| | CDR1 (SEQ ID NO: 472)- | CDR2 (SEQ ID NO: 477)-YAS |
| | GYSITSDY | CDR3 (SEQ ID NO: 478)- |
| | CDR2 (SEQ ID NO: 473)- | QDYTSPRT |
| | YSG | |
| | CDR3 (SEQ ID NO: 474)- | |
| | CVSGTYYFDYWG | |
| Cytoguide AS | DVQLQESGPGLVKPSQSL | SVVMTQTPKSLLISIGDRVTITCK |
| (U.S. Patent | SLTCTVTGYSITSDYAWN | ASQSVSSDVAWFQQKPGQSPKPL |
| Publication | WIRQFPGNKLEWMGYITY | IYYASNRYTGVPDRFTGSGYGTD |
| No. | SGITNYNPSLKSQISITRDT | FTFTISSVQAEDLAVYFCGQDYT |
| 20170119790) | SKNQFFLQLNSVTTEDTA | SPRTFGGGTKLEIKR (SEQ ID |
| | TYYCVSGTYYFDYWGQG | NO: 483) |
| | TTLTVSS (SEQ ID NO: 479) | CDR1 (SEQ ID NO: 484)- |
| | CDR1 (SEQ ID NO: 480)- | ASQSVSSDV |
| | GYSITSDY | CDR2 (SEQ ID NO: 485)-YAS |
| | CDR2 (SEQ ID NO: 481)- | CDR3 (SEQ ID NO: 486)- |
| | YSG | QDYTSPRT |
| | CDR3 (SEQ ID NO: 482)- | |
| | YYFDYWG | |

Alternatively, novel antigen-binding sites that can bind to CD163 can be identified by screening for binding to the amino acid sequence defined by SEQ ID NO:487.

SEQ ID NO: 487
MSKLRMVLLEDSGSADPRRHFVNLSPFTITVVLLLSACFVTSSLGGTD
KELRLVDGENKCSGRVEVKVQEEWGTVCNNGWSMEAVSVICNQLGCPT
AIKAPGWANSSAGSGRIWMDHVSCRGNESALWDCKHDGWGKHSNCTHQ
QDAGVTCSDGSNLEMRLTRGGNMCSGRIEIKFQGRWGTVCDDNFNIDH
ASVICRQLECGSAVSFSGSSNFGEGSGPIWFDDLICNGNESALWNCKH
QGWGKHNCDHAEDAGVICSKGADLSLRLVDGVTECSGRLEVRFQGEWG
TICDDGWDSYDAAVACKQLGCPTAVTAIGRVNASKGFGHIWLDSVSCQ
GHEPAIWQCKHHEWGKHYCNHNEDAGVTCSDGSDLELRLRGGGSRCAG
TVEVEIQRLLGKVCDRGWGLKEADVVCRQLGCGSALKTSYQVYSKIQA
TNTWLFLSSCNGNETSLWDCKNWQWGGLTCDHYEEAKITCSAHREPRL
VGGDIPCSGRVEVKHGDTWGSICDSDFSLEAASVLCRELQCGTVVSIL
GGAHFGEGNGQIWAEEFQCEGHESHLSLCPVAPRPEGTCSHSRDVGVV
CSRYTEIRLVNGKTPCEGRVELKTLGAWGSLCNSHWDIEDAHVLCQQL
KCGVALSTPGGARFGKGNGQIWRHMFHCTGTEQHMGDCPVTALGASLC
PSEQVASVICSGNQSQTLSSCNSSSLGPTRPTIPEESAVACIESGQLR
LVNGGGRCAGRVEIYHEGSWGTICDDSWDLSDAHVVCRQLGCGEAINA
TGSAHFGEGTGPIWLDEMKCNGKESRIWQCHSHGWGQQNCRHKEDAGV
ICSEFMSLRLTSEASREACAGRLEVFYNGAWGTVGKSSMSETTVGVVC
RQLGCADKGKINPASLDKAMSIPMWVDNVQCPKGPDTLWQCPSSPWEK
RLASPSEETWITCDNKIRLQEGPTSCSGRVEIWHGGSWGTVCDDSWDL
DDAQVVCQQLGCGPALKAFKEAEFGQGTGPIWLNEVKCKGNESSLWDC
PARRWGHSECGHKEDAAVNCTDISVQKTPQKATTGRSSRQSSFIAVGI
LGVVLLAIFVALFFLTKKRRQRQRLAVSSRGENLVHQIQYREMNSCLN
ADDLDLMNSSENSHESADFSAAELISVSKFLPISGMEKEAILSHTEKE
NGNL

In certain embodiments, the present disclosure provides multi-specific binding proteins that bind to the NKG2D receptor and CD16 receptor on natural killer cells, and the antigen CSF3R. Table 20 lists some exemplary peptide sequences of heavy chain variable domains and light chain variable domains that, in combination, can bind to CSF3R.

TABLE 20

| | Heavy chain variable | Light chain variable |
|---|---|---|
| Source | domain amino acid sequence | domain amino acid sequence |
| CSL Ltd. | EVQLLESGGGLVQPGGSLRL | DIQMTQSPSALSASVGDRVTIT |
| (U.S. Patent | SCAASGFTFSLYWMGWVRQ | CRASQGISSYLNWYQQKPGK |
| Publication | APGKGLEWVSSISSSGGVTP | APKLLIYYASNLQNGIPSRFSG |
| No. | YADSVKGRFTISRDNSKNTL | SGSGTDFTLTISSLQPEDFATY |
| 20170226214) | YLQMNSLRAEDTAVYYCAM | HCQQSYSTPLTFGGGTNVEIR |
| | LGELGWFDPWGQGTLVTVS | (SEQ ID NO: 492) |
| | S | CDR1 (SEQ ID NO: 493)- |
| | (SEQ ID NO: 488) | RASQGISSYLN |
| | CDR1 (SEQ ID NO: 489)- | CDR2 (SEQ ID NO: 494)- |
| | LYWMG | ASNLQN |
| | CDR2 (SEQ ID NO: 490)- | CDR3 (SEQ ID NO: 495)- |
| | SISSSGGVTPYADSVKG | QQSYSTPLT |
| | CDR3 (SEQ ID NO: 491)- | |
| | LGELGWFDP | |

Alternatively, novel antigen-binding sites that can bind to CSF3R can be identified by screening for binding to the amino acid sequence defined by SEQ ID NO:496.

SEQ ID NO: 496
MARLGNCSLTWAALIILLLPGSLEECGHISVSAPIVHLGDPITASCIIK
QNCSHLDPEPQILWRLGAELQPGGRQQRLSDGTQESIITLPHLNHTQAF
LSCCLNWGNSLQILDQVELRAGYPPAIPHNLSCLMNLTTSSLICQWEPG
PETHLPTSFTLKSFKSRGNCQTQGDSILDCVPKDGQSHCCIPRKHLLLY
QNMGIWVQAENALGTSMSPQLCLDPMDVVKLEPPMLRTMDPSPEAAPPQ
AGCLQLCWEPWQPGLHINQKCELRHKPQRGEASWALVGPLPLEALQYEL
CGLLPATAYTLQIRCIRWPLPGHWSDWSPSLELRTTERAPTVRLDTWWR
QRQLDPRTVQLFWKPVPLEEDSGRIQGYVVSWRPSGQAGAILPLCNTTE
LSCTFHLPSEAQEVALVAYNSAGTSRPTPVVFSESRGPALTRLHAMARD
PHSLWVGWEPPNPWPQGYVIEWGLGPPSASNSNKTWRMEQNGRATGFLL
KENIRPFQLYEIIVTPLYQDTMGPSQHVYAYSQEMAPSHAPELHLKHIG
KTWAQLEWVPEPPELGKSPLTHYTIFWTNAQNQSFSAILNASSRGFVLH
GLEPASLYHIHLMAASQAGATNSTVLTLMTLTPEGSELHIILGLFGLLL
LLTCLCGTAWLCCSPNRKNPLWPSVPDPAHSSLGSWVPTIMEEDAFQLP
GLGTPPITKLTVLEEDEKKPVPWESHNSSETCGLPTLVQTYVLQGDPRA
VSTQPQSQSGTSDQVLYGQLLGSPTSPGPGHYLRCDSTQPLLAGLTPSP
KSYENLWFQASPLGTLVTPAPSQEDDCVFGPLLNFPLLQGIRVHGMEAL
GSF

In certain embodiments, the present disclosure provides multi-specific binding proteins that bind to the NKG2D receptor and CD16 receptor on natural killer cells, and the antigen Siglec-9. Table 21 lists some exemplary peptide sequences of heavy chain variable domains and light chain variable domains that, in combination, can bind to Siglec-9.

TABLE 21

| | Heavy chain variable | Light chain variable |
|---|---|---|
| Source | domain amino acid sequence | domain amino acid sequence |
| Innate Pharma | DVQLQESGPGLVKPSQSL | DIVMTQSHKFMSTSVGDRVSI |
| (PCT Publication | SLTCTVTGYSITGGFAWN | TCKASQDVNTAVAWYQQKPG |
| No. | WIRQFPGNTLEWMGYIGY | QSPKLLIYSASYRYTGVPDRFT |
| WO2017153433) | GGSTSYNPSLNSRISITRDT | GSGSGTDFTFTISSVQAEDLAV |
| | SKNHFFLQFNSVTTDDSA | YYCQQHYSTPRTFGGGTKLEI |
| | TYYCARGDYLFAYWGQG | K |
| | TLVTVSA | (SEQ ID NO: 501) |
| | (SEQ ID NO: 497) | CDR1 (SEQ ID NO: 502)- |
| | CDR1 (SEQ ID NO: 498)- | KASQDVNTAVA |
| | GGFAWN | CDR2 (SEQ ID NO: 503)- |
| | CDR2 (SEQ ID NO: 499)- | SASYRYT |
| | YIGYGGSTSYNPSLNS | CDR3 (SEQ ID NO: 504)- |
| | CDR3 (SEQ ID NO: 500)- | QQHYSTPRT |
| | GDYLFAY | |
| Alector LLC | EVKLVESGGALVKPGGSL | DIQMTQSPSSLSASLGERVSLT |
| (PCT Publication | KLSCAASGFTFSNYAMSW | CRASQEISGYLGWLQQKPDGT |
| No. | VRQTPEKRLEWVATINNG | IKRLIFSTSTLDSGVPKRFSGSR |
| WO2017075432) | GSYTYYSDSVKGRFAISR | SGSDYSLTISSLESEDFADYYC |
| | DNAKNTLYLQMSNLRSE | LQYASYPPTFGGGTKLEIK |
| | DTALYYCVRRDYGTSDF | (SEQ ID NO: 509) |
| | DYVVGQGTTLTVSS | CDR1 (SEQ ID NO: 510)- |
| | (SEQ ID NO: 505) | RASQEISGYLG |
| | CDR1 (SEQ ID NO: 506)- | CDR2 (SEQ ID NO: 511)- |
| | FTFSNYAMS | STSTLDS |
| | CDR2 (SEQ ID NO: 507)- | CDR3 (SEQ ID NO: 512)- |
| | VATINNGGSYTYYS | LQYASYPPT |
| | CDR3 (SEQ ID NO: 508)- | |
| | VRRDYGTSDFDY | |

Alternatively, novel antigen-binding sites that can bind to Siglec-9 can be identified by screening for binding to the amino acid sequence defined by SEQ ID NO:513.

SEQ ID NO: 513
MLLLLLPLLWGRERAEGQTSKLLTMQSSVTVQEGLCVHVPCSFSYPSH
GWIYPGPVVHGYWFREGANTDQDAPVATNNPARAVWEETRDRFHLLGD
PHTKNCTLSIRDARRSDAGRYFFRMEKGSIKWNYKHHRLSVNVTALTH
RPNILIPGTLESGCPQNLTCSVPWACEQGTPPMISWIGTSVSPLDPST
TRSSVLTLIPQPQDHGTSLTCQVTFPGASVTTNKTVHLNVSYPPQNLT
MTVFQGDGTVSTVLGNGSSLSLPEGQSLRLVCAVDAVDSNPPARLSLS
WRGLTLCPSQPSNPGVLELPWVHLRDAAEFTCRAQNPLGSQQVYLNVS
LQSKATSGVTQGVVGGAGATALVFLSFCVIFVVVRSCRKKSARPAAGV
GDTGIEDANAVRGSASQILNHFIGFPTFLGLGFEFLLNLRDLCCHPDS
EFYVYHFSHFRLIKNIAGEIVWSLEGKILWLLDVSDFFHWPFLICVG

In certain embodiments, the present disclosure provides multi-specific binding proteins that bind to the NKG2D receptor and CD16 receptor on natural killer cells, and the antigen ITGAM. Table 22 lists some exemplary peptide sequences of heavy chain variable domains and light chain variable domains that, in combination, can bind to ITGAM.

TABLE 22

| | Heavy chain variable | Light chain variable |
|---|---|---|
| Source | domain amino acid sequence | domain amino acid sequence |
| The General | VQLVESGGGLVQPGGSLR | DIVMSQSPDSLAVSLGERVTL |
| Hospital | LSCAASGFNIKDTYIHWV | NCKSSQLLYSSNQKNYLAWY |
| Corporation | RQAPGKGLEWVARIDPAN | QQKPGQSPKLLIYWASARESG |
| (PCT Publication | DKTRYADSVKGRFTISAD | VPDRFSGSGSGTDFTLTISSVQ |
| No. | TSKNTAYLQMNSLRAEDT | AEDVAVYYCQQYYSYPLTFG |
| WO2016138538) | AVYYCSSEGHYGYDGYA | AGTKLELK |
| | MDYWGQGTLVTVSS | (SEQ ID NO: 518) |
| | (SEQ ID NO: 514) | CDR1 (SEQ ID NO: 519)- |
| | CDR1 (SEQ ID NO: 515)- | YSSNQKNY |
| | GFNIKD | CDR2 (SEQ ID NO: 520)-WAS |
| | CDR2 (SEQ ID NO: 516)- | CDR3 (SEQ ID NO: 521)- |
| | DPANDK | YYSYPL |
| | CDR3 (SEQ ID NO: 517)- | |
| | HYGYDGYA | |

Alternatively, novel antigen-binding sites that can bind to ITGAM can be identified by screening for binding to the amino acid sequence defined by SEQ ID NO:522.

SEQ ID NO: 522
MALRVLLLTALTLCHGFNLDTENAMTFQENARGFGQSVVQLQGSRVVV
GAPQEIVAANQRGSLYQCDYSTGSCEPIRLQVPVEAVNMSLGLSLAAT
TSPPQLLACGPTVHQTCSENTYVKGLCFLFGSNLRQQPQKFPEALRGC
PQEDSDIAFLIDGSGSIIPHDFRRMKEFVSTVMEQLKKSKTLFSLMQY
SEEPRIHFTFKEFQNNPNPRSLVKPITQLLGRTHTATGIRKVVRELFN
ITNGARKNAFKILVVITDGEKFGDPLGYEDVIPEADREGVIRYVIGVG
DAPRSEKSRQELNTIASKPPRDHVFQVNNPBALKTIQNQLREKIFAIE
GTQTGSSSSFEHEMSQEGFSAAITSNGPLLSTVGSYDWAGGVFLYTSK
EKSTFINMTRVDSDMNDAYLGYAAAIILRNRVQSLVLGAPRYQHIGLV
AMFRQNTGMWESNANVKGTQIGAYFGASLCSVDVDSNGSTDLVLIGAP
HYYEQTRGGQVSVCPLPRGQRARWQCDAVLYGEQGQPWGRFGAALTVL
GDVNGDKLTDVAIGAPGEEDNRGAVYLFHGTSGSGISPSHSQRIAGSK
LSPRLQYFGQSLSGGQDLTMDGLVDLTVGAQGHVLLLRSQPVLRVKAI
MEFNPREVARNVFECNDQVVKGKEAGEVRVCLHVQKSTRDRLREGQIQ
SVVTYDLALDSGRPHSRAVFNETKNSTRRQTQVLGLTQTCETLKLQLP
NCIEDPVSPIVLRLNFSLVGTPLSAFGNLRPVLAEDAQRLFTALFPFE
KNCGNDNICQDDLSITFSFMSLDCLVVGGPREFNVTVTVRNDGEDSYR
TQVTFFFPLDLSYRKVSTLQNQRSQRSWRLACESASSTEVSGALKSTS
CSINHPIFPENSEVTFNITFDVDSKASLGNKLLLKANVTSENNMPRTN
KTEFQLELPVKYAVYMVVTSHGVSTKYLNFTASENTSRVMQHQYQVSN
LGQRSLPISLVFLVPVRLNQTVIWDRPQVTFSENLSSTCHTKERLPSH
SDFLAELRKAPVVNCSIAVCQRIQCDIPFFGIQEEFNATLKGNLSFDW
YIKTSHNHLLIVSTAEILFNDSVFTLLPGQGAFVRSQTETKVEPFEVP
NPLPLIVGSSVGGLLLLALITAALYKLGFFKRQYKDMMSEGGPPGAEP
Q

In certain embodiments, the present disclosure provides multi-specific binding proteins that bind to the NKG2D receptor and CD16 receptor on natural killer cells, and the antigen CCR1. Table 23 lists some exemplary peptide sequences of heavy chain variable domains and light chain variable domains that, in combination, can bind to CCR1.

TABLE 23

| | Heavy chain variable | Light chain variable |
|---|---|---|
| Source | domain amino acid sequence | domain amino acid sequence |
| (PCT Publication | DVKLVESGEGLVKPGGS | DIQMTQSPSSLSASLGGKVTIT |
| No. | LKLSCAASGFTFSRNAMS | CKASQDINKYIAWYQHKPGQ |
| WO2017126587) | WVRQTPEKRLEWVAYIS | GPRLLIHYTSSLQPGIPSRFSGS |
| | SGSDYIYYADTVKGRFTV | GSGRDYSFSISNLEPEDIATYY |
| | SRDNARNTLYLQMTSLR | CLQYDYTMTFGGGTKLEIR |
| | SEDTAMYFCTRFSYGYG | (SEQ ID NO: 527) |
| | KNAPDYWGQGTSVTVSS | CDR1 (SEQ ID NO: 528)- |
| | (SEQ ID NO: 523) | KASQDINKYIA |
| | CDR1 (SEQ ID NO: 524)- | CDR2 (SEQ ID NO: 529)- |
| | RNAMS | YTSSLQP |
| | CDR2 (SEQ ID NO: 525)- | CDR3 (SEQ ID NO: 530)- |
| | YISSGSDYIYYADTVK | LQYDYTMT |
| | CDR3 (SEQ ID NO: 526)- | |
| | FSYGYGKNAPDY | |
| (PCT Publication | QVQLKQSGPGLVQPSQSL | DVVMTQTPRSLPVSLGDQASI |
| No. | SITCTVSGFSLNNYGVHW | SCRSRQSLIHSNGITFLHWYLQ |
| WO2017126587) | VRQPPGKGLEWLGVIWS | KAGQSPKLLIYKVSNRFSGVP |
| | AGTTVYNAAFISRLSISKD | DRFSGSGSGTDFTLRISRVEAE |
| | DSKSQVFFKMNSLQAGD | DLGVYFCSQGTHVPPTFGGGT |
| | TAIYYCAKDGSRYYTAM | KLEIK |
| | DYWGQGTSVTVSS | (SEQ ID NO: 535) |
| | (SEQ ID NO: 531) | CDR1 (SEQ ID NO: 536)- |
| | CDR1 (SEQ ID NO: 532)- | RSRQSLIHSNGITFLH |
| | NYGVH | CDR2 (SEQ ID NO: 537)- |
| | CDR2 (SEQ ID NO: 533)- | KVSNRFS |
| | VIWSAGTTVYNAAFIS | CDR3 (SEQ ID NO: 538)- |
| | CDR3 (SEQ ID NO: 534)- | SQGTHVPPT |
| | DGSRYYTAMDY | |

Alternatively, novel antigen-binding sites that can bind to CCR1 can be identified by screening for binding to the amino acid sequence defined by SEQ ID NO:539.

SEQ ID NO: 539
METPNTTEDYDTTTEFDYGDATPCQKVNERAFGAQLLPPLYSLVFVIGL
VGNILVVLVLVQYKRLKNMTSIYLLNLAISDLLFLFTLPFWIDYKLKDD
WVFGDAMCKILSGFYYTGLYSEIFFIILLTIDRYLAIVHAVFALRARTV
TFGVITSIIIWALAILASMPGLYFSKTQWEFTHHTCSLHFPHESLREWK
LFQALKLNLFGLVLPLLVMIICYTGIIKILLRRPNEKKSKAVRLIFVIM
IIFFLFWTPYNLTILISVFQDFLFTHECEQSRHLDLAVQVTEVIAYTHC
CVNPVIYAFVGERFRKYLRQLFHRRVAVHLVKWLPFLSVDRLERVSSTS
PSTGEHELSAGF

In certain embodiments, the present disclosure provides multi-specific binding proteins that bind to the NKG2D receptor and CD16 receptor on natural killer cells, and the antigen LRRC25. Antigen-binding sites that can bind to LRRC25 can be identified, for example, by screening for binding to the amino acid sequence defined by SEQ ID NO:540.

SEQ ID NO: 540
MGGTLAWTLLLPLLLRESDSLEPSCTVSSADVDWNAEFSATCLNFSGLS
LSLPHNQSLRASNVILLDLSGNGLRELPVTFFAHLQKLEVLNVLRNPLS
RVDGALAARCDLDLQADCNCALESWHDIRRDNCSGQKPLLCWDTTSSQH
NLSAFLEVSCAPGLASATIGAVVVSGCLLLGLAIAGPVLAWRLWRCRVA
RSRELNKPWAAQDGPKPGLGLQPRYGSRSAPKPQVAVPSCPSTPDYENM
FVGQPAAEHQWDEQGAHPSEDNDFYINYKDIDLASQPVYCNLQSLGQAP
MDEEEYVIPGH

In certain embodiments, the present disclosure provides multi-specific binding proteins that bind to the NKG2D receptor and CD16 receptor on natural killer cells, and the antigen PTAFR. The antigen-binding sites that can bind to PTAFR can be identified, for example, by screening for binding to the amino acid sequence defined by SEQ ID NO:541.

SEQ ID NO: 541
MEPHDSSHMDSEFRYTLFPIVYSIIFVLGVIANGYVLWVFARLYPCKKF
NEIKIFMVNLTMADMLFLITLPLWIVYYQNQGNWILPKFLCNVAGCLFF
INTYCSVAFLGVITYNRFQAVTRPIKTAQANTRKRGISLSLVIWVAIVG
AASYFLILDSTNTVPDSAGSGNVTRCFEHYEKGSVPVLIIHIFIVFSFF
LVFLIILFCNLVIIRTLLMQPVQQQRNAEVKRRALWMVCTVLAVFIICF
VPHHVVQLPWTLAELGFQDSKFHQAINDAHQVTLCLLSTNCVLDPVIYC
FLTKKFRKHLTEKFYSMRSSRKCSRATTDTVTEVVVPFNQIPGNSLKN

In certain embodiments, the present disclosure provides multi-specific binding proteins that bind to the NKG2D receptor and CD16 receptor on natural killer cells, and the antigen SIRPB1. The antigen-binding sites that can bind to SIRPB1 can be identified, for example, by screening for binding to the amino acid sequence defined by SEQ ID NO:542 (SIRPB1 isoform 1), or SEQ ID NO:543 (SIRPB1 isoform 3)

SEQ ID NO: 542
MPVPASWPHLPSPFLLMTLLLGRLTGVAGEDELQVIQPEKSVSVAAGES
ATLRCAMTSLIPVGPIMWFRGAGAGRELIYNQKEGHFPRVTTVSELTKR
NNLDFSISISNITPADAGTYYCVKFRKGSPDDVEFKSGAGTELSVRAKP
SAPVVSGPAVRATPEHTVSFTCESHGFSPRDITLKWFKNGNELSDFQTN
VDPAGDSVSYSIHSTARVVLTRGDVHSQVICEIAHITLQGDPLRGTANL
SEAIRVPPTLEVTQQPMRAENQANVTCQVSNFYPRGLQLTWLENGNVSR
TETASTLIENKDGTYNWMSWLLVNTCAHRDDVVLTCQVEHDGQQAVSKS
YALEISAHQKEHGSDITHEAALAPTAPLLVALLLGPKLLLVVGVSAIYI
CWKQKA
SEQ ID NO: 543
MPVPASWPHLPSPFLLMTLLLGRLTGVAGEEELQVIQPDKSISVAAGES
ATLHCTVTSLIPVGPIQWFRGAGPGRELIYNQKEGHFPRVTTVSDLTKR
NNMDFSIRISNITPADAGTYYCVKPRKGSPDHVEFKSGAGTELSVRAKP
SAPVVSGPAARATPQHTVSFTCESHGFSPRDITLKWFKNGNELSDFQTN
VDPAGDSVSYSIHSTAKVVLTREDVHSQVICEVAHVTLQGDPLRGTANL
SETIRVPPTLEVTQQPVRAENQVNVTCQVRKFYPQRLQLTWLENGNVSR
TETASTLTENKDGTYNWMSWLLVNVSAHRDDVKLTCQVEHDGQPAVSKS
HDLKVSAHPKEQGSNTAPGPALASAAPLLIAFLLGPKVLLVVGVSVIYV
YWKQKA

In certain embodiments, the present disclosure provides multi-specific binding proteins that bind to the NKG2D receptor and CD16 receptor on natural killer cells, and the antigen TLR2. Table 24 lists some exemplary peptide sequences of heavy chain variable domains and light chain variable domains that, in combination, can bind to TLR2.

TABLE 24

| | Heavy chain variable | Light chain variable |
|---|---|---|
| Source | domain amino acid sequence | domain amino acid sequence |
| Amgen | MSSPQSLKTLILTMGWSWIF | MESDTLLLWVLLLWVPGST |
| (U.S. Pat. No. | LFLLSGTAGVHSQVQLQQS | GDIVLTQSPASLAVSLGQRA |
| 9,458,234) | GPELVNPGASVKLSCKASG | TISCRASESVEYYGTSLMQ |
| | FTFTTYGINWVKQGPGQGL | WYQQKPGQPPKLLIFGASN |
| | EWIGWIYPRDGSTNFNENF | VESGVPVRFSGSGSGTDFSL |
| | KDKAALTVDTSSSTAYMEL | NIHPVEEDDIVMYFCQQSRK |
| | HSLTSEDSAVYFCARLTGG | LPWTFGGGTKLEIKRADAA |
| | TFLDYWGQGTTLTVSSAKT | PTVSIFPPSSE |
| | TPPSVYPLAPGSAA | (SEQ ID NO: 548) |
| | (SEQ ID NO: 544) | CDR1 (SEQ ID NO: 549)- |
| | CDR1 (SEQ ID NO: 545)- | ESVEYYGTSL |
| | GFTFTTYG | CDR2 (SEQ ID NO: 550)- |
| | CDR2 (SEQ ID NO: 546)- | GAS |
| | IYPRDGST | CDR3 (SEQ ID NO: 551)- |
| | CDR3 (SEQ ID NO: 547)- | QQSRKLPWT |
| | ARLTGGTFLDY | |
| OSPONA | QVQLVQSGSELKKPGASVK | DIVLTQSPATLSLSPGERATL |
| Therapeutic | LSCKASGFTFTTYGINWVR | SCRASESVEYYGTSLMQWY |
| (PCT Publication | AQPGQGLEWIGWIYPRDGS | QQKPGQPPKLLIFGASNVES |
| No. | TNFNENFKDRATITVDTSAS | GVPDRFSGSGSGTDFTLKIS |
| WO2011003925) | TAYMELSSLRSEDTAVYFC | RVEAEDVGMYFCQQSRKLP |
| | ARLTGGTFLDYWGQGTTVT | WTFGGGTKVEIKR |
| | VSSA | (SEQ ID NO: 556) |
| | (SEQ ID NO: 552) | CDR1 (SEQ ID NO: 557)- |
| | CDR1 (SEQ ID NO: 553)- | ES VEYYGTSLMQ |
| | GFTFTTY | CDR2 (SEQ ID NO: 558)- |
| | CDR2 (SEQ ID NO: 554)- | GASNVES |
| | YPRDGS | CDR3 (SEQ ID NO: 559)- |
| | CDR3 (SEQ ID NO: 555)- | QQSRKLPWT |
| | LTGGTFLDY | |

Alternatively, novel antigen-binding sites that can bind to TLR2 can be identified, for example, by screening for binding to the amino acid sequence defined by SEQ ID NO:560.

SEQ ID NO: 560
MPHTLWMVWVLGVIISLSKEESSNQASLSCDRNGICKGSSGSLNSIPSG
LTEAVKSLDLSNNRITYISNSDLQRCVNLQALVLTSNGINTIEEDSFSS
LGSLEHLDLSYNYLSNLSSSWFKPLSSLTFLNLLGNPYKTLGETSLFSH
LTKLQILRVGNMDTFTKIQRKDFAGLTFLEELEIDASDLQSYEPKSLKS
IQNVSHLILHMKQHILLLEIFVDVTSSVECLELRDTDLDTFHFSELSTG
ETNSLIKKFTFRNVKITDESLFQVMKLLNQISGLLELEFDDCTLNGVGN
FRASDNDRVIDPGKVETLTIRRLHIPRFYLFYDLSTLYSLTERVKRITV
ENSKVFLVPCLLSQHLKSLEYLDLSENLMVEEYLKNSACEDAWPSLQTL
ILRQNHLASLEKTGETLLTLKNLTNIDISKNSFHSMPETCQWPEKMKYL
NLSSTRIHSVTGCIPKTLEILDVSNNNLNLFSLNLPQLKELYISRNKLM
TLPDASLLPMLLVLKISRNAITTFSKEQLDSFHTLKTLEAGGNNFICSC
EFLSFTQEQQALAKVLIDWPANYLCDSPSHVRGQQVQDVRLSVSECHRT
ALVSGMCCALFLLILLTGVLCHRFHGLWYMKMMWAWLQAKRKPRKAPSR
NICYDAFVSYSERDAYWVENLMVQELENFNPPFKLCLHKRDFIPGKWII
DNIIDSIEKSHKTVFVLSENFVKSEWCKYELDFSHFRLFDENNDAAILI
LLEPIEKKAIPQRFCKLRKIMNTKTYLEWPMDEAQREGFWVNLRAAIKS

In certain embodiments, the present disclosure provides multi-specific binding proteins that bind to the NKG2D receptor and CD16 receptor on natural killer cells, and the antigen TLR4. Table 25 lists some exemplary peptide sequences of heavy chain variable domains and light chain variable domains that, in combination, can bind to TLR4.

TABLE 25

| | Heavy chain variable | Light chain variable |
|---|---|---|
| Source | domain amino acid sequence | domain amino acid sequence |
| Noviummne SA | QVTLKESGPGILQPSQTLSL | AIQMTQSTSSLSASLGDRVTIN |
| (U.S. Pat. No. | TCSFSGFSLTTYNIGVGWIR | CRASQDITNYLNWYQQKPDG |
| 10,023,646) | QPSGKGLEWLAHIWWND | TVRLLIYYTSKLHSGAPSRFSG |
| | NIYYNTVLKSRLTFSKDTS | RGSGTDYSLTISNLEQEDIATY |
| | NNQVFLKIASVDIADTATY | FCQQGNTFPWTFGGGTKLEIK |
| | YCIRMAEGRYDAMDYWG | R |
| | QGTSVTVSS | (SEQ ID NO: 565) |
| | (SEQ ID NO: 561) | CDR1 (SEQ ID NO: 566)- |
| | CDR1 (SEQ ID NO: 562)- | RASQDITNYLN |
| | TYNIGVG | CDR2 (SEQ ID NO: 567)- |
| | CDR2 (SEQ ID NO: 563)- | YTSKLHS |
| | HIWWNDNIYYNTVLKS | CDR3 (SEQ ID NO: 568)- |
| | CDR3 (SEQ ID NO: 564)- | QQGNTFPWT |
| | MAEGRYDAMDY | |
| Astellas | EVQLVESGGGLVQPGGSL | DIQMTQSPSSLSASVGDRVTIT |
| (U.S. Patent | RLSCAASGFTFDTYAMHW | CRASQSISSWLAWYQQKPGK |
| Publication No. | VRQAPGKGLEWVAGISWN | APKLLIYKASSLESGVPSRFSG |
| 20170306053) | SGNIGYADSVKGRFTISRD | SGSGTDFTLTISSLQPEDFATY |
| | NSKNTLYLQMNSLRAEDT | YCQQYSSYSWTFGQGTKVEIK |
| | AVYYCAKDWDNWNLFDY | R |
| | WGQGTLVTVSS | (SEQ ID NO: 573) |
| | (SEQ ID NO: 569) | CDR1 (SEQ ID NO: 574)- |
| | CDR1 (SEQ ID NO: 570)- | RASQSISSWLA |
| | GFTFDTYA | CDR2 (SEQ ID NO: 575)- |
| | CDR2 (SEQ ID NO: 571)- | KASSLES |
| | ISWNSGNIGYADSVKG | CDR3 (SEQ ID NO: 576)- |
| | CDR3 (SEQ ID NO: 572)- | QQYSSYSWT |
| | DWDNWNLFDY | |

Alternatively, novel antigen-binding sites that can bind to TLR4 can be identified, for example, by screening for binding to the amino acid sequence defined by SEQ ID NO:577.

SEQ ID NO: 577
MMSASRLAGTLIPAMAFLSCVRPESWEPCVEVVPNITYQCMELNFYKIP
DNLPFSTKNLDLSFNPLRHLGSYSFFSFPELQVLDLSRCEIQTIEDGAY
QSLSHLSTLILTGNPIQSLALGAFSGLSSLQKLVAVETNLASLENFPIG
HLKTLKELNVAHNLIQSFKLPEYFSNLTNLEHLDLSSNKIQSIYCTDLR
VLHQMPLLNLSLDLSLNPMNFIQPGAFKEIRLHKLTLRNNFDSLNVMKT
CIQGLAGLEVHRLVLGEFRNEGNLEKFDKSALEGLCNLTIEEFRLAYLD
YYLDDIIDLFNCLTNVSSFSLVSVTIERVKDFSYNFGWQHLELVNCKFG
QFPTLKLKSLKRLTFTSNKGGNAFSEVDLPSLEFLDLSRNGLSFKGCCS
QSDFGTTSLKYLDLSFNGVITMSSNFLGLEQLEHLDFQHSNLKQMSEFS
VFLSLRNLIYLDISHTHTRVAFNGIFNGLSSLEVLKMAGNSFQENFLPD
IFTELRNLTFLDLSQCQLEQLSPTAFNSLSSLQVLNMSHNNFFSLDTFP
YKCLNSLQVLDYSLNHIMTSKKQELQHFPSSLAFLNLTQNDFACTCEHQ
SFLQWIKDQRQLLVEVERMECATPSDKQGMPVLSLNITCQMNKTIIGVS
VLSVLVVSVVAVLVYKFYFHLMLLAGCIKYGRGENIYDAFVIYSSQDED
WVRNELVKNLEEGVPPFQLCLHYRDFIPGVAIAANIIHEGFHKSRKVIV
VVSQHFIQSRWCIFEYEIAQTWQFLSSRAGIIFIVLQKVEKTLLRQQVE
LYRLLSRNTYLEWEDSVLGRHIFWRRLRKALLDGKSWNPEGTVGTGCNW
QEATSI

In certain embodiments, the present disclosure provides multi-specific binding proteins that bind to the NKG2D receptor and CD16 receptor on natural killer cells, and the antigen CD300LB. The antigen-binding sites that can bind to CD300LB can be identified, for example, by screening for binding to the amino acid sequence defined by SEQ ID NO:578.

SEQ ID NO: 578
MWLPPALLLLSLSGCFSIQGPESVRAPEQGSLTVQCHYKQGWETYIKWW
CRGVRWDTCKILIETRGSEQGEKSDRVSIKDNQKDRTFTVTMEGLRRDD
ADVYWCGIERRGPDLGTQVKVIVDPEGAASTTASSPTNSNMAVFIGSHK
RNHYMLLVFVKVPILLILVTAILWLKGSQRVPEEPGEQPIYMNFSEPLT
KDMAT

In certain embodiments, the present disclosure provides multi-specific binding proteins that bind to the NKG2D receptor and CD16 receptor on natural killer cells, and the antigen ATP1A3. The antigen-binding sites that can bind to ATP1A3 can be identified, for example, by screening for binding to the amino acid sequence defined by SEQ ID NO:579 (ATP1A3 isoform 2), or SEQ ID NO:580 (ATP1A3 isoform 3).

SEQ ID NO: 579
MGGWEEERNRRATDKKDDKDSPKKNKGKERRDLDDLKKEVAMTEHKMSV
EEVCRKYNTDCVQGLTHSKAQEILARDGPNALTPPPTTPEWVKFCRQLF
GGFSILLWIGAILCFLAYGIQAGTEDDPSGDNLYLGIVLAAVVIITGCF
SYYQEAKSSKIMESFKNMVPQQALVIREGEKMQVNAEEVVVGDLVEIKG
GDRVPADLRIISAHGCKVDNSSLTGESEPQTRSPDCTHDNPLETRNITF
FSTNCVEGTARGVVVATGDRTVMGRIATLASGLEVGKTPIAIEIEHFIQ
LITGVAVFLGVSFPILSLILGYTWLEAVIFLIGIIVANVPEGLLATVTV
CLTLTAKRMARKNCLVKNLEAVETLGSTSTICSDKTGTLTQNRMTVAHM
WFDNQIHEADTTEDQSGTSPDKSSHTWVALSHIAGLCNRAVFKGGQDNI
PVLKRDVAGDASESALLKCIELSSGSVKLMRERNKKVAEIPFNSTNKYQ
LSIHETEDPNDNRYLLVMKGAPERILDRCSTILLQGKEQPLDEEMKEAF
QNAYLELGGLGERVLGFCHYYLPEEQFPKGFAFDCDDVNFTTDNLCFVG
LMSMIDPPRAAVPDAVGKCRSAGIKVIMVTGDHPITAKAIAKGVGIISE
GNETVEDIAARLNIPVSQVNPRDAKACVIHGTDLKDFTSEQIDEILQNH
TEIVFARTSPQQKLIIVEGCQRQGAIVAVTGDGVNDSPALKKADIGVAM
GIAGSDVSKQAADMILLDDNFASIVTGVEEGRLIFDNLKKSIAYTLTSN
IPEITPFLLFIMANIPLPLGTITILCIDLGTDMVPAISLAYEAAESDIM
KRQPRNPRTDKLVNERLISMAYGQIGMIQALGGFFSYFVILAENGFLPG
NLVGIRLNWDDRTVNDLEDSYGQQWTYEQRKVVEFTCHTAFFVSIVVVQ
WADLIICKTRRNSVFQQGMKNKILIFGLFEETALAAFLSYCPGMDVALR
MYPLKPSWWFCAFPYSFLIFVYDEIRKLILRRNPGGWVEKETYY
SEQ ID NO: 580
MGSGGSDSYRIATSQDKKDDKDSPKKNKGKERRDLDDLKKEVAMTEHKM
SVEEVCRKYNTDCVQGLTHSKAQEILARDGPNALTPPPTTPEWVKFCRQ
LFGGFSILLWIGAILCFLAYGIQAGTEDDPSGDNLYLGIVLAAVVIITG
CFSYYQEAKSSKIMESFKNMVPQQALVIREGEKMQVNAEEVVVGDLVEI
KGGDRVPADLRIISAHGCKVDNSSLTGESEPQTRSPDCTHDNPLETRNI
TFFSTNCVEGTARGVVVATGDRTVMGRIATLASGLEVGKTPIAIEIEHF
IQLITGVAVFLGVSFFILSLILGYTWLEAVIFLIGIIVANVPEGLLATV
TVCLTLTAKRMARKNCLVKNLEAVETLGSTSTICSDKTGTLTQNRMTVA
HMWFDNQIHEADTTEDQSGTSFDKSSHTWVALSHIAGLCNRAVFKGGQD
NIPVLKRDVAGDASESALLKCIELSSGSVKLMRERNKKVAEIPFNSTNK
YQLSIHETEDPNDNRYLLVMKGAPERILDRCSTILLQGKEQPLDEEMKE
AFQNAYLELGGLGERVLGFCHYYLPEEQFPKGFAFDCDDVNFTTDNLCF
VGLMSMIDPPRAAVPDAVGKCRSAGIKVIMVTGDHPITAKAIAKGVGII
SEGNETVEDIAARLNIPVSQVNPRDAKACVIHGTDLKDFTSEQIDEILQ
NHTEIVFARTSPQQKLIIVEGCQRQGAIVAVTGDGVNDSPALKKADIGV
AMGIAGSDVSKQAADMILLDDNFASIVTGVEEGRLIFDNLKKSIAYTLT
SNIPEITPFLLFIMANIPLPLGTITILCIDLGTDMVPAISLAYEAAESD
IMKRQPRNPRTDKLVNERLISMAYGQIGMIQALGGFFSYFVILAENGFL
PGNLVGIRLNWDDRTVNDLEDSYGQQWTYEQRKVVEFTCHTAFFVSIVV
VQWADLIICKTRRNSVFQQGMKNKILIFGLFEETALAAFLSYCPGMDVA
LRMYPLKPSWWFCAFPYSFLIFVYDEIRKLILRRNPGGWVEKETYY

In certain embodiments, the present disclosure provides multi-specific binding proteins that bind to the NKG2D receptor and CD16 receptor on natural killer cells, and the antigen CCR5. Table 26 lists some exemplary peptide sequences of heavy chain variable domains and light chain variable domains that, in combination, can bind to CCR5.

TABLE 26

| | Heavy chain variable | Light chain variable |
|---|---|---|
| Source | domain amino acid sequence | domain amino acid sequence |
| AbbVie | MEWSGVFIFLLSVTAGVHS | MKLPVRLLVLMFWIPASSSDI |
| (U.S. Pat. No. | EVQLVESGGGLVKPGGSL | VMTQSPLSLPVTPGEPASISCR |
| 7,851,600) | RLSCAASGYTFSNYWIGW | SSQRLLSSYGHTYLHWYLQKP |
| | VRQAPGKGLEWIGDIYPGG | GQSPQLLIYEVSNRFSGVPDRF |
| | NYIRNNEKFKDKTTLSADT | SGSGSGTDFTLKISRVEAEDVG |
| | SKNTAYLQMNSLKTEDTA | VYYCSQSTHVPLTFGQGTKVE |
| | VYYCGSSFGSNYVFAWFT | IK |
| | YWGQGTLVTVSS | (SEQ ID NO: 585) |
| | (SEQ ID NO: 581) | CDR1 (SEQ ID NO: 586)- |
| | CDR1 (SEQ ID NO: 582)- | RSSQRLLSSYGHTYLH |
| | GYTFSNYWIG | CDR2 (SEQ ID NO: 587)- |
| | CDR2 (SEQ ID NO: 583)- | EVSNRFS |
| | DIYPGGNYIRNNEKFKD | CDR3 (SEQ ID NO: 588)- |
| | CDR3 (SEQ ID NO: 584)- | SQSTHVPLT |
| | SFGSNYVFAWFTY | |
| CYTODYN/ | MEWSGVFIFLLSVTAGVHS | MKLPVRLLVLMFWIPASSSDI |
| AbbVie | QVQLVQSGPDVKKPGTSM | VMTQSPLSLPVTPGEPASISCR |
| (U.S. Pat. No. | KMSCKTSGYTFSNYWIGW | SSQRLLSSYGHTYLHWYLQKP |
| 7,851,600) | VRQAPGQGLEWIGDIYPGG | GQSPQLLIYEVSNRFSGVPDRF |
| | NYIRNNEKFKDKTTLTADT | SGSGSGTDFTLKISRVEAEDVG |
| | STSTAYMQLGSLRSEDTAV | VYYCSQSTHVPLTFGQGTKVE |
| | YYCGSSFGSNYVFAWFTY | IK |
| | WGQGTLVTVSS | (SEQ ID NO: 593) |
| | (SEQ ID NO: 589) | CDR1 (SEQ ID NO: 594)- |
| | CDR1 (SEQ ID NO: 590)- | RSSQRLLSSYGHTYLH |
| | GYTFSNYWIG | CDR2 (SEQ ID NO: 595)- |
| | CDR2 (SEQ ID NO: 591)- | EVSNRFS |
| | DIYPGGNYIRNNEKFKD | CDR3 (SEQ ID NO: 596)- |
| | CDR3 (SEQ ID NO: 592)- | SQSTHVPLT |
| | SFGSNYVFAWFTY | |

Alternatively, novel antigen-binding sites that can bind to CCR5 can be identified, for example, by screening for binding to the amino acid sequence defined by SEQ ID NO:597.

SEQ ID NO: 597
MDYQVSSPIYDINYYTSEPCQKINVKQIAARLLPPLYSLVFIFGFVGNM
LVILILINCKRLKSMTDIYLLNLAISDLFFLLTVPFWAHYAAAQWDFGN
TMCQLLTGLYFIGFFSGIPPIILLTIDRYLAVVHAVFALKARTVTFGVV
TSVITWVVAVFASLPGIIFTRSQKEGLHYTCSSHFPYSQYQFWKNFQTL
KIVILGLVLPLLVMVICYSGILKTLLRCRNEKKRHRAVRLIFTIMIVYF
LFWAPYNIVLLLNTFQEFFGLNNCSSSNRLDQAMQVTETLGMTHCCINP
IIYAFVGEKFRNYLLVFFQKHIAKRFCKCCSIFQQEAPERASSVYTRST
GEQEISVGL

In certain embodiments, the present disclosure provides multi-specific binding proteins that bind to the NKG2D receptor and CD16 receptor on natural killer cells, and the antigen B7-H4 or VTCN1 (V-Set Domain Containing T Cell Activation Inhibitor 1). Table 27 lists some exemplary peptide sequences of heavy chain variable domains and light chain variable domains that, in combination, can bind to B7-H4.

TABLE 27

| | Heavy chain variable | Light chain variable |
|---|---|---|
| Source | domain amino acid sequence | domain amino acid sequence |
| Milennium | GVQLVESGGGLVQPGGSLR | EIVMTQSPATLSVSPGERATL |
| Pharmaceuticals | LSCAASGFTISRNDMHWVR | ACRASQSVRSNLAWYQQKP |
| (U.S. Pat. No. | QATGKGLEWVSAIGTGGDT | GQAPRLLIYGASTRATGIPAR |
| 8,759,490) | YYPGSVKGRFTISRENAKNS | FSGSGSGTEFTLTISSLQSEDF |
| | LYLQMNSLRAGDTAVYYC | AVYSCQQYNNWPWTFGQ |
| | ARGHMTTFGGFIVIGNGMD | GTKVEIKR (SEQ ID NO: 602) |
| | VWVGQGTTVTVSS | CDR1: QSVRSNLA (SEQ ID |
| | (SEQ ID NO: 598) | NO: 603) |
| | CDR1: GFTISRN | CDR2: GASTRAT (SEQ ID |
| | (SEQ ID NO: 599) | NO: 604) |
| | CDR2: GTGGD (SEQ ID | CDR3: QQYNNWPWT (SEQ ID |
| | NO: 600) | NO: 605) |
| | CDR3: | |
| | GHMTTFGGFIVIGNGMDV | |
| | (SEQ IDN O: 601) | |
| ER Squibb and | QVQLQQWGAGLLKPSETLS | EIVLTQFPGTLSLSPGERATL |
| Sona LLC | LTCAVYGGSFSDYFWTWIR | SCRASQSVSSTYLAWYQQKP |
| (U.S. Pat. No. | QPPGKGLEWIGEINHSGTTN | GQAPRVLIYGASRRATGIPD |
| 9,988,453) | YNPSLKSRVTISADTSKNQF | FRSGSGSGTDFTLTISRLEPE |
| | SRLSSVTAADTAVYYCARL | DFAVYYCQQYGSSPLTFGGG |
| | SSWSNWAFEYWGQGTLVT | TKVEIK (SEQ ID NO: 610) |
| | VSS (SEQ ID NO: 606) | CDRL1: RASQSVSSTYLA |
| | CDRH1: DYFWT (SEQ ID | (SEQ ID NO: 611) |
| | NO: 607) | CDRL2: GASRRAT (SEQ ID |
| | CDRH2: | NO: 612) |
| | EINHSGTTNYNPSLKS (SEQ | CDRL3: QQYGSSPLT (SEQ |
| | ID NO: 608) | ID NO: 613) |
| | CDRS3: LSSWSNWAFEY | |
| | (SEQ ID NO: 609 | |

Alternatively, novel antigen-binding sites that can bind to B7-H4 can be identified, for example, by screening for binding to the amino acid sequence defined by SEQ ID NO:614.

SEQ ID NO: 614
MASLGQILFWSIISIIIILAGAIALIIGFGISGRHSITVTTVASAGNIG
EDGILSCTFEPDIKLSDIVIQWLKEGVLGLVHEFKEGKDELSEQDEMFR
GRTAVFADQVIVGNASLRLKNVQLTDAGTYKCYIITSKGKGNANLEYKT
GAFSMPEVNVDYNASSETLRCEAPRWFPQPTVVWASQVDQGANFSEVSN
TSFELNSENVTMKVVSVLYNVTINNTYSCMIENDIAKATGDIKVTESEI
KRRSHLQLLNSKASLCVSSFFAISWALLPLSPYLMLK

In certain embodiments, the present disclosure provides multi-specific binding proteins that bind to the NKG2D receptor and CD16 receptor on natural killer cells, and the antigen V-domain Ig suppressor of T cell activation (VISTA). Table 28 lists some exemplary peptide sequences of heavy chain variable domains and light chain variable domains that, in combination, can bind to VISTA.

TABLE 28

| | Heavy chain variable | Light chain variable |
|---|---|---|
| Source | domain amino acid sequence | domain amino acid sequence |
| MedImmune | QVQLQQSGAELVKPGAS | DIVMSQSPSSLAVSAGEKVT |
| (U.S. Patent | VKLSCKASGYTFTSHDIN | MSCKSSQSLLNSRTRKNQLA |
| Publication No. | WVRQRPELGLEWIGWIFP | WYQQKPGQSPKLLIYWAFIRE |
| US20160096891) | GDGSTKFNEKFKGKATL | SGVPDRFTGSGSGTDFTLT |
| | TTDKSSSTAYIQLSRLTSE | ISSVQAEDLAVYYCKQSYNL |
| | DSAVYFCARNSFYSMDY | RTFGGGIKLEIK (SEQ ID |
| | WGQGTSVTVSS (SEQ ID | NO: 619) |
| | NO: 615) | CDRL1: QSLLNSRTRKNQ |
| | CDH1: GYTFTSHD (SEQ | (SEQ ID NO: 620) |
| | ID NO: 616) | CDRL2: WAF (SEQ ID NO: 621) |
| | CDH2: IFPGDGST (SEQ ID | CDRL3: KQSYNLRT (SEQ ID |
| | NO: 617) | NO: 622) |
| | CDH3: ARNSFYSMDY | |
| | (SEQ ID NO: 618) | |
| Pierre Fabre | EVQLLQSGPELEKPGASV | DVLMTQTPLSLPVSLGDQASIS |
| Medicament SA | KISCKASGYSFTGYNMN | CRSSQSIVHSNGNTYLEWYLQ |
| (U.S. Pat.No. | WVKQSNGKSLEWIGNIDP | KPGQSPKLLIYKVSNRFSGVPD |
| 10,100,123) | YYDYTSYNLKFKDKATL | RFSGSGSGTDFTLKINRVEAED |
| | TVDKSSSTAYMQLKSLTS | LGVYYCFQGSHVPWTFGGGT |
| | EDSAVYYCATSTMITPFD | KLEIK (SEQ ID NO: 627) |
| | YWGQGTTLTVSS (SEQ ID | CDR1: RSSQSIVHSNGNTYLE |
| | NO: 623) | (SEQ ID NO: 628) |
| | CDH1: GYSFTGYNMN | CDR2: KVSNRFS (SEQ ID |
| | (SEQ ID NO: 624) | NO: 629) |
| | CDH2: | CDR3: FQGSHVPWT (SEQ ID |
| | NIDPYYDYTSYNLKFKD | NO: 630) |
| | (SEQ ID NO: 625) | |
| | CDH3: STMITPFDY (SEQ | |
| | ID NO: 626) | |

Alternatively, novel antigen-binding sites that can bind to VISTA can be identified, for example, by screening for binding to the amino acid sequence defined by SEQ ID NO:631.

SEQ ID NO: 631
MGVPTALEAGSWRWGSLLFALFLAASLGPVAAFKVATPYSLYVCPEGQN
VTLTCRLLGPVDKGHDVTFYKTWYRSSRGEVQTCSERRPIRNLTFQDLH
LHHGGHQAANTSHDLAQRHGLESASDHHGNFSITMRNLTLLDSGLYCCL
VVEIRHHHSEHRVHGAMELQVQTGKDAPSNCVVYPSSSQDSENITAAAL
ATGACIVGILCLPLILLLVYKQRQAASNRRAQELVRMDSNIQGIENPGF
EASPPAQGIPEAKVRHPLSYVAQRQPSESGRHLLSEPSTPLSPPGPGDV
FFPSLDPVPDSPNFEVI

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, e.g., Sondermann P et al. (2000) Nature; 406(6793):267-273.). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

The assembly of heterodimeric antibody heavy chains can be accomplished by expressing two different antibody heavy chain sequences in the same cell, which may lead to the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, U.S. Ser. No. 14/773,418, U.S. Ser. No. 12/811,207, U.S. Ser. No. 13/866,756, U.S. Ser. No. 14/647,480, and U.S. Ser. No. 14/830,336. For example, mutations can be made in the CH3 domain based on human IgG1 and incorporating distinct pairs of amino acid substitutions within a first polypeptide and a second polypeptide that allow these two chains to selectively heterodimerize with each other. The positions of amino acid substitutions illustrated below are all numbered according to the EU index as in Kabat.

In one scenario, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V.

An antibody heavy chain variable domain of the invention can optionally be coupled to an amino acid sequence at least 90% identical to an antibody constant region, such as an IgG constant region including hinge, CH2 and CH3 domains with or without CH1 domain. In some embodiments, the amino acid sequence of the constant region is at least 90% identical to a human antibody constant region, such as an human IgG1 constant region, an IgG2 constant region, IgG3 constant region, or IgG4 constant region. In some other embodiments, the amino acid sequence of the constant region is at least 90% identical to an antibody constant region from another mammal, such as rabbit, dog, cat, mouse, or horse. One or more mutations can be incorporated into the constant region as compared to human IgG1 constant region, for example at Q347, Y349, L351, S354, E356, E357, K360, Q362, S364, T366, L368, K370, N390, K392, T394, D399, 5400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, T411D, T411E, K439D, and K439E.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cκ of a human IgG1 constant region may be at amino acid E123, F116, S176, V163, S174, and/or T164.

Alternatively, amino acid substitutions could be selected from the following sets of substitutions shown in Table 29.

TABLE 29

| | First Polypeptide | Second Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 30.

TABLE 30

| | First Polypeptide | Second Polypeptide |
|---|---|---|
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following set of substitutions shown in Table 31.

TABLE 31

| | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 32.

TABLE 32

| First Polypeptide | Second Polypeptide |
|---|---|
| L351Y, D399R, D399K, S400K, | T366V, T366I, T366L, T366M, |
| S400R, Y407A, Y407I, Y407V | N390D, N390E, K392L, K392M, |
| | K392V, K392F K392D, K392E, |
| | K409F, K409W, T411D and T411E |

Alternatively, at least one amino acid substitution could be selected from the following set of substitutions in Table 33, where the position(s) indicated in the First Polypeptide column is replaced by any known negatively-charged amino acid, and the position(s) indicated in the Second Polypeptide Column is replaced by any known positively-charged amino acid.

TABLE 33

| First Polypeptide | Second Polypeptide |
|---|---|
| K392, K370, K409, or K439 | D399, E356, or E357 |

Alternatively, at least one amino acid substitution could be selected from the following set of substitutions in Table 34, where the position(s) indicated in the First Polypeptide column is replaced by any known positively-charged amino acid, and the position(s) indicated in the Second Polypeptide Column is replaced by any known negatively-charged amino acid.

TABLE 34

| First Polypeptide | Second Polypeptide |
|---|---|
| D399, E356, or E357 | K409, K439, K370, or K392 |

Alternatively, amino acid substitutions could be selected from the following set in Table 35.

TABLE 35

| First Polypeptide | Second Polypeptide |
|---|---|
| T350V, L351Y, F405A, and | T350V, T366L, K392L, and |
| Y407V | T394W |

Alternatively, or in addition, the structural stability of a hetero-multimeric protein may be increased by introducing S354C on either of the first or second polypeptide chain, and Y349C on the opposing polypeptide chain, which forms an artificial disulfide bridge within the interface of the two polypeptides.

In some embodiments, the amino acid sequence of one polypeptide chain of the antibody constant region differs from the amino acid sequence of an IgG1 constant region at position T366, and wherein the amino acid sequence of the other polypeptide chain of the antibody constant region differs from the amino acid sequence of an IgG1 constant region at one or more positions selected from the group consisting of T366, L368 and Y407.

In some embodiments, the amino acid sequence of one polypeptide chain of the antibody constant region differs from the amino acid sequence of an IgG1 constant region at one or more positions selected from the group consisting of T366, L368 and Y407, and wherein the amino acid sequence of the other polypeptide chain of the antibody constant region differs from the amino acid sequence of an IgG1 constant region at position T366.

In some embodiments, the amino acid sequence of one polypeptide chain of the antibody constant region differs from the amino acid sequence of an IgG1 constant region at one or more positions selected from the group consisting of E357, K360, Q362, S364, L368, K370, T394, D401, F405, and T411 and wherein the amino acid sequence of the other polypeptide chain of the antibody constant region differs from the amino acid sequence of an IgG1 constant region at one or more positions selected from the group consisting of Y349, E357, S364, L368, K370, T394, D401, F405 and T411.

In some embodiments, the amino acid sequence of one polypeptide chain of the antibody constant region differs from the amino acid sequence of an IgG1 constant region at one or more positions selected from the group consisting of Y349, E357, S364, L368, K370, T394, D401, F405 and T411 and wherein the amino acid sequence of the other polypeptide chain of the antibody constant region differs from the amino acid sequence of an IgG1 constant region at one or more positions selected from the group consisting of E357, K360, Q362, S364, L368, K370, T394, D401, F405, and T411.

In some embodiments, the amino acid sequence of one polypeptide chain of the antibody constant region differs from the amino acid sequence of an IgG1 constant region at one or more positions selected from the group consisting of L351, D399, S400 and Y407 and wherein the amino acid sequence of the other polypeptide chain of the antibody constant region differs from the amino acid sequence of an IgG1 constant region at one or more positions selected from the group consisting of T366, N390, K392, K409 and T411.

In some embodiments, the amino acid sequence of one polypeptide chain of the antibody constant region differs from the amino acid sequence of an IgG1 constant region at one or more positions selected from the group consisting of T366, N390, K392, K409 and T411 and wherein the amino acid sequence of the other polypeptide chain of the antibody constant region differs from the amino acid sequence of an IgG1 constant region at one or more positions selected from the group consisting of L351, D399, S400 and Y407.

In some embodiments, the amino acid sequence of one polypeptide chain of the antibody constant region differs from the amino acid sequence of an IgG1 constant region at one or more positions selected from the group consisting of Q347, Y349, K360, and K409, and wherein the amino acid sequence of the other polypeptide chain of the antibody constant region differs from the amino acid sequence of an IgG1 constant region at one or more positions selected from the group consisting of Q347, E357, D399 and F405.

In some embodiments, the amino acid sequence of one polypeptide chain of the antibody constant region differs from the amino acid sequence of an IgG1 constant region at one or more positions selected from the group consisting of Q347, E357, D399 and F405, and wherein the amino acid sequence of the other polypeptide chain of the antibody constant region differs from the amino acid sequence of an IgG1 constant region at one or more positions selected from the group consisting of Y349, K360, Q347 and K409.

In some embodiments, the amino acid sequence of one polypeptide chain of the antibody constant region differs from the amino acid sequence of an IgG1 constant region at one or more positions selected from the group consisting of K370, K392, K409 and K439, and wherein the amino acid sequence of the other polypeptide chain of the antibody constant region differs from the amino acid sequence of an IgG1 constant region at one or more positions selected from the group consisting of D356, E357 and D399.

In some embodiments, the amino acid sequence of one polypeptide chain of the antibody constant region differs from the amino acid sequence of an IgG1 constant region at one or more positions selected from the group consisting of D356, E357 and D399, and wherein the amino acid sequence of the other polypeptide chain of the antibody constant region differs from the amino acid sequence of an IgG1 constant region at one or more positions selected from the group consisting of K370, K392, K409 and K439.

In some embodiments, the amino acid sequence of one polypeptide chain of the antibody constant region differs from the amino acid sequence of an IgG1 constant region at one or more positions selected from the group consisting of L351, E356, T366 and D399, and wherein the amino acid sequence of the other polypeptide chain of the antibody constant region differs from the amino acid sequence of an IgG1 constant region at one or more positions selected from the group consisting of Y349, L351, L368, K392 and K409.

In some embodiments, the amino acid sequence of one polypeptide chain of the antibody constant region differs from the amino acid sequence of an IgG1 constant region at one or more positions selected from the group consisting of Y349, L351, L368, K392 and K409, and wherein the amino acid sequence of the other polypeptide chain of the antibody constant region differs from the amino acid sequence of an IgG1 constant region at one or more positions selected from the group consisting of L351, E356, T366 and D399.

In some embodiments, the amino acid sequence of one polypeptide chain of the antibody constant region differs from the amino acid sequence of an IgG1 constant region by an S354C substitution and wherein the amino acid sequence of the other polypeptide chain of the antibody constant region differs from the amino acid sequence of an IgG1 constant region by a Y349C substitution.

In some embodiments, the amino acid sequence of one polypeptide chain of the antibody constant region differs from the amino acid sequence of an IgG1 constant region by a Y349C substitution and wherein the amino acid sequence of the other polypeptide chain of the antibody constant region differs from the amino acid sequence of an IgG1 constant region by an S354C substitution.

In some embodiments, the amino acid sequence of one polypeptide chain of the antibody constant region differs from the amino acid sequence of an IgG1 constant region by K360E and K409W substitutions and wherein the amino acid sequence of the other polypeptide chain of the antibody constant region differs from the amino acid sequence of an IgG1 constant region by Q347R, D399V and F405T substitutions.

In some embodiments, the amino acid sequence of one polypeptide chain of the antibody constant region differs from the amino acid sequence of an IgG1 constant region by Q347R, D399V and F405T substitutions and wherein the amino acid sequence of the other polypeptide chain of the antibody constant region differs from the amino acid sequence of an IgG1 constant region by K360E and K409W substitutions.

In some embodiments, the amino acid sequence of one polypeptide chain of the antibody constant region differs from the amino acid sequence of an IgG1 constant region by a T366W substitution and wherein the amino acid sequence of the other polypeptide chain of the antibody constant region differs from the amino acid sequence of an IgG1 constant region by T366S, T368A, and Y407V substitutions.

In some embodiments, the amino acid sequence of one polypeptide chain of the antibody constant region differs from the amino acid sequence of an IgG1 constant region by T366S, T368A, and Y407V substitutions and wherein the amino acid sequence of the other polypeptide chain of the antibody constant region differs from the amino acid sequence of an IgG1 constant region by a T366W substitution.

In some embodiments, the amino acid sequence of one polypeptide chain of the antibody constant region differs from the amino acid sequence of an IgG1 constant region by T350V, L351Y, F405A, and Y407V substitutions and wherein the amino acid sequence of the other polypeptide chain of the antibody constant region differs from the amino acid sequence of an IgG1 constant region by T350V, T366L, K392L, and T394W substitutions.

In some embodiments, the amino acid sequence of one polypeptide chain of the antibody constant region differs from the amino acid sequence of an IgG1 constant region by T350V, T366L, K392L, and T394W substitutions and wherein the amino acid sequence of the other polypeptide chain of the antibody constant region differs from the amino acid sequence of an IgG1 constant region by T350V, L351Y, F405A, and Y407V substitutions.

The multi-specific binding proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the immunoglobulin light chain can be cloned into a third expression vector; and the first, second, and third expression vectors can be stably transfected together into host cells to produce the multimeric proteins.

To achieve the highest yield of the multi-specific binding protein, different ratios of the first, second, and third expression vector can be explored to determine the optimal ratio for transfection into the host cells. After transfection, single clones can be isolated for cell bank generation using methods known in the art, such as limited dilution, ELISA, flow cytometry, microscopy, or Clonepix.

Clones can be cultured under conditions suitable for bio-reactor scale-up and maintained expression of the multi-specific binding protein. The multi-specific binding proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### II. Characteristics of Multi-Specific Binding Proteins

In certain embodiments, the multi-specific binding proteins described herein, which include an NKG2D-binding domain and a binding domain for DLL3, MUC1 (or MUC1-C), Plexin-AL TNFRSF10B, STEAP1, CDCP1, PTK7, AXL, ERBB-3, EDNRB, TYRP1, OLR1, ADAM12, PLAUR, CCR6, or EPHA4, bind to cells expressing human NKG2D. In certain embodiments, the multi-specific binding proteins bind to cells expressing NKG2D and/or CD16, such as NK cells, and tumor cells expressing DLL3, MUC1 (or MUC1-C), Plexin-AL TNFRSF10B, STEAP1, CDCP1, PTK7, AXL, ERBB-3, EDNRB, TYRP1, OLR1, ADAM12, PLAUR, CCR6, or EPHA4, simultaneously. Binding of the multi-specific binding proteins to NK cells can enhance the cytotoxic activity of NK cells leading to destruction of the tumor cells.

In certain embodiments, the multi-specific binding proteins described herein bind to a tumor-associated antigen at a comparable level to that of a corresponding monoclonal antibody having the same tumor-associated antigen binding site. In certain embodiments, the multi-specific binding proteins described herein may be more effective in reducing tumor growth and killing cancer cells expressing a tumor-associated antigen selected from DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, or EPHA4, than a corresponding monoclonal antibody having the same tumor-associated antigen binding site.

In certain embodiments, the multi-specific binding proteins described herein, which include an NKG2D-binding domain and a tumor-associated antigen binding domain for DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, or EPHA4, can activate primary human NK cells when co-cultured with tumor cells expressing DLL3, MUC1 (or MUC1-C), Plexin-AL TNFRSF10B, STEAP1, CDCP1, PTK7, AXL, ERBB-3, EDNRB, TYRP1, OLR1, ADAM12, PLAUR, CCR6, or EPHA4. NK cell activation is marked by the increase in CD107a expression, degranulation, and IFNγ cytokine production. Furthermore, compared to a corresponding monoclonal antibody having the same tumor-associated antigen binding site, the multi-specific binding proteins described herein show superior activation of human NK cells in the presence of tumor cells expressing DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, or EPHA4.

In certain embodiments, the multi-specific binding proteins described herein, which include an NKG2D-binding domain and a binding domain for DLL3, MUC1 (or MUC1-C), Plexin-AL TNFRSF10B, STEAP1, CDCP1, PTK7, AXL, ERBB-3, EDNRB, TYRP1, OLR1, ADAM12, PLAUR, CCR6, or EPHA4, can enhance the activation of resting and IL-2-activated human NK cells in the presence of tumor cells expressing DLL3, MUC1 (or MUC1-C), Plexin-AL TNFRSF10B, STEAP1, CDCP1, PTK7, AXL, ERBB-3, EDNRB, TYRP1, OLR1, ADAM12, PLAUR, CCR6, or EPHA4.

In certain embodiments, the multi-specific binding proteins described herein, which include an NKG2D-binding domain and a tumor-associated antigen binding domain for DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, or EPHA4, can enhance the cytotoxic activity of resting and IL-2-activated human NK cells in the presence of tumor cells expressing DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, or EPHA4. In certain embodiments, compared to corresponding monoclonal antibodies having the same tumor-associated antigen binding site, the multi-specific binding proteins described herein can have greater cytotoxic activity against tumor cells having medium and low expression of DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, or EPHA4.

In certain embodiments, the multi-specific binding proteins described herein can be advantageous in treating cancers with high expression of Fc receptor (FcR), or cancers residing in a tumor microenvironment with high levels of FcR expression, compared to corresponding DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, or EPHA4 monoclonal antibodies having the same tumor-associated antigen binding site. Monoclonal antibodies exert their effects on tumor growth through multiple mechanisms including, for example, initiation of antibody-dependent cellular cytotoxicity (ADCC), cell-dependent cytotoxicity (CDC), phagocytosis, and signal blockade, amongst others. Amongst FcγRs, CD16 has the lowest affinity for IgG Fc; FcγRI (CD64) is the high-affinity FcR, which binds about 1000 times more strongly to IgG Fc than CD16. CD64 is normally expressed on cells of many hematopoietic lineages, such as the myeloid lineage, and can also be expressed on cancer cells derived from these lineages, such as in acute myeloid leukemia (AML) Immune cells infiltrating into a tumor, such as MDSCs and monocytes, also express CD64 and are known to contribute to the tumor microenvironment. Expression of CD64 by tumor cells or by cells in the tumor microenvironment can have a detrimental effect on monoclonal antibody therapy. Expression of CD64 in the tumor microenvironment makes it difficult for monoclonal antibodies to engage CD16 on the surface of NK cells, as they preferentially bind to the high-affinity CD64 Fc-receptor. By targeting two activating receptors on the surface of NK cells, the multi-specific binding proteins of the present invention can overcome the detrimental effect of CD64 expression (either on tumors or in the tumor microenvironment). Regardless of CD64 expression on the tumor cells, the multi-specific binding proteins described herein are able to induce human NK cell responses against tumor cells, because dual targeting of two activating receptors (i.e., NKG2D and CD16) on NK cells provides stronger specific binding to and activation of NK cells.

In some embodiments, the multi-specific binding proteins described herein can provide a better safety profile through reduced on-target, off-tumor, side effects. Natural killer cells and CD8 T cells are both able to directly lyse tumor cells, although the mechanisms through which NK cells and CD8 T cells recognize normal, healthy, cells from tumor cells differ. The activity of NK cells is regulated by the balance of signals from activating (e.g., NCRs, NKG2D, CD16, etc.) and inhibitory (e.g., KIRs, NKG2A, etc.) receptors. The presence of stressed, virally-infected, or transformed self-cells stimulates activating receptors and shifts the balance towards NK cell activation. In contrast, normal, healthy, self-cells activate inhibitory receptors which shift the balance towards NK cell tolerance, thereby protecting normal, healthy tissues from potentially damaging NK cell activity. By coupling NK cell activation with tumor-associated antigen binding, or binding of antigens in the tumor microenvironment, multi-specific binding proteins described herein can avoid off-tumor side effects, and/or have an increased therapeutic window.

Unlike NK cells, T cells require recognition of a specific peptide presented by MHC/HLA molecules for activation and effector functions. T cells have been the primary target of current immunotherapies, and many strategies have been developed to redirect T cell responses against the tumor. T cell bispecifics, checkpoint inhibitors, and CAR-T cells have all been approved by the FDA, but these approaches often suffer from dose-limiting toxicities. T cell bispecifics and CAR-T cell technologies are based upon the TCR-MHC recognition system and use binding domains to target antigens on the surface of tumor cells, and engineered signaling domains to transduce the activation signals into an effector cell. Although effective at eliciting an anti-tumor immune response, these therapies are often coupled with cytokine release syndrome (CRS), and on-target, off-tumor, side effects. In contrast, the multi-specific binding proteins of the present invention will not “override” the natural activating and inhibiting systems of NK cells but will instead provide additional activation signals to NK cells, while maintaining NK tolerance to normal, healthy self cells.

In some embodiments, a multi-specific binding protein described herein can delay progression of a tumor more effectively than a corresponding monoclonal antibody having the same tumor-associated antigen-binding domain. In some embodiments, a multi-specific binding protein described herein ismore effective at inhibiting cancer metastasis than a corresponding monoclonal antibody having the same tumor-associated antigen-binding domain.

In certain other embodiments, the multi-specific binding proteins described herein include an NKG2D-binding site, a CD16-binding site, and a binding site for CD14, CD163, CSF3R, Siglec-9, ITGAM, VISTA, B7-H4, CCR1, LRRC25, PTAFR, SIRPB1, TLR2, TLR4, CD300LB, ATP1A3, or CCR5. In some embodiments, the multi-specific binding proteins further include an additional binding site for a tumor-associated antigen. In some embodiments, the multi-specific binding proteins bind to cells expressing NKG2D and/or CD16, such as NK cells, and cells expressing any one of the above antigens, such as MDSCs and/or TAMs simultaneously. In some other embodiments, the multi-specific binding proteins bind to cells expressing NKG2D and/or CD16, such as NK cells; cells expressing any one of the above antigens, such as MDSCs and/or TAMs; and tumor cells expressing a tumor-associated antigen, simultaneously. Binding of the multi-specific binding proteins to NK cells can enhance the activity of the NK cells toward destruction of the MDSCs and/or TAMs in a tumor environment, and promote an immune response against the tumor cells in the same tumor environment. In some embodiments, the multi-specific binding proteins that include an additional tumor-associated antigen-binding site enhance the activity of the NK cells toward destruction of the tumor cells that express the tumor-associated antigen as well as the MDSCs and/or TAMs in the tumor environment.

In some embodiments, the multi-specific binding proteins of the present invention, which include an additional tumor-associated antigen-binding site, bind to the tumor cells expressing the tumor-associated antigen with a similar affinity to that of a monoclonal antibody having the same antigen-binding site. The multi-specific binding proteins can be more effective in killing tumor cells than the corresponding monoclonal antibodies having the same antigen-binding site.

In certain embodiments, the multi-specific binding proteins described herein bind to the antigen CD14, CD163, CSF3R, Siglec-9, ITGAM, VISTA, B7-H4, CCR1, LRRC25, PTAFR, SIRPB1, TLR2, TLR4, CD300LB, ATP1A3, or CCR5 with a similar affinity to that of a corresponding monoclonal antibody having the same antigen-binding site. In some embodiments, the multi-specific binding proteins of the present invention are more effective at de-repressing/enhancing the immune response within a tumor microenvironment, and in killing the tumor cells residing therein than the corresponding monoclonal antibodies having the same antigen-binding site.

In certain embodiments, the multi-specific binding proteins described herein, which include an NKG2D-binding site and a binding site for CD14, CD163, CSF3R, Siglec-9, ITGAM, VISTA, B7-H4, CCR1, LRRC25, PTAFR, SIRPB1, TLR2, TLR4, CD300LB, ATP1A3, or CCR5, activate primary human NK cells when co-cultured with cells expressing CD14, CD163, CSF3R, Siglec-9, ITGAM, VISTA, B7-H4, CCR1, LRRC25, PTAFR, SIRPB1, TLR2, TLR4, CD300LB, ATP1A3, or CCR5. NK cell activation is marked by an increase in CD107a expression, degranulation, and IFN-γ cytokine production. Furthermore, compared to a corresponding monoclonal antibody having the same antigen-binding site, the multi-specific binding proteins disclosed herein may elicit superior activation of human NK cells in the presence of cells expressing the antigen CD14, CD163, CSF3R, Siglec-9, ITGAM, VISTA, B7-H4, CCR1, LRRC25, PTAFR, SIRPB1, TLR2, TLR4, CD300LB, ATP1A3, or CCR5. In some embodiments, the cells expressing one or more of these antigens are MDSCs and/or TAMs.

In certain embodiments, the multi-specific binding proteins described herein, which include an NKG2D-binding site and a binding site for CD14, CD163, CSF3R, Siglec-9, ITGAM, VISTA, B7-H4, CCR1, LRRC25, PTAFR, SIRPB1, TLR2, TLR4, CD300LB, ATP1A3, or CCR5, enhance the activation of resting and IL-2-activated human NK cells co-cultured with cells expressing CD14, CD163, CSF3R, Siglec-9, ITGAM, VISTA, B7-H4, CCR1, LRRC25, PTAFR, SIRPB1, TLR2, TLR4, CD300LB, ATP1A3, or CCR5. In some embodiments, the cells expressing one or more of these antigens are MDSCs and/or TAMs.

In certain embodiments, compared to a corresponding monoclonal antibody that binds to CD14, CD163, CSF3R, Siglec-9, ITGAM, VISTA, B7-H4, CCR1, LRRC25, PTAFR, SIRPB1, TLR2, TLR4, CD300LB, ATP1A3, or CCR5, the multi-specific binding proteins disclosed herein can have greater cytotoxic activity against cells having medium and low expression of CD14, CD163, CSF3R, Siglec-9, ITGAM, VISTA, B7-H4, CCR1, LRRC25, PTAFR, SIRPB1, TLR2, TLR4, CD300LB, ATP1A3, or CCR5. In some embodiments, the cells expressing medium and low levels of CD14, CD163, CSF3R, Siglec-9, ITGAM, VISTA, B7-H4, CCR1, LRRC25, PTAFR, SIRPB1, TLR2, TLR4, CD300LB, ATP1A3, or CCR5 are MDSCs. In some embodiments, the cells expressing medium and low levels of CD14, CD163, CSF3R, Siglec-9, ITGAM, VISTA, B7-H4, CCR1, LRRC25, PTAFR, SIRPB1, TLR2, TLR4, CD300LB, ATP1A3, or CCR5 are TAMs.

### III. Therapeutic Applications

The invention provides methods for treating cancer using a multi-specific binding protein described herein and/or a pharmaceutical composition described herein. The methods may be used to treat a variety of cancers which express DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, or EPHA4 by administering to a patient in need thereof a therapeutically effective amount of a multi-specific binding protein described herein.

The cancer to be treated can be characterized according to the presence of a particular antigen expressed on the surface of the cancer cell. In certain embodiments, the cancer cell can express one or more of the following in addition to DLL3, MUC1 (or MUC1-C), Plexin-A1, TNFRSF10B, STEAP1, CDCP1, PTK7, AXL, ERBB-3, EDNRB, TYRP1, OLR1, ADAM12, PLAUR, CCR6, or EPHA4: CD2, CD19, CD20, CD30, CD38, CD40, CD52, CD70, EGFR/ERBB-1, IGF1R, HER3/ERBB-3, HER4/ERBB-4, MUC1, TROP2, cMET, SLAMF7, PSCA, MICA, MICB, TRAILR1, TRAILR2, MAGE-A3, B7.1, B7.2, CTLA4, and PD1.

The methods may also be used to treat a variety of cancers, which co-exist with CD14, CD163, CSF3R, Siglec-9, ITGAM, VISTA, B7-H4, CCR1, LRRC25, PTAFR, SIRPB1, TLR2, TLR4, CD300LB, ATP1A3, or CCR5-expressing MDSCs and/or TAMs in the tumor microenvironment.

The therapeutic method can be characterized according to the cancer to be treated. Exemplary cancers to be treated may be acral lentiginous melanoma, actinic keratoses, acute lymphoblastic leukemia, acute lymphocytic leukemia, acute myeloid leukemia, acute myeloid leukemia, adenocarcinoma, adenoid cystic carcinoma, adenosarcoma, adenosquamous carcinoma, anal canal cancer, anaplastic large cell lymphoma, angioimmunoblastic T-cell lymphoma, angiosarcoma, anorectal cancer, astrocytic tumor, bartholin gland carcinoma, basocellular carcinomas (e.g., skin), B-cell lymphoma, biliary tract cancer, bladder cancer, bone cancer, bone marrow cancer, brain cancer, breast cancer, bronchial cancer, bronchial gland carcinoma, Burkitt lymphoma, carcinoid, cervical cancer, cholangiocarcinoma, chondrosarcoma, choroid plexus papilloma/carcinoma, chronic lymphocytic leukemia, chronic myeloid leukemia, chronic neutrophilic leukemia, clear cell carcinoma, colon cancer, colorectal cancer, connective tissue cancer, cutaneous T-cell lymphoma, cystadenoma, diffuse large B-cell lymphoma, digestive system cancer, duodenum cancer, endocrine system cancer, endodermal sinus tumor, endometrial cancer/hyperplasia, endometrial stromal sarcoma, endometrioid adenocarcinoma, endothelial cell cancer, enteropathy type T-cell lymphoma, ependymal cancer, epithelial cell cancer, esophageal cancer, Ewing sarcoma, extranodal marginal zone B-cell lymphoma, extranodal natural killer/T-cell lymphoma, eye and orbit cancer, female genital cancer, focal nodular hyperplasia, follicular lymphoma, gall bladder cancer, gastric antrum cancer, gastric cancer, gastric fundus cancer, gastrinoma, glioblastoma, glioma, glucagonoma, hairy cell leukemia, head and neck cancer, heart cancer, hemangioblastoma, hemangioendothelioma, hemangiomas, hematological tumors, hepatic adenoma, hepatic adenomatosis, hepatocellular carcinoma, heptobilliary cancer, Hodgkin's disease, ileum cancer, insulinoma, intraepithelial neoplasia, intraepithelial squamous cell neoplasia, intrahepatic bile duct cancer, invasive squamous cell carcinoma, jejunum cancer, joint cancer, Kaposi's sarcoma, kidney cancer, large cell carcinoma, large intestine cancer, leiomyosarcoma, lentigo maligna melanomas, leukemia, liver cancer, lung cancer, lymphoma, lymphoplasmacytic lymphoma, male genital cancer, malignant melanoma, malignant mesotheilial tumors, mantle cell lymphoma, marginal zone B-cell lymphoma, medulloblastoma, medulloepithelioma, melanoma, meningeal cancer, mesothelial cancer, mesothelioma, metastatic carcinoma, mouth cancer, mucoepidermoid carcinoma, multiple myeloma, muscle cancer, myelodysplastic neoplasms, myeloproliferative neoplasms, nasal tract cancer, nervous system cancer, neuroblastoma, neuroepithelial adenocarcinoma, nodal marginal zone B-cell lymphoma, nodular melanoma, non-epithelial skin cancer, non-Hodgkin's lymphoma, oat cell carcinoma, oligodendroglial cancer, oral cavity cancer, osteosarcoma, ovarian cancer, pancreatic cancer, papillary serous adenocarcinoma, parotid, pelvic cancer, penile cancer, peripheral T-cell lymphoma, pharynx cancer, pituitary tumors, plasmacytoma, precursor T-lymphoblastic lymphoma, primary central nervous system lymphoma, primary mediastinal B-cell lymphoma, prostate cancer, pseudosarcoma, pulmonary blastoma, rectal cancer, renal cancer, renal cell carcinoma, respiratory system cancer, retinoblastoma, rhabdomyosarcoma, sarcoma, serous carcinoma, sinus cancer, skin cancer, small cell carcinoma, small intestine cancer, small lymphocytic lymphoma, smooth muscle cancer, soft tissue cancer, somatostatin-secreting tumor, spine cancer, splenic marginal zone B-cell lymphoma, squamous cell carcinoma (e.g., skin), striated muscle cancer, subcutaneous panniculitis-like t-cell lymphoma, submesothelial cancer, superficial spreading melanoma, T cell leukemia, T cell lymphoma, testicular cancer, thyroid cancer, tongue cancer, undifferentiated carcinoma, ureter cancer, urethra cancer, urinary bladder cancer, uterine cancer, uterine corpus cancer, uveal melanoma, vaginal cancer, verrucous carcinoma, VIPoma, vulva cancer, well-differentiated carcinoma, or Wilms tumor.

### IV. Combination Therapy

Another aspect of the invention provides for combination therapy. Multi-specific binding proteins described herein can be used in combination with additional therapeutic agents to treat a cancer.

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

An additional class of agents that may be used as part of a combination therapy in treating cancer is immune checkpoint inhibitors. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAGS, (v) B7-H3, (vi) B7-H4, and (vii) TIM3. The CTLA4 inhibitor ipilimumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK inhibitor, an ATR inhibitor, an A2A antagonist, a base excision repair inhibitor, a Bcr-Abl tyrosine kinase inhibitor, a Bruton's tyrosine kinase inhibitor, a CDCl₇ inhibitor, a CHK1 inhibitor, a Cyclin-dependent kinase inhibitor, a DNA-PK inhibitor, an inhibitor of both DNA-PK and mTOR, a DNMT1 inhibitor, a DNMT1 inhibitor plus 2-chloro-deoxyadenosine, an HDAC inhibitor, a Hedgehog signaling pathway inhibitor, an IDO inhibitor, a JAK inhibitor, an mTOR inhibitor, a MEK inhibitor, a MELK inhibitor, a MTH1 inhibitor, a PARP inhibitor, a phosphoinositide 3-kinase inhibitor, an inhibitor of both PARP1 and DHODH, a proteasome inhibitor, a topoisomerase-II inhibitor, a tyrosine kinase inhibitor, a VEGFR inhibitor, and a WEE1 inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

Proteins of the invention can also be used as an adjunct to surgical removal of the primary lesion.

The amount of multi-specific binding protein and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a multi-specific binding protein may be administered during a time when the additional therapeutic agent(s) exerts its prophylactic or therapeutic effect, or vice versa.

### V. Pharmaceutical Compositions

The present disclosure also features pharmaceutical compositions that contain a therapeutically effective amount of a protein described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, 17th Ed. Mack Publishing Company, Easton, Pa. (1985). For a brief review of methods for drug delivery, see, e.g., Langer R. (1990) Science; 249(4976):1527-1533.

The intravenous drug delivery formulation of the present disclosure may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may be freeze-dried (lyophilized) and contained in about 12-60 vials. In certain embodiments, the formulation may be freeze-dried and 45 mg of the freeze-dried formulation may be contained in one vial. In certain embodiments, the about 40 mg to about 100 mg of freeze-dried formulation may be contained in one vial. In certain embodiments, freeze dried formulation from 12, 27, or 45 vials are combined to obtain a therapeutic dose of the protein in the intravenous drug formulation. In certain embodiments, the formulation may be a liquid formulation and stored as about 250 mg/vial to about 1000 mg/vial. In certain embodiments, the formulation may be a liquid formulation and stored as about 600 mg/vial. In certain embodiments, the formulation may be a liquid formulation and stored as about 250 mg/vial.

This present disclosure could exist in a liquid aqueous pharmaceutical formulation including a therapeutically effective amount of the multi-specific binding protein in a buffered solution.

The compositions disclosed herein may be sterilized by conventional sterilization techniques, or may be filter-sterilized. The resulting aqueous solutions may be packaged for use as-is, or lyophilized, wherein the lyophilized preparation is combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides a formulation with an extended shelf life including the multi-specific binding protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including the multi-specific binding protein of the present disclosure in a pH-buffered solution. The buffer of this invention may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g., sodium acetate), succinate (e.g., sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/ml of sodium citrate (e.g., 0.305 mg/mL), about 1.5 mg/mL of disodium phosphate dihydrate (e.g., 1.53 mg/mL), about 0.9 mg/mL of sodium dihydrogen phosphate dihydrate (e.g., 0.86 mg/mL), and about 6.2 mg/mL of sodium chloride (e.g., 6.165 mg/mL). In certain embodiments, the buffer system includes 1-1.5 mg/mL of citric acid, 0.25 to 0.5 mg/mL of sodium citrate, 1.25 to 1.75 mg/mL of disodium phosphate dihydrate, 0.7 to 1.1 mg/mL of sodium dihydrogen phosphate dihydrate, and 6.0 to 6.4 mg/mL of sodium chloride. In certain embodiments, the pH of the formulation is adjusted with sodium hydroxide.

A polyol, which acts as a tonicifier and may stabilize an antibody, may also be included in the formulations described herein. The polyol is added to a formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (e.g., trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to 15 mg/mL. In certain embodiments, the concentration of mannitol may be about 10-14 mg/mL. In certain embodiments, the concentration of mannitol may be about 12 mg/mL. In certain embodiments, the polyol sorbitol may be included in the formulation.

A detergent or surfactant may also be added to the formulations of the present invention. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see, e.g., Fiedler H. P., Lexikon der Hifsstoffe für Pharmazie, Kosmetik and andrenzende Gebiete, 4^{th} Ed., Editio Cantor, Aulendorf, Germany (1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In certain embodiments, the multi-specific binding protein product of the present disclosure is formulated as a liquid formulation. The liquid formulation may be present at a 10 mg/mL concentration in either a USP/Ph Eur type I 50R vial closed with a rubber stopper and sealed with an aluminum crimp seal closure. The stopper may be made of elastomer complying with USP and Ph Eur. In certain embodiments vials may be filled with 61.2 mL of the multi-specific binding protein product solution in order to allow an extractable volume of 60 mL. In certain embodiments, the liquid formulation may be diluted with 0.9% saline solution.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be a disaccharide, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In addition to aggregation, deamidation is a common product variation of peptides and proteins that may occur during fermentation, harvest/cell clarification, purification, drug substance/drug product storage, and sample analysis. Under physiological conditions, deamidation is the loss of ammonia (NH₃) from an asparagine residue of a protein, resulting in a 17 dalton descrease in mass and formation of a succinimide intermediate. Subsequent hydrolysis of succinimide results in an 18 dalton mass increase and formation of aspartic acid or isoaspartic acid. The parameters affecting the rate of deamidation include pH, temperature, solvent dielectric constant, ionic strength, primary sequence, local polypeptide conformation and tertiary structure. The amino acid residues adjacent to Asn in the peptide chain may also affect deamidation rates, e.g., Gly and Ser residues following an Asn residue results in a higher susceptibility to deamidation.

In certain embodiments, the liquid formulation of the present disclosure may be preserved under conditions of pH and humidity to prevent deamidation of the protein product.

The aqueous carrier of interest herein is one which is pharmaceutically acceptable (i.e., safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

Intravenous (IV) formulations may be the preferred administration route in particular instances, such as when a patient is in the hospital after transplantation receiving all drugs via the IV route. In certain embodiments, the liquid formulation is diluted with 0.9% sodium chloride solution before administration. In certain embodiments, the diluted drug product for injection is isotonic and suitable for administration by intravenous infusion.

In certain embodiments, salt or buffer components may be added in amounts of about 10 mM to about 200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterions.

A preservative may be optionally added to the formulations herein to reduce bacterial action. The addition of a preservative may, for example, facilitate the production of a multi-use (i.e., multiple-dose) formulation.

The aqueous carrier of interest herein is one which is pharmaceutically acceptable (i.e., safe and non-toxic for administration to a human) and is useful for the preparation of a liquid formulation. Illustrative carriers include SWFI, BWFI, a pH buffered solution (e.g., phosphate-buffered saline), sterile saline solution, Ringer's solution or dextrose solution.

This present disclosure could exist in a lyophilized formulation including the proteins and a lyoprotectant. The lyoprotectant may be a sugar, e.g., a disaccharide. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be from 1:2 to 1:5.

In certain embodiments, the pH of the lyophilized formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing the protein of the present disclosure may be adjusted between 6 to 8. In certain embodiments, the pH range for the lyophilized drug product may be from 7 to 8.

In certain embodiments of the lyophilized formulation, salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In certain embodiments, a “bulking agent” may be added to the lyophilized formulation. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

A preservative may be optionally added to the lyophilized formulations herein to reduce bacterial action. The addition of a preservative may, for example, facilitate the production of a multi-use (i.e., multiple-dose) formulation.

In certain embodiments, the lyophilized drug product may be reconstituted with an aqueous diluent. The aqueous diluent of interest herein is one which is pharmaceutically acceptable (e.g., safe and non-toxic for administration to a human) and is useful for the preparation of a reconstituted liquid formulation, after lyophilization. Illustrative diluents include SWFI, BWFI, a pH buffered solution (e.g., phosphate-buffered saline), sterile saline solution, Ringer's solution or dextrose solution.

In certain embodiments, the lyophilized drug product of the current disclosure is reconstituted with either SWFI, USP or 0.9% sodium chloride for injection, USP. During reconstitution, the lyophilized powder dissolves into a solution.

In certain embodiments, the lyophilized protein product of the instant disclosure is reconstituted to about 4.5 mL in SWFI and diluted with 0.9% saline solution (sodium chloride solution).

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (see, e.g., Schmitz et al. (2001) Clinica Chimica Acta; 308: 43-53; Steimer et al. (2001) Clinica Chimica Acta; 308: 33-41.). In general, dosages based on body weight are from about 0.01 μg to about 100 mg per kg of body weight, such as about 0.01 μg to about 100 mg/kg of body weight, about 0.01 μg to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, or about 50 mg to about 100 mg/kg of body weight.

Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present invention can be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, or once or more times annually.

The description above describes multiple aspects and embodiments of the invention. The patent application specifically contemplates all combinations and permutations of the aspects and embodiments.

### EXAMPLES

The invention now being generally described, will be more readily understood by reference to the following examples, which are included merely for purposes of illustration of certain aspects and embodiments of the present invention, and are not intended to limit the invention.

### Example 1—NKG2D-Binding Domains Bind to NKG2D

### NKG2D-Binding Domains Bind to Purified Recombinant NKG2D

The nucleic acid sequences of human, mouse or cynomolgus NKG2D ectodomains were fused with nucleic acid sequences encoding human IgG1 Fc domains and introduced into mammalian cells to be expressed. After purification, NKG2D-Fc fusion proteins were adsorbed to wells of microplates. After blocking the wells with bovine serum albumin to prevent non-specific binding, NKG2D-binding domains were titrated and added to the wells pre-adsorbed with NKG2D-Fc fusion proteins. Primary antibody binding was detected using a secondary antibody which was conjugated to horseradish peroxidase and specifically recognizes a human kappa light chain to avoid Fc cross-reactivity. 3,3′,5,5′-Tetramethylbenzidine (TMB), a substrate for horseradish peroxidase, was added to the wells to visualize the binding signal, whose absorbance was measured at 450 nM and corrected at 540 nM. An NKG2D-binding domain clone, an isotype control or a positive control (comprising heavy chain and light chain variable domains selected from SEQ ID NOs:101-104, or anti-mouse NKG2D clones MI-6 and CX-5, eBioscience, San Diego, Calif.) was added to each well.

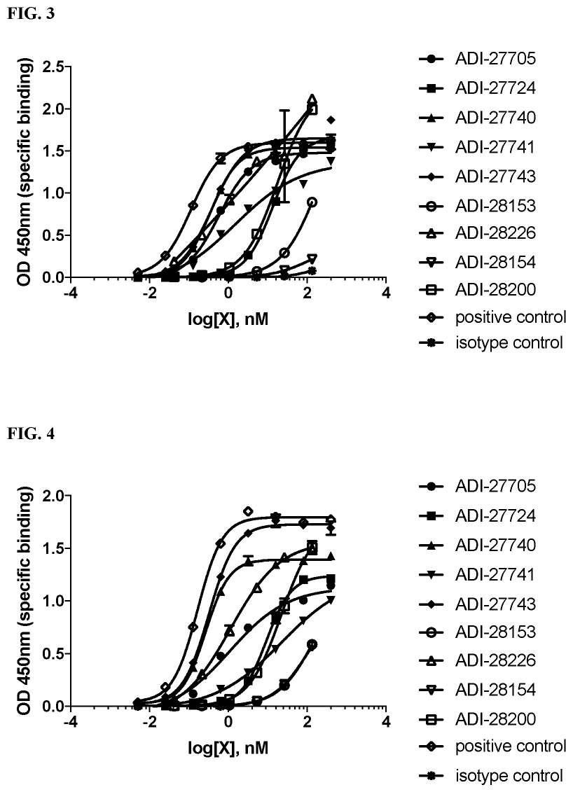

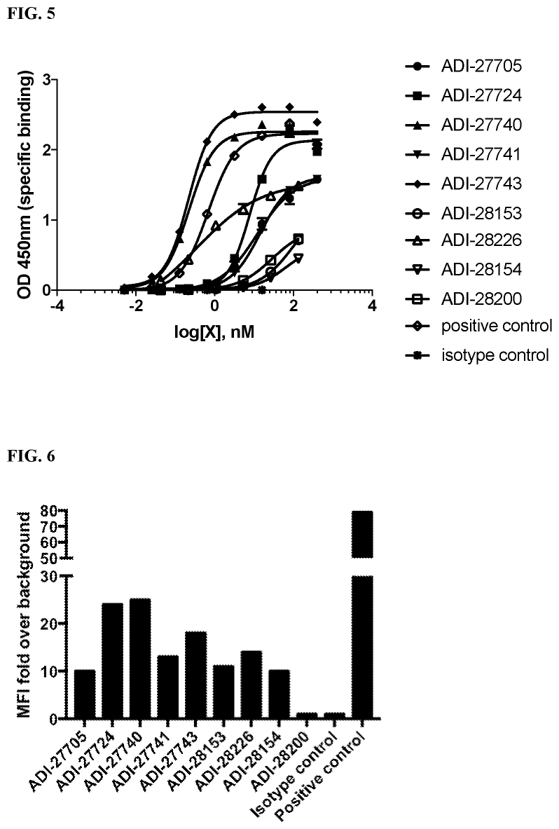

The isotype control showed minimal binding to recombinant NKG2D-Fc proteins, while the positive control bound strongest to the recombinant antigens. NKG2D-binding domains produced by all clones demonstrated binding across human, mouse, and cynomolgus recombinant NKG2D-Fc proteins, although with varying affinities from clone to clone. Generally, each anti-NKG2D clone bound to human (FIG. 3) and cynomolgus (FIG. 4) recombinant NKG2D-Fc with similar affinity, but with lower affinity to mouse (FIG. 5) recombinant NKG2D-Fc.

### NKG2D-Binding Domains Bind to Cells Expressing NKG2D

EL4 mouse lymphoma cell lines were engineered to express human or mouse NKG2D-CD3 zeta signaling domain chimeric antigen receptors. An NKG2D-binding clone, an isotype control, or a positive control was used at a 100 nM concentration to stain extracellular NKG2D expressed on the EL4 cells. The antibody binding was detected using fluorophore-conjugated anti-human IgG secondary antibodies. Cells were analyzed by flow cytometry, and fold-over-background (FOB) was calculated using the mean fluorescence intensity (MFI) of NKG2D-expressing cells compared to parental EL4 cells.

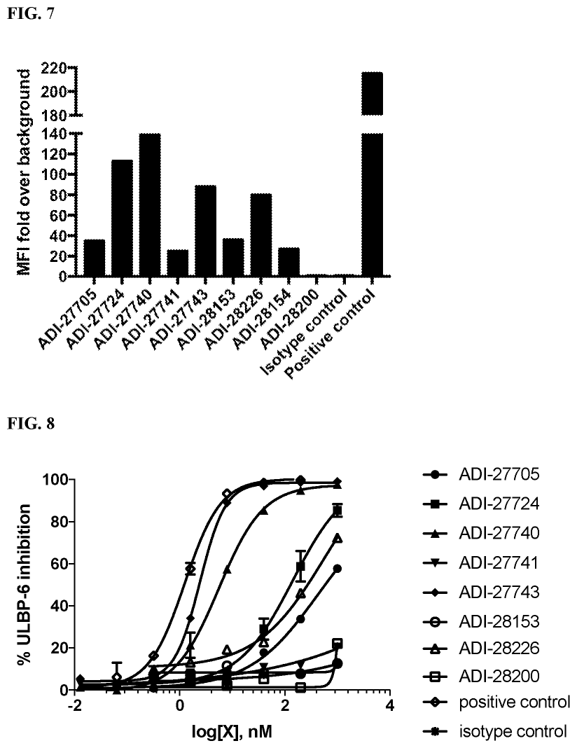

NKG2D-binding domains produced by all clones bound to EL4 cells expressing human and mouse NKG2D. Positive control antibodies (comprising heavy chain and light chain variable domains selected from SEQ ID NOs:101-104, or anti-mouse NKG2D clones MI-6 and CX-5, eBioscience, San Diego, Calif.) gave the best FOB binding signal. The NKG2D-binding affinity for each clone was similar between cells expressing human NKG2D (FIG. 6) and mouse (FIG. 7) NKG2D.

### Example 2—NKG2D-Binding Domains Block Natural Ligand Binding to NKG2D

Competition with ULBP-6

Recombinant human NKG2D-Fc proteins were adsorbed to wells of a microplate, and the wells were blocked with bovine serum albumin to reduce non-specific binding. A saturating concentration of ULBP-6-His-biotin was added to the wells, followed by addition of the NKG2D-binding domain clones. After a 2-hour incubation, wells were washed and ULBP-6-His-biotin that remained bound to the NKG2D-Fc coated wells was detected by streptavidin-conjugated to horseradish peroxidase and TMB substrate. Absorbance was measured at 450 nM and corrected at 540 nM. After subtracting background, specific binding of NKG2D-binding domains to the NKG2D-Fc proteins was calculated from the percentage of ULBP-6-His-biotin that was blocked from binding to the NKG2D-Fc proteins in wells. The positive control antibody (comprising heavy chain and light chain variable domains selected from SEQ ID NOs:101-104) and various NKG2D-binding domains blocked ULBP-6 binding to NKG2D, while isotype control showed little competition with ULBP-6 (FIG. 8).

ULBP-6 sequence is represented by SEQ ID NO:632.

MAAAAIPALLLCLPLLFLLFGWSRARRDDPHSLCYDITVIPKFRPGPRWCAVQGQVD
EKTFLHYDCGNKTVTPVSPLGKKLNVTMAWKAQNPVLREVVDILTEQLLDIQLENY
TPKEPLTLQARMSCEQKAEGHSSGSWQFSIDGQTFLLFDSEKRMWTTVHPGARKMK
EKWENDKDVAMSFHYISMGDCIGWLEDFLMGMDSTLEPSAGAPLAMSSGTTQLRA
TATTLILCCLLIILPCFILPGI (SEQ ID NO: 632)

Competition with MICA

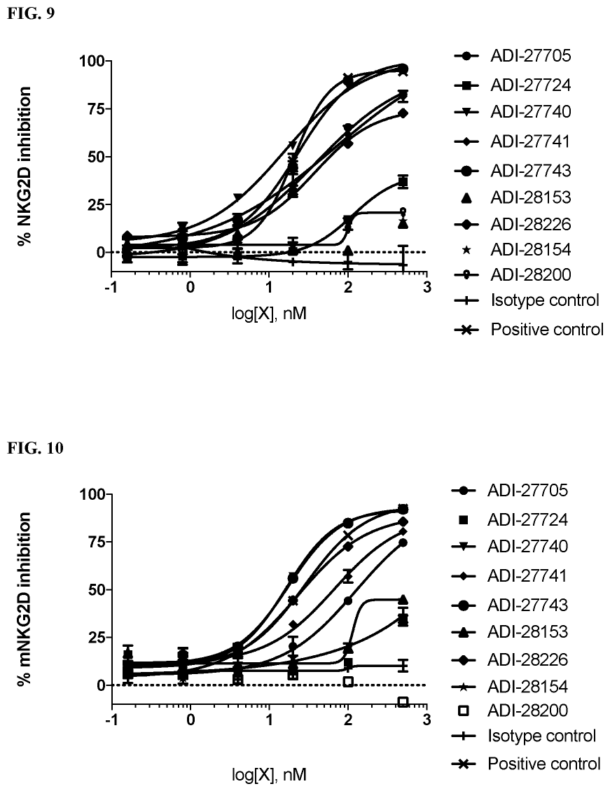

Recombinant human MICA-Fc proteins were adsorbed to wells of a microplate, and the wells were blocked with bovine serum albumin to reduce non-specific binding. NKG2D-Fc-biotin was added to wells followed by NKG2D-binding domains. After incubation and washing, NKG2D-Fc-biotin that remained bound to MICA-Fc coated wells was detected using streptavidin-HRP and TMB substrate. Absorbance was measured at 450 nM and corrected at 540 nM. After subtracting background, specific binding of NKG2D-binding domains to the NKG2D-Fc proteins was calculated from the percentage of NKG2D-Fc-biotin that was blocked from binding to the MICA-Fc coated wells. The positive control antibody (comprising heavy chain and light chain variable domains selected from SEQ ID NOs:101-104) and various NKG2D-binding domains blocked MICA binding to NKG2D, while isotype control showed little competition with MICA (FIG. 9).

Competition with Rae-1 Delta

Recombinant mouse Rae-1 delta-Fc (R&D Systems, Minneapolis, Minn.) was adsorbed to wells of a microplate, and the wells were blocked with bovine serum albumin to reduce non-specific binding. Mouse NKG2D-Fc-biotin was added to the wells followed by NKG2D-binding domains. After incubation and washing, NKG2D-Fc-biotin that remained bound to Rae-1delta-Fc coated wells was detected using streptavidin-HRP and TMB substrate. Absorbance was measured at 450 nM and corrected at 540 nM. After subtracting background, specific binding of NKG2D-binding domains to the NKG2D-Fc proteins was calculated from the percentage of NKG2D-Fc-biotin that was blocked from binding to the Rae-1delta-Fc coated wells. The positive control (comprising heavy chain and light chain variable domains selected from SEQ ID NOs:101-104, or anti-mouse NKG2D clones MI-6 and CX-5, eBioscience, San Diego, Calif.) and various NKG2D-binding domain clones blocked Rae-1 delta binding to mouse NKG2D, while the isotype control antibody showed little competition with Rae-1delta (FIG. 10).

### Example 3—NKG2D-Binding Domain Clones Activate NKG2D

Nucleic acid sequences of human and mouse NKG2D were fused to nucleic acid sequences encoding a CD3 zeta signaling domain to obtain chimeric antigen receptor (CAR) constructs. The NKG2D-CAR constructs were then cloned into a retrovirus vector using Gibson assembly and transfected into expi293 cells for retrovirus production. EL4 cells were infected with viruses containing NKG2D-CAR together with 8 μg/mL polybrene. 24 hours after infection, the expression levels of NKG2D-CAR in the EL4 cells were analyzed by flow cytometry, and clones which express high levels of the NKG2D-CAR on the cell surface were selected.

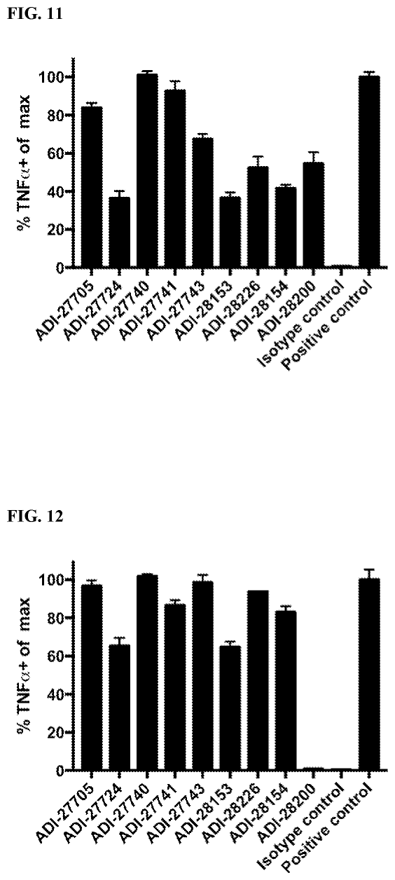

To determine whether NKG2D-binding domains activate NKG2D, they were adsorbed to wells of a microplate, and NKG2D-CAR EL4 cells were cultured on the antibody fragment-coated wells for 4 hours in the presence of brefeldin-A and monensin. Intracellular TNF-alpha production, an indicator for NKG2D activation, was assayed by flow cytometry. The percentage of TNF-alpha positive cells was normalized to the cells treated with the positive control. All NKG2D-binding domains activated both human NKG2D (FIG. 11) and mouse NKG2D (FIG. 12).

### Example 4—NKG2D-Binding Domains Activate NK Cells

### Primary Human NK Cells

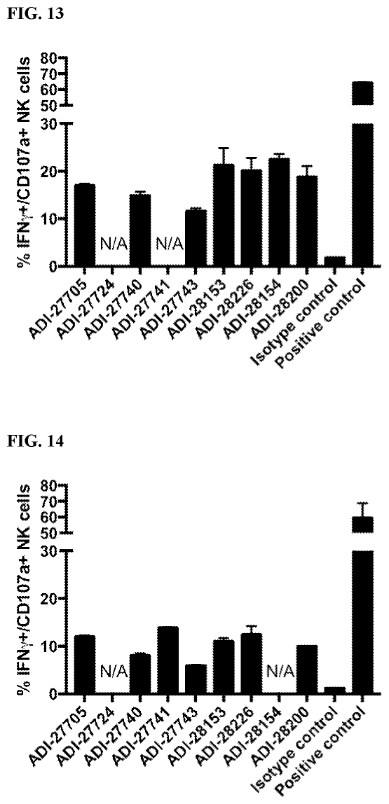

Peripheral blood mononuclear cells (PBMCs) were isolated from human peripheral blood buffy coats using density gradient centrifugation. NK cells (CD3⁻CD56⁺) were isolated using negative selection with magnetic beads from PBMCs, and the purity of the isolated NK cells was typically >95%. Isolated NK cells were then cultured in media containing 100 ng/mL IL-2 for 24-48 hours before they were transferred to the wells of a microplate to which the NKG2D-binding domains were adsorbed, and cultured in media containing fluorophore-conjugated anti-CD107a antibody, brefeldin-A, and monensin. Following culture, NK cells were assayed by flow cytometry using fluorophore-conjugated antibodies against CD3, CD56 and IFNγ. CD107a and IFNγ staining were analyzed in CD3⁻ CD56⁺ cells to assess NK cell activation. The increase in CD107a/IFNγ double-positive cells is indicative of better NK cell activation through engagement of two activating receptors rather than one receptor. NKG2D-binding domains and the positive control (comprising heavy chain and light chain variable domains selected from SEQ ID NOs:101-104) showed a higher percentage of NK cells becoming CD107a⁺ and IFNγ⁺ than the isotype control (FIG. 13 and FIG. 14 represent data from two independent experiments, each using a different donor's PBMCs for NK cell preparation).

### Primary Mouse NK Cells

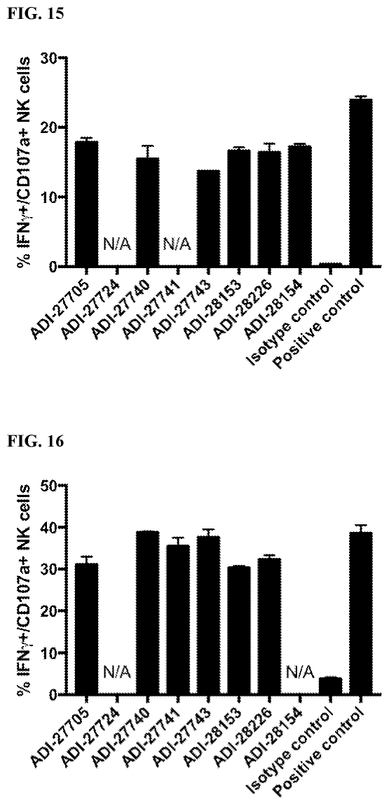

Spleens were obtained from C57Bl/6 mice and crushed through a 70 μm cell strainer to obtain a single cell suspension. Cells were pelleted and resuspended in ACK lysis buffer (Thermo Fisher Scientific #A1049201, Carlsbad, Calif.; 155 mM ammonium chloride, 10 mM potassium bicarbonate, 0.01 mM EDTA) to remove red blood cells. The remaining cells were cultured with 100 ng/mL hIL-2 for 72 hours before being harvested and prepared for NK cell isolation. NK cells (CD3⁻NK1.1⁺) were then isolated from spleen cells using a negative depletion technique with magnetic beads which typically yields NK cell populations having >90% purity. Purified NK cells were cultured in media containing 100 ng/mL mIL-15 for 48 hours before they were transferred to the wells of a microplate to which the NKG2D-binding domains were adsorbed, and cultured in media containing fluorophore-conjugated anti-CD107a antibody, brefeldin-A, and monensin. Following culture in NKG2D-binding domain-coated wells, NK cells were assayed by flow cytometry using fluorophore-conjugated antibodies against CD3, NK1.1 and IFNγ. CD107a and IFNγ staining were analyzed in CD3⁻NK1.1⁺ cells to assess NK cell activation. The increase in CD107a/IFNγ double-positive cells is indicative of better NK cell activation through engagement of two activating receptors rather than one receptor. NKG2D-binding domains and the positive control (selected from anti-mouse NKG2D clones MI-6 and CX-5, eBioscience, San Diego, Calif.) showed a higher percentage of NK cells becoming CD107a⁺ and IFNγ⁺ than the isotype control (FIG. 15 and FIG. 16 represent data from two independent experiments, each using a different mouse for NK cell preparation).

### Example 5—NKG2D-Binding Domains Enhance Cytotoxicity Against Target Tumor Cells

Human and mouse primary NK cell activation assays demonstrate increased cytotoxicity markers on NK cells after incubation with NKG2D-binding domains. To address whether this translates into increased tumor cell lysis, a cell-based assay was utilized where each NKG2D-binding domain was developed into a monospecific antibody. The Fc region was used as one targeting arm, while the Fab region (NKG2D-binding domain) acted as another targeting arm to activate NK cells. THP-1 cells, which are of human origin and express high levels of Fc receptors, were used as a tumor target and a Perkin Elmer DELFIA® Cytotoxicity Kit (Waltham, Mass.) was used. THP-1 cells were labeled with BATDA reagent, and resuspended at 10⁵ cells/mL in culture media. Labeled THP-1 cells were then combined with NKG2D antibodies and isolated mouse NK cells in wells of a microtiter plate at 37° C. for 3 hours. After incubation, 20 μl of the culture supernatant was removed, mixed with 200 μl of Europium solution and incubated with shaking for 15 minutes in the dark. Fluorescence was measured over time by a PHERAstar® plate reader equipped with a time-resolved fluorescence module (Excitation 337 nm, Emission 620 nm) and specific lysis was calculated according to the kit instructions.

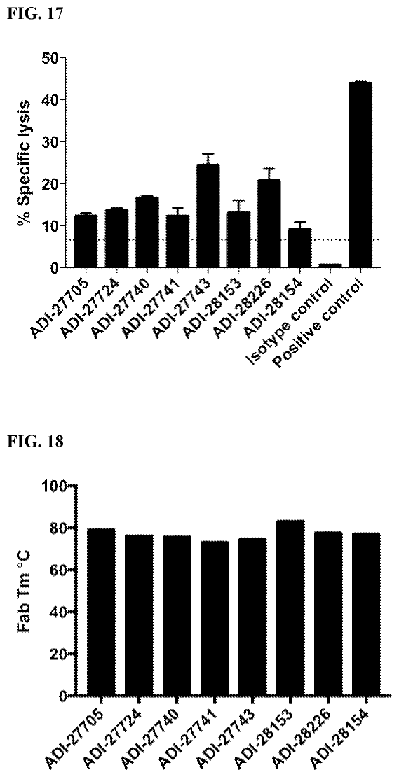

The positive control, ULBP-6, a natural ligand for NKG2D, showed increased specific lysis of THP-1 target cells by mouse NK cells. NKG2D antibodies also increased specific lysis of THP-1 target cells, while isotype control antibody showed reduced specific lysis. The dotted line indicates specific lysis of THP-1 cells by mouse NK cells without antibody added (FIG. 17).

### Example 6—NKG2D Antibodies have High Thermostability

Melting temperatures of NKG2D-binding domains were assayed using differential scanning fluorimetry. The extrapolated apparent melting temperatures of NKG2D-binding domains were high relative to typical IgG1 antibodies (FIG. 18).

### Example 7—Synergistic Activation of Human NK Cells by Cross-Linking NKG2D and CD16

### Primary Human NK Cell Activation Assay

Peripheral blood mononuclear cells (PBMCs) were isolated from peripheral human blood buffy coats using density gradient centrifugation. NK cells were purified from PBMCs using negative selection magnetic beads (StemCell Technologies, Vancouver, Canada; Cat #17955). NK cells were >90% CD3⁻CD56⁺ as determined by flow cytometry. Cells were then expanded 48 hours in media containing 100 ng/mL hIL-2 (PeproTech, Inc., Rocky Hill, N.J.; Cat #200-02) before use in activation assays. Antibodies were coated onto a 96-well flat-bottom plate at a concentration of 2 μg/ml (anti-CD16, BioLegend, San Diego, Calif.; Cat #302013) and 5 μg/mL (anti-NKG2D, R&D Systems, Minneapolis, Minn.; Cat #MAB139) in 100 μl sterile PBS overnight at 4° C. followed by washing the wells thoroughly to remove excess antibody. For the assessment of degranulation, IL-2-activated NK cells were resuspended at 5×10⁵ cells/ml in culture media supplemented with 100 ng/mL hIL2 and 1 μg/mL APC-conjugated anti-CD107a mAb (BioLegend Cat #328619). 1×10⁵ cells/well were then added onto antibody coated plates. The protein transport inhibitors Brefeldin A (BFA, BioLegend, San Diego, Calif.; Cat #420601) and Monensin (BioLegend, San Diego, Calif.; Cat #420701) were added at a final dilution of 1:1000 and 1:270 respectively. Plated cells were incubated for 4 hours at 37° C. in 5% CO₂. For intracellular staining of IFNγ, NK cells were labeled with anti-CD3 (BioLegend, San Diego, Calif.; Cat #300452) and anti-CD56 mAb (BioLegend, San Diego, Calif.; Cat #318328) and subsequently fixed and permeabilized and labeled with anti-IFNγ mAb (BioLegend, San Diego, Calif.; Cat #506507). NK cells were analyzed for expression of CD107a and IFNγ by flow cytometry after gating on live CD56⁺CD3⁻cells.

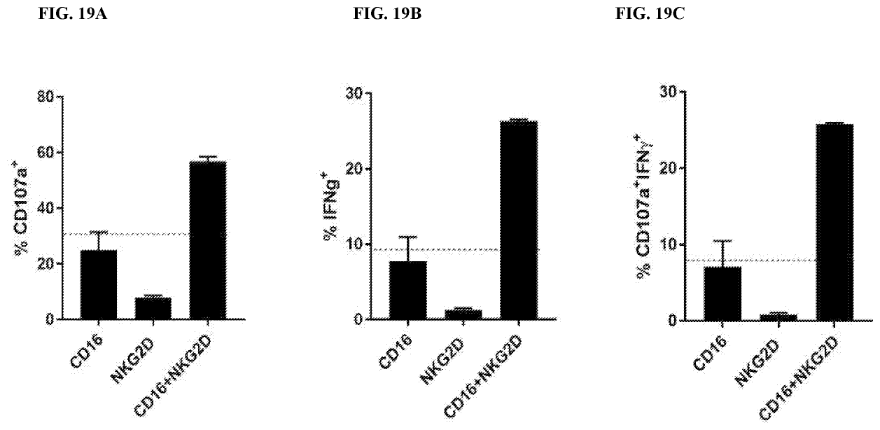

To investigate the relative potency of receptor combination, crosslinking of NKG2D or CD16 and co-crosslinking of both receptors by plate-bound stimulation was performed. As shown in FIG. 19, expression of CD107a and intracellular IFNγ of IL-2-activated NK cells was analyzed after 4 hours of plate-bound stimulation with anti-CD16, anti-NKG2D, or a combination of both monoclonal antibodies. Combined stimulation of CD16 and NKG2D resulted in percentages of CD107a⁺ cells (FIG. 19A) and IFNγ⁺ cells (FIG. 19B) that were greater than the additive effect of individual stimulations of CD16 or NKG2D alone (as indicated by the dotted line). Similarly, combined stimulation of CD16 and NKG2D resulted in a greater percentage of CD107a⁺IFNγ⁺ double-positive cells as compared to the additive effect of individual of each receptor alone (FIG. 19C). Bar graphs show the mean (n=2)±SD and are representative of five independent experiments using five different healthy donors.

### Example 8—Binding Kinetics of DLL3 Antibodies to Different Variants of DLL3

The extracellular domain (ECD) of human DLL3 was (AdipoGen Life Sciences, San Diego, Calif.) further purified using size exclusion chromatography. Recombinant His-tagged proteins of different domains of human DLL3 (N-terminal, EGF2-6, EGF2-6, EGF4-6, EGF5-6) were expressed in a cell line and purified using size exclusion chromatography.

The binding kinetics of different anti-DLL3 antibodies to recombinant proteins of different domains of human DLL3 were studied by surface plasmon resonance (SPR) using a Biacore™ 8K instrument. These anti-DLL3 antibodies were produced from mouse hybridomas, and each included a heavy chain variable region and light chain variable region described herein. Briefly, antibodies recognizing human IgG Fc and antibodies recognizing mouse IgG Fc were immobilized on different channels of a Biacore™ 8K chip to allow simultaneous analysis of human and murine anti-DLL3 antibodies. Murine anti-DLL3 antibodies were captured on the anti-mouse Fc channel of the Biacore chip. The human anti-DLL3 antibody from Stemcentrx (San Francisco, Calif.) was used as a control and was captured onto anti-human Fc channel of the Biacore™ chip. Different concentrations of DLL3 ECD, DLL3 N-terminal domain, EGF2-6, EGF3-6, EGF4-6, or EGF5-6 domains of DLL3 were injected. Experiments were performed at 37° C. Biacore™ 8K evaluation software was used for all data analysis. To obtain kinetic rate constants double-referenced data were fit to a 1:1 interaction model using Biacore™ 8K Evaluation software (GE Healthcare, Marlborough, Mass.). The equilibrium binding constant K_{D} was determined by the ratio of binding rate constants k_{d}/kₐ.

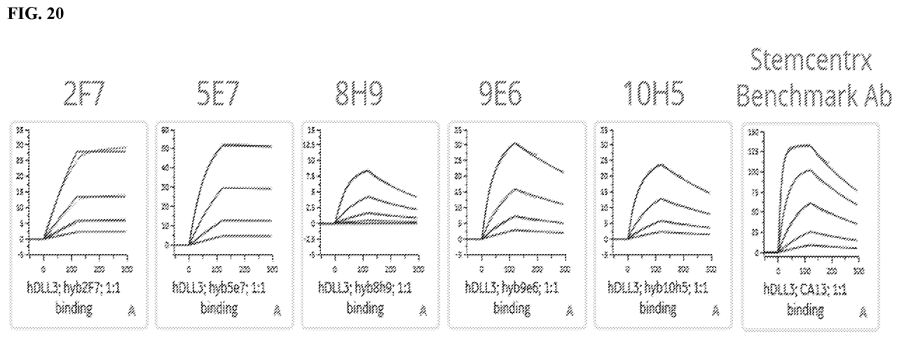

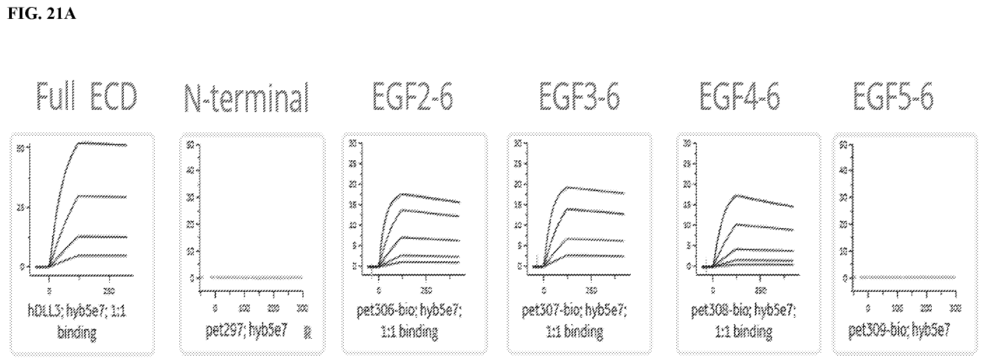

FIG. 20 shows binding profiles of murine anti-DLL3 antibodies to the ECD of human DLL3 (AdipoGen Life Sciences, San Diego, Calif.), obtained by SPR analysis at 37° C. Table 36 lists the calculated binding kinetics (K_{D}). The antibodies demonstrate a range of binding affinities to the DLL3 ECD from <0.011 to 8.44 nM. Stemcentrx anti-DLL3 antibody was used as a control.

TABLE 36

| Antibody | Construct | kₐ (1/Ms) | k_{d} (1/s) | K_{D} (nM) |
|---|---|---|---|---|
| 2F7 | hDLL3 ECD | 7.21e+4 | ~1.15e−6 | <0.011* |
| 5E7 | hDLL3 ECD | 3.19e+5 | 6.47e−5 | 0.203 |
| 8H9 | hDLL3 ECD | 5.42e+5 | 4.58e−3 | 8.44 |
| 9E6 | hDLL3 ECD | 4.08e+5 | 2.08e−3 | 5.11 |
| 10H5 | hDLL3 ECD | 4.46e+5 | 2.74e−3 | 6.1 |
| Stemcentrx | hDLL3 ECD | 1.64e+6 | 3.14e−3 | 1.92 |
| Benchmark Ab | | | | |

*Kinetic data for 2F7 are approximate - off rate beyond the instrument sensitivity

### Mapping of Antibody Binding Epitope on DLL3

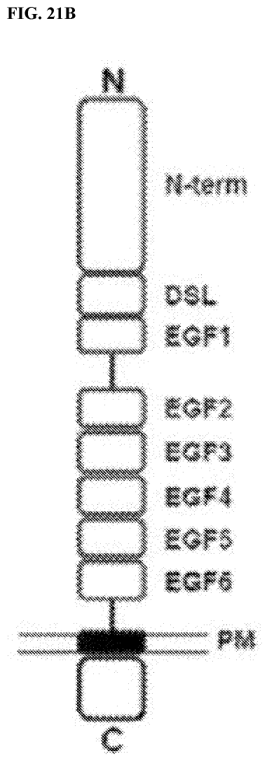

The binding epitope on DLL3 by the anti-DLL3 antibody which included a heavy chain variable region and light chain variable region of clone 5E7 (see Table 2) was identified by SPR analysis at 37° C. FIG. 21A shows the kinetics of the anti-DLL3 antibody binding to different DLL3 domains (N-terminus, EGF2-6, EGF3-6, EGF4-6, or EGF5-6) illustrated in FIG. 21B. The antibody binds to constructs incorporating EGF2-6, EGF3-6, and EGF4-6, but shows no binding to the N-terminal domain or EGF5-6 domain of DLL3, indicating that EGF4 is involved in binding to 5E7. Binding kinetics (K_{D}) of the antibody to different DLL3 domains are listed in Table 37.

TABLE 37

| Antibody | Construct | kₐ (1/Ms) | k_{d} (1/s) | K_{D} (nM) |
|---|---|---|---|---|
| 5E7 | hDLL3 ECD | 3.19e+5 | 6.47e−5 | 0.203 |
| 5E7 | N-terminal | No binding | | |
| 5E7 | EGF2-6 | 5.7e+5 | 3.82e−4 | 0.669 |
| 5E7 | EGF3-6 | 1.45e+6 | 2.64e−4 | 0.184 |
| 5E7 | EGF4-6 | 5.46e+5 | 6.10e−4 | 1.12 |

| | | |
|---|---|---|
| 5E7 | EGF5-6 | No binding |

### Example 9—Epitope Binning of Anti-DLL3 Antibodies

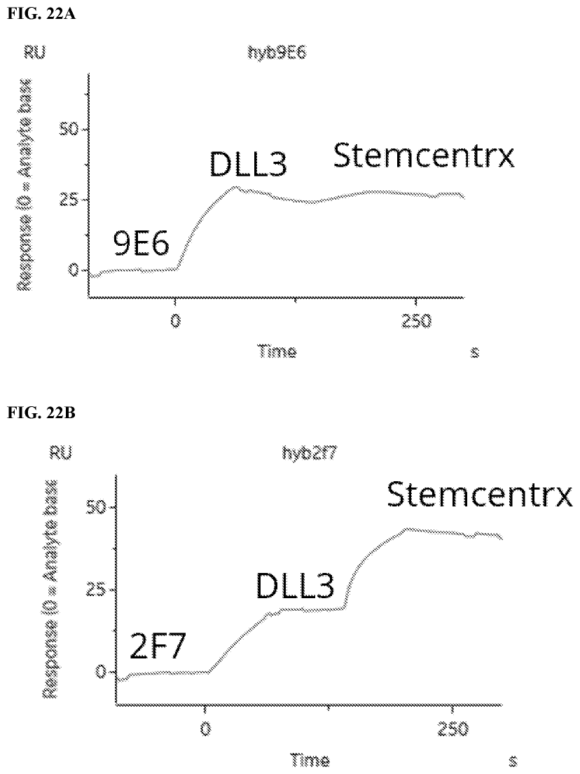

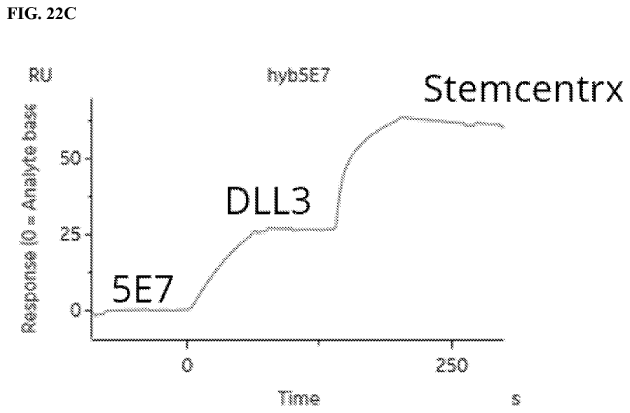

Binning of different anti-DLL3 antibodies against Stemcentrx antibody was performed by SPR using a Biacore™ 8K instrument. Briefly, murine anti-DLL3 antibodies were captured using an anti-mouse Fc antibody immobilized on a CMS chip. This was followed by injections of human DLL3 ECD and the Stemcentrx anti-DLL3 antibody (San Francisco, Calif.) consecutively. Experiments were performed at 25° C. Biacore™ 8K evaluation software was used for all data analysis. FIGS. 22A-22C show the binning profiles of anti-DLL3 antibodies corresponding to 2F7 (FIG. 22B), 5E7 (FIG. 22C), and 9E6 (FIG. 22A) clones to the ECD of DLL3. The antibody including the 9E6 clone binds to an epitope on DLL3 overlapping with the epitope to which the Stemcentrx anti-DLL3 antibody binds (FIG. 22A). The antibody including the 2F7 or 5E7 clone did not block the binding of the Stemcentrx antibody to the DLL3 ECD, suggesting that the antibodies corresponding to 2F7 (FIG. 22B) and 5E7 (FIG. 22C) bind to epitopes on DLL3 different from the one bound by the Stemcentrx antibody.

### Example 10—Determination of the Melting Temperatures of Anti-DLL3 Antibodies

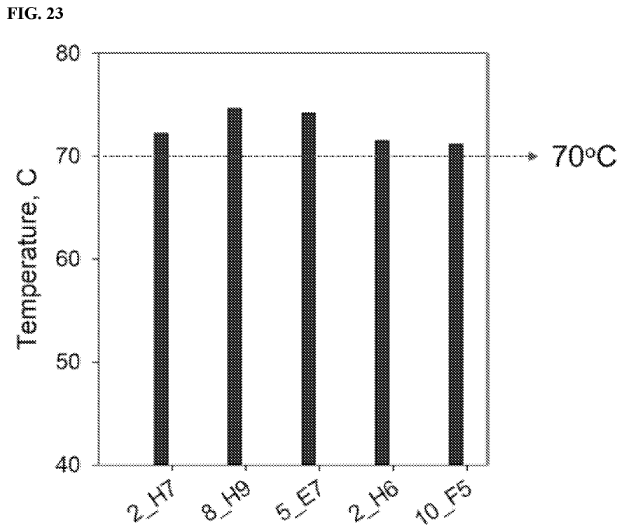

Determination of the melting temperature of anti-DLL3 antibodies was done by differential scanning fluorimetry analysis using an Applied Biosystems QuantStudio3™ instrument (Thermo Fisher, Waltham, Mass.) in a temperature range of 15−95° C. All samples were run in duplicate. Results were analyzed using Applied Biosystems Protein Thermal Shift™ Software Version 13 (Thermo Firsher, Waltham, Mass.). As shown in FIG. 23, anti-DLL3 antibodies corresponding to clones 2H7, 8H9, 5E7, 2H6, and 10F5 showed melting temperatures above 70° C.

### Example 11—Assessment of Binding to Recombinant Human DLL3 and Cross-Reactivity with DLL1/DLL4

To investigate the binding of anti-DLL3 antibodies to human DLL3, wells of high binding flat-bottom plates were coated with recombinant human DLL3 diluted to 0.5 μg/ml. To assess cross-reactivity of the antibodies to DLL1/DLL4, which are closely related family members to DLL3, plates were coated with recombinant human DLL1 diluted to 1 μg/ml, or human DLL4 diluted to 0.5 μg/ml. After blocking the plates with PBS containing 1% BSA, a test anti-DLL3 antibody and a positive control antibody for each of DLL3 (R&D Systems, Minneapolis, Minn., Cat #MAB4315), DLL1 (BioLegend, San Diego, Calif., Cat #MHD1-314) and DLL4 (BioLegend, San Diego, Calif., Cat #MHD4-46), respectively, were diluted serially starting from 10 μg/ml and added to the wells. Binding was detected using anti-mouse IgG-HRP and 3,3′,5,5′-Tetramethylbenzidine (TMB) substrate. Signals were normalized to the corresponding positive control antibody.

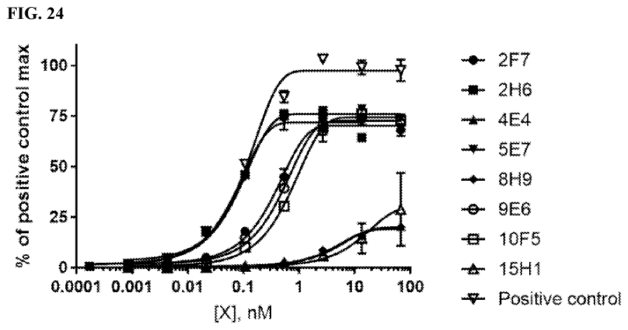

As shown in FIG. 24, robust binding of anti-DLL3 antibodies to DLL3 in a dose-dependent manner was observed. Table 38 lists the calculated EC₅₀ based on the binding data from FIG. 24. Asterisks indicate anti-DLL3 antibodies that did not reach saturation of binding to DLL3; the corresponding EC₅₀ values are therefore estimates.

TABLE 38

| | ELISA |
|---|---|
| DLL3 clone | EC₅₀ (nM) |
| 2F7 | 0.30 |
| 2H6 | 0.06 |
| 4E4 | 4.93* |
| 5E7 | 0.07 |
| 8H9 | 4.58* |
| 9E6 | 0.45 |
| 10F5 | 0.64 |
| 15H1 | 76.1* |
| Positive** | 0.10 |

*does not reach saturation, value is estimate
**commercial reagent mAb

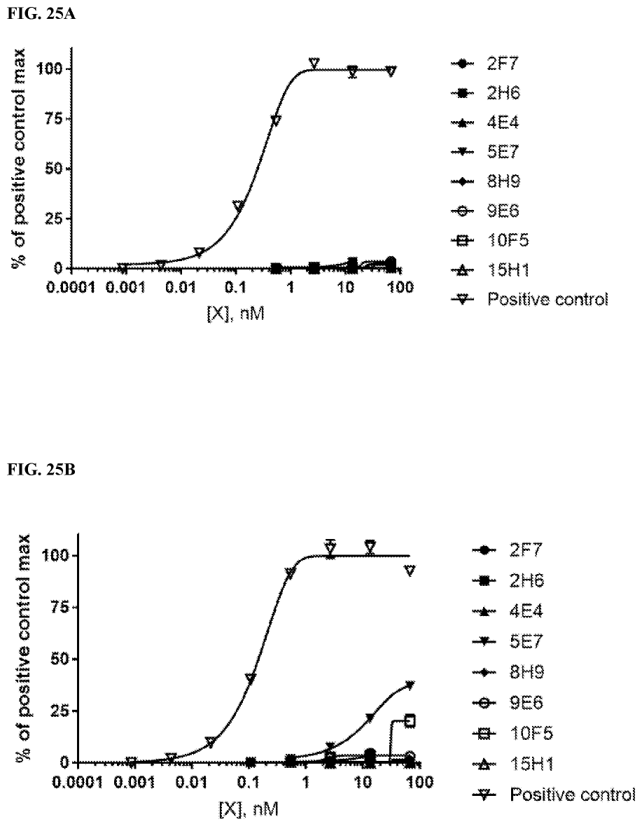

As shown in FIG. 25A, the anti-DLL3 antibodies displayed little cross-reactivity with human DLL1. As shown in FIG. 25B, the anti-DLL3 antibodies corresponding to clones 2F7, 2H6, 4E4, 8H9, 9E6, and 15H1 also displayed little cross-reactivity with human DLL4. Two antibodies corresponding to clones 5E7 and 10F5 showed weak cross-reactive binding to DLL4 at very high concentrations measured by ELISA. Binding signals were normalized to the corresponding positive control anti-DLL3 antibody and the positive control anti-DLL4 antibody.

### Example 12—Assessment of Antibody Binding to Cell-Expressed Human DLL3

DLL3-expressing human small cell lung cancer line NCI-H82 was used to assess the binding of anti-DLL3 antibodies to DLL3. Antibodies were serially diluted starting from 2 μg/mL and then incubated with the cells. Binding was detected using a fluorophore-conjugated anti-mouse IgG secondary antibody. Cells were analyzed by flow cytometry, and binding was expressed as mean fluorescence intensity (MFI) relative to the signal from secondary antibody-only control.

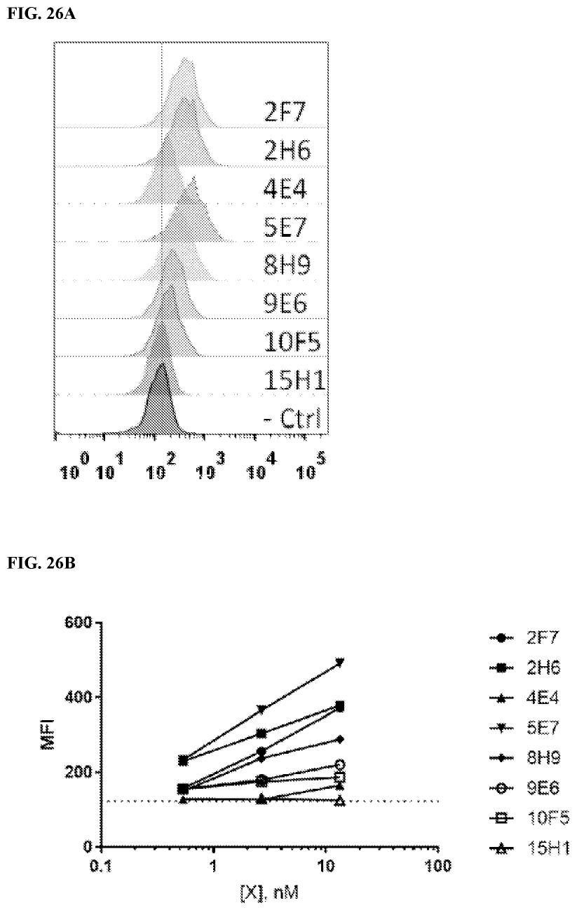

As shown in FIG. 26A, the anti-DLL3 antibodies corresponding to clones 2F7, 2H6, 4E4, 5E7, 8H9, 9E6, 10F5, and 15H1 (2 μg/mL) bound to DLL3 expressed on NCI-H82 cells. Binding of anti-DLL3 antibodies corresponding to clones 5E7, 2H6, 2F7, and 8H9 to DLL3 on NCI-H82 cells was dose-dependent, binding of antibodies corresponding to clones 9E6 and 10F5 to DLL3 was dose-dependent above a 5 nM dose, and binding of antibodies corresponding to clone 15H1 to DLL3 on NCI-H82 cells was independent of dose (FIG. 26B).

### Example 13—Determination of Extent of Antibody Internalization by DLL3⁺ SCLC Lines

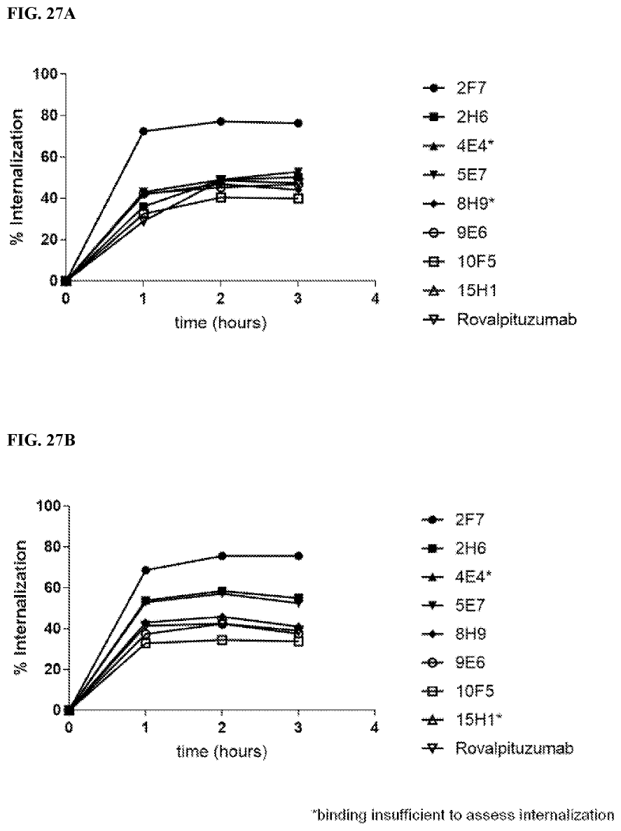

DLL3-expressing human small cell lung cancer cell lines SHP-77 and DMS-79 were used to assess internalization of anti-DLL3 antibodies upon binding to DLL3 on the surface of the cells. Antibodies were diluted to 10 μg/mL and incubated with the cells at 37° C. for 1, 2 or 3 hours, or on ice for 20 minutes. The remaining surface-bound antibodies were then detected using a fluorophore-conjugated anti-mouse IgG secondary antibody. Cells were analyzed by flow cytometry, and the antibody internalization was calculated as a percentage loss of mean fluorescence intensity (MFI) in comparison with the corresponding control condition, when the cells were incubated with the antibody on ice. As shown in FIG. 27, significant internalization of the anti-DLL3 antibodies on SHP-77 cells (FIG. 27A) and DMS-79 cells (FIG. 27B) was observed.

### Example 14—Binding of Multi-Specific Binding Proteins to DLL3-Positive Cells

The human myeloma cell line RPMI-8226 was transduced to express either the full-length (DLL3-D1-6) or a truncated (DLL3-D1-2) form of the extracellular portion of DLL3. An anti-DLL3 multi-specific binding protein and monoclonal antibody were diluted and incubated with DLL3⁺ RPMI-8226 cells. Binding was detected using a fluorophore conjugated anti-human IgG secondary antibody, and cells were analyzed by flow cytometry.

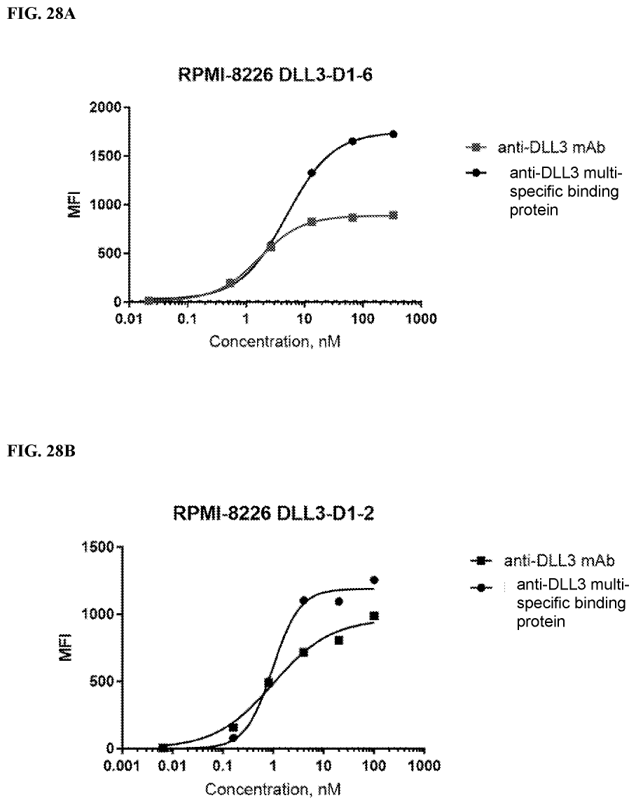

As shown in FIG. 28A and FIG. 28B, an anti-DLL3 monoclonal antibody and an anti-DLL3 multi-specific binding protein having the same DLL3-binding Fab bind with a similar dose-response to RPMI-8226 cells transduced to express DLL3-D1-6 or DLL3-D1-2, but anti-DLL3 multi-specific binding protein bind to a higher maximum.

### Example 15—Activation of NK Cells by Multi-Specific Binding Proteins

Peripheral blood mononuclear cells (PBMCs) were isolated from human peripheral blood buffy coats using density gradient centrifugation. Isolated PBMCs were washed and NK cells were isolated using a negative selection technique with magnetic beads. The purity of isolated CD3⁺CD56⁺ NK cells was typically >90%. Isolated NK cells were cultured overnight in media containing 100 ng/mL IL-2.

RPMI-8226 cells transduced to express DLL3 were harvested from culture, pelleted, and re-suspended in culture media to a concentration of 10⁶ cells/mL. 50 μl of cell suspension was added to individual wells of a 96-well plate. Anti-DLL3 multi-specific binding protein or anti-DLL3 monoclonal antibody was diluted in culture media, and 50 μl added to duplicate wells containing cell suspension. To enable detection of CD107a and intracellular IFNγ, 50 μl of activation cocktail lBrefeldin A (BioLegend, San Diego, Calif.; Cat #420601), Monensin (BioLegend, San Diego, Calif.; Cat #420701), 100 ng/mL IL-2, and fluorophore-conjugated anti-CD107a (BioLegend, San Diego, Calif.; Cat #328620)] was added to each well.

Isolated primary NK cells were harvested from overnight culture, washed, and re-suspended at in culture media to a concentration of 10⁶ cells/mL. 50 μl of isolated primary NK cell suspension was added to RPMI-8226 cell-containing wells and incubated at 37° C., 5% CO₂ for 4 hours.

Following co-culture, cells were stained for surface markers (CD107a and/or CD69), fixed, permeabilized with permeabilization/wash buffer (BioLegend, San Diego, Calif.; Cat #421002), stained for intracellular IFNγ, and analyzed by flow cytometry.

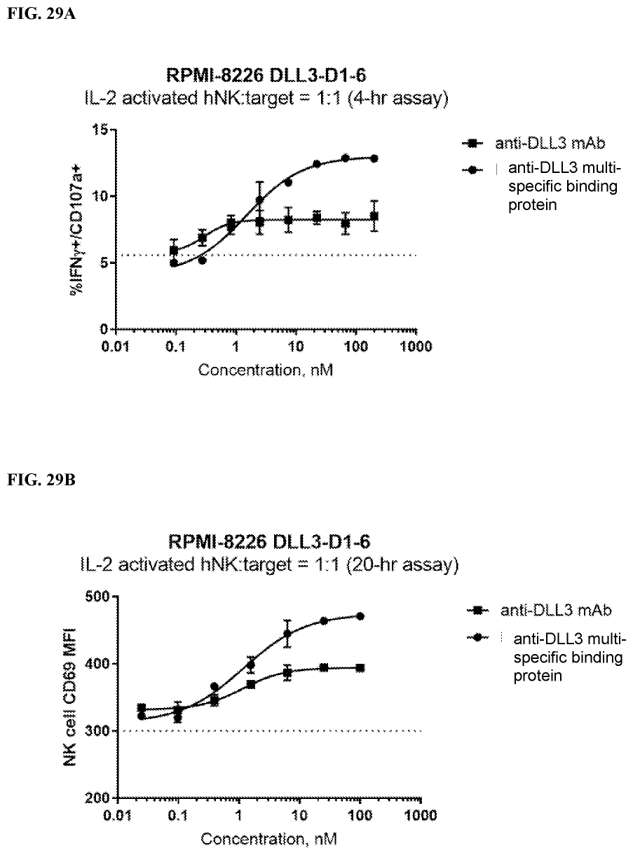

As shown in FIG. 29A and FIG. 29B, anti-DLL3 monoclonal antibody and anti-DLL3 multi-specific binding protein activate purified human NK cells co-cultured with DLL3-expressing target cells. As detected by flow cytometry, expression of CD107a and accumulation of intracellular IFNγ were comparable in NK cells treated with anti-DLL3 multi-specific binding protein and anti-DLL3 monoclonal antibody (FIG. 29A). Following a 20-hour co-culture with DLL3⁺ target cells, activation marker CD69 was upregulated to a greater extent on NK cells treated with the anti-DLL3 multi-specific binding protein as compared to the anti-DLL3 monoclonal antibody (FIG. 29B).

### Example 16—Enhanced NK Cell-Mediated Lysis of DLL3⁺ Target Cells by Multi-Specific Binding Proteins

### DELFIA Cytotoxicity Assay

RPMI-8226 cells transduced to express DLL3 were harvested from culture, pelleted, and re-suspended in culture media to a concentration of 10⁶ cells/mL for labeling with BATDA reagent (Perkin Elmer, Waltham, Mass., Cat #AD0116) in accordance with the manufacturer's instructions. After labeling, cells were washed 3× with HEPES buffered saline, re-suspended at a concentration of 5×10⁴ cells/mL in culture media, and 100 ul of BATDA labeled cells were added to each well of the 96-well plate. Designated wells were reserved to measure for spontaneous release from target cells, and all other wells were prepared for max lysis of target cells by addition of 1% Triton-X.

50 μl of diluted anti-DLL3 monoclonal antibody or anti-DLL3 multi-specific binding protein was added to designated wells. IL-2 activated, isolated NK cells (prepared as previously described in Example 15) were harvested from culture, washed, and re-suspended at a concentration of 5×10⁵ cells/mL. 50 μl of NK cell suspension was added to designated wells of the 96-well plate to make a total of 200 μl culture volume and to achieve a final NK cell to RPMI-8226 target cell ratio of 5:1. Plates were incubated at 37° C., 5% CO₂ for 2-3 hours.

Following co-culture, cells were pelleted by centrifugation at 200 g for 5 minutes. 20 μL of culture supernatant was transferred to a clean microplate and 200 μL of room temperature europium solution was added to each well. The microplate was protected from light and incubated on a plate shaker at 250 rpm for 15 minutes. The microplate was read using either Victor3™ (Perkin Elmer, Waltham, Mass.) or SpectraMax i3X (Molecular Devices, San Jose, Calif.) instruments. % Specific lysis was calculated as follows:

% Specific lysis=[(Experimental release−Spontaneous release)/(Maximum release−Spontaneous release)]×100%

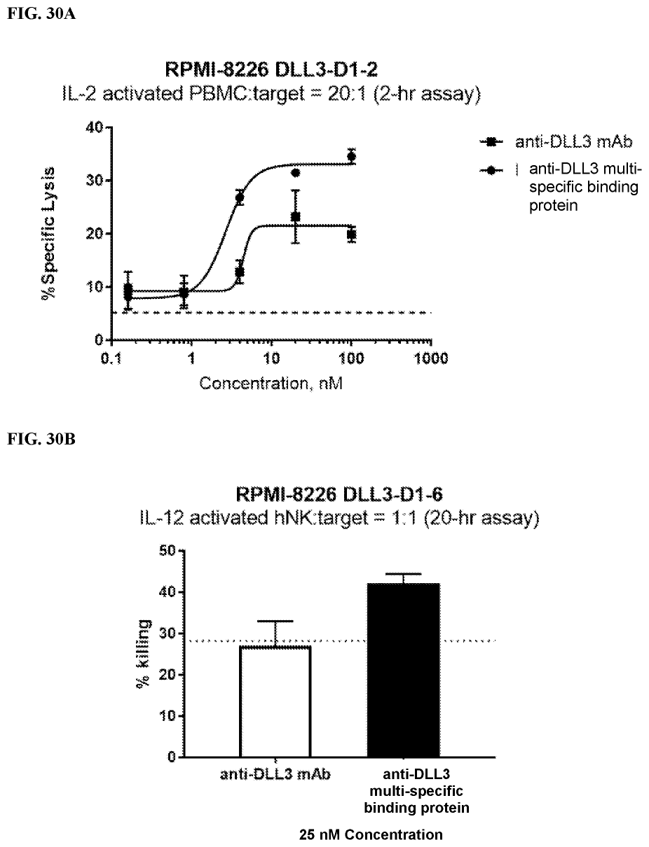

As shown in FIG. 30A, IL-2-activated human PBMC effector cells have limited basal cytotoxic activity against RPMI-8226 cells expressing DLL3-D1-2 after 2 hours co-culture (dotted line). Introduction of anti-DLL3 multi-specific binding protein in co-culture results in a 30% increase in specific lysis as compared to an anti-DLL3 monoclonal antibody having the same Fab arms.

### Flow Cytometry Cytotoxicity Assay

RPMI-8226 cells transduced to express DLL3 were harvested from culture, pelleted, and re-suspended at a concentration of 10⁵ cells/mL in culture media and 100 μl of cell suspension was added to each well of a 96-well plate. Anti-DLL3 multi-specific binding protein or anti-DLL3 monoclonal antibody was diluted in culture media and 50 ul of each was added to duplicate wells of the 96-well plate. Purified human NK cells (prepared as previously described in Example 15) were harvested from culture, washed, and re-suspended at 2×10⁵ cells/mL in culture media. 50 μl of NK cell suspension was added to wells except for RPMI-8226-only controls. The 96-well plate was incubated at 37° C., 5% CO₂ for 20 hours. Following co-culture, cells were stained, fixed, and analyzed by flow cytometry.

As shown in FIG. 30B, IL-2-activated purified human NK cells exhibited basal lysis of RPMI-8226 cells expressing DLL3-D1-6 after 20 hours (dotted line). Co-cultures in the presence of anti-DLL3 monoclonal antibody did not result in a significant increase in cell lysis. However, co-cultures in the presence of anti-DLL3 multi-specific binding protein exhibited enhanced NK cell-mediated killing of RPMI-8226 target cells.

### Example 17—Humanization of Murine Antibody 5E7

Humanization of mouse 5E7 was accomplished by grafting mouse CDRs to appropriate human frameworks using molecular operating environment (MOE) protein modelling software. The CDR grafting was based on combination of best sequence match to human frameworks and by homology model. Human germline VH1-3 was selected as the most appropriate acceptor framework for variable heavy chain. For maintaining binding and structural integrity of the VH domain, three residues of the selected human framework were mutated back to the original mouse framework residues. Human germline VK1-39 was selected as the most appropriate acceptor framework for variable light chain. For maintaining binding and structural integrity of the VL domain, three residues of the selected human framework were mutated back to the original mouse frame work residues. From this effort the best variant (clone h5E7) was selected.

The three residues in the VH mutated back to the original mouse framework residues were at Kabat positions 44, 71, and 76, as bolded and underlined in the h5E7 VH sequence below. The heavy chain CDR sequences were also identified and are underlined below.

h5E7 VH sequence
[SEQ ID NO: 668]
QVQLVQSGAEVKKPGASVKVSCKASGFNIKDDYIHWVRQAPGQGLEWMG
WIDSENGDTEYASKFQGRVTITADTSANTAYMELSSLRSEDTAVYYCAT
SSYYSYDLFVYWGQGTLVTVSS

The three residues in the VL mutated back to the original mouse framework residues were at Kabat positions 2, 36, and 42, as bolded and underlined in the h5E7 VL sequence below. The light chain CDR sequences were also identified and are underlined below.

h5E7 VL sequence
[SEQ ID NO: 669]
DVQMTQSPSSLSASVGDRVTITCKSSQSLLHSNGKTYLNWLQQKPGQAP
KLLLYLVSKLESGVPSRFSGSGSGTDYTLTISSLQPEDFATYYCLQTTH
LYTFGQGTKLEIK

### Example 18—Affinity Maturation of h5E7 by CDRH3 Mutation

A CDRH3 focused library with single, double and triple mutants of h5E7 was displayed as single chain variable fragment (scFv) on the surface of Saccharomyces cerevisiae. The starting library diversity was estimated to be around 10⁶. Three rounds of selections were carried out. The first round of selection was performed by magnetic activated cell sorting (MACS) and enriched clones that bound to 100 nM human DLL3 ECD (ECD of DLL3 was purchased from Adipogen (AG-40B-0151) and further purified in house using size exclusion chromatography before use in this experiment). The second and third rounds of selection were carried out on a fluorescence activated cell sorter (FACS). During the second round of selection, biotinylated human DLL3 was titrated down to 1 nM and variants in the library that bound better than parent h5E7 were gated and collected. The third round of selection was focused on enriching binders that have slower off-rate (kd) than the parent h5E7 clone. This was achieved by competing off bound biotinylated human DLL3 from relatively faster kd variants with excess of unbiotinylated hDLL3 or with the murine 5E7 monoclonal antibody (mAb). The clones enriched from the second and third rounds included h5E7-YD-C6, h5E7-YD-F3, h5E7-YD-A6, and h5E7-YD-B5, the sequence of which are shown in Table 2. Consensus sequences of the humanized 5E7 variants are also provided in Table 2.

The murine 5E7 and all the humanized versions were cloned and expressed as IgG1 mAbs. All heavy chain variable regions (including mouse 5E7) were cloned into the N-terminus of human IgG1 CH1-CH2-CH3 constant region. All light chain variable regions (including mouse 5E7) were cloned into N-terminus of human constant Kappa region. All clones were expressed in the EXPI293 system and purified using protein A MabSelect SuRe resin. When necessary, an additional step of SEC purification was performed.

The kinetics of binding of different humanized variants and parental murine 5E7 antibody to human DLL3-His was studied by Surface Plasmon Resonance using a Biacore 8K instrument. Briefly, anti-hFc and anti-mFc IgG proteins were covalently immobilized onto different channels of CMS chip to allow simultaneous analysis of human and murine antibodies. m5E7 was captured on the anti-mouse Fc channel and humanized variants were captured on the anti-human Fc channel at a desired capture level of −56 RU in HBS-EP+ buffer supplemented with 0.1% BSA. Three buffer blanks and human DLL3-His (analyte) at concentrations 0.411-300 nM (in three fold dilutions) were injected over the surface with captured murine or humanized 5E7 for 300 second association time and let dissociate for 600 second at a flow rate of 30 μL/min. The surfaces were subjected to regeneration with three 20-second pulses of 10 mM glycine pH 1.70 at a flow rate of 100 μL/min between every concentration of analyte. The experiment was conducted at 37° C. Data were double referenced and a 1:1 fit model was applied to the sensorgrams in the Biacore Insight Evaluation software.

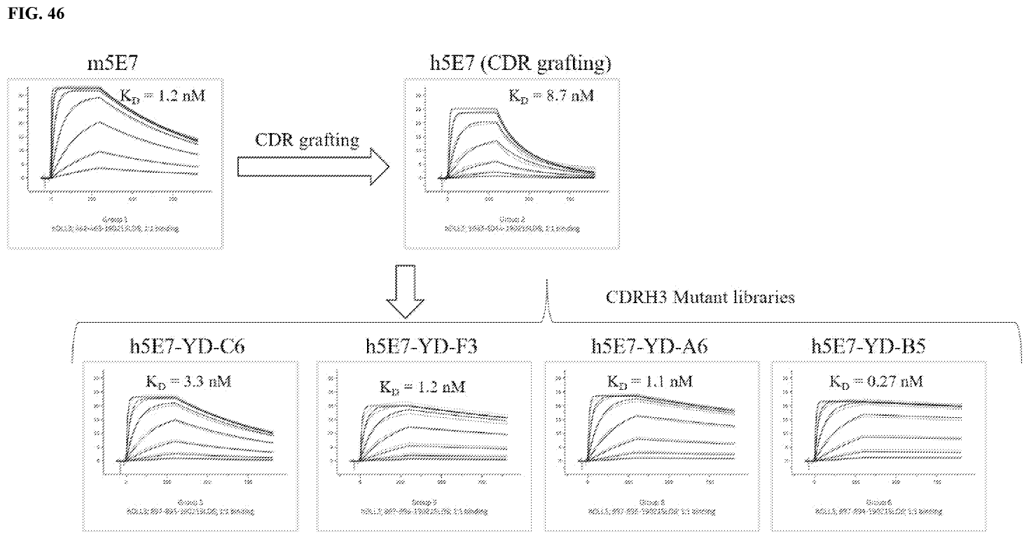

As shown in FIG. 46, the murine 5E7 and the humanized variants all bound human DLL3-His. The kinetic parameters of human DLL3-His binding to murine and humanized variants of 5E7 antibody variants were calculated and shown in Table 39.

TABLE 39

| Antibody clone | Antigen | kₐ (1/Ms) | k_{d} (1/s) | K_{D} (nM) | CDRH3 |
|---|---|---|---|---|---|
| 5E7 | hDLL3 | 1.19 × 10⁶ | 1.45 × 10⁻³ | 1.22 | SSYYSYDLFVY |
| h5E7 | hDLL3 | 7.99 × 10⁵ | 6.97 × 10⁻³ | 8.72 | SSYYSYDLFVY |
| h5E7-YD-C6 | hDLL3 | 4.09 × 10⁵ | 1.37 × 10⁻³ | 3.35 | SEYYSYDLFVY |
| h5E7-YD-F3 | hDLL3 | 3.12 × 10⁵ | 4.05 × 10⁻⁴ | 1.29 | SSYWSYDLLVY |
| h5E7-YD-A6 | hDLL3 | 3.96 × 10⁵ | 4.34 × 10⁻⁴ | 1.10 | SSYWSYDLFVY |
| h5E7-YD-B5 | hDLL3 | 4.74 × 10⁵ | 1.30 × 10⁻⁴ | 0.27 | STYWSYDLFVY |

To assess the binding of the mAbs to DLL3 positive cells, human myeloma cell line RPMI-8226 was transduced to express the full-length extracellular portion of DLL3. Anti-DLL3 mAbs were diluted and incubated with DLL3⁺ RPMI-8226 cells. The cells were analyzed by flow cytometry and binding of a mAb was detected using a fluorophore conjugated anti-human IgG secondary antibody.

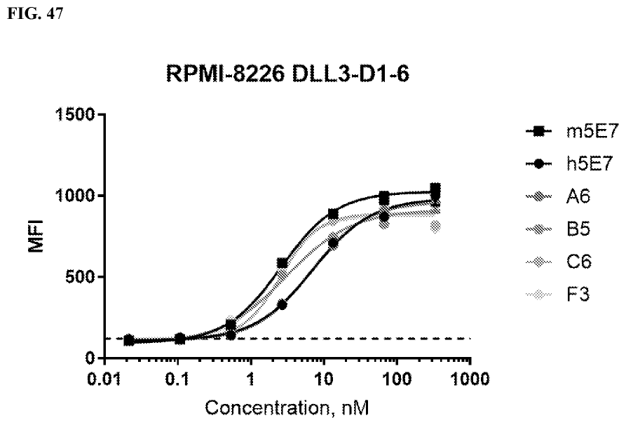

As shown in FIG. 47, conversion to a human backbone reduced the binding affinity of 5E7 to DLL3 presented on cells. After introducing mutations in heavy chain CDR3, the C6 and F3 variants of h5E7 showed similar binding affinities to cell surface DLL3 as the murine 5E7. The A6 variant exhibited similar binding affinity as h5E7, and the B5 variant bound with an intermediate affinity.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A protein comprising:
(a) a first antigen-binding site that binds NKG2D;
(b) a second antigen-binding site that binds Delta like canonical Notch ligand 3 (DLL3); and
(c) an antibody fragment crystallizable (Fc) domain or a portion thereof sufficient to bind CD16, or a third antigen-binding site that binds CD16.

2. The protein according to claim 1, wherein:
(a) the heavy chain variable domain of the second antigen-binding site comprises an amino acid sequence at least 90% identical to SEQ ID NO: 679, 668, 671, 673, 675, 677, or 130, and the light chain variable domain of the second antigen-binding site comprises an amino acid sequence at least 90% identical to SEQ ID NO: 669 or 131;
(b) the heavy chain variable domain of the second antigen-binding site comprises an amino acid sequence at least 90% identical to SEQ ID NO:114 and the light chain variable domain of the second antigen-binding site comprises an amino acid sequence at least 90% identical to SEQ ID NO:115;
(c) the heavy chain variable domain of the second antigen-binding site comprises an amino acid sequence at least 90% identical to SEQ ID NO:122 and the light chain variable domain of the second antigen-binding site comprises an amino acid sequence at least 90% identical to SEQ ID NO:123; or
(d) the heavy chain variable domain of the second antigen-binding site comprises an amino acid sequence at least 90% identical to SEQ ID NO:138 and the light chain variable domain of the second antigen-binding site comprises an amino acid sequence at least 90% identical to SEQ ID NO:139.

3. The protein according to claim 1, wherein the second antigen-binding site comprises:
(a) a heavy chain variable domain comprising a complementarity-determining region 1 (CDR1) amino acid sequence of SEQ ID NO:132; a complementarity-determining region 2 (CDR2) amino acid sequence of SEQ ID NO:133; and a complementarity-determining region 3 (CDR3) amino acid sequence of SEQ ID NO: 680, 670, 672, 674, 676, 678, or 134; and a light chain variable domain comprising a CDR1 amino acid sequence of SEQ ID NO:135; a CDR2 amino acid sequence of SEQ ID NO:136; and a CDR3 amino acid sequence of SEQ ID NO:137;
(b) a heavy chain variable domain comprising a CDR1 amino acid sequence of SEQ ID NO:116, a CDR2 amino acid sequence of SEQ ID NO:117, and a CDR3 amino acid sequence of SEQ ID NO:118, and a light chain variable domain comprising a CDR1 amino acid sequence of SEQ ID NO:119, a CDR2 amino acid sequence of SEQ ID NO:120, and a CDR3 amino acid sequence of SEQ ID NO:121;
(c) a heavy chain variable domain comprising a CDR1 amino acid sequence of SEQ ID NO:124, a CDR2 amino acid sequence of SEQ ID NO:125, and a CDR3 amino acid sequence of SEQ ID NO:126; and a light chain variable domain comprising a CDR1 amino acid sequence of SEQ ID NO:127, a CDR2 amino acid sequence of SEQ ID NO:128; and a CDR3 amino acid sequence of SEQ ID NO:129; or
(d) a heavy chain variable domain comprising a CDR1 amino acid sequence of SEQ ID NO:140, a CDR2 amino acid sequence of SEQ ID NO:141, and a CDR3 amino acid sequence of SEQ ID NO:142; and a light chain variable domain comprising a CDR1 amino acid sequence of SEQ ID NO:143, a CDR2 amino acid sequence of SEQ ID NO:144, and a CDR3 amino acid sequence of SEQ ID NO:145.

4-13. (canceled)

14. A protein comprising:
(a) a first antigen-binding site that binds NKG2D;
(b) a second antigen-binding site that binds mucin 1 (MUC1 or MUC1-C), Plexin-A1, tumor necrosis factor receptor superfamily member 10B (TNFRSF10B), six-transmembrane epithelial antigen of prostate member 1 (STEAP1), CUB domain-containing protein 1 (CDCP1), tyrosine-protein kinase-like 7 (PTK7), AXL receptor tyrosine kinase (AXL), receptor tyrosine-protein kinase ERBB-3 (ERBB-3), endothelin receptor type B (EDNRB), tyrosinase related protein-1 (TYRP1), or oxidized low-density lipoprotein receptor 1 (OLR1); and
(c) an antibody Fc domain or a portion thereof sufficient to bind CD16, or a third antigen-binding site that binds CD16.

15-37. (canceled)

38. A protein comprising:
(a) a first antigen-binding site that binds NKG2D;
(b) a second antigen-binding site that binds disintegrin and metalloproteinase domain-containing protein 12 (ADAM12), urokinase plasminogen activator receptor (PLAUR), C-C motif chemokine receptor 6 (CCR6), or ephrin type-A receptor 4 (EPHA4); and
(c) an antibody Fc domain or a portion thereof sufficient to bind CD16, or a third antigen-binding site that binds CD16.

39-44. (canceled)

45. A protein comprising:
(a) a first antigen-binding site that binds NKG2D;
(b) a second antigen-binding site that binds CD14, CD163, colony stimulating factor 3 receptor (CSF3R), sialic acid-binding Ig-like lectin 9 (Siglec-9), integrin alpha M (ITGAM), V-type immunoglobulin domain-containing suppressor of T-cell activation (VISTA), V-set domain-containing T-cell activation inhibitor 1(B7-H4), C-C chemokine receptor type 1 (CCR1), leucine rich repeat containing 25 (LRRC25), platelet activating factor receptor (PTAFR), signal regulatory protein beta 1 (SIRPB1), Toll-like receptor 2 (TLR2), Toll-like receptor 4 (TLR4), CD300 molecule like family member b (CD300LB), ATPase Na+/K+ transporting subunit alpha 3 (ATP1A3), or C-C chemokine receptor type 5 (CCR5); and
(c) an antibody Fc domain or a portion thereof sufficient to bind CD16, or a third antigen-binding site that binds CD16.

46-65. (canceled)

66. The protein according to claim 45, wherein the protein further comprises a site that binds a tumor-associated antigen.

67. The protein according to claim 66, wherein the tumor-associated antigen is selected from the group consisting of human epidermal growth factor receptor 2 (HER2), CD20, prostate-specific membrane (PSMA), DLL3, ganglioside GD2 (GD2), CD123, anoctamin-1 (Ano1), mesothelin, carbonic anhydrase IX (CAIX), tumor-associated calcium signal transducer 2 (TROP2), claudin-18.2, receptor tyrosine kinase-like orphan receptor 1 (ROR1), trophoblast glycoprotein (5T4), glycoprotein nonmetastatic melanoma protein B (GPNMB), folate receptor-alpha (FR-alpha), pregnancy-associated plasma protein A (PAPP-A), CD37, epithelial cell adhesion molecule (EpCAM), CD2, CD30, CD38, CD40, CD52, CD70, CD79b, glypican 3 (GPC3), B7 homolog 6 (B7H6), C-C chemokine receptor type 4 (CCR4), C-X-C motif chemokine receptor 4 (CXCR4), receptor tyrosine kinase-like orphan receptor 2 (ROR2), CD133, epidermal growth factor receptor (EGFR/ERBB-1), insulin-like growth factor 1-receptor (IGF1R), human epidermal growth factor receptor 3 (HER3)/ERBB-3, human epidermal growth factor receptor 4 (HER4)/ERBB-4, MUC1, signaling lymphocytic activation molecule F7 (SLAMF7), prostate stem cell antigen (PSCA), MHC class I polypeptide-related sequence A (MICA), MHC class I polypeptide-related sequence B (MICB), TNF-related apoptosis inducing ligand receptor 1 (TRAILR1), TNF-related apoptosis inducing ligand receptor 2 (TRAILR2), melanoma associated antigen 3 (MAGE-A3), B-lymphocyte activation antigen B7.1 (B7.1), B-lymphocyte activation antigen B7.2 (B7.2), cytotoxic T-lymphocyte associated protein 4 (CTLA4), programmed cell death protein 1 (PD1), programmed cell death 1 ligand 1 (PD-L1), and CD25.

68. The protein according to claim 1, wherein the first antigen-binding site binds to NKG2D in humans and non-human primates.

69. (canceled)

70. The protein according to claim 1, wherein the first antigen-binding site comprises a heavy chain variable domain and a light chain variable domain present on the same polypeptide.

71. (canceled)

72. The protein according to claim 70, wherein the second antigen-binding site also comprises a heavy chain variable domain and a light chain variable domain, and wherein the light chain variable domain of the first antigen-binding site has an amino acid sequence identical to the amino acid sequence of the light chain variable domain of the second antigen-binding site.

73-74. (canceled)

75. A protein according to claim 1, wherein the first antigen-binding site comprises a heavy chain variable domain at least 90% identical to an amino acid sequence selected from: SEQ ID NO:1, SEQ ID NO:41, SEQ ID NO:49, SEQ ID NO:57, SEQ ID NO:59, SEQ ID NO:61, SEQ ID NO:69, SEQ ID NO:77, SEQ ID NO:85, SEQ ID NO:650, SEQ ID NO:653, SEQ ID NO:656, SEQ ID NO:659, SEQ ID NO:662, SEQ ID NO:665, and SEQ ID NO:93.

76. The protein according to claim 1, wherein the first antigen-binding site comprises:
a. a heavy chain variable domain comprising an amino acid sequence at least 90% identical to SEQ ID NO:41 and a light chain variable domain comprising an amino acid sequence at least 90% identical to SEQ ID NO:42;
b. a heavy chain variable domain comprising an amino acid sequence at least 90% identical to SEQ ID NO:49 and a light chain variable domain comprising an amino acid sequence at least 90% identical to SEQ ID NO:50;
c. a heavy chain variable domain comprising an amino acid sequence at least 90% identical to SEQ ID NO:57 and a light chain variable domain comprising an amino acid sequence at least 90% identical to SEQ ID NO:58;
d. a heavy chain variable domain comprising an amino acid sequence at least 90% identical to SEQ ID NO:59 and a light chain variable comprising an amino acid sequence domain at least 90% identical to SEQ ID NO:60;
e. a heavy chain variable domain comprising an amino acid sequence at least 90% identical to SEQ ID NO:61 and a light chain variable domain comprising an amino acid sequence at least 90% identical to SEQ ID NO:62;
f. a heavy chain variable domain comprising an amino acid sequence at least 90% identical to SEQ ID NO:69 and a light chain variable domain comprising an amino acid sequence at least 90% identical to SEQ ID NO:70;
g. a heavy chain variable domain comprising an amino acid sequence at least 90% identical to SEQ ID NO:77 and a light chain variable domain comprising an amino acid sequence at least 90% identical to SEQ ID NO:78;
h. a heavy chain variable domain a comprising an amino acid sequence at least 90% identical to SEQ ID NO:85, SEQ ID NO:650, SEQ ID NO:653, SEQ ID NO:656, SEQ ID NO:659, SEQ ID NO:662, or SEQ ID NO:665, and a light chain variable domain comprising an amino acid sequence at least 90% identical to SEQ ID NO:86;
i. a heavy chain variable domain comprising an amino acid sequence at least 90% identical to SEQ ID NO:93 and a light chain variable domain comprising an amino acid sequence at least 90% identical to SEQ ID NO:94;
j. a heavy chain variable domain comprising an amino acid sequence at least 90% identical to SEQ ID NO:101 and a light chain variable domain comprising an amino acid sequence at least 90% identical to SEQ ID NO:102; or
k. a heavy chain variable domain comprising an amino acid sequence at least 90% identical to SEQ ID NO:103 and a light chain variable domain comprising an amino acid sequence at least 90% identical to SEQ ID NO:104.

77-86. (canceled)

87. The protein according to claim 1, wherein the first antigen-binding site is a single-domain antibody.

88. The protein according to claim 87, wherein the single-domain antibody is a V_{H}H fragment or a V_{NAR} fragment.

89. The protein according to claim 1, wherein the second antigen-binding site comprises a heavy chain variable domain and a light chain variable domain present on the same polypeptide.

90. (canceled)

91. The protein according to claim 1, wherein the second antigen-binding site is a single-domain antibody.

92. The protein of claim 91, wherein the second antigen-binding site is a V_{H}H fragment or a V_{NAR} fragment.

93. The protein according to claim 1, wherein the protein comprises a portion of an antibody Fc domain sufficient to bind CD16, wherein the antibody Fc domain comprises hinge and CH2 domains of a human IgG1 antibody.

94. (canceled)

95. The protein according to claim 93, wherein the Fc domain comprises an amino acid sequence at least 90% identical to amino acids 234-332 of a human IgG1 antibody.

96. The protein according to claim 95, wherein the Fc domain comprises amino acid sequence at least 90% identical to the Fc domain of human IgG1 and differs at one or more positions selected from the group consisting of Q347, Y349, L351, S354, E356, E357, K360, Q362, S364, T366, L368, K370, N390, K392, T394, D399, S400, D401, F405, Y407, K409, T411, and K439.

97. A formulation comprising a protein according to claim 1 and a pharmaceutically acceptable carrier.

98. A cell comprising one or more nucleic acids encoding a protein according to claim 1.

99. A method of directly and/or indirectly enhancing tumor cell death, the method comprising exposing a tumor microenvironment and natural killer cells to a protein according to claim 1.

100. A method of treating a cancer, wherein the method comprises administering a protein according to claim 1 to a patient.

101. A method of treating a cancer, wherein the method comprising administration to a patient in need thereof a protein according to claim 1 or a formulation comprising a protein according to claim 1 and a pharmaceutically acceptable carrier, wherein the cancer is selected from the group consisting of small cell lung cancer, large cell neuroendocrine carcinoma, glioblastoma, Ewing sarcoma, and cancers with a neuroendocrine phenotype.

102-119. (canceled)
